(19) United States
(12) Patent Application Publication
(10) Pub. No.: US 2019/0127755 A1
(43) Pub. Date: May 2, 2019

(54) CONSTRUCT AND VECTOR FOR INTRAGENIC PLANT TRANSFORMATION

(71) Applicant: Nexgen Plants Pty Ltd, Brisbane (AU)

(72) Inventors: Peer Martin Philipp Schenk, Tennyson (AU); Ekaterina Nowak, Ormiston (AU)

(73) Assignee: Nexgen Plants Pty Ltd, Brisbane (AU)

(21) Appl. No.: 16/172,228

(22) Filed: Oct. 26, 2018

Related U.S. Application Data
(63) Continuation-in-part of application No. PCT/AU2017/050383, filed on Apr. 27, 2017.

(30) Foreign Application Priority Data
Apr. 27, 2016 (AU) 2016901547

(51) Int. Cl. C12N 15/82

(52) U.S. Cl. CPC C12N 15/8283; C12N 15/827; C12N 15/8281; C12N 15/8282; C12N 15/8286; C12N 15/8205

(57) ABSTRACT

Genetic constructs are provided at least a fragment of which are insertable into the genetic material of a plant, wherein at least a fragment of the genetic construct comprises, consists essentially of, or consists of one or more nucleotide sequences derived from one or more plants. Also provided is use of the genetic construct for the production of genetically improved plants, and improved plants improved thereby. The improved plants may have desirable disease resistance, abiotic stress tolerance, or nutritional, palatability, or morphological properties.

### CROSS-REFERENCE TO RELATED APPLICATIONS

This application is a continuation-in-part of PCT/AU2017/050383 filed Apr. 27, 2017, which International Application was published by the International Bureau in English on Nov. 2, 2017, and claims priority from Australian Application 2016901547, filed Apr. 27, 2016, which applications are hereby incorporated by reference in their entirety in this application.

### TECHNICAL FIELD

THIS invention relates to plant transformation. More specifically, the invention relates, but is not limited, to a genetic construct for intragenic plant transformation, and methods of use of this construct.

### BACKGROUND

Gene technology for the production of new crop varieties offers significant advantages compared to conventional breeding methods, for example time and cost savings, elimination of genetic drag, and the obviation of crossing between crops and their wild relatives with partial fertility. However, a major obstacle to the progress of genetic improvement of crops by means of gene technology is the lack of public acceptance of transgenic varieties. This is due, at least in part, to the perception that the transfer of genetic material between organisms belonging to taxonomically distant groups is ‘unnatural’.

Plants produced using genetic technologies involving the transfer of genetic material between varieties of the same plants, or its sexually compatible relatives, are generally considered more acceptable to the public than transgenic crops. These processes can be considered genetic recombination where parts of a plants' genome (or that of its sexually compatible relative) is partially re-arranged and recombined to give rise to genetic diversity. Genetic recombination is an important process in nature so that individuals from a population with a diverse gene pool can adapt to changing environments. The mimicking of genetic recombination can be achieved with molecular biology tools by two approaches that are currently being explored, termed ‘cisgenic’ and ‘intragenic’.

The cisgenic approach is relatively conservative, permitting only the transfer of unmodified genomic versions of genes, complete with introns and regulatory elements from the same plants, or its sexually compatible relatives. By comparison, the intragenic approach broadens opportunities by transferring nucleic acids comprising sequences derived from multiple areas within the genome of a plant, and/or from multiple individual plants of the same species, or its sexually compatible relatives.

### SUMMARY

The present invention is broadly directed to plant transformation using plant-derived nucleotide sequences.

It is a preferred object of the invention to provide a recombinant genetic construct at least a fragment of which is insertable into the genetic material of a plant, wherein at least a fragment of the genetic construct consists of one or more nucleotide sequences derived from one or more plants. The invention is also broadly directed to the use of said genetic construct for the production of genetically improved plants.

In a first aspect, the invention provides a recombinant genetic construct comprising one or more nucleic acid fragments insertable into the genetic material of a plant, wherein said one or more nucleic acid fragments comprise, consist of, or consist essentially of a plurality of nucleotide sequences of at least 15 nucleotides in length, or preferably at least 20 nucleotides in length, derived from one or more plants.

Preferably, said nucleotide sequences derived from one or more plants are derived from one plant. Suitably, in embodiments wherein said nucleotide sequences are derived from more than one plant, said plants are inter-fertile and/or of the same species.

Preferably, the total length of the one or more nucleic acid fragments of the genetic construct that are insertable into the genetic material of a plant is at least 100 base pairs; at least 500 base pairs; at least 1000 base pairs; at least 2000 base pairs; at least 2500 base pairs; or at least 3000 base pairs.

Preferably, the one or more nucleic acid fragments of the genetic construct of this aspect that are insertable into the genetic material of a plant comprise one or more nucleotide sequences for expression. Preferably, said one or more nucleotide sequences are suitable for expression in a plant.

Preferably, said one or more nucleotide sequences for expression in a plant are adapted for expression in the plant to alter or modify a trait of the plant.

In certain preferred embodiments, one or more of said nucleotide sequences for expression in a plant comprise protein coding nucleotide sequences. In one preferred embodiment, said protein coding nucleotide sequences comprises a nucleotide sequence set forth in SEQ ID NOS:38-46, 76, 78, or 98, or a fragment or variant thereof.

In certain preferred embodiments, one or more of said nucleotide sequences suitable for expression in a plant are non-protein-coding nucleotide sequences. Preferably, said non-protein-coding nucleotide sequences comprise one or more small RNA nucleotide sequences. In one preferred embodiment, said nucleotide sequences for expression comprising one or more small RNA nucleotide sequences comprise a nucleotide sequence set forth in SEQ ID NOS:12-26, 64-66, 80-81, 83-92, 94, or 96-101, or a fragment or variant thereof.

In a preferred embodiment, said one or more nucleotide sequences for expression in a plant comprise one or more selectable marker nucleotide sequences. In one preferred embodiment, said selectable marker nucleotide sequences comprise a nucleotide sequence encoding an amino acid sequences set forth in SEQ ID NOS:38-46, or the nucleotide sequence set forth in SEQ ID NO:119, or a fragment or variant thereof.

Preferably, the one or more nucleic acid fragments of the genetic construct of this aspect that are insertable into the genetic material of a plant comprise one or more regulatory nucleotide sequences.

Suitably, the expressible nucleotide sequences of the nucleic acid fragments of the genetic construct that are insertable into the genetic material of a plant are operably connected with one or more of said regulatory nucleotide sequences.

Preferably, said regulatory nucleotide sequences comprise one or more promoter sequences. In one preferred embodiment, said promoter nucleotide sequences comprise a nucleotide sequence set forth in SEQ ID NOS:4-7, 67, 73, 74, 76, 78, or 98, or a fragment or variant thereof.

Preferably, said regulatory sequences comprise one or more terminator sequences. In one preferred embodiment, said terminator nucleotide sequences comprise a nucleotide sequence set forth in SEQ ID NOS:8-11, 106, 108, 111, or 112, or a fragment or variants thereof.

Suitably, the recombinant genetic construct of this aspect may comprise flanking sequences of or surrounding the one or more fragments insertable into the genetic material of a plant. In some preferred embodiments, the flanking sequences, or a portion thereof, are derived from the one or more plants.

In some preferred embodiments, the flanking sequences comprise restriction digest sites. In certain particularly preferred embodiments, one or more of the flanking sequences comprise a nucleotide sequence set forth in SEQ ID NOS:102, 103, 109, 110, 115, 116, 117, 118, 120, or 121, or a fragment or variant thereof.

In certain particularly preferred embodiments, the recombinant genetic construct of this aspect comprises a nucleotide sequence set forth in SEQ ID NOS:1-35, 49, 51-56, 66-68, 71-92, or 94-101, or a nucleic acid encoding an amino acid sequence set forth in SEQ ID NOS:38-46, or a fragment or variant thereof.

In certain preferred embodiments of the first aspect, the flanking sequences of the recombinant genetic construct comprise border sequences. In preferred such embodiments, the recombinant genetic construct comprises:

a first border nucleotide sequence;

a second border nucleotide sequence; and

one or more additional nucleotide sequences located between the first border nucleotide sequence and the second border nucleotide sequence,

wherein said additional nucleotide sequences, and at least a portion of said first border nucleotide sequence that is adjacent to said additional nucleotide sequences, is derived from one or more plants species.

In these embodiments, optionally, at least a portion of said second border nucleotide sequence that is adjacent to said one or more additional nucleotide sequences may be derived from one or more plants. Suitably, said one or more plants are the same plants from which the additional nucleotide sequences and the at least a portion of the first border sequence is derived.

In these preferred embodiments of the first aspect, preferably the one or more nucleic acid fragments insertable into the genetic material of a plant that consist of a plurality of nucleotide sequences of at least 15 nucleotides in length, or preferably at least 20 nucleotides in length, derived from one or more plants consist of:

(i) the at least a portion of the first border sequence derived from one or more plants;

(ii) the one or more additional nucleotide sequences derived from one or more plants; and, optionally

(iii) at least a portion of the second border sequence derived from one or more plants.

In certain embodiments, (i) and an additional nucleotide sequence of (ii) are derived from the same nucleotide sequence of a plant that is at least 15, or preferably at least 20, nucleotides in length.

In certain embodiments, (iii) and an additional nucleotide sequence of (ii) are derived from the same nucleotide sequence of a plant that is at least 15, or preferably at least 20, nucleotides in length.

Preferably, the first border nucleotide sequence of the genetic construct of these embodiments is of an Agrobacterium Right Border nucleotide sequence.

Preferably, the second border nucleotide sequence of the genetic construct of these embodiments is of an Agrobacterium Left Border nucleotide sequence.

Suitably, the additional nucleotide sequences of these embodiments may include the nucleotide sequences for expression and/or the regulatory nucleotide sequences.

In certain preferred embodiments, the additional nucleotide sequences comprising the regulatory sequence comprise a promoter sequence located adjacent to the second border nucleotide sequence of the genetic construct, and operably connected with a selectable marker nucleotide sequence.

In some particularly preferred embodiments wherein the recombinant genetic construct comprising border sequences, the genetic construct comprises a nucleotide sequence set forth in SEQ ID NOS:1-35, 49, 51-66, 81, 94, or 100, and/or a nucleotide sequence encoding the amino acid sequences set forth in SEQ ID NOS:38-46, or fragments or variants thereof.

In a second aspect, the invention provides a method for producing a recombinant genetic construct, the method including the step of deriving one or more nucleic acid fragments insertable into the genetic material of a plant from one or more plants, wherein said one or more nucleic acid fragments consist of a plurality of nucleotide sequences of at least 15 nucleotides in length, or preferably at least 20 nucleotides in length, to thereby produce the recombinant genetic construct.

In certain preferred embodiments of the second aspect, the method includes the step of adding a first border nucleotide sequence and a second border nucleotide sequence to respective ends of one or more additional nucleotide sequences, wherein the one or more additional nucleotide sequences and at least a portion of the first border nucleotide sequence are derived from one or more plants.

In a third aspect, the invention provides a genetic construct produced according to the method of the second aspect. In particularly preferred embodiments, said genetic construct comprises a nucleotide sequence set forth in SEQ ID NOS:1-35, 49, 51-56, 66-68, 71-92, or 94-101, or a nucleic acid encoding an amino acid sequence set forth in SEQ ID NOS:38-46, or a fragment or variant thereof.

Preferably the one or more plants of the first to third aspects is or includes a monocotyledonous plant or a dicotyledonous plant.

More preferably said one or more plants is or includes a grass of the Poaceae family; a cereal including sorghum, rice, wheat, barley, oats, and maize; a leguminous species including beans and peanut; a solanaceous species including tomato and potato; a brassicaceous species including cabbage and oriental mustard; a cucurbitaceous plants including pumpkin and zucchini; a rosaceous plants including rose; an asteraceous plants including lettuce and sunflower or a relative of any of the preceding plants.

In certain particularly preferred embodiments, said one or more plants is or includes tomato or a relative of tomato. In certain particularly preferred embodiments, said one or more plants is or includes rice, or a relative of rice. In certain particularly preferred embodiments, said one or more plants is or includes sorghum, or a relative or sorghum.

In a fourth aspect, the invention provides a vector comprising the recombinant genetic construct of the first or third aspects. Suitably, the vector further comprises a backbone nucleotide sequence. In one preferred embodiment, said vector backbone nucleotide sequence comprises SEQ ID NO:50, or a fragment or variant thereof.

Preferably, the backbone nucleotide sequence of the vector of this aspect comprises a backbone insertion marker nucleotide sequence. In certain preferred embodiments the backbone insertion marker nucleotide sequence comprises SEQ ID NO:36 or SEQ ID NO:37, or a fragment or variant thereof.

In certain preferred embodiments of this aspect, the vector comprises a further genetic construct.

In certain embodiments, the further genetic construct comprises one or more nucleotide sequences for insertion into the genetic material of a plant that are not of or derived from a plant. In these embodiments, preferably said one or more nucleotide sequences comprise a selectable marker nucleotide sequence. Said one or more nucleotide sequences may comprise a regulatory nucleotide sequence. In one particularly preferred such embodiment, said further genetic construct comprises the nucleotide sequence set forth in SEQ ID NO:69, or a fragment or variant thereof.

In some particularly preferred embodiments, the vector of the fourth aspect comprises a nucleotide sequence set forth in SEQ ID NO:47, 48, 63, 70, 82, 93, or 95.

In a fifth aspect, the invention provides a host cell comprising the recombinant genetic construct of the first or third aspect, or the vector of the fourth aspect.

In a sixth aspect, the invention provides a method of genetically improving a plant, including the step of inserting at least a nucleic acid fragment of the recombinant genetic construct of the first or third aspects into the genetic material of a plant cell or plant tissue.

In some preferred embodiments, said at least a nucleic acid fragment of the genetic construct is inserted into the genetic material of the plant cell or plant tissue via bacteria-mediated transformation of the plant cell or plant tissue. In said embodiments, said at least a fragment of the genetic construct is preferably inserted into the genetic material of the plant cell or plant tissue via Agrobacterium-mediated transformation of the plant cell or plant tissue, preferably using a vector of the fourth aspect.

In some preferred embodiments, said at least a nucleic acid fragment of the genetic construct is inserted into the genetic material the plant cell or plant tissue via direct transformation, such as particle bombardment.

It is particularly preferred according to this aspect that the at least a nucleic acid fragment of the genetic construct of the first or third aspect that is introduced into the genetic material of the plant cell or plant tissue is the one or more nucleic acid fragments insertable into the genetic material of a plant, wherein said one or more nucleic acid fragments consist of a plurality of nucleotide sequences of at least 15 nucleotides in length, or preferably at least 20 nucleotides in length, derived from one or more plants.

Suitably, the plant that is genetically improved according to this aspect is of a plant that is inter-fertile with and/or of the same species as said one or more plants.

Preferably, the method of this aspect includes the further step of selecting a genetically improved plant wherein one or more traits of said plant are altered as a result of insertion of the at least a fragment of the genetic construct into the genetic material of the plant.

Preferably, in embodiments of the method of this aspect including said step, the trait is altered according to the expression of one or more of the nucleotide sequences for expression of the genetic construct, in the plant.

In a preferred embodiment, said one or more altered traits is relative increased abiotic stress tolerance. In a preferred embodiment, said one or more altered traits is relatively increased disease resistance. In a preferred embodiment, said one or more altered traits is a relatively improved nutritional and/or palatability property. In a preferred embodiment, said one or more altered traits is a relatively improved morphological property.

Preferably, said one or more nucleotide sequences for expression are at least 15, or more preferably at least 20, nucleotides in length.

In some preferred embodiments, said one or more nucleotide sequences for expression comprise a protein coding nucleotide sequence.

In some preferred embodiments, said one or more nucleotide sequences for expression comprise small RNA sequences.

In one particularly preferred embodiment of this aspect, disease resistance of the plant is relatively improved or increased by the expression of said one or more isolated nucleic acids comprising one or more small RNA sequences, wherein said isolated nucleic acids are capable of altering the expression, translation and/or replication of one or more nucleic acids of a plant pathogen.

Preferably the plant pathogen is a viral plant pathogen.

In certain embodiments of the method of the sixth aspect, the method includes the further steps of:

inserting a nucleic acid fragment of a further genetic construct into the genetic material of the plant;

producing a population of plants from the plant wherein the nucleic acid fragment of the genetic construct of the first aspect and the nucleic acid fragment of the further genetic construct have been inserted into the genetic material; and

selecting a plant from said population of plants, wherein the genetic material of said plant comprises the nucleic acid fragment of the genetic construct of the first aspect, but not the nucleic acid fragment of the further genetic construct.

Preferably, the nucleic acid fragment of the further genetic construct that is inserted into the genetic material of the plant comprises a selectable marker nucleotide sequence.

In some preferred such embodiments, the genetic construct of the first aspect and the further genetic construct are of a vector of the fourth aspect.

In additional or alternative such embodiments, the further genetic construct is of a further vector.

In a seventh aspect the invention provides a genetically improved plant or plant part produced according to the method of the sixth aspect.

In a preferred embodiment, the plant or plant part of this aspect has relatively improved disease resistance. Preferably said relatively improved disease resistance is or comprises resistance to a viral pathogen.

In a preferred embodiment, the plant or plant part of this aspect has a relatively improved abiotic stress tolerance. Preferably, said abiotic stress tolerance is salt tolerance.

In a preferred embodiment, the plant or plant part of this aspect has a relatively improved nutritional and/or palatability property.

In a preferred embodiment, the plant or plant part of this aspect has a relatively improved morphological property.

In an eighth aspect the invention provides a plant wherein at least a nucleic acid fragment of a recombinant genetic construct has been inserted into the genetic material of the plant, wherein said recombinant genetic construct comprises one or more nucleic acid fragments insertable into the genetic material of a plant, wherein said one or more nucleic acid fragments consist of a plurality of nucleotide sequences of at least 15 nucleotides in length, or preferably at least 20 nucleotides in length, derived from one or more plants.

Preferably, the at least a nucleic acid fragment of the recombinant genetic construct that has been inserted into the genetic material of the plant is the one or more nucleic acid fragments consisting of a plurality of nucleotide sequences of at least 15 nucleotides in length, or preferably at least 20 nucleotide in length, derived from one or more plants.

Suitably, the plant into which the at least a nucleic acid fragment of the genetic construct has been inserted is of the same species and/or inter-fertile with the one or more plants from which said one or more nucleotide sequences are derived.

Preferably a plant of the sixth to eighth aspect is a monocotyledonous plant or a dicotyledonous plant.

More preferably said plant is or includes a grass of the Poaceae family; a cereal including rice, sorghum, wheat, barley, oats, and maize; a leguminous species including beans and peanut; a solanaceous species including tomato and potato; a brassicaceous species including cabbage and oriental mustard; a cucurbitaceous plants including pumpkin and zucchini; a rosaceous plants including rose; an asteraceous plants including lettuce and sunflower or a relative of any of the preceding plants.

In certain particularly preferred embodiments, said one or more plants is or includes tomato or a relative of tomato. In certain particularly preferred embodiments, said one or more plants is or includes rice, or a relative of rice. In certain particularly preferred embodiments, said one or more plants is or includes sorghum, or a relative or sorghum.

It will be appreciated that the indefinite articles “a” and “an” are not to be read as singular indefinite articles or as otherwise excluding more than one or more than a single target to which the indefinite article refers. For example, “a” nucleotide sequence includes one nucleotide sequence, one or more nucleotide sequences or a plurality of nucleotide sequences.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures, wherein:

FIG. 1 sets forth a diagrammatic illustration of a genetic construct of the invention, and a vector (pIntR 2) of the invention comprising said genetic construct. The nucleotide sequence of this genetic construct is set forth in SEQ ID NO:1.

FIG. 2 sets forth a diagrammatic illustration of a genetic construct of the invention, and a vector of the invention comprising said genetic construct.

FIG. 3 sets forth a diagrammatic illustration of a genetic construct of the invention, and a vector of the invention comprising said genetic construct.

FIG. 4 sets forth results of transient transformation of tomato mesophyll protoplasts with a pRbcS3C:sGFP:tRbcS3C construct and p35S:sGFP:tNOS as a control.

FIG. 5 sets forth results of pRbcS3C:sGFP:tRbcS3C expression in tomato leaves in vascular tissue and stomata.

FIG. 6 sets forth a comparison of GFP expression driven by promoter-terminator pairs belonging to tomato ACTIN (Act7), CYCLOPHILIN (CyP40) and UBIQUITIN (Ubi3) genes by transient expression in agroinfiltrated Nicotiana benthamiana leaves.

FIG. 7 sets forth a comparison of GFP expression driven by promoter-terminator pairs belonging to tomato ACTIN (Act7; left column), CaMV 35S (middle column) and RUBISCO subunit 3C (RbcS3C) genes (right column) by transient expression in agroinfiltrated N. benthamiana leaves.

FIG. 8 sets forth results of regeneration from tomato cotyledons transformed with intragenic pRbcS3C:GS1G245C:tRbcS3C construct on selective 1 mg/L GA medium for 2 weeks; two plates on the left are control concurrent cotyledons which were not co-incubated with construct-harbouring Agrobacterium.

FIG. 9 sets forth results of initial regeneration from tomato cotyledons transformed with intragenic pRbcS3C:GS1G245C:tRbcS3C construct on selective 1 mg/L GA medium for 4 weeks.

FIG. 10 sets forth results of the use of tomato derived amiRNA constructs to target Cucumber mosaic virus sequences. Shown are dual LUC assays following agroinfiltration of N. benthamiana leaves. N=6; Error bars represent the standard error of the mean.

FIG. 11 sets forth CMV symptom development in five wild-type vs five ami10 (SEQ ID NO:15) expressing plants. A: CMV symptom development in wild-type (Top panel) vs ami10 (Bottom panel) plants 3 weeks post CMV inoculation. B: CMV symptom development in wild-type (left) vs ami10 (right) 3 weeks post CMV inoculation.

FIG. 12 sets forth CMV viral load quantification by qRT-PCR in five wild-type vs five ami10 (SEQ ID NO:15) expressing plants. Relative expression ratios were calculated based on the geometric averages of relative ratios of two reference genes, ACTIN and GAPDH.

FIG. 13 demonstrates the process of designing the RNAi construct with nucleotide sequence set forth in SEQ ID NO:18 using tomato (cultivar Moneymaker) sequences, which were used and brought together bioinformatically to create SEQ ID NO:18, where each plant-derived sequence is at least 20 nucleotides in length.

FIG. 14 sets forth results of the use of a tomato derived RNAi construct to target Cucumber mosaic virus sequences. Shown are results of dual LUC assays following agroinfiltration of N. benthamiana leaves. N=6; Error bars represent the standard error of the mean; t-tests showed highly significant differences.

FIG. 15 sets forth the sequence of the genetic construct (SEQ ID NO:1) contained within the basic intragenic cloning vector pIntR 2, which is depicted diagrammatically in FIG. 1, showing: first and second border nucleotide sequences comprising Agrobacterium RB and LB (in bold); tomato RbcS3C promoter and terminator (underlined); and restriction enzyme sites used for insertion of a gene and additional intragenic expression cassettes (in bold). Note that the first border nucleotide sequence (RB sequence) is depicted at the 5′ end of the sequence, and the second border nucleotide sequence (LB sequence) is depicted at the 3′ end of the sequence.

FIG. 16 sets forth virus resistance of Agrobacterium-mediated T-DNA insertional mutant plants (med18) (A); and suppression of tomato MED18 using tomato-derived amiRNA sequences.

FIG. 17 sets forth SEQ ID NOS:1-66, 68, 71-72, 75, 77, 80, 83-89, 93, and 95 in FASTA format.

FIG. 18 sets forth the nucleotide sequence and structure of pIntrA (SEQ ID NO:67), a preferred cloning construct of the invention. BbvCI restriction enzyme site (SEQ ID NO:102); SphI restriction enzyme site (SEQ ID NO:103); RB (SEQ ID NO:104); LB (SEQ ID NO:105); HpaI restriction enzyme site; PmlI restriction enzyme site; nucleotides added to create cloning sites; PARTIAL ACTIN7 promoter (SEQ ID NO:106) and PARTIAL ACTIN7 terminator (SEQ ID NO:107) are indicated by highlighting and/or underlining.

FIG. 19 sets forth the nucleotide sequence (SEQ ID NO NO:69) and structure of a construct comprising a selectable marker gene that is not of or derived from a plants (nptII), for use in co-transformation together with genetic constructs of the invention. RB; LB, nptII selection marker; double 35S promoter; nos terminator, ANT1 Solanum chilense anthocyanin gene; tomato ACTIN7 promoter; and tomato RbcS3C terminator are indicated by highlighting and/or underlining.

FIG. 20 sets forth the nucleotide sequence (SEQ ID NO:70) and structure of a vector of the invention comprising a preferred genetic construct of the invention together with a further genetic construct comprising a selectable marker gene that is not of or derived from a plants (nptII), for use in co-transformation according to the invention. HpaI restriction enzyme site; PmlI restriction enzyme site; RB; LB, nptII selection marker; visual selection ANT1 marker, and partial ACTIN promoter and terminator are indicated by highlighting and/underlining.

FIG. 21 sets forth pSbiUbi1 (SEQ ID NO:73), a preferred cloning construct of the invention comprising a Ubi1 promoter and terminator from Sorghum bicolor; a CTGCAG PstI restriction enzyme site; and a ggcGCC SfoI restriction enzyme site. Ubi1 promoter and terminator from Sobic 004G050000 (SEQ ID NO:108); CTGCAG PstI restriction enzyme site (SEQ ID NO:109); and ggcGCC SfoI restriction enzyme site (SEQ ID NO:110) are indicated by highlighting and/or underlining.

FIG. 22 sets forth pSbiUbi2 (SEQ ID NO:74), a preferred cloning construct of the invention comprising a Ubi2 promoter from Sorghum bicolor; a Ubi1 terminator from Sorghum bicolor; a CTGCAG PstI restriction enzyme site; and a ggcGCC SfoI restriction enzyme site. Ubi2 promoter from Sobic.004G049900 (SEQ ID NO:111) and Ubi1 terminator from Sobic.004G050000; and CTGCAG PstI restriction enzyme site; ggcGCC SfoI restriction enzyme site are indicated by highlighting and/or underlining.

FIG. 23 sets forth pOsaAPX (SEQ ID NO:76), a preferred cloning construct of the invention comprising an Oryza sativa APX promoter and terminator; and a gagcTCCGGATTAtaa multiple cloning site consisting of SacI or Eco53kI and blunt cutter PsiI; GAACGt and cGATTC: XmnI restriction enzyme sites. APX promoter (SEQ ID NO:112); APX terminator (SEQ ID NO:113); gagcTCCGGATTAtaa multiple cloning site consisting of SacI or Eco53kI and blunt cutter PsiI (SEQ ID NO:114); GAACGt (SEQ ID NO:115) and cGATTC (SEQ ID NO:116): and XmnI restriction enzyme sites are indicated by highlighting and/or underlining.

FIG. 24 sets forth tomato plants expressing SEQ ID NO:69, displaying increased anthocyanin levels (purple stem, roots, veins and part of the leaves).

FIG. 25 sets forth tomato plants co-transformed with the vector of the invention set forth in SEQ ID NO:69 (left), showing strong anthocyanin production, as compared to control tomato plants (right).

FIG. 26 sets forth an ACTIN1:DREB1A:DREB1A genetic construct of the invention (SEQ ID NO:78) comprising nucleotide sequence of an Oryza sativa DREB1A gene; an Oryza sativa Actin1 promoter, and an Oryza sativa DREB1A terminator. The genetic construct further comprises NheI and PmlI restriction digest sites for excision and cloning. NheI (SEQ ID NO:117) and PmlI (SEQ ID NO:118) restriction sites; DREB1A coding sequence (SEQ ID NO:119); and added GTGTT sequence at the 3′ end of the DREB1A coding sequence are indicated using highlighting and/or underline.

FIG. 27 sets forth an NCED3:DREB1A:NCED3 genetic construct of the invention (SEQ ID NO:79) comprising nucleotide sequence of an Oryza sativa DREB1A gene; and an Oryza sativa NCED3 promoter and terminator. Additional TGC (SEQ ID NO:120) and GCA (SEQ ID NO:121) nucleotides; NCED3 promoter (SEQ ID NO:122); and NCED3 terminator (SEQ ID NO:123); and DREB1A coding sequence are indicated by the use of highlighting and/or underlining.

FIG. 28 sets forth regeneration of rice callus transformed with ACTIN1:DREB1A:DREB1A (left) or NCED3:DREB1A:NCED3 (right) on medium containing 100 mM NaCl.

FIG. 29 sets forth CMV inoculated ami11-I T1 plants and CMV inoculated wild type control tomato plants. All wild type plants display “shoestring” symptoms in new growth (right-hand side). Most ami11-I plants appear symptom-free (left-hand side).

FIG. 30 sets forth ELISA assessment of CMV load in WT, T1 azygous, and ami11-I T1 tomato plants.

FIG. 31 sets forth ELISA assessment of CMV load in WT, T1 azygous, and ami11-II T1 tomato plants.

FIG. 32 sets forth assessment of CMV severity and plant height in ami11-I and ami11-II tomato plants.

FIG. 33 sets forth fruit number and exemplary fruit morphology from ami11-I and ami11-II lines infected with CMV.

FIG. 34 sets forth nucleotide sequence of a ‘double’ anti-CMV amiRNA insert with tomato-derived anti-CMV ami10 and ami11, and assessment of RNA targeting of the insert.

FIG. 35 sets forth nucleotide sequence (SEQ ID NO:81) and structure of a preferred genetic construct of the invention comprising CMV amiRNA 10 and amiRNA 11. LB; Actin promoter; CMV amiRNA 10 in Sly-miR156b; amiRNA 11 in Sly-miR156a; Actin terminator; and RB are indicated by highlighting/text colour.

FIG. 36 sets forth nucleotide sequence (SEQ ID NO:82) and structure of a preferred vector of the invention comprising the genetic construct set forth in FIG. 35 in conjunction with the selectable marker-containing genetic construct set forth in FIG. 19. Components of the vector are indicated by highlighting/text colour.

FIG. 37 sets forth intragenic TSWV-targeting amiRNA 7 sequence (SEQ ID NO:83); an assessment of RNA targeting by this sequence using dual LUC assays following agroinfiltration of N. benthamiana leaves (error bars represent the standard error of the mean); and exemplary morphology of a tomato plant transformed to express this sequence.

FIG. 38 sets forth nucleotide sequences (SEQ ID NOS:84-85) of sorghum-derived amiRNAs (amiRNA 3 and amiRNA 6) targeting conserved regions of MDMV and SCMV, assessment of RNA targeting by these sequences, and regenerating sorghum plants. Successful transformants are expected to have a MDMV/SCMV resistance phenotype.

FIG. 39 sets forth nucleotide sequences (SEQ ID NOS:86-89) of sorghum-derived amiRNAs (amiRNA 2, amiRNA 4, amiRNA 5, and amiRNA 7) targeting JGMV, assessment of RNA targeting by these sequences, and regenerating sorghum plants. Successful transformants are expected to have a JGMV resistance phenotype.

FIG. 40 sets forth nucleotide sequence (SEQ ID NO:90) and structure of a genetic construct of the invention comprising a sorghum Ubi1 promoter and terminator, and three sorghum-derived amiRNAs (amiRNA 4, amiRNA 5, and amiRNA 2) targeting JGMV. Components of the construct are indicated by text colour.

FIG. 41 sets forth nucleotide sequence (SEQ ID NO:91) and structure of a preferred genetic construct of the invention comprising a sorghum Ubi2 promoter and a sorghum Ubi1 terminator, and three sorghum-derived amiRNAs (amiRNA 4, amiRNA 5, and amiRNA 2) targeting JGMV. Components of the construct are indicated by text colour.

FIG. 42 sets forth nucleotide sequence (SEQ ID NO:92) of a rice-derived RTSV amiRNA 1.

FIG. 43 sets forth design of a tomato-derived hairpin RNAi construct targeting TSWV (SEQ ID NO:94). The full nucleotide sequence of the RNAi vector is set forth in SEQ ID NO:95.

FIG. 44 sets forth assessment of RNA targeting by the construct set forth in FIG. 43; exemplary phenotype of tomato plants transformed using the construct set forth in FIG. 43; and TSWV load in tomato plants transformed using the construct set forth in FIG. 43 as compared to wild type tomato plants, when challenged with TSWV.

FIG. 45 sets forth targeting of MED18 by tomato-derived amiRNA27; expression of amiRNA27 and MED18 in transformed tomato plants as compared to wilt type controls; and CMV load in WT as compared to amiRNA27 transformed plants (labelled med18).

FIG. 46 sets forth results of detached leaf P. syringae assays in control (labelled W or WT) as compared to amiRNA27 transformed (labelled A or MED18) tomato plants; and abundance of P. syringae in control as compared to amiRNA27 transformed lines as measured by qPCR of P. syringae Gyrase.

FIG. 47 sets forth regeneration and growth rice plants transformed with ACTIN1:DREB1A:DREB1A on media containing 100 mM NaCl.

FIG. 48 sets forth regeneration and growth of rice plants transformed with NCED3:DREB1A:NCED3 on media containing 100 mM NaCl.

FIG. 49 sets forth a comparison of morphology of tomato plants transformed with tomato-derived amiRNA27 as compared to wild type control lines.

FIG. 50 sets forth nucleotide sequence of tomato derived amiRNA6 targeting MED25; assessment of targeting of MED25 by amiRNA6; and expression of amiRNA6 and MED25 in tomato lines transformed with amiRNA6 as compared to wild type control lines.

FIG. 51 sets forth anthocyanin expression in tomato lines transformed using the construct set forth in SEQ ID NO:69 (left); and anthocyanin expression in regenerating rice plants transformed using the rice-derived construct set forth in SEQ ID NO:98 (right).

FIG. 52 sets forth the nucleotide sequence (SEQ ID NO:100) and structure of a tomato derived hairpin RNAi construct targeting a tomato gene encoding the γ-subunit of the type B heterotrimeric G protein (GGB1); and an exemplary transformed tomato plant co-transformed with said construct and the construct set forth in SEQ ID NO:69, and expressing anthocyanin.

FIG. 53 sets forth developing rice plants produced by particle bombardment using a rice-derived RNAi construct targeting rice BADH2. Successful transformants are expected to have a fragrant phenotype.

FIG. 54 sets forth the nucleotide sequence (SEQ ID NO:96) of a tomato MED25 gene.

FIG. 55 sets forth a visual representation and the nucleotide sequence (SEQ ID NO:98) of a rice derived R1G1B:OSB2:R1G1B construct (SEQ ID NO:98).

FIG. 56 sets forth the nucleotide sequence (SEQ ID NO:99) of a tomato GGB1 transcript.

FIG. 57 sets forth a visual representation and the nucleotide sequence (SEQ ID NO:101) of a rice derived RNAi construct targeting rice BADH2.

### BRIEF DESCRIPTION OF THE SEQUENCE LISTING

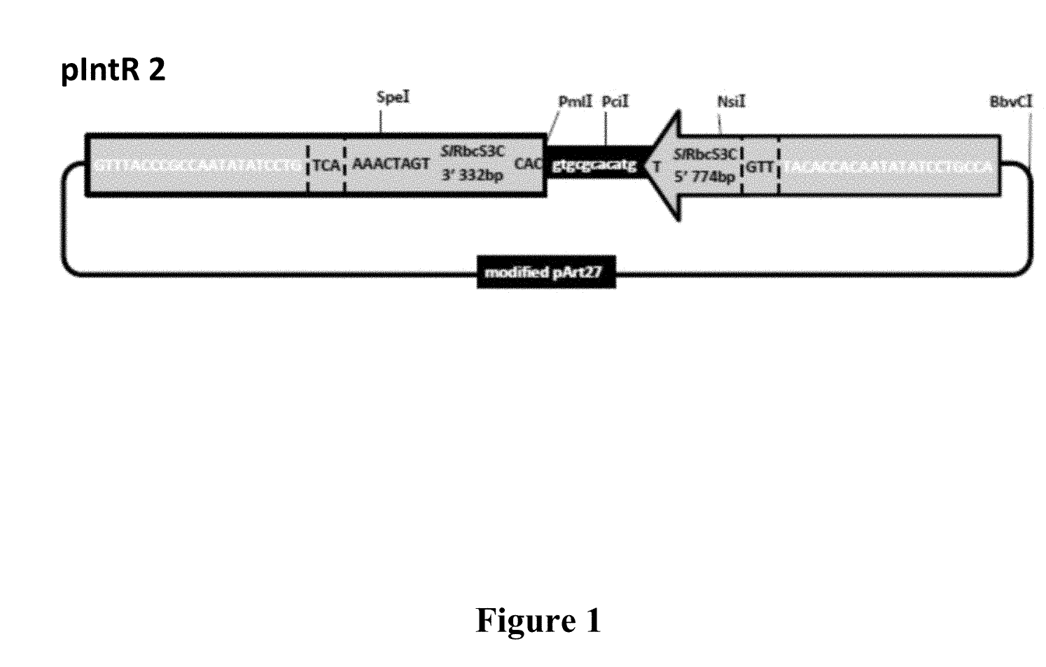

SEQ ID NO:1 Nucleotide sequence of the genetic construct of the invention contained within the basic intragenic cloning vector pIntR 2 of the invention (shown diagrammatically in FIG. 1).
SEQ ID NO:2 Nucleotide sequence of a portion of the first border sequence in certain preferred genetic constructs of the invention.
SEQ ID NO:3 Nucleotide sequence of a portion of the second border sequence in certain preferred genetic constructs of the invention.
SEQ ID NO:4 Nucleotide sequence of the promoter of a RUBISCO subunit 3C (RbS3C) gene of cultivated tomato (Solanum lycopersicum).
SEQ ID NO:5 Nucleotide sequence of the promoter of an ACTIN gene of cultivated tomato.
SEQ ID NO:6 Nucleotide sequence of the promoter of a UBIQUITIN gene of cultivated tomato.
SEQ ID NO:7 Nucleotide sequence of the promoter of a CYCLOPHILIN gene of cultivated tomato.
SEQ ID NO:8 Nucleotide sequence of the terminator of a RUBISCO subunit 3C (RbS3C) gene of cultivated tomato.
SEQ ID NO:9 Nucleotide sequence of the terminator of an ACTIN gene of cultivated tomato.
SEQ ID NO:10 Nucleotide sequence of the terminator of a UBIQUITIN gene of cultivated tomato.
SEQ ID NO:11 Nucleotide sequence of the terminator of a CYCLOPHILIN gene of cultivated tomato.
SEQ ID NO:12 Nucleotide sequence of the tomato miR156b gene. Mature miRNA capitalized.
SEQ ID NO:13 Nucleotide sequence of a tomato-derived amiRNA construct based on SEQ ID NO:12 targeting Cucumber mosaic virus (CMV) K segment 1 replicase (nucleotides 2665-2685). Mature miRNA capitalized.
SEQ ID NO:14 Nucleotide sequence of a tomato-derived amiRNA construct based on SEQ ID NO:12 targeting K segment 2 orf3 (nucleotides 198-218). Mature miRNA capitalized.
SEQ ID NO:15 Nucleotide sequence of a tomato-derived amiRNA construct based on SEQ ID NO:12 targeting CMV K segment 3 orf1 (nucleotides 56-76). Mature miRNA capitalized.
SEQ ID NO:16 Nucleotide sequence of a tomato-derived amiRNA construct based on SEQ ID NO:12 targeting CMV K segment 1 replicase (nucleotides 1437-1457). Mature miRNA capitalized. Mature miRNA capitalized.
SEQ ID NO:17 Nucleotide sequence of a tomato-derived amiRNA construct based on SEQ ID NO:12 targeting CMV K segment 3 orf1 (nucleotides 707-727). Mature miRNA capitalized.
SEQ ID NO:18 Nucleotide sequence of a tomato-derived RNAi construct targeting CMV.
SEQ ID NO:19 Nucleotide sequence of a fragment of SEQ ID NO:18 highly similar to CMV K segment 1 replicase nucleotides 751-896.
SEQ ID NO:20 Nucleotide sequence of a fragment of SEQ ID NO:18 highly similar to CMV K segment 1 replicase nucleotides 1235-1358.
SEQ ID NO:21 Nucleotide sequence of a fragment of SEQ ID NO:18 highly similar to CMV K segment 3 orf2 (coat protein) nucleotides 250-375.
SEQ ID NO:22 Nucleotide sequence of a tomato-derived RNAi construct targeting Tomato spotted wilt virus (TSWV).
SEQ ID NO:23 Nucleotide sequence of a fragment of SEQ ID NO:22 highly similar to TSWV QLD1 segment L RDRP nucleotides 1918-2155.
SEQ ID NO:24 Nucleotide sequence of a fragment of SEQ ID NO:22 highly similar to TSWV QLD1 segment L RDRP nucleotides 8429-8639.
SEQ ID NO:25 Nucleotide sequence of a fragment of SEQ ID NO:22 highly similar to TSWV QLD1 segment M orf1 nucleotides 187-360.
SEQ ID NO:26 Nucleotide sequence of a fragment of SEQ ID NO:22 highly similar to TSWV QLD1 segment M orf2 nucleotides 297-510.
SEQ ID NO:27 Nucleotide sequence of a tomato Betaine Aldehyde Dehydrogenase (BADH) cDNA (gi 209362342).
SEQ ID NO:28 Nucleotide sequence of a tomato Sorbitol Dehydrogenase (SDH) cDNA (gi 78183415).
SEQ ID NO:29 Nucleotide sequence of a tomato Osmotin CDS (gi 460400210).
SEQ ID NO:30 Nucleotide sequence of a tomato Glutamine Synthetase (GTS) cDNA (gi 460409535).
SEQ ID NO:31 Nucleotide sequence of a tomato Phytoene Desaturase cDNA (gi 512772532).
SEQ ID NO:32 Nucleotide sequence of a tomato 5-Enolpyruvyl-3-Phosphoshikimate cDNA (gi 822092668).
SEQ ID NO:33 Nucleotide sequence of a tomato Acetolactate Synthase cDNA (gi 723680771).
SEQ ID NO:34 Nucleotide sequence of a tomato Protoporphyrinogen Oxidase cDNA (gi 723658549).
SEQ ID NO:35 Nucleotide sequence of a Solanum chilense Anthocyanin 1 (ANT1) cDNA (gi 126653934).
SEQ ID NO:36 Nucleotide sequence of a tomato Chlorophyll Synthase cDNA (gi 460401624).
SEQ ID NO:37 Nucleotide sequence of a Barnase suicide construct codon-optimised for Solanum expression, with an intron from a potato ST-LS1 gene.
SEQ ID NO:38 Amino acid sequence of Betaine Aldehyde Dehydrogenase encoded by SEQ ID NO:27.
SEQ ID NO:39 Amino acid sequence of tomato Sorbitol Dehydrogenase protein encoded by SEQ ID NO:28.
SEQ ID NO:40 Amino acid sequence of tomato Osmotin protein encoded by SEQ ID NO:29.
SEQ ID NO:41 Amino acid sequence of tomato Glutamine Synthetase protein encoded by SEQ ID NO:30.
SEQ ID NO:42 Amino acid sequence of tomato Phytoene Desaturase protein encoded by SEQ ID NO:31.
SEQ ID NO:43 Amino acid sequence of tomato 5-Enolpyruvyl-3-Phosphoshikimate protein encoded by SEQ ID NO:32.
SEQ ID NO:44 Amino acid sequence of tomato Acetolactate Synthase protein encoded by SEQ ID NO:33.
SEQ ID NO:45 Amino acid sequence of tomato ProtOx protein encoded by SEQ ID NO:34.
SEQ ID NO:46 Amino acid sequence of Solanum chilense Anthocyanin 1 protein encoded by SEQ ID NO:35.
SEQ ID NO:47 Nucleotide sequence of basic intragenic cloning vector pIntR2 diagrammatically depicted in FIG. 1.
SEQ ID NO:48 Nucleotide sequence of the vector ‘pIntR2 GS1 G245C CML18’ of the invention.
SEQ ID NO:49 Nucleotide sequence of a Glutamine Synthetase 1 (GS1) G245C marker gene operably linked to native GS1 promoter and terminator sequences.
SEQ ID NO:50 Nucleotide sequence of a modified pArt27 backbone of the invention.
SEQ ID NO:51 Nucleotide sequence of CDS of tomato GS1 G733T gene encoding G245C protein.
SEQ ID NO:52 CDS nucleotide sequence of tomato GS1 C745T CDS encoding H249Y protein.
SEQ ID NO:53 Nucleotide sequence of tomato GS1 promoter.
SEQ ID NO:54 Nucleotide sequence of tomato GS1 terminator.
SEQ ID NO:55 Nucleotide sequence of tomato Phytoene Desaturase promoter.
SEQ ID NO:56 Nucleotide sequence of tomato Phytoene Desaturase terminator.
SEQ ID NO:57 Nucleotide sequence of tomato Acetolactate Synthase promoter.
SEQ ID NO:58 Nucleotide sequence of tomato Acetolactate Synthase terminator.
SEQ ID NO:59 Nucleotide sequence of tomato 5-enolpyruvylshikimate-3-phosphate synthase promoter.
SEQ ID NO:60 Nucleotide sequence of tomato 5-enolpyruvylshikimate-3-phosphate synthase terminator.
SEQ ID NO:61 Nucleotide sequence of tomato ProtOx promoter.
SEQ ID NO:62 Nucleotide sequence of tomato ProtOx terminator.
SEQ ID NO:63 Nucleotide sequence of intragenic cloning vector pIntR 2 (SEQ ID NO:1) that is removed upon digestion with PmlI and PciI restriction enzymes, and facilitates ligation of nucleotide sequences into pIntR 2.
SEQ ID NO:64 Nucleotide sequence of the MED18 gene from tomato (gi|723704094|ref|XM_010323502.1).
SEQ ID NO:65 Nucleotide sequence of an amiRNA sequence (MED18 ami3) targeting tomato MED18.
SEQ ID NO:66 Nucleotide sequence of an amiRNA sequence (MED18ami27) targeting tomato MED18.
SEQ ID NO:67 Nucleotide sequence of basic intragenic cloning construct of pIntrA.
SEQ ID NO:68 Nucleotide sequence of removable sequence containing restriction digest sites of pIntrA.
SEQ ID NO:69 Nucleotide sequence of construct comprising a selectable marker gene that is not of or derived from a plants (nptII), for use in co-transformation together with genetic constructs of the invention.
SEQ ID NO:70 Nucleotide sequence of a vector of the invention comprising a preferred genetic construct of the invention together with a further genetic construct comprising a selectable marker gene that is not of or derived from a plants (nptII), for use in co-transformation according to the invention.
SEQ ID NO:71 Nucleotide sequence of a portion of the first border sequence in certain preferred genetic constructs of the invention.
SEQ ID NO:72 Nucleotide sequence of a portion of the second border sequence in certain preferred genetic constructs of the invention.
SEQ ID NO:73 Nucleotide sequence of pSbiUbi1.
SEQ ID NO:74 Nucleotide sequence of pSbiUbi2.
SEQ ID NO:75 Nucleotide sequence of spacer at pSbiUbi1 and pSbiUbi2 cloning sites.
SEQ ID NO:76 Nucleotide sequence of pOsaAPX construct.
SEQ ID NO:77 Nucleotide sequence of spacer at pOsaAPX cloning site.
SEQ ID NO:78 Nucleotide sequence of rice ACTIN1:DREB1A:DREB1A construct.
SEQ ID NO:79 Nucleotide sequence of rice NCED3:DREB1A:NCED3 construct.
SEQ ID NO:80 Nucleotide sequence of tomato-derived double anti-CMV amiRNA insert.
SEQ ID NO:81 Nucleotide sequence of intragenic tomato-derived construct comprising SEQ ID NO:80.
SEQ ID NO:82 Nucleotide sequence of vector comprising SEQ ID NO:81.
SEQ ID NO:83 Nucleotide sequence of tomato-derived anti-TSWV amiRNA 7.
SEQ ID NO:84 Nucleotide sequence of sorghum-derived amiRNA 3 targeting a conserved region of MDMV and SCMV.
SEQ ID NO:85 Nucleotide sequence of sorghum-derived amiRNA 6 targeting a conserved region of MDMV and SCMV.
SEQ ID NO:86 Nucleotide sequence of sorghum-derived amiRNA 2 targeting JGMV.
SEQ ID NO:87 Nucleotide sequence of sorghum-derived amiRNA 4 targeting JGMV.
SEQ ID NO:88 Nucleotide sequence of sorghum-derived amiRNA 5 targeting JGMV.
SEQ ID NO:89 Nucleotide sequence of sorghum-derived amiRNA 7 targeting JGMV.
SEQ ID NO:90 Nucleotide sequence of sorghum-derived triple anti-JGMV amiRNA construct in pSbiUbi1.
SEQ ID NO:91 Nucleotide sequence of sorghum-derived triple anti-JGMV amiRNA construct in pSbiUbi2.
SEQ ID NO:92 Nucleotide sequence of rice-derived amiRNA 1 targeting RTSV.
SEQ ID NO:93 Nucleotide sequence of vector comprising SEQ ID NO:92.
SEQ ID NO:94 Nucleotide sequence of tomato derived hairpin RNAi targeting TSWV.
SEQ ID NO:95 Nucleotide sequence of vector comprising SEQ ID NO:94.
SEQ ID NO:96 Nucleotide sequence of a tomato MED25 gene.
SEQ ID NO:97 Nucleotide sequence of tomato-derived amiRNA6 targeting MED25.
SEQ ID NO:98 Nucleotide sequence of a rice derived R1G1B:OSB2:R1G1B construct.
SEQ ID NO:99 Nucleotide sequence of a tomato GGB1 gene.
SEQ ID NO:100 Nucleotide sequence of a tomato derived hairpin RNAi construct targeting a tomato gene encoding the γ-subunit of the type B heterotrimeric G protein (GGB1).
SEQ ID NO:101 Nucleotide sequence of a rice-derived RNAi construct targeting BADH2.
SEQ ID NO:102 Nucleotide sequence of BbvCI restriction enzyme site.
SEQ ID NO:103 Nucleotide sequence of SphI restriction enzyme site.
SEQ ID NO:104 Nucleotide sequence of RB sequence.
SEQ ID NO:105 Nucleotide sequence of LB sequence.
SEQ ID NO:106 Nucleotide sequence partial ACTIN7 promoter.
SEQ ID NO:107 Nucleotide sequence of partial ACTIN7 terminator.
SEQ ID NO:108 Nucleotide sequence of sorghum Ubi1 promoter and terminator.
SEQ ID NO:109 Nucleotide sequence of PstI restriction site.
SEQ ID NO:110 Nucleotide sequence of SfoI restriction site.
SEQ ID NO:111 Nucleotide sequence of sorghum Ubi2 promoter.
SEQ ID NO:112 Nucleotide sequence of rice APX promoter.
SEQ ID NO:113 Nucleotide sequence of rice APX terminator.
SEQ ID NO:114 Nucleotide sequence of multiple cloning site of pOsaAPX.
SEQ ID NO:115 Nucleotide sequence of XmnI restriction site.
SEQ ID NO:116 Nucleotide sequence of XmnI restriction site.
SEQ ID NO:117 Nucleotide sequence of NheI restriction site.
SEQ ID NO:118 Nucleotide sequence of PmlI restriction site.
SEQ ID NO:119 Nucleotide sequence of rice DREB1A coding sequence with 3′ GTGTT addition.
SEQ ID NO:120 Nucleotide sequence of FspI restriction site.
SEQ ID NO:121 Nucleotide sequence of FspI restriction site.
SEQ ID NO:122 Nucleotide sequence of rice NCED3 promoter.
SEQ ID NO:123 Nucleotide sequence of rice NCED3 terminator.
SEQ ID NO:124 Nucleotide sequence of tomato-derived anti-CMV amiRNA10 in Sly-miR156b.
SEQ ID NO:125 Nucleotide sequence of tomato-derived anti-CMV amiRNA11 in Sly-miR156a.
SEQ ID NOS:126-153 Nucleotide sequence of primers set forth in this specification.

### DETAILED DESCRIPTION

The present invention is at least partly predicted on the realisation that there is a demand for genetic improvement of plants, wherein the introduction of nucleotide sequences that are not derived or derivable from a plant into the genetic material of the plant is avoided.

This invention therefore broadly provides means for the production of genetically improved plants using recombinant genetic constructs comprising nucleotide sequences derived from one or more plants. In one preferred embodiment, said one or more nucleotide sequences are derived from a single plant. Suitably, in embodiments wherein said one or more nucleotide sequences are derived from more than one plants, said plants are of the same species and/or inter-fertile.

It will be appreciated that the genetic alteration that occurs as a result of insertion of a nucleic acid fragment of preferred genetic constructs of the invention into the genetic material of a plant can be the same, or at least similar, as genetic recombination that occurs in nature, e.g. natural genetic recombination that serves to increase diversity of the gene pool in a plant population to increase its survival changes under changing environmental conditions.

It will be further appreciated, as hereinbelow described, that it is preferred that nucleotide sequence that is inserted into a plant using preferred genetic constructs of the invention comprises at least 15, or preferably at least 20 plant-derived nucleotides. It has been realised for the invention that this length of nucleotide sequence is typically the minimum length of nucleotide sequence that is understood to be functional in plants.

As used herein, the term “plant” will be understood to include:

“Embryophyta” or “land plants”, with reference to Margulis, L (1971) Evolution, 25: 242-245 (incorporated herein by reference) and inclusive of liverworts, hornworts, mosses, and vascular plants;

“Viridiplantae” or “green plants”, with reference to Copeland, H F (1956) Palo Alto: Pacific Books, p. 6 (incorporated herein by reference) and inclusive of land plants and green algae.

“Archaeplastida” with reference to Cavalier-Smith, T (1981) BioSystems 14: 461-481 (incorporated by reference) and inclusive of land plants, green plants, Rhodophyta (red algae) and Glaucophyta (glaucophyte algae); and

“Vegetabilia” with reference to Linnaeus, C (1751) Philosophia botanica, 1st ed, p. 37 (incorporated by reference) and inclusive of land plants, green plants, Archaeplastida, and diverse algae and fungi, such as edible fungi including mushrooms.

As used herein, a “genetic construct” will be understood to mean an artificially created segment of genetic material comprising one or more isolated nucleic acids.

As used herein, a nucleotide sequence that is “derived” or “derivable” from a plant will be understood to mean a nucleotide sequence that is substantially the same as a nucleotide sequence found within the native or endogenous genetic material of a plant. It will be readily appreciated that an isolated nucleic acid that comprises a nucleotide sequence that is derived or derivable from a plant need not be obtained from the plant, but can be obtained in any suitable manner, with reference to the detail hereinbelow provided.

It is preferred that a nucleotide sequence that is “derived” or “derivable” from a plant is identical to a native or endogenous plant nucleotide sequence. Suitably, at least wherein the plant derived or plant derivable nucleotide sequence is a protein-coding sequence, the derived or derivable nucleotide sequence will encode an amino acid sequence that is substantially identical, or preferably identical, to a corresponding native or endogenous amino acid sequence. It will be understood however that, while plant derived or plant derivable nucleotide sequences that are identical to a native or endogenous plant nucleotide sequence are preferred, in certain alternative embodiments, the nucleotide sequence may comprise synonymous nucleotide substitutions providing that a protein encoded by the nucleotide sequence is substantially identical, or preferably identical, to a corresponding native or endogenous plant protein.

A used herein in the context of genetic material including genetic constructs, “recombinant”, will be understood to mean genetic material derived from multiple sources. It will be understood that, although parts, portions, or fragments of genetic material that is “recombinant” may comprise nucleotide sequence corresponding to a native nucleotide sequence of the genetic material of a biological organism (such as a plant), the arrangement of the nucleotide sequence within the recombinant genetic material will not occur in the genetic material of the biological organism.

It will be appreciated that recombinant genetic constructs of the invention are designed to facilitate genetic improvement of a plant, wherein at least a nucleic acid fragment of the genetic construct consisting of one or nucleotide sequences that are derived, or derivable, from a plant is inserted into the genetic material of a plant.

Suitably, the production of genetically improved plants comprising nucleotide sequence that is not derived from one or more plants is avoided, or at least substantially minimised, using a genetic construct of the invention.

Suitably, the nucleic acid fragment of the genetic construct that is inserted into a plant as per the invention consists of one or more nucleotide sequence of at least 15, or preferably at least 20, nucleotides in length, that are derived or derivable from one or more plants, wherein said one or more plants are inter-fertile with said plant.

In embodiments, the one or more plants from which the nucleotide sequences of a genetic construct of the invention are derived or derivable is or includes an organism of the classification Vegetabilia as hereinabove described.

In preferred embodiments, the one or more plants from which the nucleotide sequences of a genetic construct of the invention are derived or derivable is or includes an organism of the classification Archaeplastida as hereinabove described.

More preferably, the one or more plants from which the nucleotide sequences of a genetic construct of the invention are derived or derivable is or includes an organism of the classification Viridiplantae as hereinabove described.

Even more preferably, the one or more plants from which the nucleotide sequences of a genetic construct of the invention are derived or derivable is or includes an organism of the classification Embryophyta as hereinabove described.

In some embodiments, the plant is an algae inclusive of microalgae and macroalgae.

In some embodiments, the plant is an edible fungi, inclusive of mushrooms.

Preferably, the plant is monocotyledonous plant or a dicotyledonous plant.

More preferably said one or more plants is or includes a grass of the Poaceae family such as sugar cane; a Gossypium species such as cotton; a berry such as strawberry; a tree species inclusive of fruit trees such as apple and orange and nut trees such as almond; an ornamental plant such as an ornamental flowering plant, inclusive of rosaceous plants such as rose; a vine inclusive of fruit vines such as grapes; a cereal including sorghum, rice, wheat, barley, oats, and maize; a leguminous species including beans such as soybean and peanut; a solanaceous species including tomato and potato; a brassicaceous species including cabbage and oriental mustard; a cucurbitaceous plants including pumpkin and zucchini; a rosaceous plants including rose; an asteraceous plants including lettuce, chicory, and sunflower, or a relative of any of the preceding plants.

In some particularly embodiments, said plant is or includes tomato.

In some particularly preferred embodiments, said plant is or includes sorghum.

In some particularly preferred embodiments, said plant is or includes rice, inclusive of wild rice.

### Isolated Nucleic Acids and Proteins

For the purposes of this invention, by “isolated” is meant material that has been removed from its natural state or otherwise been subjected to human manipulation.

Isolated material may be substantially or essentially free from components that normally accompany it in its natural state, or may be manipulated so as to be in an artificial state together with components that normally accompany it in its natural state. Isolated material may be in native, chemical synthetic or recombinant form.

The term “nucleic acid” as used herein designates single- or double-stranded DNA and RNA. DNA includes genomic DNA and cDNA. RNA includes mRNA, RNA, sRNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylcytosine, methylinosine, methyladenosine and/or thiouridine, although without limitation thereto.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

As used herein, by “protein” is meant an amino acid polymer, comprising natural and/or non-natural amino acids, including L- and D-isomeric forms as are well understood in the art.

In certain embodiments, an isolated nucleic acid of, or an isolated protein encoded by, a genetic construct of the invention is a fragment nucleic acid or protein, respectively.

In certain embodiments, a “fragment” nucleic acid comprises a nucleotide sequence which constitutes less than 100%, but at least 20%, preferably at least 30%, more preferably at least 80% or even more preferably at least 90%, 95%, 96%, 97%, 98% or 99% of a nucleotide sequence set forth in SEQ ID NOS:1-35, 49, 51-56, 66-68, 71-92, or 94-101.

In certain embodiments, a “fragment” protein comprises an amino acid sequence which constitutes less than 100%, but at least 20%, preferably at least 30%, more preferably at least 80% or even more preferably at least 90%, 95%, 96%, 97%, 98% or 99% of an amino acid sequence set forth in SEQ ID NOS:38-46.

In one preferred embodiment a fragment of the genetic construct of the invention comprises no more than 10, 12, 15, 20, 30, 40, 50, 60, 70, 80, 90, 100, 120, 150, 200, 250, 300, 350, 400, 500, 600, 700, 800, 900, 1000, 1500, 2000, 2500, or 3000 contiguous nucleotides of a nucleotide sequence set forth in SEQ ID NOS:1, 67, 73-74, 76, 81, 95, 98, 100, or 101.

An isolated nucleic acid of, an isolated protein encoded by, or a nucleotide sequence that leads to transcriptional or translational silencing or enhancement by the genetic construct of the invention may be a “variant” nucleic acid or protein, respectively, in which one or more nucleotides or amino acids, respectively have been deleted or substituted by different nucleotides or amino acids, respectively.

Variants include naturally occurring (e.g., allelic) variants, orthologs (e.g. from other plants) and synthetic variants, such as produced in vitro using mutagenesis techniques.

In some embodiments, nucleic acid variants include isolated nucleic acids having at least 75%, 80%, 85%, 90% or 95%, 96%, 97%, 98% or 99% nucleotide sequence identity with a nucleotide sequence set forth in SEQ ID NOS:1-35, 49, 51-56, 66-68, 71-92, or 94-101.

In some embodiments, protein variants include proteins having at least 75%, 80%, 85%, 90% or 95%, 96%, 97%, 98% or 99% amino acid sequence identity with an amino acid sequence set forth in SEQ ID NOS:38-46.

Terms used generally herein to describe sequence relationships between respective nucleotide sequences and amino acid sequences include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence.

The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for Windows; available from Hitachi Software engineering Co., Ltd., South San Francisco, Calif., USA).

A detailed discussion of sequence analysis can be found in Chapter 19.3 of Ausubel et al., supra.

It will be appreciated that, without limitation, nucleic acid and protein variants can be created by mutagenizing a protein or an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference. Mutagenesis may also be induced by chemical means, such as ethyl methane sulphonate (EMS) and/or irradiation means, such as fast neutron irradiation of seeds as known in the art (Carroll et al., 1985, Proc. Natl. Acad. Sci. USA 82 4162; Carroll et al., 1985, Plant Physiol. 78 34; Men et al., 2002, Genome Letters 3 147).

### Genetic Constructs

An aspect of the invention provides a recombinant genetic construct comprising one or more nucleic acid fragments insertable into the genetic material of a plant, wherein said one or more nucleic acid fragments comprise, consist of, or consist essentially of a plurality of nucleotide sequences of at least 15 nucleotides in length, or preferably at least 20 nucleotides in length, derived from one or more plants.

As used in this context, a nucleic acid fragment that “consists essentially of” nucleotide sequence derived or derivable from one or more plants, will be understood to include no more than 1, 2, 3, or 4 nucleotides that are not derived or derivable from a plant.

Preferably, the one or more nucleic acid fragments insertable into the genetic material of a plant consist of plant-derived or plant-derivable nucleotide sequences.

In certain preferred embodiments said one or more nucleic acid fragments of the recombinant genetic construct that are insertable into the genetic material of a plant consist of a plurality of nucleotide sequences of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 nucleotides in length.

In certain preferred embodiments said plurality of nucleotide sequences are at least 100, 200, 300, 400, 500, 600, 700, 800, 900, or 1000 nucleotides in length.

In one preferred embodiment, said one or more nucleotide sequences are derived from one plant.

Suitably, in embodiments wherein said one or more nucleotide sequences are derived from more than one plant, said plants are inter-fertile, such as sexually compatible relatives, and/or of the same species.

Preferably, the total length of the one or more nucleic acid fragments of the genetic construct that are insertable into the genetic material of a plant is at least: 100, 200, 300, 400, 500, 600, 700, 800, 900, 1000, 1500, 2000, 2500, 3000, or 3500 base pairs.

With reference to the Examples, it will be appreciated that the preferred recombinant genetic construct pIntR 2 of this aspect comprises 1110 base pairs that are adapted for insertion into the genetic material of a plant, and that this construct is a cloning construct designed to receive further plant-derived nucleotide sequences for insertion or incorporation into the genetic material of a plant. Similarly, the preferred recombinant genetic construct pIntRA of this aspect comprises 1787 base pairs that are adapted for insertion into the genetic material of a plant, and that this construct is a cloning construct designed to receive further plant-derived nucleotide sequences for insertion or incorporation into the genetic material of a plant. Furthermore, the preferred recombinant genetic constructs set forth in SEQ ID NOS:78, 79, 81, 98, and 100 comprise 2387, 3369, 2084, 3304, and 3071 base pairs adapted for insertion into the genetic material of a plant, respectively.

### Sequence of Genetic Constructs

Recombinant genetic constructs of this aspect will suitably comprise one or more nucleotide sequences which can be categorised as follows.

### Sequences for Expression

Preferably, the recombinant genetic construct of this aspect comprises one or more nucleotide sequences for expression. Suitably, said nucleotide sequences for expression are of the one or more nucleic acid fragments of the genetic construct of this aspect that are insertable into the genetic material of a plant.

As used herein in the context of a recombinant genetic construct of the invention, a nucleotide sequence “for expression” will be understood to mean a nucleotide sequence of the genetic construct that is capable of being expressed in a host cell or host organism, such as a plant. Preferably, the sequence for expression is a sequence for expression in a plant.

Preferably, the genetic construct of the invention comprises one or more additional nucleotide sequences for expression, wherein said nucleotide sequences are suitable for expression in a plant to alter or modify a trait of the plant. With reference to the Examples, it will be appreciated that the expression of certain preferred nucleotide sequences has been demonstrated to alter or modify traits including abiotic stress tolerance, nutritional properties, and disease resistance, in plants.

In certain preferred embodiments, one or more of said nucleotide sequences for expression in a plant comprise protein coding nucleotide sequences. The protein coding sequence for expression can be any suitable protein coding sequence. Preferably, the nucleotide sequence encodes a protein associated with a desirable or beneficial plant trait or characteristic, as are well known in the art. By way example, expression of nucleotide sequences encoding proteins including DREB1A, associated with abiotic stress tolerance including salt tolerance, and ANT1, associated with anthocyanin production, has been demonstrated herein.

In certain particularly preferred embodiments, said protein coding nucleotide sequences comprise a nucleotide sequence set forth in SEQ ID NOS:38-46, 76, 78, or 98, or a fragment or variant thereof.

In some preferred embodiments, the genetic construct comprises one or more sequences comprising one or more non-coding nucleotide sequences suitable for expression in a plant to alter or modify a trait of the plant.

Preferably, said non-coding sequences comprise small RNA sequences.

As used herein, “small RNA” will be understood to refer to small, non-coding RNA molecules that have the capacity to bind to and regulate the expression, translation and/or replication of other nucleic acid molecules. The skilled person is directed to Ipsaro, J. J., & Joshua-Tor, L., 2015, Nature Struc. & Mol. Biol. 22 20; and Axtell, J. M., 2013, Ann. Rev. Plant Biol. 64, 137-159, incorporated herein by reference, for summaries of small, non-coding RNA molecules, and such molecules in plant, respectively.

It will be understood that, as used herein, the term small RNA encompasses all such molecules, regardless of the particular name that may be used by the scientific community. By way of non-limiting example, the skilled person will readily appreciate that, as used herein, the term small RNA encompasses small non-coding RNA molecules referred to as ‘miRNA’ and ‘siRNA’.

It will be further understood that small RNA molecules generally have a high degree of nucleotide sequence identity with a nucleic acid molecule for which they have the capacity to bind to and regulate the expression, translation, and/or replication of. However, it will also be understood that a small RNA molecule need not necessarily have 100% identity to such a sequence.

In certain embodiments, a small RNA of the invention has at least 85%, at least 90%, or at least 91%, 92%, 93%, 94%, 95%, 96%, 97%, 98%, or 99% identity to a nucleic acid molecule for which it has the capacity to bind to and regulate the expression, translation, and/or replication.

It will be appreciated that mature small RNAs generally have a length of 18-40 nucleotides. Typically, mature plant small RNAs have a length of 19-26 nucleotides, particularly 19-24 nucleotides. Accordingly, the nucleotide sequence of a small RNA nucleotide sequence for expression of the genetic construct may be 19, 20, 21, 22, 23, or 24 nucleotides in length.

The small RNA sequence may be of a small RNA precursor sequence. As will be readily understood by those skilled in the art, small RNA precursors comprise longer nucleotide sequences than mature small RNAs. When expressed in a plant, small RNA precursors are processed into mature small RNAs. Typically, although without limitation thereto, processing of the small RNA precursors into mature small RNAs is mediated by Dicer-Like Proteins such as DCL-1, DCL-2, DCL-4, and/or Argonaute protein-1 (AGO1).

In certain preferred embodiments, the nucleotide sequence for expression of the genetic construct comprising one or more microRNA sequences comprises a miRNA precursor (pre-miRNA) (e.g. SEQ ID NO:12), or an artificial miRNA (amiRNA) construct comprising a modified pre-miRNA (e.g. SEQ ID NOS:13-17).

It will be readily understood by the skilled person that, in plants, pre-miRNAs are non-protein coding sequences from which mature small RNA sequences are produced. Typically, pre-miRNA sequences are between approximately 60 nucleotides and approximately 100 nucleotides in length, although it will be appreciated that they can be greater than several hundred nucleotides in length. These pre-miRNA sequences form secondary ‘stem loop’ structures, prior to processing into one or more mature miRNAs; see Axtell, J. M., supra.

Suitably, amiRNA constructs comprising modified pre-miRNA sequences can be used in which the one or more small RNA sequence of the pre-miRNA sequences are replaced with one or more small RNA sequences of interest (e.g. SEQ ID NOS:13-17).

In certain other preferred embodiments, the sequence for expression comprising one or more small RNA sequences comprises a ‘double stranded RNA’ (‘dsRNA’) or ‘RNAi’ construct (e.g. SEQ ID NO:18 and SEQ ID NO:22).

It will be readily understood that dsRNA or RNAi constructs are designed to express RNA sequences that form double stranded RNA ‘hairpin’ structures. By way of example, the skilled person is directed to Miki, D, & Shimamoto, K, 2004, Plant and Cell Physiology 45 490. Generally, said hairpin structures are up to several hundred base pairs in length. It will be readily understood that when expressed in a plant, said hairpin structures are processed into small RNAs as hereinabove described.

In some preferred embodiments, the one or more small RNA sequences of a nucleotide sequence for expression of the genetic construct are capable of altering the expression, translation and/or replication of one or more nucleic acids of a plant pathogen.

In certain particularly preferred embodiments, said small RNA is capable of inhibiting the replication of a nucleic acid of a plant virus. In other particularly preferred embodiments, said small RNA is capable of inhibiting infection and/or replication of a bacterial plant pathogen. Additionally or alternatively, said small RNA may be capable of inhibiting infection and/or replication of a fungal plant pathogen, and/or a plant infecting or infesting oomycete, nematode, and/or insect.

In particularly preferred embodiment, said non-coding nucleotide sequence for expression that comprises a small RNA sequence comprises a nucleotide sequence set forth in SEQ ID NOS:12-26, 80, 81, 83-92, or 94-101, or a fragment or variants thereof.

The one or more nucleotide sequences of the genetic construct of this aspect that are sequences for expression may additionally or alternatively comprise one or more selectable marker nucleotide sequences.

As used herein, a “selectable marker” nucleotide sequence refers to a nucleotide sequence suitable for expression in a plant cell, plant tissue, or plant, and adapted to facilitate identification of a plant cell, plant tissue, or plant wherein the genetic construct of the invention, or a fragment thereof, has been inserted into the genetic material of said plant cell, plant tissue, or plant.

In particularly preferred embodiments, said one or more selectable marker nucleotide sequences comprise one or more of SEQ ID NOS:27-35 or 119, or fragments or variants thereof, or one or more nucleotide sequences encoding the amino acid sequence set forth in any one of SEQ ID NOS:38-46, respectively, or fragments or variants thereof.

By way of non-limiting example, a selectable marker nucleotide sequence of the one or more additional nucleotide sequences for expression of the genetic construct may be of a gene which, when expressed in a plant, increases the plants tolerance to a toxic metabolite, or increases the plants ability to utilise alternative nutrient sources, as compared to a corresponding wild type plant.

In this respect, it will be recognised that the nucleotide sequence set forth in SEQ ID NO:27, encoding the amino acid sequence set forth in SEQ ID NO:38, is of a betaine aldehyde dehydrogenase gene. The skilled person will recognise that expression of a selectable marker that comprises the nucleotide sequence of a betaine aldehyde dehydrogenase gene, or fragment or variant thereof, can increase the tolerance of a plant to the chemical betaine aldehyde, facilitating selection by application of exogenous betaine aldehyde.

By way of another non-limiting example, a selectable marker nucleotide sequence of the one or more additional nucleotide sequences for expression may be of a gene which confers herbicide tolerance. By way of non-limiting example, it will be recognised that a selectable marker nucleotide sequence encoding a photosynthesis-related or other enzyme target of herbicide action comprising an introduced mutation conferring herbicide tolerance can be used.

In this respect, it will be recognised that the nucleotide sequence set forth in SEQ ID NO:30, encoding the amino acid sequence set forth in SEQ ID NO:41, is of a glutamine synthetase gene.

The skilled person will recognise that expression of a selectable marker nucleotide sequence that encodes a glutamine synthetase protein comprising one or more mutations as compared to a corresponding wild type protein can confer tolerance of a plant to herbicide (e.g. glufosinate ammonium) facilitating selection by application of an exogenous herbicide. In this regard, the skilled person is directed to Tischer, E., DasSarma, S., & Goodman, H. M., 1986, Mol. Gen. Genet. 203 221; and Pornprom, T., Prodmatee, N., & Chatchawankanphanich, O., 2009, Pest Management Sci. 65 216, incorporated herein by reference.

By way of yet another non-limiting example, a selectable marker nucleotide sequence of the one or more additional nucleotide sequences for expression may be a gene which facilitates visual selection.

In this respect, the nucleotide sequence SEQ ID NO:35, encoding the amino acid sequence set forth in SEQ ID NO:46, is of a anthocyanin 1 gene.

The skilled person will appreciate that expression of a selectable marker that comprises the nucleotide sequence of an anthocyanin 1 gene, or a fragment or variant thereof, can facilitate visual selection of plants transformed with a genetic construct of the invention, or fragment thereof.

It will be readily understood that a range of other suitable selectable markers known to those skilled in the art can be used according to this embodiment of the invention.

It will be appreciated that, in some embodiments, a selectable marker nucleotide sequence of the genetic construct of the invention may also be a nucleotide sequence suitable for expression in a plant to alter or modify a trait of the plant.

By way of non-limiting example, the skilled person will appreciate that the expression of SEQ ID NO:27, encoding the amino acid sequence set forth in SEQ ID NO:38, of a betaine aldehyde dehydrogenase gene (as hereinabove described), can confer increased tolerance to drought and/or salt stress in a plant.

It will be further appreciated with reference to the Examples that it has been demonstrated herein that the expression of DREB1A can confer salt tolerance, which has enabled the production of intragenic transformed plants to be selected via regeneration on salt-containing medium.

By way of another non-limiting example, the skilled person will appreciate that the expression of SEQ ID NO:35, encoding the amino acid sequence set forth in SEQ ID NO:46, of a anthocyanin 1 gene (as hereinabove described), can increase stress tolerance in a plant, and increase the nutritional properties of a plant for human consumption.

In at least certain embodiments, the use of a nucleotide sequence for expression that both confers a desirable trait and can act as a selectable marker can be highly advantageous. It has been demonstrated herein that this approach can facilitate efficient selection of intragenic transformants without the need for the use of other selectable markers.

### Regulatory Sequences

The recombinant genetic construct of this aspect preferably comprises one or more regulatory nucleotide. Suitably, the one or more regulatory sequences are of the nucleic acid fragments of the genetic construct of this aspect that are insertable into the genetic material of a plant. Suitably, the nucleotide sequences for expression of the genetic construct are operably connected with one or more of said regulatory nucleotide sequences.

As used herein, a “regulatory sequence” is a nucleotide sequence that is capable of controlling or otherwise facilitating, enabling, or modifying transcription and/or translation of one or more other nucleotide sequences with which the regulatory sequence is operably connected.

By “operably connected” or “operably linked” is meant that said regulatory nucleotide sequence(s) is/are suitably positioned relative to said one or more nucleotide sequences in order to achieve said control or modification of transcription and/or translation.

Suitably, a regulatory sequence of the additional sequences of the genetic construct is capable of controlling or modifying transcription and/or translation of one or more nucleotide sequences for expression of the recombinant genetic construct, with which the regulatory sequence is operably connected.

A wide range of regulatory sequences are known to those skilled in the art, and may include, without limitation: promoter sequences; leader or signal sequences; ribosomal binding sites; transcriptional start and stop sequences, translational start and stop sequences; enhancer or activator sequences; and terminator sequences.

Preferably, the one or more regulatory nucleotide sequences comprise a promoter sequence.

Preferably, the one or more regulatory sequences comprise a terminator sequence.

It will be appreciated that regulatory sequences that facilitate, by way of non-limiting example, constitutive expression; tissue specific expression; developmental stage-specific expression, or inducible expression (e.g. in response to environmental stimuli) can be used according to the invention.

In certain preferred embodiments, native regulatory elements of one or more plants, or fragments or variants thereof, may be selected for use in a genetic construct of the invention based on the endogenous expression of plant genes or non-coding sequences with which they are operably connected.

In preferred embodiments, the regulatory sequences comprise a promoter comprising a nucleotide sequence set forth SEQ ID NOS:4-7, 53, 55, 57, 59, 61, 67, 73, 74, 76, 78, or 98 or a fragments or variant thereof.

In preferred embodiments, the regulatory sequences comprise a terminator comprising a nucleotide sequence set forth in SEQ ID NOS:8-11, :54, 56, 58, 60, 62, 106, 108, 111, or 112, or a fragment or variant thereof.

### Other Sequences

A genetic construct of this aspect may comprise further nucleotide sequences as described below. It will be appreciated that said other sequences may, but need not necessarily, be of the one or more nucleic acid fragments of the recombinant genetic construct of this aspect that are insertable into the genetic material of a plant. It will be further appreciated that said other sequences may be of the one or more nucleotide sequences for expression, and/or the one or more regulatory sequences of the recombinant genetic construct.

Preferably, the genetic construct comprises nucleotide sequences comprising one or more restriction digest or restriction enzyme sites. Suitably, the restriction digest sites facilitate addition and/or removal of nucleotide sequences of a genetic construct of the invention.

In certain particularly preferred embodiments, the recombinant genetic construct of this aspect comprises flanking sequences of or surrounding nucleic acid fragments insertable into the genetic material of a plant. In some embodiments, the flanking sequences, or portions thereof, are derived from one or more plants. Preferably, the flanking sequences comprise restriction digest sites. In certain particularly preferred embodiments, one or more of the flanking sequences comprise a nucleotide sequence set forth in SEQ ID NOS:102, 103, 109, 110, 115, 116, 117, 118, 120, or 121, or a fragment or variant thereof.

Suitably, flanking sequences comprising restriction digest sites facilitate removal or excision of one or more fragments of the recombinant genetic construct of this aspect consisting of plant derived sequences from a larger construct and/or vector. With reference to the Examples, it will be appreciated by way of example that the preferred genetic constructs set forth in SEQ ID NOS:73-74, 78, 79, 98, and 101 comprise such flanking sequences facilitating removal of fragments of the recombinant genetic construct consisting of plant derived sequences.

Such embodiments may be particularly desirable for transformation approaches using genetic constructs of this aspect involving direct transformation, e.g. particle bombardment. It will be appreciated that removal or excision of a fragment consisting of plant-derived nucleotide sequences facilitates application of this fragment for transformation of a plant, such that no non-plant derived sequence of the genetic construct is expected to be transferred to the genetic material of the plant.

Furthermore, in certain embodiments, the genetic construct of the invention may comprise one or more “spacer” nucleotide sequences. Preferably, the function of nucleotide sequences of the genetic construct that are expressed nucleotide sequences or regulatory nucleotide sequences are unaffected, or substantially unaffected, by said spacer sequences.

By way of non-limiting example, the one or more spacer nucleotide sequences may comprise an extended regulatory sequence, intergenic sequence and/or intron sequence. The recombinant genetic construct comprise spacer sequences at any suitable location, such as between multiple other additional nucleotide sequences of the genetic construct, although without limitation thereto.

### Border Sequences

In certain preferred embodiments of this aspect, the recombinant genetic construct comprises flanking sequences that are “border” nucleotide sequences.

As used in this context, a “border” nucleotide sequence will be understood to refer to a sequence recognised during bacteria-mediated transformation of a plant, plant cell, or plant tissue. More specifically, in a recombinant genetic construct of the invention, the border nucleotide sequences facilitate transfer of at least a fragment of the genetic construct into the genetic material of a plant, via bacteria-mediated transformation. As will be understood by the skilled person, bacteria-mediated plant transformation is commonly performed using Agrobacterium. In this respect, the skilled person is directed to Banta L. M., Montenegro M., 2008, “Agrobacterium and plant biotechnology,” in AGROBACTERIUM: FROM BIOLOGY TO BIOTECHNOLOGY Eds. Tzfira T., Citovsky V., (New York, N.Y.: Springer).

Suitably, in embodiments wherein the recombinant genetic construct comprises border sequences, the construct comprises a first border nucleotide sequence; a second border nucleotide sequence; and one or more additional nucleotide sequences located between the first border nucleotide sequence and the second border nucleotide sequence, wherein said additional nucleotide sequences, and at least a portion of said first border nucleotide sequence that is adjacent to said additional nucleotide sequences, is derived or derivable from one or more plants.

In some embodiments, at least a portion of the second border nucleotide sequence that is adjacent to the additional nucleotide sequences is derived from one or more plants. Preferably, said one or more plants are the same plants from which the additional nucleotide sequences and the at least a fragment of the first border nucleotide sequence are derived.

It will be appreciated that during Agrobacterium transformation of a plant, border sequences, generally referred to as ‘right border’ (RB) and ‘left border’ (LB) nucleotide sequences, enable the insertion of a nucleotide sequence located between the RB and LB sequences, generally referred to as ‘T-DNA’, into the genetic material of a plant. Generally, said RB and LB sequences are approximately 25 nucleotides in length, although without limitation thereto.

Preferably, the first border nucleotide sequence of the genetic construct of the invention comprises an Agrobacterium RB sequence. Preferably the second border nucleotide sequence of the genetic construct of the invention comprises an Agrobacterium LB sequence. It will be appreciated that in these preferred embodiments, the one or more additional nucleotide sequences of the recombinant genetic construct according to these embodiments can function as a T-DNA during Agrobacterium-mediated transformation of a plant.

It will be further appreciated that during Agrobacterium-mediated transformation of a plant, that often a 2 or 3-nucleotide portion of the RB sequence located adjacent to the T-DNA sequence is inserted into the genetic material of the plant (Thomas and Jones, 2007, Molecular Genetics and Genomics 278 411). For example, in Arabidopsis, the RB after integration is frequently (36%) truncated between the second and fifth bases from the canonical T-DNA insertion site, and for tomato three or less bases of the RB typically remain after integration.

Therefore, as set forth above, in preferred genetic constructs of this aspect comprising border sequence at least a portion of the first border nucleotide sequence located adjacent to the one or more additional nucleotide sequences will be derived from one or more plants. Suitably, the at least a portion of the first border nucleotide sequence that is adjacent to the additional nucleotide sequences is at least 3 nucleotides in length. In certain preferred embodiments, the at least a portion of the first border nucleotide sequence that is adjacent to the additional nucleotide sequences comprises the sequence set forth in SEQ ID NO:2 or SEQ ID NO:71. It will be appreciated that these sequences can be derived from any suitable plants and that these sequences can form part of the adjacent larger plant-derived T-DNA sequences with desirable functions.

It will also be appreciated that, in the majority of cases (e.g. 76% in Arabidopsis; 100% in tomato), during Agrobacterium-mediated transformation of a plant, part or all of the LB sequence itself; and in some cases the sequence up to 100 nucleotides, or even greater, upstream of the LB sequence (i.e. towards to RB sequence), is truncated and therefore not inserted into the genetic material of the plant (Thomas and Jones, supra; Brunaud et al., 2002, EMBO Rep. 3 1152). In some plants the LB sequence is frequently completely truncated after T-DNA integration (Thomas and Jones, supra; 98% of the cases in tomato). Therefore, it is not essential for preferred genetic constructs of this aspect that comprise border sequences that a portion of the second border nucleotide sequence is derived from one or more plants.

However, it will also be appreciated that during Agrobacterium-mediated transformation of a plant, in some circumstances, a portion of the LB sequence can nevertheless be inserted into the genetic material of the plant. At least in certain plants, such as Arabidopsis, when a portion of the LB sequence is inserted into genetic material of a plant during Agrobacterium-mediated transformation, said portion is typically between 1 nucleotide and 22 nucleotides in length (see, Brunaud et al., supra). Therefore, in certain embodiments, at least a portion of the second border nucleotide sequence that is adjacent to the additional nucleotide sequences is derived from one or more plants. Preferably, said portion of the border nucleotide sequence is at least 2 nucleotides in length. In some embodiments said portion of the second border nucleotide sequence is at least 22 nucleotides in length.

The presence of said portion of the second border nucleotide sequence that is derived from a plants can be advantageous in circumstances wherein a portion of said border sequence is inserted into the genetic material of the plant, as this should reduce the likelihood that any nucleotide sequence of the genetic construct that is not derived from a plants is inserted into the genetic material of the plant in these circumstances. In certain preferred embodiments, the at least a portion of the second border nucleotide sequence that is adjacent to the additional nucleotide sequences, and derived from one or more plants, comprises the sequence set forth in SEQ ID NO:3 or SEQ ID NO:72. It will be appreciated that these sequences can be derived from any suitable plants and that these sequences can form part of the adjacent larger plant-derived T-DNA sequences with desirable functions.

In particularly preferred embodiments wherein the recombinant genetic construct comprises border sequences, preferably the one or more nucleic acid fragments of the recombinant genetic construct that are insertable into the genetic material of a plant consisting of a plurality of nucleotide sequences of at least 15 nucleotides in length, or preferably at least 20 nucleotides in length, derived from one or more plants consist of:

(i) the at least a portion of the first border sequence derived from one or more plants;

(ii) the one or more additional nucleotide sequences derived from one or more plants; and, optionally

(iii) at least a portion of the second border sequence derived from one or more plants.

It will be appreciated that, in certain said preferred embodiments, a single at least 15, or preferably at least 20, nucleotide sequence may form the portion of the first border sequence comprising a nucleotide sequence derived from a plant and the additional nucleotide sequence located adjacent to said first border sequence. Similarly, it will be appreciated that, in certain said preferred embodiments, a single at least 15, or preferably at least 20, nucleotide sequence may form the portion of the second border sequence comprising a nucleotide sequence derived from a plant and the additional nucleotide sequence located adjacent to said second border sequence.

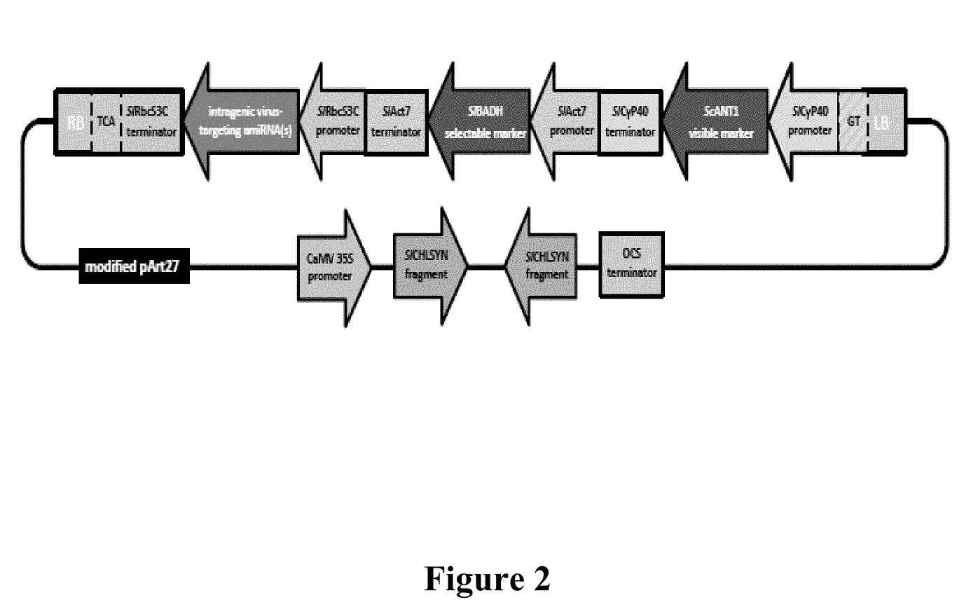

By way of example, in the genetic construct set forth in FIG. 2, it will be appreciated that a single plant-derived nucleotide sequence of a tomato RbcS3C terminator forms the 3-nucleotide portion of the first border sequence that is derived from a plants and the additional nucleotide sequence located adjacent to the first border sequence; and that a single plant-derived nucleotide sequence of a tomato RbcS3C promoter forms the 3-nucleotide portion of the second border sequence that is derived from a plants and the additional nucleotide sequence located adjacent to the second border sequence.

In some preferred embodiments of this aspect wherein the recombinant genetic construct comprises border nucleotide sequence, the genetic construct further comprises a spacer sequence, as hereinabove described, located adjacent to the second border nucleotide sequence.

As hereinabove described, when the genetic construct of the invention, or a fragment thereof, is inserted into the genetic material of a plant via Agrobacterium-mediated transformation, generally the second border sequence and at least a portion of the one or more additional sequences of the genetic construct located towards the second border sequence, is truncated and not inserted into the genetic material of the plant.

Therefore, the location of a spacer sequence adjacent to the second border nucleotide sequence can be advantageous, as this can result in a portion of the one or more additional nucleotide sequences which comprises all other of the additional nucleotide sequences of the genetic construct being inserted into the genetic material of a plant, wherein truncation of a portion of the one or more additional nucleotide sequences consisting of said spacer sequence occurs.

In certain preferred embodiments of this aspect wherein the recombinant genetic construct comprises border sequence, the genetic construct comprises a regulatory sequence that is a promoter sequence, located adjacent to the second border nucleotide sequence and operably connected with a selectable marker sequence.

As hereinabove described, it will be appreciated that when the genetic construct, or a nucleic acid fragment thereof, is inserted into the genetic material of a plant via Agrobacterium-mediated transformation, generally the second border sequence, and at least a portion of the one or more additional sequences of the genetic construct located substantially towards the second border sequence, is truncated and not inserted into the genetic material of the plant. However, in some circumstances, at least a portion of the second border sequence may be inserted into the genetic material of the plant.

Therefore, the location of a promoter sequence that is operably connected with a selectable marker nucleotide sequence adjacent to the second border nucleotide sequence can be advantageous, as this can facilitate identification of genetically improved plants produced according the invention, wherein the second border nucleotide sequence of the genetic construct of the invention may be likely to have been inserted into the genetic material of the plant.

Particularly in embodiments of the invention wherein the second border nucleotide sequence does not comprise a portion of plant-derived nucleotide sequence located adjacent to the one or more nucleotide sequences, the nucleotide sequence of the genetic construct that is inserted into the plant may comprise at least a fragment of the second border sequence which is not derived from one or more plants, which is not desirable according to the invention, as hereinabove described.

Therefore, the inclusion of a selectable marker sequence that is operably connected to a promoter sequence located adjacent to the second border sequence can be advantageous, as expression of said selectable marker sequence in a plant will indicate that the second border nucleotide sequence of the genetic construct may have been inserted into the genetic material of the plant. This can indicate that the plant may not be desirable for further use according to the invention, or that it may be beneficial to perform further analysis of the plant to determine whether nucleotide sequence of the second border sequence that is not derived from one or more plants has been inserted into the genetic material of the plant.

In one preferred embodiment, said promoter sequence located adjacent to the second border nucleotide sequence is operably connected with a selectable marker nucleotide sequence encoding the amino acid sequence set forth in SEQ ID NO:46, or a fragment or variant thereof, which sequence is of a anthocyanin 1-encoding gene, as hereinbefore described.

### Vectors

According to another aspect, the invention provides a vector, wherein the vector comprises a recombinant genetic construct of the invention as hereinabove described. Certain preferred examples of the nucleotide sequence of a vector comprising a genetic construct of the invention are set forth in SEQ ID NOS: 47, 48, 63, 70, 82, 93, and 95.

Suitably, the vector further comprises a vector backbone sequence. One preferred example of a vector backbone sequence of a vector of the invention is set forth in SEQ ID NO:50. However, it will be appreciated that a range of suitable vectors comprising a range of suitable backbone sequences can be used, as are well known in the art.

In preferred embodiments wherein the recombinant genetic construct comprises border sequences, the vector of the invention is adapted for transformation of a plant with a genetic construct of the invention, or a nucleic acid fragment thereof, via bacteria-mediated plant transformation. Preferably, said bacteria-mediated transformation is Agrobacterium-mediated plant transformation.

As will be readily understood by the skilled person, Agrobacterium-mediated plant transformation is generally facilitated by ‘binary’ vector systems. For an overview of binary vector systems for Agrobacterium-mediated plant transformation, the skilled person is directed to Gartland & Davey, 1995, Agrobacterium Protocols (Humana Press Inc. NJ USA); and Lee, L. Y., & Gelvin, S. B., 2008, Plant Physiol., 146 325, incorporated herein by reference.

Briefly, a binary vector typically comprises a T-DNA sequence flanked by RB and LB sequences, as hereinabove described, and additional elements located on a vector backbone sequence which facilitate replication and selection of the vector in certain common laboratory strains of bacteria (e.g. E. coli strains), and Agrobacterium.

Suitably, a binary vector can be transferred to an Agrobacterium strain comprising a separate vector (often referred to as a ‘helper plasmid’) which comprises elements (often referred to as ‘virulence’ elements), which facilitate the transfer of the T-DNA sequence to the genetic material of the plant via Agrobacterium-mediated plant transformation using the Agrobacterium strain.

In these embodiments, preferably the vector is a binary vector.

In certain preferred embodiments, the backbone sequence of the vector comprises a backbone insertion marker.

As used herein, the term “backbone insertion marker” will be understood to refer to a nucleotide sequence that facilitates distinguishing plant cells, tissues, or plants transformed using a vector of the invention wherein the vector backbone has been introduced into the genetic material of a plant, from plant cells, tissues, or plants transformed using a vector of the invention wherein the vector backbone has not been introduced into the genetic material of the plant.

It will be appreciated that, in usual circumstances, as a result of Agrobacterium mediated-transformation of a plant using a vector of the invention, the vector backbone is not transferred to the genetic material of the plant. However, in some circumstances, for example due to incorrect processing of a genetic construct of the invention, the backbone may be inserted into the genetic material of the plant. It will be further appreciated that, although preferred genetic constructs of the invention that are adapted for direct transformation of a plant are designed to allow excision of a fragment consisting of plant-derived sequences for transformation, it is possible (e.g. due to technical error) that a vector backbone may be incorporated into the plant genetic material via direct transformation.

Such circumstances are generally undesirable for the invention; for example the vector backbone sequence may comprise sequence that is not derived from one or more plants, and or is unnecessary or undesirable for the expression of one or more additional sequences that are sequence for expression of a genetic construct of the invention in a plant. Therefore, the inclusion of a backbone insertion marker may be desirable, as this can allow for plants carrying vector backbone sequence to be identified and avoided for further development according to the invention.

A backbone insertion marker of the invention may take any suitable form. In certain embodiments, a backbone insertion marker may facilitate screening of a plant transformed by the application of a chemical or by visual screening, similar to as hereinabove described in relation to selectable markers of the genetic construct of the invention.

In one preferred embodiment, a backbone insertion marker comprises a nucleotide sequence of a small RNA capable of inhibiting or reducing the expression of a gene encoding a chlorophyll synthase protein, such as set forth in SEQ ID NO:36.

It will be appreciated that inhibition or reduction of the expression of a gene encoding a chlorophyll synthase protein by a backbone insertion marker of a vector of the invention in a plant can allow for visual screening of plants transformed using a vector of the invention, wherein reduced or absent chlorophyll pigmentation is indicative of transformation wherein the vector backbone has been inserted into the genetic material of the plant. Suitably, such plants can be avoided for further development according to the invention.

In certain preferred embodiments, the backbone insertion marker is a ‘lethal’ or ‘negative selection’ marker. Suitably, in these embodiments, transformation wherein the backbone is inserted into the genetic material of a plant results in death, or substantially inhibited growth and development, of the transformed plant.

By way of non-limiting example, a negative selection backbone insertion marker may comprise the sequence set forth in SEQ ID NO:37, or a fragment or variant thereof, which is of a Barnase suicide gene.

By way of another non-limiting example, a negative selection backbone insertion marker of a vector of the invention may comprise a small RNA sequence capable of inhibiting or reducing the expression or translation of one or more plant genes or non-protein-coding sequences that are important for survival and/or growth and development of the plant.

### Host Cells

The invention also provides host cells or organisms comprising a genetic construct or vector of the invention. Said host cell or organism may be prokaryotic or eukaryotic.

In certain preferred embodiments, said host cell may by a bacterial cell (e.g. and E. coli cell) capable of propagation of a genetic construct or vector of the invention.

In one preferred embodiments said host cell is an Agrobacterium cell capable of transformation of a plant cell using a vector of the invention, as hereinbefore described.

In one preferred embodiments said host cell is a plant cell or plant tissue (e.g. Nicotiana benthamiana) capable of transiently testing transformation constructs or RNA binding ability of intragenic sequence of the invention, as hereinbefore described.

### Method of Genetically Improving a Plant

Another aspect of the invention provides a method of genetically improving a plant, including the step of introducing at least a fragment of the genetic construct of the invention, or a fragment thereof, into the genetic material of a plant cell or plant tissue.

As hereinabove described, it is particularly desirable for the invention that the at least a nucleic acid fragment of the genetic construct that is introduced into the genetic material of a plant cell or plant tissue according to the method of this aspect is, or is of, a fragment of the genetic construct that consists of one or more nucleotide sequences derived from one or more plants. Suitably, said at least a nucleic acid fragment of the genetic construct that is introduced into the genetic material of the plant consists of the one or more fragments of the genetic construct that consisting of plant-derived nucleotide sequences of at least 15 nucleotides in length, or preferably at least 20 base pairs in length, that are insertable into the genetic material of a plant.

It is particularly preferred according to this aspect that the plant that is genetically improved is of a species that is the same as, and/or inter-fertile with, the one or more plants from which said one or more nucleotide sequences of are derived.

In one embodiment, the method of this aspect includes the steps of:

(i) transforming a plant cell or plant tissue using a genetic construct of the invention or a vector of the invention comprising a genetic construct of the invention; and

(ii) selectively propagating a genetically improved plant from a plant cell or plant tissue transformed in step (i), wherein at least a fragment of the genetic construct has been inserted into the genetic material of the plant cell or plant tissue.

Suitably, a plant cell or plant tissue used for step (i) may be a leaf disk, callus, meristem, hypocotyl, root, leaf spindle or whorl, leaf blade, stem, shoot, petiole, axillary bud, shoot apex, internode, cotyledonary-node, flower stalk or inflorescence tissue, although without limitation thereto.

Suitably, for step (ii), the transformed plant material may, by way non-limiting example, be cultured in shoot induction medium followed by shoot elongation media as is well known in the art. Shoots may be cut and inserted into root induction media to induce root formation as is well known in the art.

In certain preferred embodiments of this aspect, transformation of the plant cell or plant tissue according to step (i) is bacteria-mediated transformation. It is particularly preferred that transformation of the plant cell or plant tissue according to step (i) is Agrobacterium-mediated transformation.

Preferably, in embodiments wherein the transformation of the plant cell or plant tissue is bacteria-mediated transformation, the genetic construct used for the transformation comprises border sequences. Preferably, a vector of the invention is used for said Agrobacterium-mediated transformation. Preferably the vector is a binary vector as hereinabove described.

In certain preferred embodiments, transformation of the plant cell or plant tissue according to step (i) is direct transformation, such as particle bombardment transformation as is well known in the art. Persons skilled in the art will be aware of a variety of plant transformation methods including microprojectile bombardment (Franks & Birch, 1991, Aust. J. Plant. Physiol., 18 471; Bower et al., 1996, Molecular Breeding, 2 239; Nutt et al., 1999, Proc. Aust. Soc. SugarCane Technol. 21 171), liposome-mediated (Ahokas et al., 1987, Heriditas 106 129), laser-mediated (Guo et al., 1995, Physiologia Plantarum 93 19), silicon carbide or tungsten whiskers-mediated (U.S. Pat. No. 5,302,523; Kaeppler et al., 1992, Theor. Appl. Genet. 84 560), virus-mediated (Brisson et al., 1987, Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791), all of which are incorporated herein by reference. In embodiments, transformation according to step (i) of this aspect may be by any of the aforementioned approaches.

In embodiments wherein transformation of the plant cell or plant tissue according to step (i) is direct transformation, preferably the genetic construct used for the transformation comprises flanking sequence for excision of a fragment consisting of plant derived sequences, as hereinabove described, prior to use of said fragment for transformation.

In a preferred embodiment of this aspect, the expression of an additional nucleotide sequence of the genetic construct of the invention that is a selectable marker, as hereinabove described, facilitates selective propagation of a genetically improved plant according to step (ii).

In certain preferred embodiments said selectable marker nucleotide sequence facilitates selection by increasing the tolerance of a genetically improved plant tolerance to a toxic metabolite, or increases the plants ability to utilise alternative nutrient sources, as compared to a corresponding wild type plant. In one preferred embodiment, said selectable marker comprises a nucleotide sequence encoding the amino acid sequence set forth in SEQ ID NO:38, which is of a betaine aldehyde dehydrogenase gene, as hereinabove described.

In certain other preferred embodiments, said selectable marker sequence facilitates selection by conferring herbicide tolerance to a genetically improved plant. In one preferred embodiment, said selectable marker comprises a nucleotide sequence encoding the amino acid sequence set forth in SEQ ID NO:41, which is of a glutamine synthetase gene, as hereinabove described.

In certain other preferred embodiments, said selectable marker sequence facilitates selection by conferring salinity tolerance to a genetically improved plant. In one preferred embodiment, said selectable marker comprises the nucleotide sequence set forth in SEQ ID NO:119, which is of a DREB1A gene, as hereinabove described.

In certain embodiments of the method of this aspect, the method includes the further steps of:

inserting a nucleic acid fragment of a further genetic construct into the genetic material of the plant;

producing a population of plants from the plant wherein the nucleic acid fragment of the genetic construct of the first aspect and the nucleic acid fragment of the further genetic construct have been inserted into the genetic material; and

selecting a plant from said population of plants, wherein the genetic material of said plant comprises the nucleic acid fragment of the genetic construct of the first aspect, but not the nucleic acid fragment of the further genetic construct.

Preferably, the nucleic acid fragment of the further genetic construct that is inserted into the genetic material of the plant comprises a selectable marker nucleotide sequence.

With reference to the Examples, it will be appreciated that these embodiments are particularly desirable in circumstances wherein incorporation of selectable marker of the further genetic construct into the genetic material of the a plant is desirable for facilitating initial selection of a transformed plant, however it is desirable to ultimately produce plants wherein the genetic material of the plants do not contain said selectable marker.

By way of Example, it has been demonstrated herein that the further construct set forth in SEQ ID NO:69 can be beneficial to use according to these embodiments to facilitate selection of transformants. However, it will be appreciated that said construct is adapted for incorporation of a nucleic acid fragment into the genetic material of a plant wherein said fragment comprises inter alia an NPTII selectable marker gene that is not of or derived from one or more plant species. Accordingly, it is desirable to remove this fragment from transformed plants ultimately selected according to the method of this aspect.

In some preferred such embodiments involving the use of a further genetic construct, the genetic construct of the first aspect and the further genetic construct are of a vector of the fourth aspect. With reference to the Examples, such an vector comprising both the genetic construct of the first aspect and the further genetic construct is exemplified and set forth in SEQ ID NO:70.

In additional or alternative such embodiments, the further genetic construct is of a further vector.

The method of this aspect may further include the step of selecting a genetically improved plant wherein the vector backbone has not been inserted into the genetic material of a plant. Suitably, the expression of a backbone insertion marker of a vector of the invention, as hereinabove described, facilitates selection of a genetically improved plant according to this step.

In certain embodiments, said backbone insertion marker is a visual marker. Suitably, in these embodiments, when the vector backbone has been inserted into the genetic material of a plant, the plant exhibits a visual alteration relative to a corresponding wild type plant. Suitably, in these embodiments, only plants which do not exhibit the visual marker are selected according to this step.

In one preferred embodiment that includes this step, the backbone insertion marker comprises a nucleotide sequence of a small RNA capable of inhibiting or reducing the expression of a gene encoding a chlorophyll synthase protein, such as set forth in SEQ ID NO:36. Suitably, according to this embodiment, when the vector backbone has been inserted into the genetic material of the plant, the plant exhibits substantially altered chlorophyll expression as compared to a corresponding wild type plant. Suitably, according to this embodiment, only plants which do not exhibit substantially altered chlorophyll expression are selected according to this step.

In certain other embodiments of the method that include this further step, the backbone insertion marker is a ‘lethal’ or ‘negative selection’ marker. Suitably, according to these embodiments, when the vector backbone has been inserted into the genetic material of a plant, the plant will not survive, or will exhibit growth and development that is substantially impeded as compared to a corresponding wild type plant. Suitably, according to these embodiments, only surviving plants and/or those plants which do not exhibit substantially impeded growth and development are selected according to this step.

In one particularly preferred embodiment that includes this further step, selection of a genetically improved plant according to this step is facilitated by expression of a backbone insertion marker comprising the nucleotide sequence set forth in SEQ ID NO:37, or a fragment or variant thereof, which is of a Barnase suicide gene, as hereinabove described.

The method of this aspect may further include the step of identifying a genetically improved plant wherein there is an increased likelihood that at least a portion of the second border nucleotide sequence of the genetic construct has been incorporated into the genetic material of the plant.

Suitably, identification of a genetically improved plant according to this step is facilitated by the expression of an additional sequence of the genetic construct that is a selectable marker nucleotide sequence that is operably connected with an additional sequence of the genetic construct that is a promoter nucleotide sequence, wherein said promoter sequence is located adjacent to the second border of the genetic construct, as hereinabove described.

Suitably, according to this embodiment, plants expressing the selectable marker nucleotide sequence are identified as possessing an increased likelihood that at least a portion of the second border nucleotide sequence of the genetic construct has been incorporated into the genetic material of the plant.

In one particularly preferred embodiment of the method of this aspect that includes said further step, selection of a genetically improved plant according to this step is facilitated by the expression of a selectable marker nucleotide sequences comprising the nucleotide sequences set forth in SEQ ID NO:46, or a fragment or variant thereof, which sequence is of an anthocyanin 1 protein, as hereinabove described.

Suitably, according to these embodiments, plants displaying a substantially increased level of anthocyanin as compared to a corresponding wild type plant are identified according to this step.

Genetically Improved Plants with Modified Traits

Preferably, the method of this aspect includes the further step of selecting a genetically improved plant comprising one or more altered, modified, or improved traits relative to a corresponding wild type plant.

Preferably, the one or more traits are altered according to the expression of one or more additional nucleotide sequences of the genetic construct that are suitable for expression in a plant to alter or modify a trait of the plant.

In certain preferred embodiments of this aspect, said one or more nucleotide sequences comprise small RNA nucleotide sequences.

In certain preferred embodiments of this aspect, said one or more nucleotide sequences may comprise protein-coding nucleotide sequences.

Certain non-limiting examples of a trait that may be modified in a plant according to the method of this aspect include: nutritional qualities (including seed or grain quality properties and/or nutritional or palatability qualities of vegetative parts of a plant); stress tolerance, for example abiotic stress tolerance such as drought or salt resistance; plant yield (including seed or grain yield and/or or the yield of vegetative parts of a plant); vigour; plant stature; and seed or grain dormancy; biotic stress resistance such as resistance to disease; and nutrient use and/or efficiency. Disease resistance may include viral, bacterial, fungal, nematode, and/or insect resistance.

It will be further appreciated that the trait may be a morphological trait, such as improved ornamental properties, or desirable shape of fruit, foliage, or any other plant part.

It will be further appreciated that intragenic transformation of plants to express particular desired agents, such as in the context of pharmaceutical and/or nutraceutical production, can be considered trait improvement.

In one preferred embodiment, the trait is a disease resistance trait.

In one preferred embodiment, the trait is an abiotic stress tolerance trait.

In one preferred embodiment, the trait is a nutritional and/or palatability quality trait.

In one preferred embodiment, the trait is a morphological trait.

In certain preferred embodiments of this aspect, the trait of the plant is relatively improved or increased or otherwise positively altered by the expression or one or more protein-coding genes. With reference to the Examples, it has been demonstrated that expression of DREB1A according to the method of this aspect can confer abiotic stress tolerance, and in particular salt tolerance.

In certain preferred embodiment of this aspect, a trait of the plant is relatively improved or increased or otherwise positively altered by the expression of one or more additional nucleotide sequences of the genetic construct that are small RNA sequences.

In a preferred such embodiment, disease resistance in the plant is improved or increased, wherein said small RNA sequences are capable of altering the expression, translation and/or replication of one or more nucleic acids of a plant pathogen.

It will be appreciated that, without limitation, the expression of one or more small RNA sequences that are capable of altering the expression and/or replication of one or more nucleic acids of a plant pathogen may relatively improve or enhance disease resistance in a genetically improved plant of this aspect by attenuating, inhibiting, or eliminating the expression of genes or non-protein-coding sequences of the plant pathogen that facilitate infection of the plant.

It will be further appreciated that, without limitation, the expression of one or more small RNA sequences that are capable of altering the expression and/or replication of one or more nucleic acids of a plant pathogen may relatively improve or enhance disease resistance in a genetically improved plant of this aspect by attenuating, inhibiting, or eliminating the replication or reproduction of the plant pathogen in the plant.

In certain preferred embodiments, the plant pathogen is a viral plant pathogen.

In one preferred embodiment, the expression of one or more small RNA sequences that are capable of altering the expression and/or replication of one or more nucleic acids of a plant virus is capable of attenuating, inhibiting, or eliminating the replication of the plant virus in the plant.

In certain particularly preferred embodiments, the viral plant pathogen is a tomato virus such as Cucumber mosaic virus (CMV) and/or Tomato spotted wilt virus (TSWV). In certain particularly preferred embodiments, the viral plant pathogen is a cereal virus, such as a sorghum virus or a rice virus. Particularly preferred cereal plant viruses according to these embodiments include Maize dwarf mosaic virus (MDMV), Sugarcane mosaic virus (SCMV), and Johnsongrass mosaic virus (JGMV).

In certain preferred embodiments, the plant pathogen is a bacterial plant pathogen. In particularly preferred such embodiments the bacterial plant pathogen is Pseudomonas syringae.

In certain embodiments, the plant pathogen is a fungal plant pathogen. The fungal plant pathogen may be a biotrophic, necrotrophic, or hemibiotrophic fungal plant pathogen.

In other embodiments, a trait of a plant may be improved, increased, or otherwise positively altered by the expression of one or more additional nucleotide sequences of the genetic construct that are small RNA sequences, wherein the small RNA sequences decrease, inhibit, or remove expression of an endogenous gene in the plant.

In certain preferred such embodiments, the trait is a nutritional and/or palatability trait. With reference to the Examples, it will be appreciated that production of fragrant rice using a strategy according to the method of this aspect is being explored.

In certain preferred such embodiments, the trait is a morphological trait. With reference to the Examples, it will be appreciated that production of ‘heart shaped’ tomatoes using a strategy according to the method of this aspect is being explored.

### Alternative Methods of Selection

Although in certain preferred embodiments of the method of this aspect the expression of an additional nucleotide sequence of the genetic construct of the invention that is a selectable marker facilitates selective propagation of a genetically improved plant according to step (ii), as hereinabove described, it will be appreciated that, additionally or alternatively, a separate selection construct may be included at step (i), which comprises a separate selectable marker.

By way of non-limiting example, suitable such selectable markers may include neomycin phosphotransferase II which confers kanamycin and geneticin/G418 resistance (nptII; Raynaerts et al., In: Plant Molecular Biology Manual A9:1-16. Gelvin & Schilperoort Eds (Kluwer, Dordrecht, 1988), bialophos/phosphinothricin resistance (bar; Thompson et al., 1987, EMBO J. 6 1589), streptomycin resistance (aadA; Jones et al., 1987, Mol. Gen. Genet. 210 86) paromomycin resistance (Mauro et al., 1995, Plant Sci. 112 97), β-glucuronidase (gus; Vancanneyt et al., 1990, Mol. Gen. Genet. 220 245) and hygromycin resistance (hmr or hpt; Waldron et al., 1985, Plant Mol. Biol. 5 103; Perl et al., 1996, Nature Biotechnol. 14 624).

As hereinabove described, in preferred embodiments involving the use of a separate selectable marker that comprises nucleotide sequence that is not derived or derivable from a plant, the method includes further steps resulting in the ultimate selection of plants that do not comprise said nucleotide sequence within their genetic material.

Additionally, it will be understood that selection of a genetically improved plant according to step (ii) need not necessarily require the use of a selectable marker.

For example, selection of genetically improved plants produced according to this aspect may be performed by screening for the presence of a nucleotide sequence of a genetic construct of the invention, or fragment thereof, within the genetic material of the plant, by any of a range of methods known to those skilled in the art. By way of non-limiting example, Southern hybridization and/or PCR may be employed to detect DNA of a genetic construct, or fragment thereof, inserted into the genetic material of a plant genetically improved according to this aspect, using appropriate nucleotide sequence-specific primers.

Furthermore, in embodiments wherein the genetic construct comprises one or more protein-encoding nucleotide sequences, selection of a genetically improved plant produced according to this aspect may be performed by screening for expression of a protein encoded by said nucleotide sequence in a plant, for example by using an antibody specific for said protein:

(i) in an ELISA such as described in Chapter 11.2 of CURRENT PROTOCOLS IN MOLECULAR BIOLOGY Eds. Ausubel et al. (John Wiley & Sons Inc. NY, 1995) which is herein incorporated by reference; or

(ii) by Western blotting and/or immunoprecipitation such as described in Chapter 12 of CURRENT PROTOCOLS IN PROTEIN SCIENCE Eds. Coligan et al. (John Wiley & Sons Inc. NY, 1997), which is herein incorporated by reference.

Protein-based techniques such as mentioned above may also be found in Chapter 4.2 of PLANT MOLECULAR BIOLOGY: A Laboratory Manual, supra, which is herein incorporated by reference.

It will also be appreciated that, in embodiments wherein the genetic construct comprises one or more nucleotide sequences for expression, selection of a genetically improved plant produced according to the method of this aspect may be performed by screening for the expression of said nucleic acids by, for example, RT-PCR (including quantitative RT-PCR), Northern hybridization, and/or microarray analysis.

For examples of RNA isolation and Northern hybridization methods, the skilled person is referred to Chapter 3 of PLANT MOLECULAR BIOLOGY: A Laboratory Manual, supra, which is herein incorporated by reference. Southern hybridization is described, for example, in Chapter 1 of PLANT MOLECULAR BIOLOGY: A Laboratory Manual, supra, which is incorporated herein by reference.

It will be readily understood that, while a selectable marker as described herein can be advantageous to increase the number of positive transformants during plant transformation, identification of genetically improved plants by PCR and other high throughput type systems (e.g., microarrays, high-throughput sequencing) can enable selection of genetically improved plants without use of a selectable marker due to a large number of samples that may be easily tested.

By way of non-limiting example, PCR may be performed on thousands of samples using primers specific for the transgene or part thereof, the amplified PCR product may be separated by gel electrophoresis, coated onto multi-well plates and/or dot blotting onto a membrane and hybridised with a suitable probe, for example probes described herein including radioactive and fluorescent probes to identify the genetically improved plants.

A related aspect of the invention provides a genetically improved plant produced according to the method of the preceding aspect. Preferably, said plant has an altered or modified trait, relative to a corresponding wild type plant.

In embodiments a plant according to this aspect, or genetically improved plant according to the directly preceding aspect is an organism of the classification Vegetabilia as hereinabove described.

In preferred embodiments, said plant is an organism of the classification Archaeplastida as hereinabove described.

More preferably, said plant is an organism of the classification Viridiplantae as hereinabove described.

Even more preferably, said plant is an organism of the classification Embryophyta as hereinabove described.

In some embodiments, the plant is an algae inclusive of microalgae and macroalgae.

In some embodiments, the plant is an edible fungi, inclusive of mushroom.

Preferably, the plant is monocotyledonous plant or a dicotyledonous plant.

More preferably said plants is a grass of the Poaceae family such as sugar cane; a Gossypium species such as cotton; a berry such as strawberry; a tree species inclusive of fruit trees such as apple and orange and nut trees such as almond; an ornamental plant such as an ornamental flowering plant, inclusive of rosaceous plants such as rose; a vine inclusive of fruit vines such as grapes; a cereal including sorghum, rice, wheat, barley, oats, and maize; a leguminous species including beans such as soybean and peanut; a solanaceous species including tomato and potato; a brassicaceous species including cabbage and oriental mustard; a cucurbitaceous plants including pumpkin and zucchini; a rosaceous plants including rose; an asteraceous plants including lettuce, chicory, and sunflower, or a relative of any of the preceding plants.

In some particularly embodiments, said plant is tomato or a relative of tomato.

In some particularly preferred embodiments, said plant is sorghum or a relative or sorghum.

In some particularly preferred embodiments, said plant is rice or a relative of rice.

### EXAMPLES

### Example 1. Preferred Genetic Constructs and Vectors

This Example sets forth details of certain preferred genetic constructs that have been designed for the invention, and preferred vectors comprising these genetic constructs.

These preferred genetic constructs and vectors have been designed to facilitate genetic modification of a plant via Agrobacterium-mediated transformation wherein a fragment of the genetic construct that consists of a plurality of nucleotide sequences derived from one or more plants is inserted into the genetic material of the plant. Each of said plurality of nucleotide sequences derived from one or more plants is at least 20 nucleotide sequences in length. It will be readily appreciated however, that direct gene transfer (e.g. by using biolistics) can also be used for plant transformation using genetic constructs and/or vectors described herein.

### Basic Cloning Constructs and Vectors: Tomato

A schematic diagram of one preferred genetic construct, and vector comprising this genetic construct (pIntR 2), is set forth in FIG. 1. The complete nucleotide sequence of this genetic construct is set forth in SEQ ID NO:1. The complete nucleotide sequence of the vector is set forth in SEQ ID NO:47.

The backbone sequence of the vector set forth in FIG. 1 is the backbone sequence of the binary vector pArt27.

The genetic construct comprises: a first border sequence that is of an Agrobacterium RB sequence; a second border sequence that is of an Agrobacterium LB sequence; and a plurality of additional sequences located between the RB sequence and the LB sequence. The additional nucleotide sequences and respective portions of the RB sequence and the LB sequence are derived from cultivated tomato (Solanum lycopersicum).

The portion of the RB sequence derived from tomato is the 3-nucleotides of the RB sequence adjacent to the additional nucleotide sequences of the genetic construct, comprising the sequence set forth in SEQ ID NO:2. The portion of the LB sequence derived from tomato is the 3-nucleotides of the second border sequence adjacent to the additional nucleotide sequences, comprising the sequence set forth in SEQ ID NO:3.

The additional nucleotide sequences of the genetic construct comprise:

(i) the regulatory sequence set forth in SEQ ID NO:4 that is of the promoter of a tomato RbcS3C gene, located adjacent to the LB sequence;

(ii) the regulatory sequence set forth in SEQ ID NO:8 that is of the terminator of a tomato RbcS3C gene, located adjacent to the RB sequence;

(iii) a spacer sequence.

It will be appreciated that the 3-nucleotide portion of the LB sequence is a fragment of the promoter sequence of the tomato RbcS3C gene of (i), such that this portion of the LB sequence and (i) are of a single plant-derived nucleotide sequence.

Similarly, it will be appreciated that the 3-nucleotide portion of the RB sequence is a fragment of the terminator sequence of the tomato RbcS3C gene of (ii), such that this portion of the RB sequence and (ii) are of a single plant-derived nucleotide sequence.

The spacer sequence of the genetic construct is in the form of an ‘extended’ portion of the promoter nucleotide sequence of (i) located adjacent to the LB sequence. The nucleotide sequence of (i) has been designed such that truncation of this spacer sequence should not substantially compromise the promoter function of (i).

The genetic construct of this example further comprises the restriction enzyme sites SpeI, PmiI, PciI, and NsiI. The restriction enzyme sites SpeI is of the RbcS3C terminator sequence; and the restriction enzyme site NsiI is of the RbcS3C promoter sequence. The restriction enzyme site PciI is of the nucleotide sequence GTGCGCACATG (SEQ ID NO:63), located between the RbcS3C promoter sequence and the RbcS3C terminator sequence. The restriction enzyme site PmlI is formed from the 3 base pairs (CAC) of the nucleotide sequence of the RbcS3C terminator sequence and three base pairs (GTG) of SEQ ID NO:63.

It will be understood that SEQ ID NO:63 as per this genetic construct need not necessarily be derived or derivable from one or more plants. Rather, the sequence and location of SEQ ID NO:63 as per the genetic construct of this example has been designed to facilitate introduction of one or more nucleotide sequences derived from tomato, or a relative of tomato, into the genetic construct of this example, by digestion and ligation using the abovementioned PmlI and PciI restriction enzyme sites.

It will be appreciated that after digestion and ligation using the PmlI and PciI restriction sites, and insertion of the one or more nucleotide sequences derived from tomato or a wild relative of tomato, SEQ ID NO:63 is removed from the genetic construct.

Suitably, after introduction of said one or more nucleotide sequences derived from tomato or a wild relative of tomato into the genetic construct, a fragment of the genetic construct of this Example consists of a plurality of nucleotide sequences of at least 15, or preferably at least 20, nucleotides in length derived from one or more plants, wherein said fragment consists of:

(i) the 3-nucleotide portion of the LB sequence that is a fragment of the promoter sequence of the tomato RbcS3C gene;

(ii) the promoter of the tomato RbcS3C gene, located adjacent to the LB sequence;

(iii) the one or more nucleotide sequences derived from tomato, or a wild relative of tomato, introduced into the genetic construct;

(iv) the terminator of the tomato RbcS3C gene, located adjacent to the RB sequence; and

(v) the portion of the RB sequence that is a fragment of the terminator sequence of the tomato RbcS3C gene.

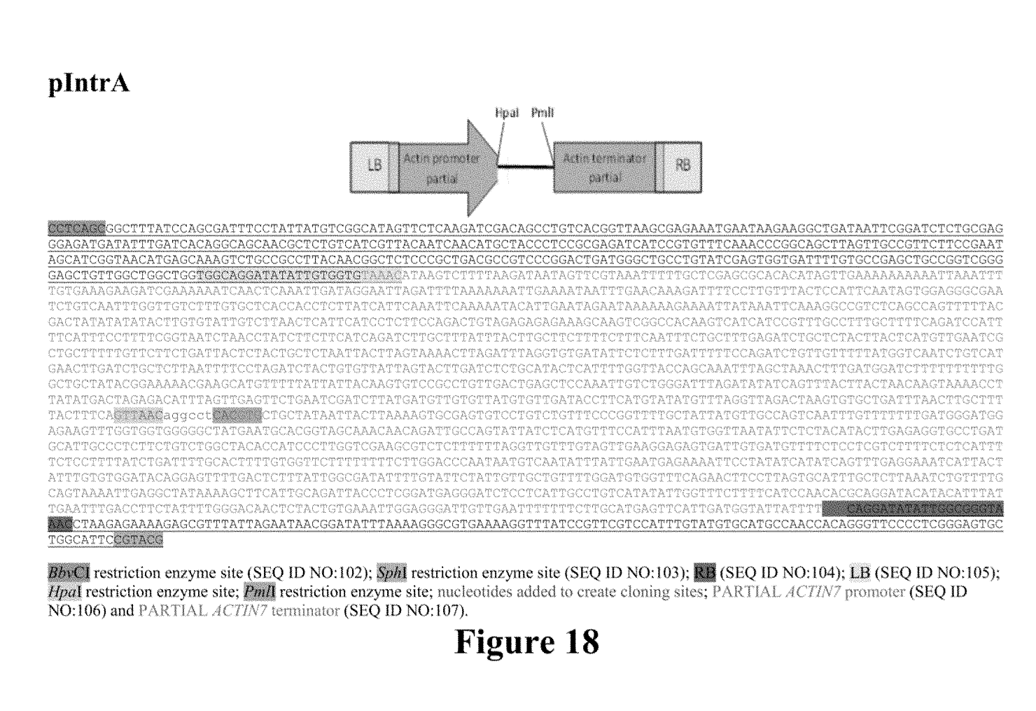

A schematic diagram of another preferred genetic construct of the invention is set forth in FIG. 18. The complete nucleotide sequence of this genetic construct (pIntrA) is set forth in SEQ ID NO:67.

Similar to pIntR 2, the backbone sequence of the vector for pIntrA is the backbone sequence of the binary vector pArt27. It was developed by removing a segment within the RB and LB from blank pArt27 with AseI enzyme (to remove some repeating restriction enzyme sites), re-ligating the remaining portion and substituting the fragment between BbvCI and, now unique, SphI sites with a synthesised sequence containing removed parts of the backbone, RB, LB and tomato ACTIN7 promoter and terminator with cloning sites, HpaI and PmlI, between them. The sequence of synthesised fragment including nucleotides added to create cloning sites between the partial ACTIN7 promoter and partial ACTIN7 terminator is set forth in SEQ ID NO:67.

This genetic construct comprises: a first border sequence that is of an Agrobacterium RB sequence; a second border sequence that is of an Agrobacterium LB sequence; and a plurality of additional sequences located between the RB sequence and the LB sequence. The additional nucleotide sequences and respective portions of the RB sequence and the LB sequence are derived from cultivated tomato (Solanum lycopersicum).

The portion of the RB sequence derived from tomato is the 3-nucleotides of the RB sequence adjacent to the additional nucleotide sequences of the genetic construct, comprising the sequence set forth in SEQ ID NO:2. The portion of the LB sequence derived from tomato is the 5-nucleotides of the second border sequence adjacent to the additional nucleotide sequences, comprising the sequence set forth in SEQ ID NO:3.

The additional nucleotide sequences of the genetic construct comprise:

(i) regulatory sequence that is of the promoter of a tomato ACTIN7 gene, located adjacent to the LB sequence;

(ii) regulatory sequence that is of the terminator of a tomato ACTIN7 gene, located adjacent to the RB sequence;

(iii) a spacer sequence.

It will be appreciated that the 5-nucleotide portion of the LB sequence is a fragment of the promoter sequence of the tomato ACTIN7 gene of (i), such that this portion of the LB sequence and (i) are of a single plant-derived nucleotide sequence.

Similarly, it will be appreciated that the 3-nucleotide portion of the RB sequence is a fragment of the terminator sequence of the tomato ACTIN7 gene of (ii), such that this portion of the RB sequence and (ii) are of a single plant-derived nucleotide sequence.

The spacer sequence of the genetic construct is in the form of an ‘extended’ portion of the promoter nucleotide sequence of (i) located adjacent to the LB sequence. The nucleotide sequence of (i) has been designed such that truncation of this spacer sequence should not substantially compromise the promoter function of (i).

The genetic construct of this example comprises the restriction enzyme sites HpaI and PmlI that are located between the ACTIN7 promoter sequence and the ACTIN7 terminator sequence. The restriction enzyme site HpaI is formed from the 3′ base pairs (GTT) from the ACTIN7 promoter and three base pairs (AAC) are added that are lost after DNA restriction and insertion of a desirable DNA. Similarly, the restriction enzyme site PmlI is formed from the 5′ base pairs (GTG) from the ACTIN7 terminator and three base pairs (CAC) are added that are lost after DNA restriction and insertion of a desirable DNA.

It will be understood that SEQ ID NO:68 between ACTIN7 promoter and terminator in SEQ ID NO:67 as per the genetic construct of this invention need not necessarily be derived or derivable from one or more plants. Rather, the sequence and location of SEQ ID NO:68 as per the genetic construct of this example has been designed to facilitate introduction of one or more nucleotide sequences derived from tomato, or a relative of tomato, into the genetic construct of this example, by digestion and ligation using the abovementioned HpaI and PmlI restriction enzyme sites.

It will be appreciated that after digestion and ligation using the HpaI and PmlI restriction sites, and insertion of the one or more nucleotide sequences derived from tomato or a wild relative of tomato, SEQ ID NO:68 is removed from the genetic construct.

Suitably, after introduction of said one or more nucleotide sequences derived from tomato or a wild relative of tomato into the genetic construct, a fragment of the genetic construct of this Example consists of a plurality of nucleotide sequences of at least 15, or preferably at least 20, nucleotides in length derived from one or more plants, wherein said fragment consists of:

(i) the 5-nucleotide portion of the LB sequence (SEQ ID NO: 71) that is a fragment of the promoter sequence of the tomato ACTIN7 gene;

(ii) the promoter of the tomato ACTIN7 gene, located adjacent to the LB sequence;

(iii) the one or more nucleotide sequences derived from tomato, or a wild relative of tomato, introduced into the genetic construct;

(iv) the terminator of the tomato ACTIN7 gene, located adjacent to the RB sequence; and

(v) the portion of the RB sequence that is a fragment of the terminator sequence of the tomato ACTIN7 gene (SEQ ID NO:72).

Cloning of sequences into pIntrA uses the unique blunt end cloning restriction enzyme sites that must be complemented with the insert, to which the nucleotides are added with primers used to amplify the insert, and these primers also must be 5′ phosphorylated to enable blunt end ligation, i.e:

Forward primer: 5′PhosGATTAAAA[start insert sequence]

Reverse primer: 5′PhosC[reverse complement of end of insert sequence).

T-DNA constructs like those mentioned above (pInR 2 and pIntrA), become completely intragenic (plant genome-derived) when integrated in the plant genome, when only the 3 bases of the 5′ end of the RB remain after integration, while the LB often gets truncated during integration (often removing parts of the adjacent sequence; Thomas and Jones, supra). The adjacent promoter sequences have therefore been chosen to be large enough so that promoter function should not be compromised, even if parts of the promoters at the 5′ end are truncated during integration.

Constructs and Vectors with Sequences for Expression: Tomato

A schematic diagram of another genetic construct and vector comprising said genetic construct, is set forth in FIG. 2.

The backbone sequence of the vector set forth FIG. 2 is modified from the backbone sequence of the binary vector pArt27, and is set forth in SEQ ID NO:50. The modified pArt27 backbone sequence comprises a backbone insertion marker sequence operably linked to a suitable promoter sequence (e.g. a CaMV 35S promoter sequence as depicted in FIG. 2, although this can be varied as desired) and a suitable terminator sequence.

The genetic construct comprises: a first border sequence that is of an Agrobacterium RB sequence; a second border sequence that is of an Agrobacterium LB sequence; and a plurality of additional sequences located between the RB sequence and the LB sequence. The additional nucleotide sequences and respective portions of the RB sequence and the LB sequence are derived from cultivated tomato (Solanum lycopersicum) or Solanum chilense, a wild relative of cultivated tomato.

The portion of the RB sequence derived from tomato is the 3-nucleotides of the RB sequence adjacent to the additional nucleotide sequences of the genetic construct comprising the sequence set forth in SEQ ID NO:2. The portion of the LB sequence derived from tomato is the 3-nucleotides of the second border sequence adjacent to the additional nucleotide sequences comprising the sequence set forth in SEQ ID NO:3.

The additional nucleotide sequences of the genetic construct comprise:

(i) the regulatory nucleotide sequence set forth in SEQ ID NO:7 that is of the promoter sequence of a tomato CyP40 gene, located adjacent to the LB sequence and operably connected with (ii);

(ii) the selectable marker nucleotide sequence set forth in SEQ ID NO:35 that is of a Solanum chilense ANT1 anthocyanin gene;

(iii) the regulatory sequence set forth in SEQ ID NO:11 that is of the terminator of a tomato CyP40 gene, operably connected with (ii);

(iv) the regulatory nucleotide sequence set forth in SEQ ID NO:5 that is of the promoter sequence of a tomato ACTIN gene, operably connected with (v);

(v) the selectable marker nucleotide sequence set forth in SEQ ID NO:27 that is of a tomato betaine aldehyde dehydrogenase gene;

(vi) the regulatory nucleotide sequence set forth SEQ ID NO:9 that is of the terminator of a tomato ACTIN gene, operably connected with (v);

(vii) the regulatory nucleotide sequence set forth in SEQ ID NO:4 that is of the promoter of a tomato RbcS3C gene, operably connected with (viii);

(viii) a nucleotide sequence for expression that comprises one or more small RNA nucleotide sequences capable of modifying the expression and/or replication of one or more nucleic acids of a plant virus;

(ix) the regulatory nucleotide sequence set forth in SEQ ID NO:8 that is of the terminator sequence of a tomato RbcS3C gene, located adjacent to the RB sequence and operably connected with (viii).

It will be appreciated that the 3-nucleotide portion of the LB sequence is a fragment of the promoter sequence of the tomato CyP40 gene of (i), such that this portion of the LB sequence portion and (i) are of a single plant-derived nucleotide sequence.

Similarly, it will be appreciated that the 3-nucleotide portion of the RB sequence is a fragment of the terminator sequence of the tomato RbcS3C gene of (ix), such that this portion of the RB sequence and (ix) are of a single plant-derived nucleotide sequence.

The sequence of (i) has been designed such that substantial truncation of the CyP40 promoter sequence will ablate or substantially compromise the promoter function of (i), such that the ability of (i) to drive the expression of the selectable marker sequence (ii) that is of the Solanum chilense ANT1 anthocyanin gene will be eliminated or substantially reduced.

It will be understood that the fragment of the genetic construct of this Example consisting of the abovementioned 3-nucleotide portions of the LB and RB sequences, and all sequence in between, consists of a plurality of nucleotide sequences of at least 20 nucleotide sequences in length derived from Solanum lycopersicum or Solanum chilense.

Constructs and Vectors with Sequences for Expression: Generic

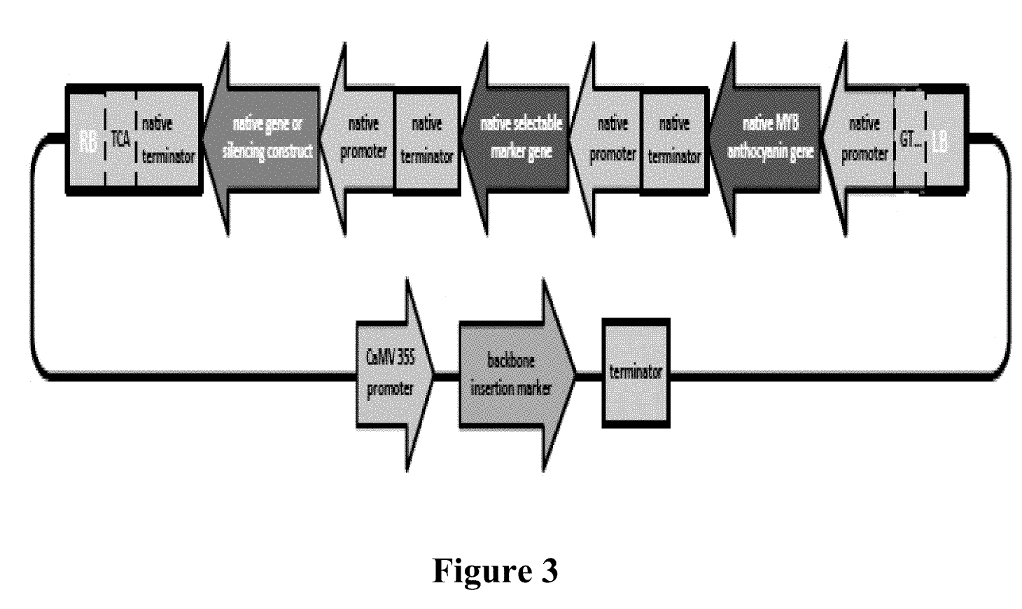

A schematic diagram of yet another preferred genetic construct, and a preferred vector comprising said genetic construct, is set forth in FIG. 3.

The preferred vector comprising the genetic construct further comprises a backbone sequence. The backbone sequence comprises a backbone insertion marker sequence operably linked to a suitable promoter sequence (e.g. a CaMV 35S promoter sequence as depicted in FIG. 3, although this can be varied as desired) and a suitable terminator sequence (e.g. an OCS terminator as depicted in FIG. 3, although this can be varied as desired). As depicted in FIG. 3 the backbone insertion marker is a Barnase suicide gene, however this can be varied as desired.

The genetic construct of the vector set forth in FIG. 3 comprises: a first border sequence that of an Agrobacterium RB sequence; a second border sequence that is of an Agrobacterium LB sequence; and a plurality of additional sequences located between the RB sequence and the LB sequence.

The additional nucleotide sequences and respective portions of the RB sequence and the LB sequence are derived from one or more plants. Said plants can be any suitable plants. In embodiments wherein the additional sequences are derived from a plurality of plants, suitably, said plants are inter-fertile.

The portion of the RB sequence derived from a plants is adjacent to the additional nucleotide sequences of the genetic construct. The portion of the LB sequence derived a plants is adjacent to the additional nucleotide sequences of the genetic construct.

The additional nucleotide sequences of the genetic construct comprise:

(i) a regulatory nucleotide sequence that is of a promoter operably connected with (ii);

(ii) a selectable marker sequence. As depicted in the FIG. 3, said selectable marker sequence is of an anthocyanin gene, but this can be varied as desired;

(iii) a regulatory sequence that is of a terminator operably connected with (ii);

(iv) a further regulatory nucleotide sequence that is of a promoter, operably connected with (v);

(v) a further selectable marker sequence, preferably wherein said sequence is different from the sequence of (ii);

(vi) a regulatory nucleotide sequence that is of a terminator, operably connected with (v);

(vii) a regulatory nucleotide sequence that is of a promoter operably connected with (viii);

(viii) one or more nucleotide sequences for expression, wherein said nucleotide sequences are suitable for expression in a plant to alter or modify a trait of the plant;

(ix) a regulatory nucleotide sequence that is of a terminator operably connected with (viii).

Optionally, the portion of the LB sequence that is derived from one or more plants is a fragment of the promoter sequence of (i), such that this portion of the LB sequence and (i) are of a single plant-derived nucleotide sequence.

Optionally, the portion of the RB sequence that is derived from one or more plants is a fragment of the terminator sequence of (ix), such that this portion of the LB sequence and (ii) are of a single plant-derived nucleotide sequence.

The sequence of (i) should be designed such that substantial truncation of the promoter sequence of (i) will ablate or substantially compromise the promoter function of (i), such that the ability of (i) to drive the expression of the selectable marker sequence (ii) will be eliminated or substantially reduced.

Suitably, at least the fragment of the genetic construct of this Example consisting of the abovementioned portions of the LB and RB sequences derived from one or more plants, and all sequence in between, consists of a plurality of nucleotide sequences of at least 20 nucleotides in length derived from (a) one plants; or (b) two or more inter-fertile plants.

The genetic construct as set forth in this Example is designed to be used for transformation of a plants such that the fragment (or a portion thereof) of the genetic construct consisting of a plurality of nucleotide sequences of at least 20 nucleotides in length derived from one or more plants is inserted into the genetic material of the plant, wherein the transformed plants is the same, or inter-fertile with, the one or more plants from the nucleotide sequences of said fragment of the genetic construct are derived.

Constructs and Vectors with Sequences for Expression: Sorghum

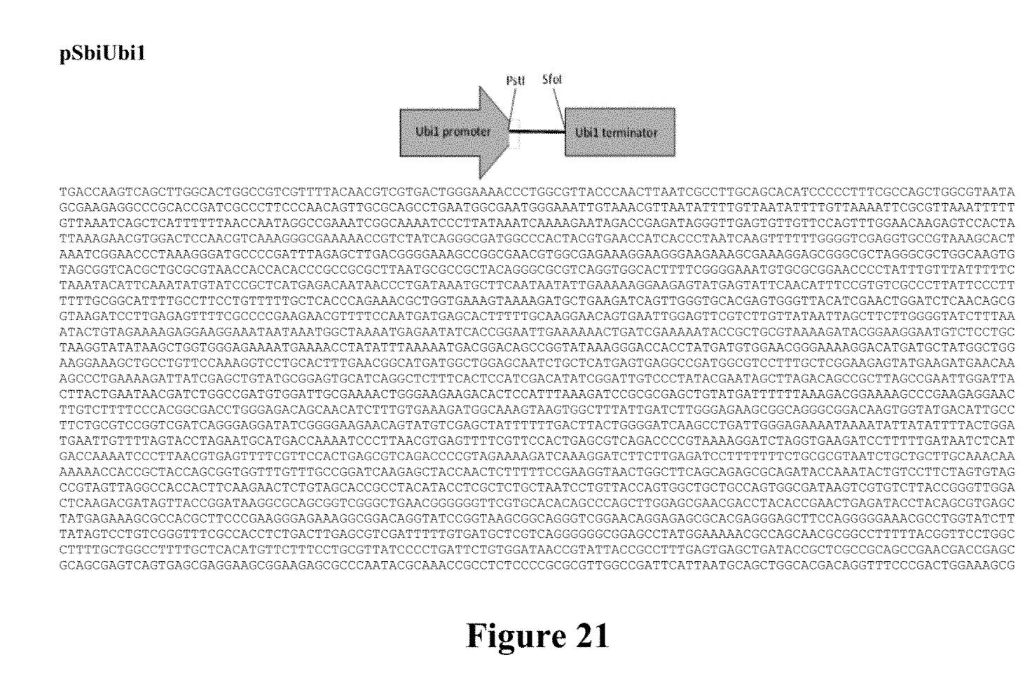

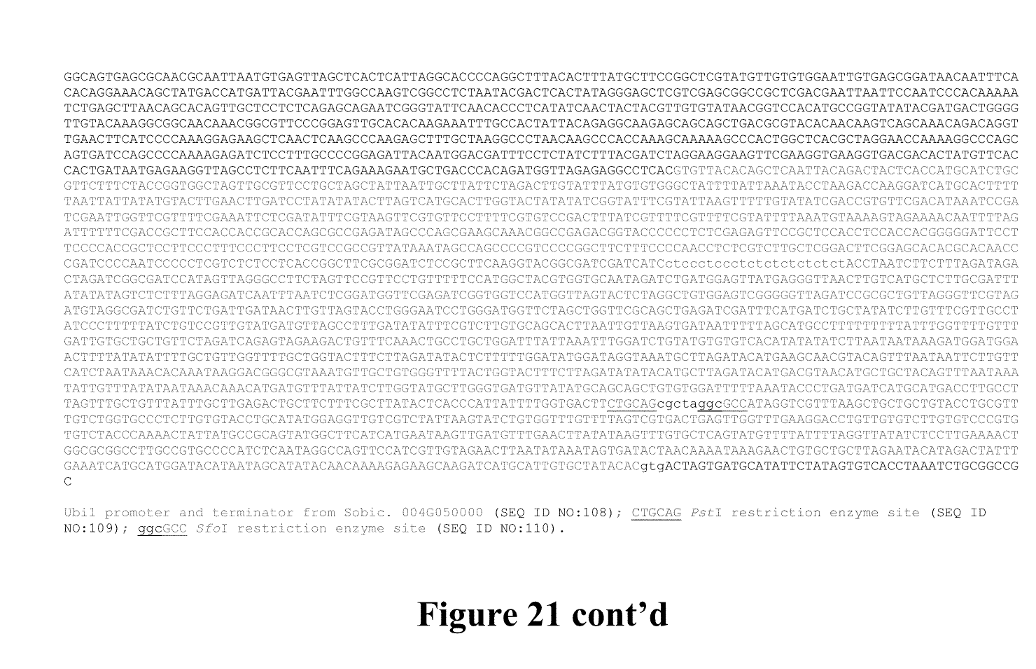

A preferred method for sorghum transformation is by direct gene transfer using biolistics. To ensure that only sorghum genome-derived sequences are used, a vector is used where the linear DNA fragment for direct gene transfer can be easily excised prior to biolistics. A schematic diagram of such a preferred genetic construct (pSbiUbi1) is set forth in FIG. 21. The complete nucleotide sequence of this genetic construct is set forth in SEQ ID NO:73.

The backbone sequence of this vector is the backbone sequence of the vector pKannibal. It contains the promoter sequence of the Sorghum biocolor UBIQUITIN1 gene (Sobic.004G049900) and the terminator of the Sorghum biocolor UBIQUITIN2 gene (Sobic.004G050000). It was developed by making use of the natural PstI site at the 3′ end of the Ubi1 promoter which was amplified from sorghum gDNA with primers F 5′Phos cctcacGTGTTACACAGCTCAATTACAGACTACTCACC (SEQ ID NO:126) (adding 3 nucleotides to the start of the promoter to create a blunt-cutter site PmlI to enable excision of the intragenic cassette prior to direct gene transfer) and R tccCTGCAGAAGTCACCAAAATAATGGGT (SEQ ID NO:127). The fragments were digested with PstI and ligated into vector pKannibal opened up with StuI and PstI. Terminator Ubi1 was amplified with primers F tccCTGCAGcgctaggcGCCATAGGTCGTTTAAGCTGCTG (SEQ ID NO:128) (adding 3 nucleotides to start of the terminator to create a blunt-cutter cloning site SfoI) and R tccCACTAGTcacGTGTATAGCACAATGCATGATCTTGCT (SEQ ID NO:129) (adding 3 nucleotides to end of the terminator to create a blunt-cutter site PmlI for excision of the intragenic cassette, and a SpeI site for insertion in the previous vectors). The fragment was digested with PstI and SpeI and ligated into two previously obtained intermediate vectors opened up with the same enzymes.

This vector (pSbiUbi1) is suitable to express a sequence of interest in sorghum, by amplifying the insert with primers F CTGCAG[start of insert sequence] and R 5′Phos[reverse complement of end of insert sequence]. The fragment is then digested with PstI and ligated into pSbiUbi1 opened up with PstI and SfoI restriction enzymes.

It will be appreciated that after excision, the sequence for direct gene transfer consists of plant-derived nucleotide sequences.

It will be appreciated that after digestion and ligation using the PstI and SfoI restriction sites, and insertion of the one or more nucleotide sequences derived from sorghum or a wild relative of sorghum, spacer SEQ ID NO:75 is removed from the genetic construct.

Suitably, after introduction of said one or more nucleotide sequences derived from sorghum or a wild relative of sorghum into the genetic construct, a fragment of the genetic construct of this Example consists of a plurality of nucleotide sequences of at least 15, or preferably at least 20, nucleotides in length derived from one or more plants, wherein said fragment consists of:

(i) the promoter of the sorghum UBIQUITIN1 gene,

(ii) the one or more nucleotide sequences derived from sorghum, or a wild relative of sorghum, introduced into the genetic construct;

(iii) the terminator of the sorghum UBIQUITIN1 gene

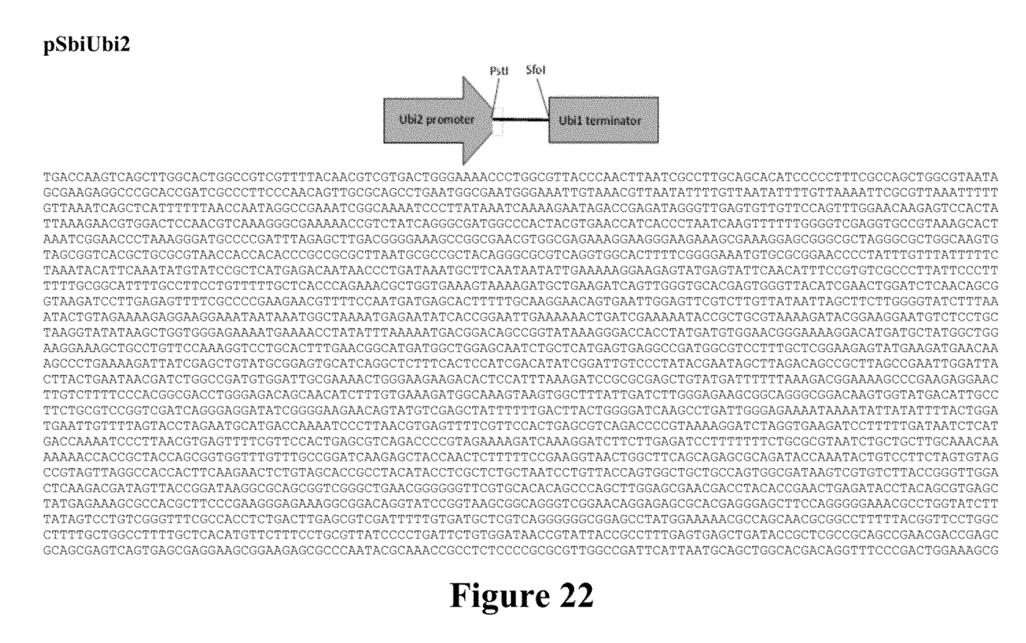

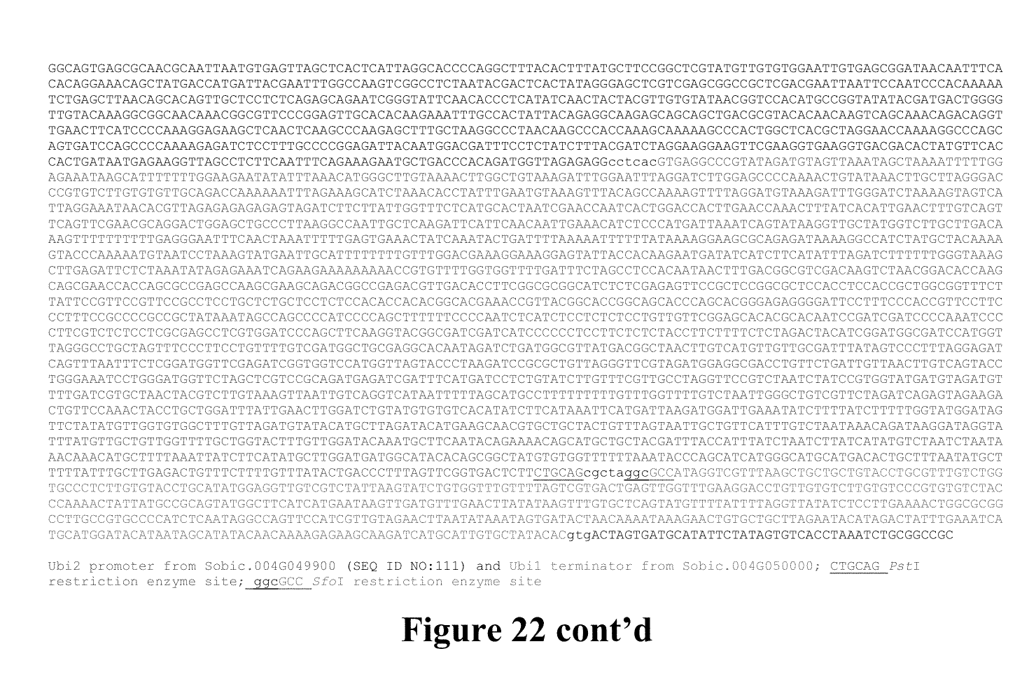

A schematic diagram of another preferred genetic construct (pSbiUbi2) is set forth in FIG. 22. The complete nucleotide sequence of this genetic construct is set forth in SEQ ID NO:74.

The backbone sequence of this vector is the backbone sequence of the vector pKannibal. It contains the promoter and terminator sequence of the Sorghum biocolor UBIQUITIN2 gene (Sobic.004G050000). It was developed by making use of the natural PstI site at the 3′ end of the Ubi2 promoter which was amplified from sorghum gDNA with primers F 5Phos/cctcacGTGAGGCCCGTATAGATGTA GTTAAATAGCTAAA (SEQ ID NO:130) (adding 3 nucleotides to the start of the promoter to create a blunt-cutter site PmlI to enable excision of the intragenic cassette) and R tccCTGCAGAAGAGTCACCGAACTAAAGG (SEQ ID NO:131). The fragments were digested with PstI and ligated into vector pKannibal digested with StuI and PstI. Terminator Ubi1 was amplified and cloned as described above for pSbiUbi1.

This vector (pSbiUbi2) is suitable to express a sequence of interest in sorghum, by amplifying the insert with primers F CTGCAG[start of insert sequence] and R 5′Phos[reverse complement of end of insert sequence]. The fragment is then digested with PstI and ligated into pSbiUbi1 opened up with PstI and SfoI restriction enzymes.

It will be appreciated that after excision, the sequence for direct gene transfer consists of plant-derived nucleotide sequences.

It will be appreciated that after digestion and ligation using the PstI and SfoI restriction sites, and insertion of the one or more nucleotide sequences derived from sorghum or a wild relative of sorghum, spacer SEQ ID NO:75 is removed from the genetic construct.

Suitably, after introduction of said one or more nucleotide sequences derived from sorghum or a wild relative of sorghum into the genetic construct, a fragment of the genetic construct of this Example consists of a plurality of nucleotide sequences of at least 15, or preferably at least 20, nucleotides in length derived from one or more plants, wherein said fragment consists of:

(i) the promoter of the sorghum UBIQUITIN2 gene,

(ii) the one or more nucleotide sequences derived from sorghum, or a wild relative of sorghum, introduced into the genetic construct;

(iii) the terminator of the sorghum UBIQUITIN1 gene

Constructs and Vectors with Sequences for Expression: Rice

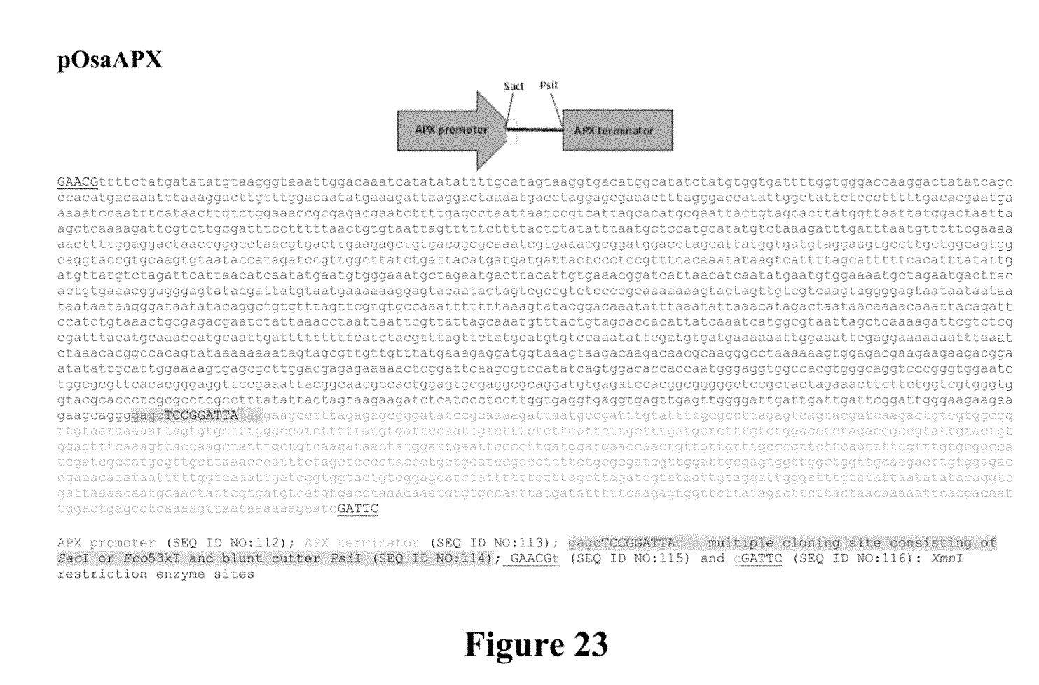

A preferred method for rice transformation is by direct gene transfer using biolistics. To ensure that only rice genome-derived sequences are used, a vector is used where the linear DNA fragment for direct gene transfer can be easily excised prior to biolistics. A schematic diagram of such a preferred genetic construct (pOsaAPX) is set forth in FIG. 23. The complete nucleotide sequence of this genetic construct is set forth in SEQ ID NO:76.

The backbone sequence of this vector is the backbone sequence of the vector pUC57-KAN. It contains the promoter and terminator sequence of the Oryza sativa APX gene. It was developed by ligating the synthesised sequence of SEQ ID NO:76 into the cut Eco53kI site of pUC57-KAN. The APX gene promoter was chosen for its constitutive throughout the plant and its strong expression in leaves.

This vector (pOsaAPX) is suitable to express a sequence of interest in rice, by amplifying the insert with primers F GAGCTC[start of insert sequence] and R 5′Phos[reverse complement of end of insert sequence]. The fragment is then digested with SacI (or Eco53kI) and ligated into pOsaAPX1 opened up with SacI (or Eco53kI) and PsiII restriction enzymes.

It will be appreciated that after excision, the sequence for direct gene transfer that consists of plant-derived nucleotide sequences.

It will be appreciated that after digestion and ligation using the SacI (or Eco53kI) and PsiI restriction sites, and insertion of the one or more nucleotide sequences derived from rice or a wild relative of rice, spacer SEQ ID NO:77 is removed from the genetic construct.

Suitably, after introduction of said one or more nucleotide sequences derived from rice or a wild relative of rice into the genetic construct, a fragment of the genetic construct of this Example consists of a plurality of nucleotide sequences of at least 15, or preferably at least 20, nucleotides in length derived from one or more plants, wherein said fragment consists of:

(i) the promoter of the rice APX gene,

(ii) the one or more nucleotide sequences derived from rice, or a wild relative of rice, introduced into the genetic construct;

(iii) the terminator of the rice APX gene

### Example 2. Assessment of Regulatory Sequences for Use in Genetic Constructs of the Invention

The use of intragenic regulatory sequences, such as promoters and terminators is important to achieve the desired expression in plants. For example, this can achieve strong constitutive expression throughout the plant, expression in various plant organs or cell types, expression during certain developmental stages, and/or expression upon induction with a signalling compound (e.g. a plant hormone).

Apart from the specificity and expression pattern throughout the plant, in preferred embodiments of constructs of the present invention, intragenic regulatory sequence(s) such as promoters and terminators are chosen that come from the same or a related species as a sequence for expression using the construct.

Furthermore, in preferred embodiments wherein the construct comprises border sequences and is optimized for Agrobacterium-mediated transformation, regulatory sequence(s) containing parts of an LB or RB sequence are used. Additionally, in preferred embodiments wherein the constructs are optimized for transformation that does is not Agrobacterium-mediated transformation (e.g. direct gene transfer methods), regulatory sequence(s) containing at least partial restriction sites are used, to facilitate excision of the plant-derived fragment to be transferred to the genetic material of a plant, in the absence of any surrounding non-plant-derived sequences.

For the present invention, several tomato regulatory sequences were isolated and tested with reporter genes, such as the green fluorescent protein (GFP) encoding gene, to investigate their potential as regulatory nucleotide sequences for genetic constructs of the invention.

The nucleotide sequence set forth in SEQ ID NO:4 of the promoter of the tomato RUBISCO subunit 3C (RbcS3C) gene was tested together with the nucleotide sequence set forth in SEQ ID NO:8 of the terminator belonging to the same gene, by transient expression of GFP in tomato mesophyll protoplasts, and stable Agrobacterium-mediated transformation of tomato plants.

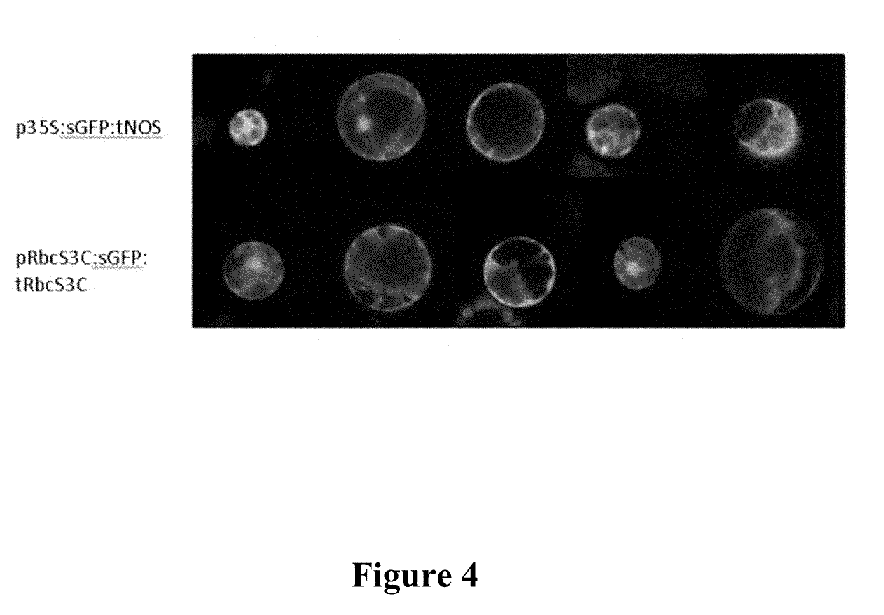

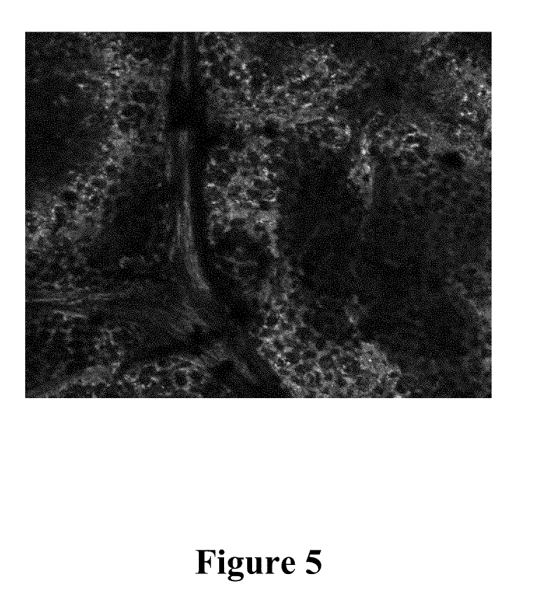

Strong GFP expression, comparable to that driven by the widely-used Cauliflower mosaic virus (CaMV) 35S promoter, was obtained in protoplasts, confirming the functionality of the RbcS3C terminator (FIG. 4). One of the purposes of the stable transformation experiment was to establish the pattern of RbcS3C-driven expression. While it was hypothesised that expression of the reported gene regulated by RbcS3C regulatory elements would be limited to the green parts of the plant, GFP fluorescence was observed in the roots, as well as in some cell types in leaves (FIG. 5). This may be explained by the fact that only 763 nucleotides of the RbcS3C promoter were used.

To identify other candidate regulatory elements for use in genetic constructs of the invention, information on expression levels of common tomato housekeeping genes was derived from Mascia, T. et al., 2010, Molecular Plant Pathology, 11 805, incorporated herein by reference.

Among those with the highest and most stable expression in both shoots and roots, ACTIN (gi 460378622) UBIQUITIN (gi 19396) and CYCLOPHILIN (gi 225312116) genes stood out particularly. Transient expression of GFP driven by these regulatory genes in agroinfiltrated N. benthamiana leaves was then performed to assess their ability to regulate expression.

Sequences of approximately 1000 nucleotides upstream of the start codon and a few to several hundred nucleotides downstream of the stop codon of the genes were amplified from tomato genomic DNA (cultivar Moneymaker) by polymerase chain reaction (PCR) using specific primers and used as promoters and terminators in GFP constructs. The GFP expression cassettes were then inserted into the binary vector pArt27 and introduced into A. tumifaciens strain GV3101 by triparental mating including E. coli strain harbouring pHelper plasmid. Overnight A. tumifaciens cultures harbouring the binary vectors were centrifuged at 4000×g for 15 min and pellets were resuspended in 10 mM magnesium chloride supplemented with 200 mM acetosyringone to OD600 of 1.0. The suspensions were incubated at room temperature for 4 hours and infiltrated into young leaves of 4-6 week-old Nicotiana benthamiana using needleless syringes.

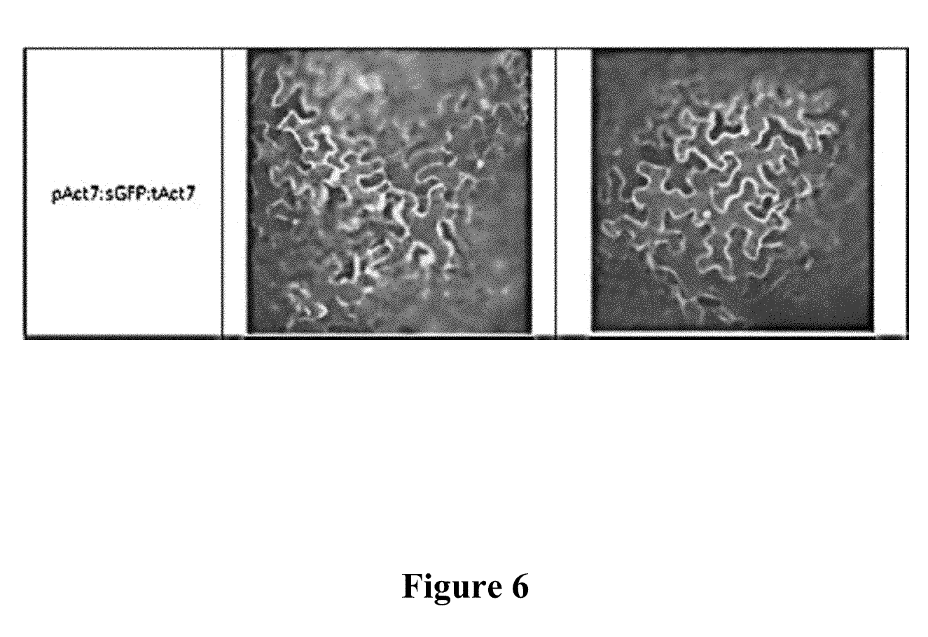

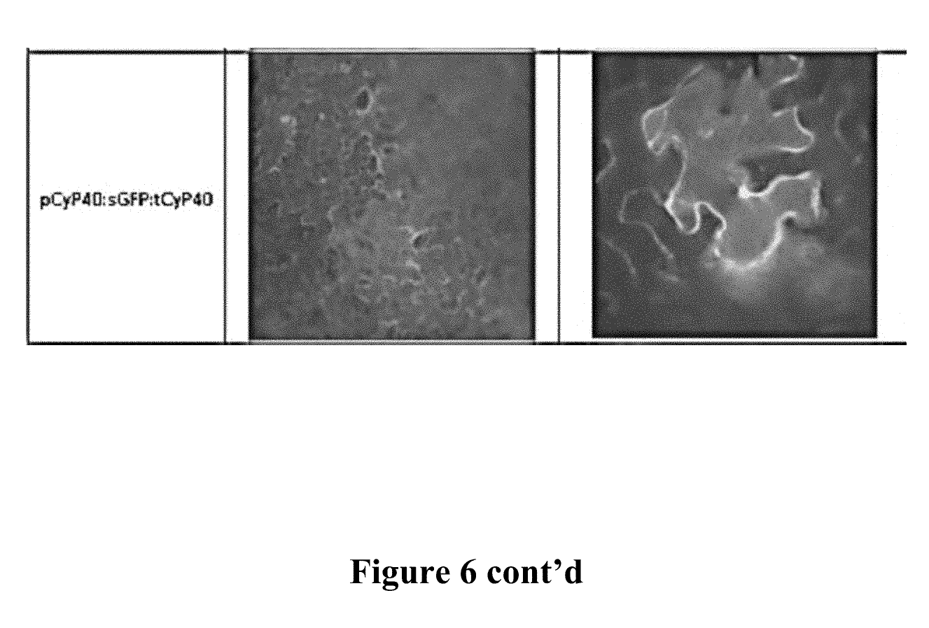

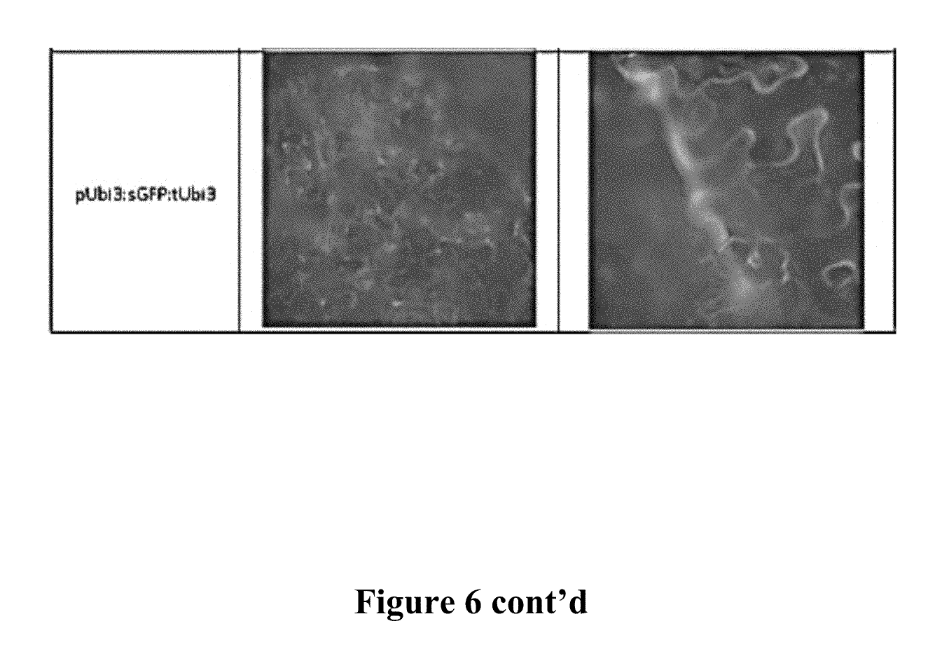

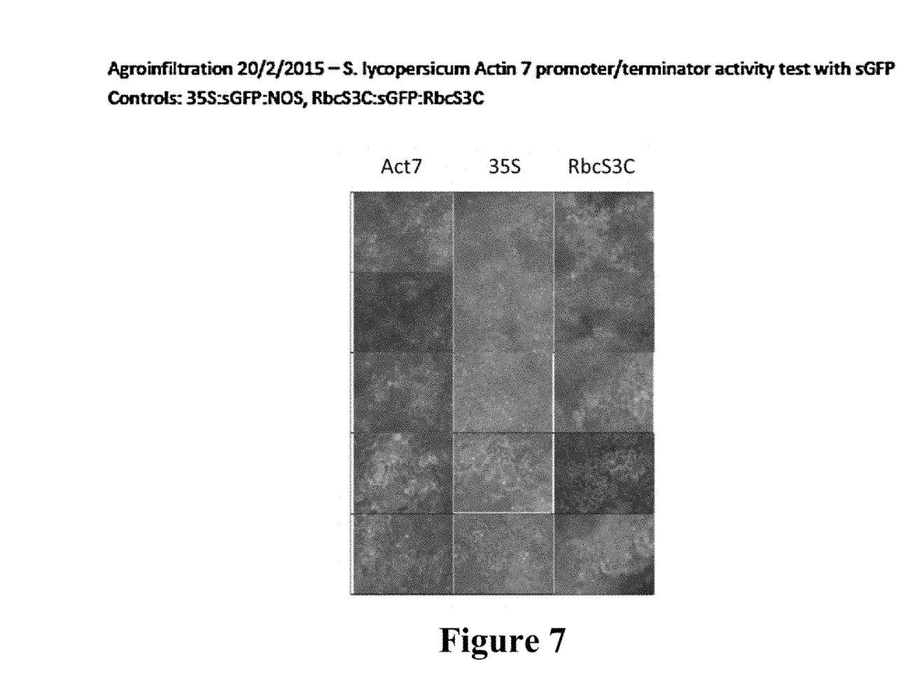

GFP expression was observed using a fluorescence microscope following 3 days post-infiltration. All three promoter-terminator pairs were able to drive the expression of GFP in transient leaf agroinfiltration assays in N. benthamiana. The best level of GFP expression was observed for the ACTIN promoter, both in terms of brightness of expression and extensive size of leaf areas containing expressing cells (FIG. 6). In another agroinfiltration test, the activity of tomato ACTIN promoter-terminator combination was compared with that of tomato RbcS3C and CaMV 35S, where the ACTIN gene regulatory elements performed as well, or possibly better, than the traditionally used promoters in terms of brightness and uniformity (FIG. 7).

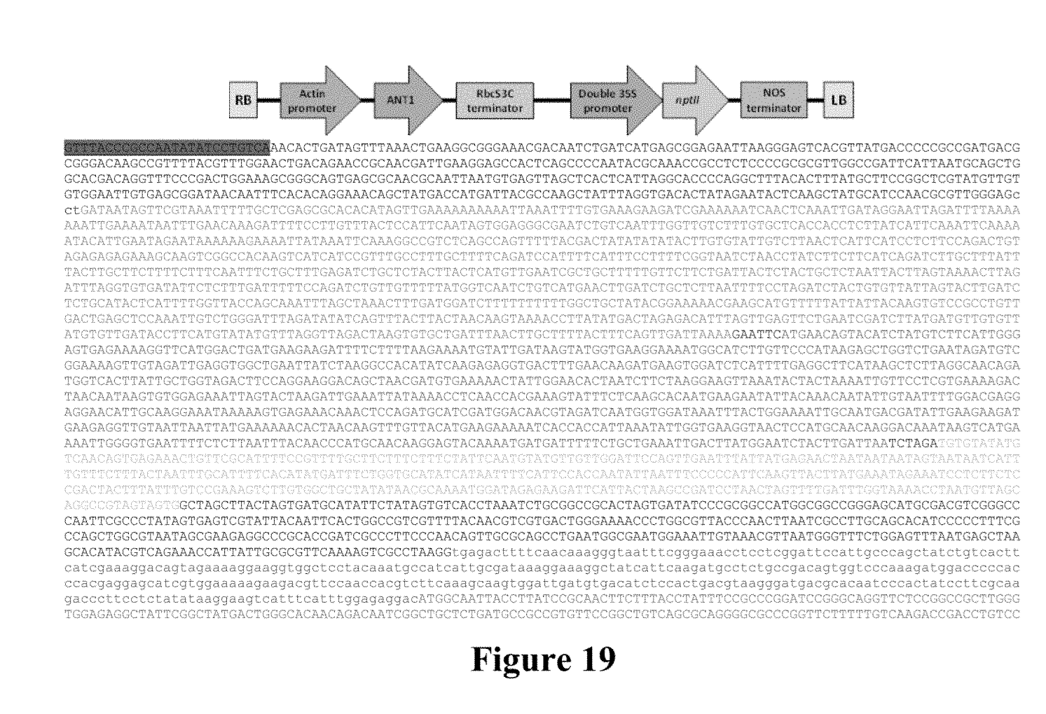

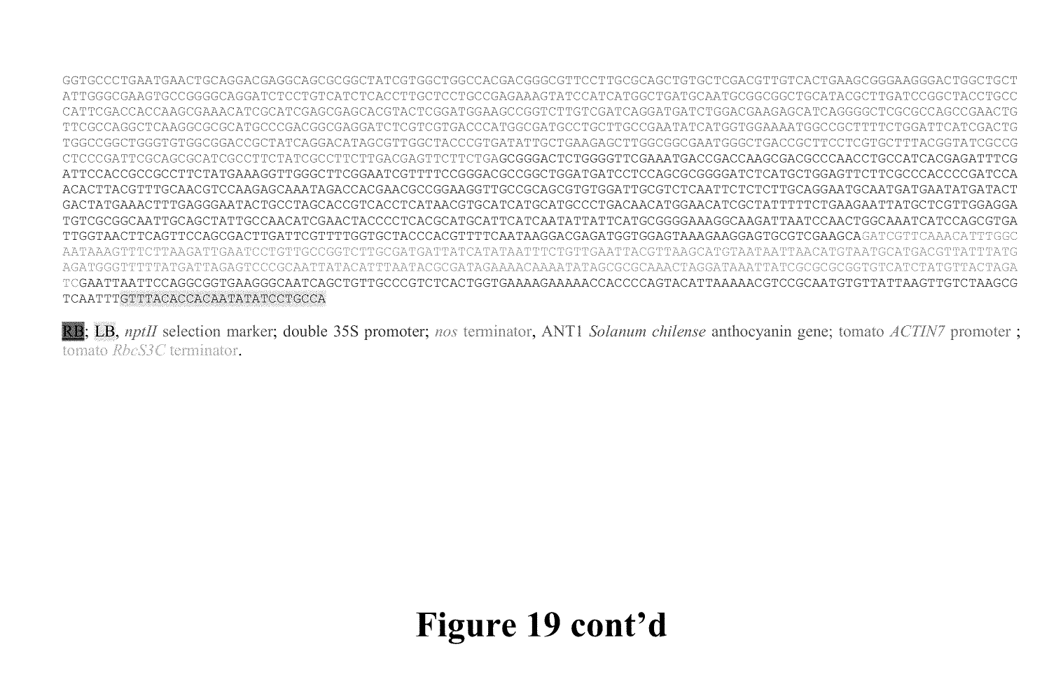

To test whether the tomato ACTIN promoter and RbcS3C terminator also perform well in stably transformed plants, a promoter-reporter-terminator cassette was constructed that was inserted into pArt27. This cassette contained the ACTIN7 promoter, the ANT1 gene and the RbcS3C terminator (pArt27 ACT:ANT1:RbcS3C 35S:nptII:NOS). The construction of this cassette and its vector has been described in Example 1 and is set forth in FIG. 19. The sequence of this reporter gene construct is set forth in SEQ ID NO:69.

Next, tomato plants were produced by Agrobacterium-mediated transformation (following the method by Subramaniam et al., 2016, Plant Physiology, 170 1117) with pArt27 ACT:ANT1:RbcS3C 35S:nptII:NOS. Their transformed status was confirmed by quantitative real-time PCR (qPCR) and their ANT1 expression was confirmed by quantitative real-time reverse transcriptase PCR (qRT-PCR).

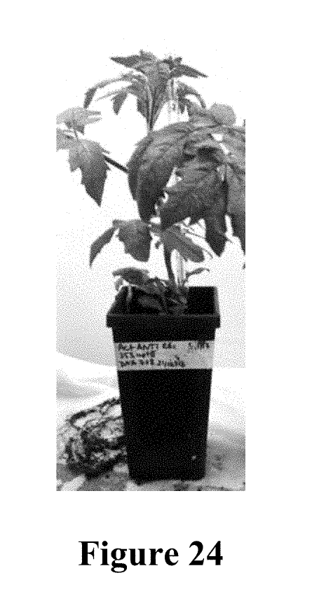

As set forth in FIG. 24, these plants expressing SEQ ID NO:69 displayed increased anthocyanin levels (purple stem, roots, veins and part of the leaves) as compared to corresponding wild type tomato plants. This demonstrates functionality of the tomato ACTIN7 promoter and the RbcS3C terminator for near-constitutive gene expression and the intragenic cassette included in FIG. 24 and SEQ ID NO:69.

Similarly, the functionalities of other intragenic plant promoters and terminators were also established. This includes the rice ACTIN1 promoter in combination with the rice DREB1A terminator (see Example 7), and the abscisic acid (ABA) inducible promoter and terminator of the ABA biosynthesis gene NCED3, the R1G1B promoter and terminator, and the APX promoter and terminator (FIG. 23; SEQ ID NO:76). All promoters and terminators were tested in combination with the rice DREB1A gene in intragenic constructs (see Example 7) that also serves as a selectable marker (see Example 3).

The rice ACTIN1 promoter is well established as a functional constitutive promoter in rice (McElroy et al., 1991, Molecular and General Genetics, 231 150). The rice NCED3 promoter and terminator were chosen as examples for inducible regulatory sequences, as the corresponding NCED3 gene is ABA inducible. The rice R1G1B promoter and terminator were chosen as they are expected to express highly throughout the plant, in particular in the endosperm (Park et al., 2010, Journal of Experimental Botany, 61 2459) and were therefore used to express traits that express in the rice grain (e.g. fragrant rice; see Example 9, and anthocyanin production). The rice APX promoter and terminator were chosen based on the expected strong and constitutive expression in rice. Construction of these intragenic DNA fragments and their sequences are set forth in Examples 2, 3, and 9, for APX, ACTIN1/DREB1A/NCED3, and R1G1B, respectively.

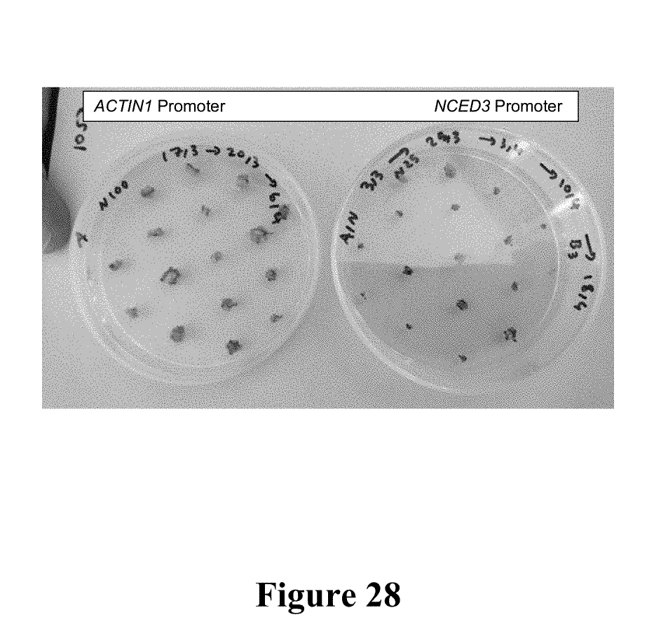

Rice calli (Oryza sativa cultivar Reiziq) were produced and used for direct gene transfer of excised linear DNA (for details on rice somatic embryogenesis and transformation see Example 7). Functionality of the rice ACTIN1 constitutive promoter in combination with the DREB1A terminator was confirmed as 9% of the transformed rice calli survived on high salinity (100 mM NaCl) medium during regeneration (see Example 3 and FIG. 28).

Functionality and inducibility of the rice NCED3 ABA-inducible promoter and terminator were confirmed as 19% of the transformed rice calli survived on high salinity (100 mM NaCl) medium during regeneration (see Example 3 and FIG. 28).

Functionality and inducibility of the rice R1G1B promoter and terminator were confirmed as 21% of the transformed rice calli survived on high salinity (100 mM NaCl) medium during regeneration.

Furthermore, the functionalities of sorghum intragenic plant promoters and terminators were established. This includes the previously untested sorghum UBIQUITIN1 (Ubi1) promoter and terminator from Sobic.004G050000, as well as the previously used UBIQUITIN2 promoter (REF), that was also tested with the UBIQUITIN1 terminator. Construction of these two cloning cassettes has been described in Example 2 and is set forth in FIGS. 21 and 22, and SEQ ID NO:74 and SEQ ID NO:77, respectively.

### Example 3. Use of Native Genes as Selectable Markers for Transformation

The use of selectable markers during plant transformation facilitates efficient selection of transformed plants. For this purpose, it is advantageous that genetic constructs of the invention comprise one or more additional nucleotide sequences that are selectable marker nucleotide sequences, derived from one or plants.

For the present invention, several native tomato genes were assessed for potential to act as selectable markers nucleotide sequence in the genetic construct of the invention.

A gene with homology to betaine aldehyde dehydrogenase in tomato was identified (gi 209362342), comprising nucleotide sequence set forth in SEQ ID NO:27, and tested by stable Agrobacterium-mediated transformation with transgenic cassettes comprising this gene under the control of 35S or tomato RbcS3C promoters. Among the shoots regenerated on selective media containing 5 mM BA, 18% contained the integrated p35S:BADH cassette. No pRbcS3C:BADH regenerants were obtained. The p35S:BADH transformants developed normally in vitro and were planted in soil, where they grew healthily and produced morphologically normal flowers.

Additionally, a gene homologous to alfalfa and soybean cytoplasmic Glutamine Synthetase 1 (GS1) was identified in tomato (gi 460409536), comprising nucleotide sequence set forth in SEQ ID NO:30 which has over 90% similarity to both in amino acid sequence and over 80% identity in coding sequence. Mutations of this tomato GS1 that have been described to confer tolerance to herbicides in alfalfa (Tischer, E., et al., supra; U.S. Pat. No. 4,975,374 A) and soybean (Pornprom, T., et al., supra) were introduced by site-directed mutagenesis.

Specifically, the two mutants produced were G245C (encoded by the nucleotide sequence set forth in SEQ ID NO:51) and H249Y (encoded by the nucleotide sequence set forth in SEQ ID NO:52). The tomato GS1 variants were cloned in first transgenic and later intragenic binary vectors under the control of tomato RbcS3C promoter and terminator as hereinbefore described. By way of example, the full nucleotide sequence of the intragenic binary vector encoding the G245C variant is set forth in SEQ ID NO:48.

Tomato cotyledon explants treated with Agrobacterium harbouring the vectors were cultivated on shoot-regenerating media containing 1 mg/L Glufosinate Ammonium (GA). 86% of the multiple shoots regenerated from transgenic transformation with GS1 G245C were PCR-positive for the marker. Regeneration of shoots from transformation with GS1 H249Y was considerably less efficient.

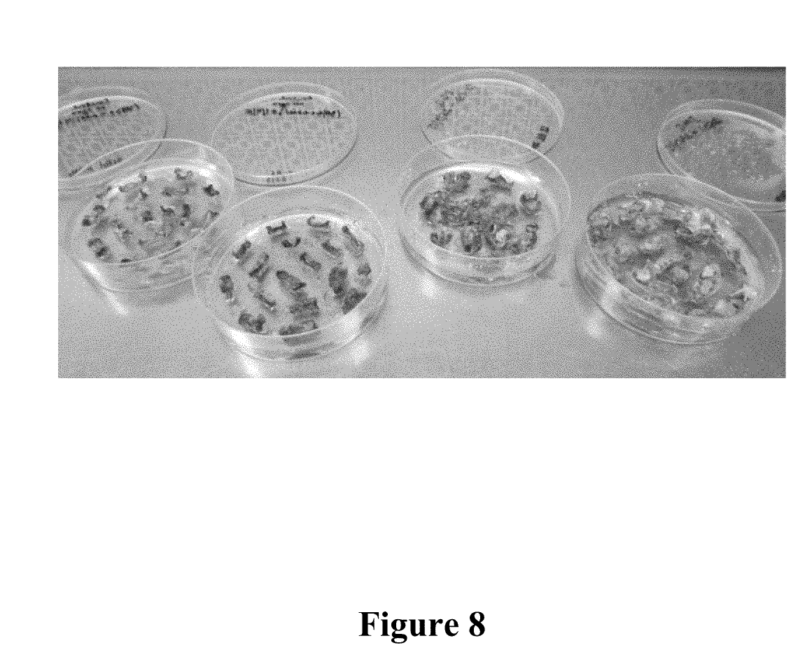

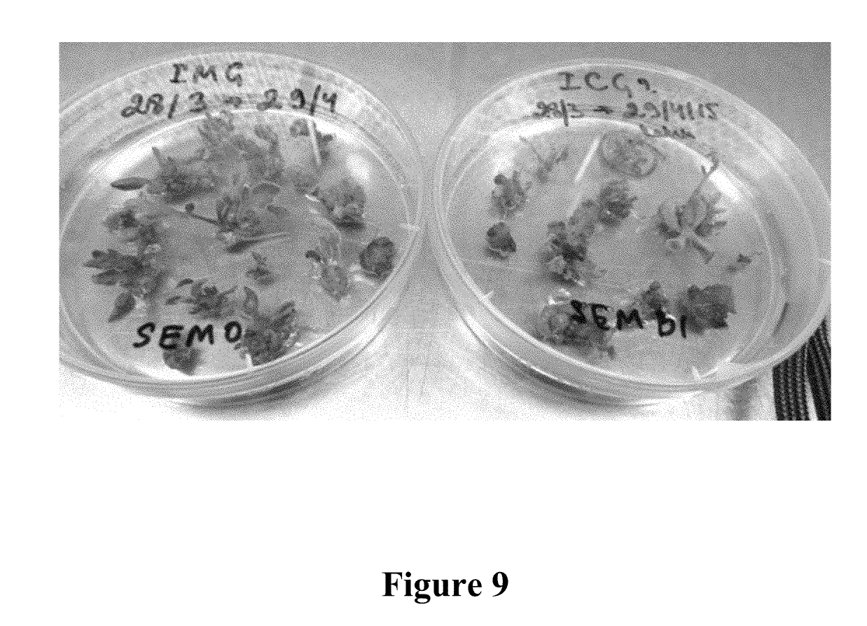

Test transformations with intragenic vectors containing two expression cassettes, of which the pRbcS3C: GS1 G245C was situated with the start of the promoter immediately adjacent to the left border, produced vigorous shoot growth in contrast with none obtained from non Agrobacterium co-cultivated control explants on the same regeneration medium (FIGS. 8 and 9). However, only a small proportion of the shoots were PCR-positive for the integration of pRbcS3C: GS1 G245C cassette, with considerably more cases of integration of the second expression cassette only.

In both cases of transgenic and intragenic test transformations so far there have been some difficulties with regeneration of initially quickly formed, vigorous shoots containing the integrated pRbcS3C: GS1 G245C to the stage ready for planting out in soil, due mainly to uneven growth patterns. As a potential solution, different amino acid substitutes at the clearly important position 245 may be tested, e.g. G245S or G245R by analogy with those naturally occurring in GA-tolerant alfalfa, along with the employment of alternative native promoters, e.g. from the number of those already tested.

For the purpose of this invention, the usefulness of anthocyanin as a visual selectable marker was also tested. As set forth in FIG. 24, tomato plants expressing SEQ ID NO:69 displayed increased anthocyanin levels (purple stem, roots, veins and part of the leaves) as compared to corresponding wild type tomato plants. This demonstrates functionality of the ANT1 gene as a suitable visual marker gene, in principle, and the intragenic cassette included in FIG. 24 and SEQ ID NO:69.

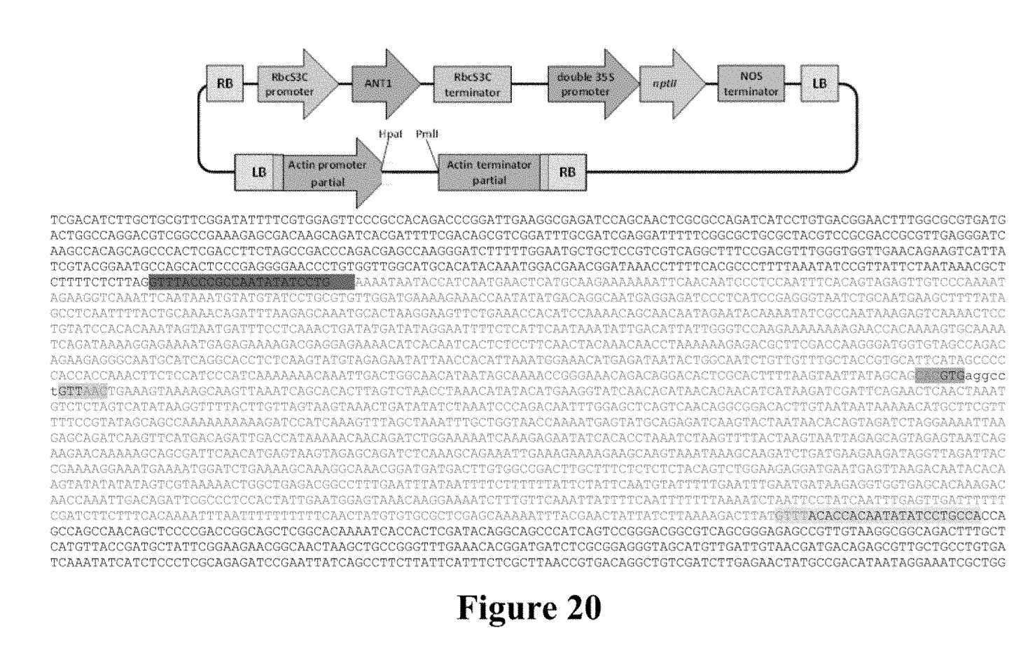

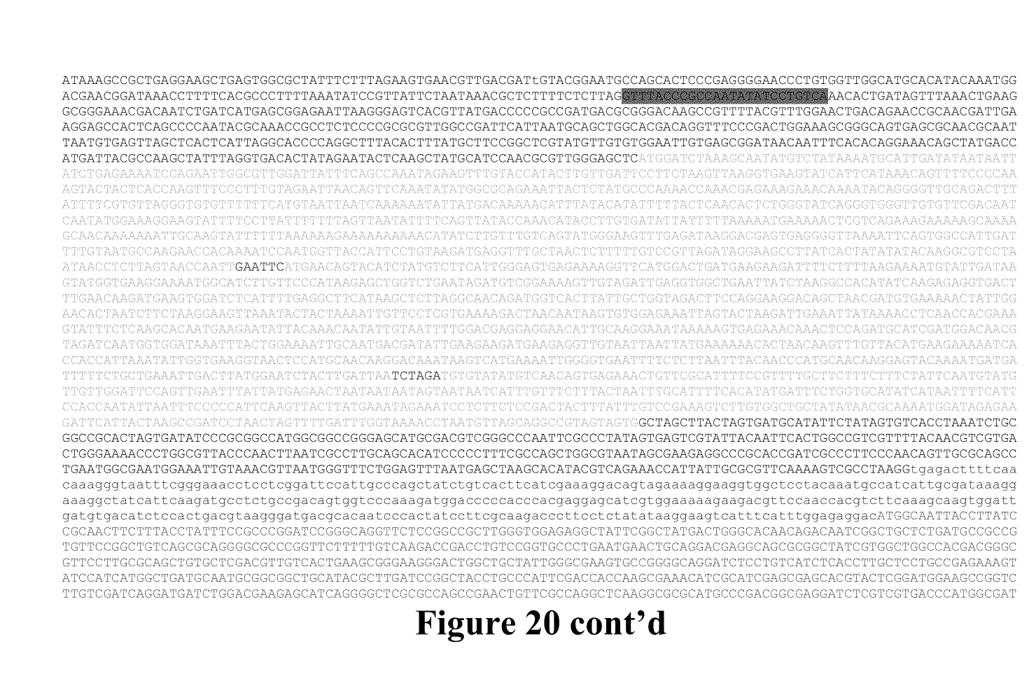

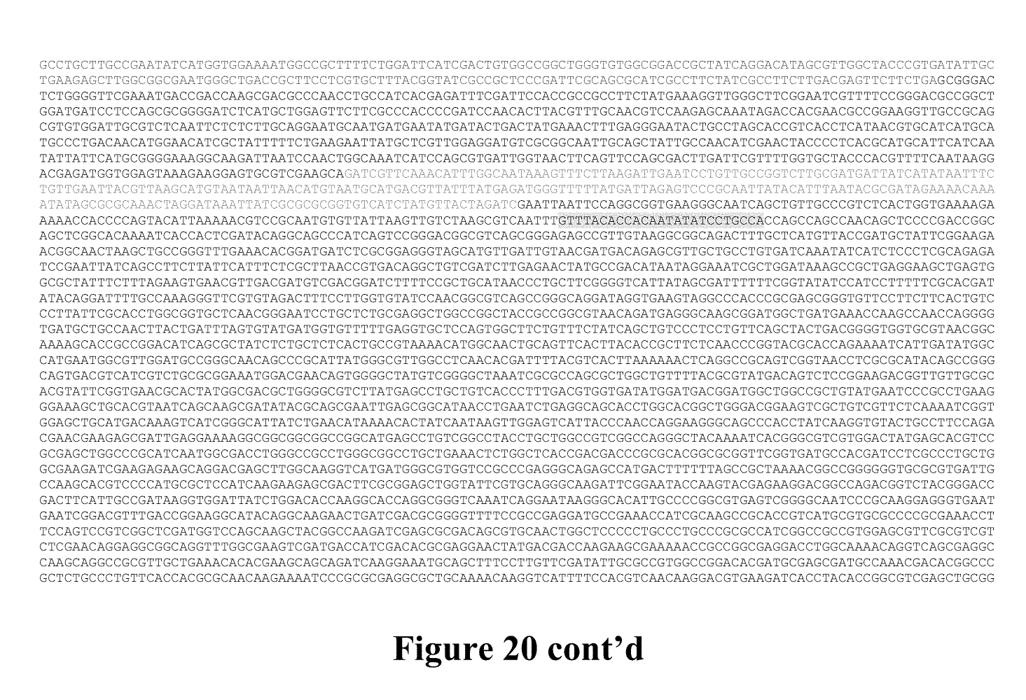

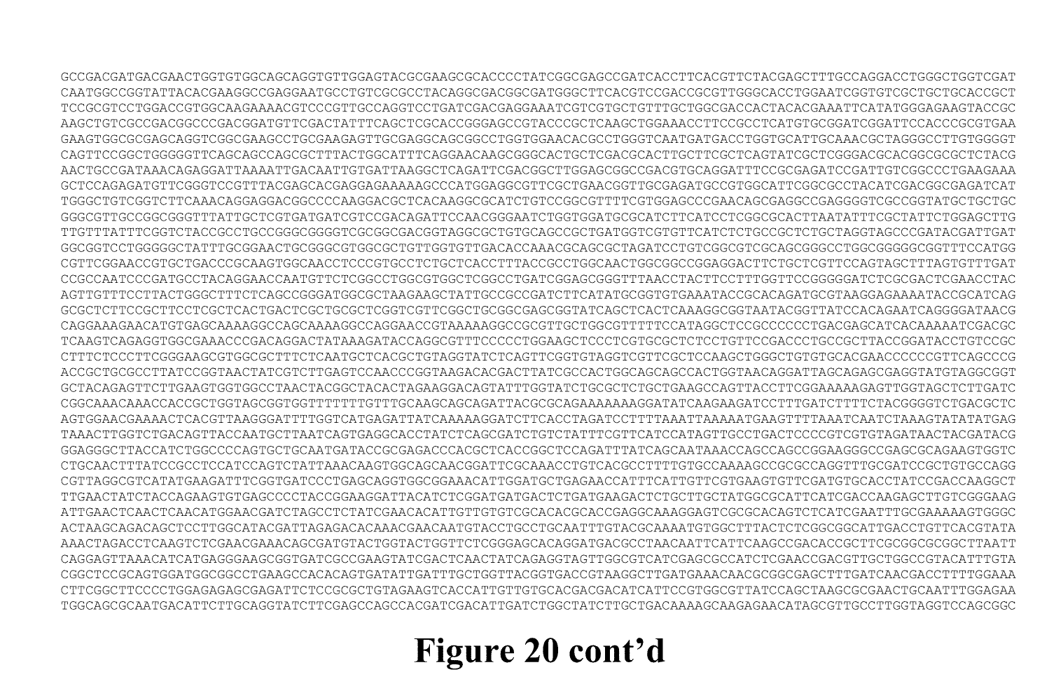

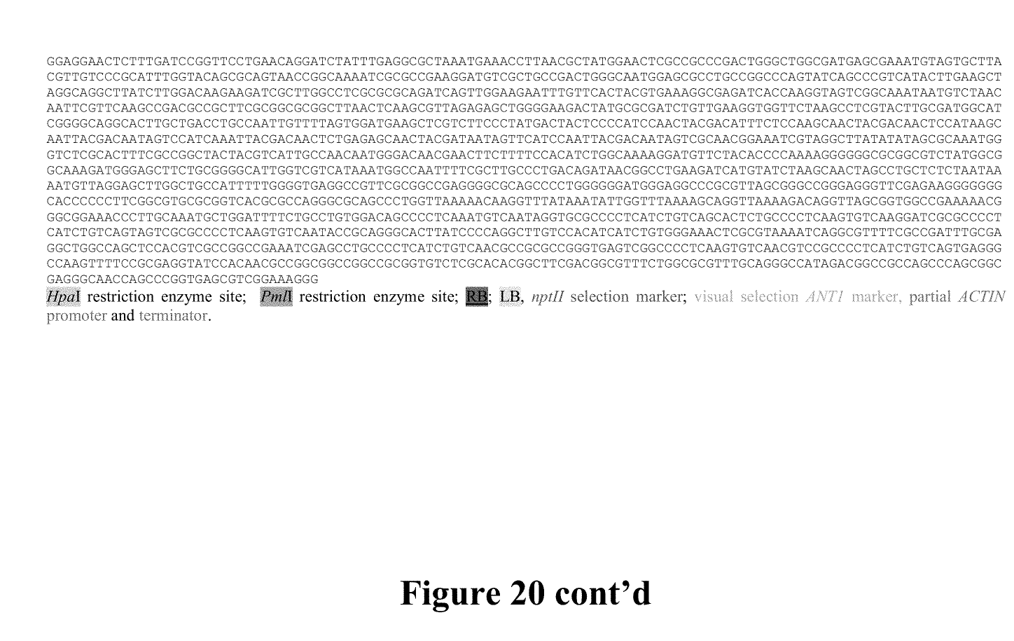

However, the use of anthocyanin as the sole selectable marker can be laborious and may require many transformation events, as there is only visual but not physiologically active selection against non-transformed cells. Hence there is the conventional option to separately transform with a transgenic selectable marker gene, such as the NPTII gene that confers gentamycin or kanamycin resistance. This separately transformed gene cassette would undergo independent integration into the plant's genome at a different locus that can be later crossed out (e.g. by back crosses). The use of anthocyanin as a visual marker can greatly assist here to rapidly screen for those plants where the selectable marker has putatively been removed. To evaluate this approach, constructs for two options were prepared as set forth in Example 1. In option 1, a selectable marker cassette with ANT1 is provided as a separate vector making use of co-transformation (FIG. 19; SEQ ID NO:69), and in option 2, a selectable marker cassette with ANT1 is included on the same plasmid but that is integrated independently by providing its own LB and RB sequences (FIG. 20; SEQ ID NO:70).

Next, tomato plants were produced by Agrobacterium-mediated transformation (following the method by Subramaniam et al., 2016, Plant Physiology, 170 1117) with pArt27 ACT:ANT1:RbcS3C 35S:nptII:NOS (FIG. 19) co-transformed with a construct conferring the desirable trait of heart-shaped tomatoes (for details see Example 9; Figure X). Their transformed status was confirmed by quantitative real-time PCR (qPCR) and their ANT1 expression was confirmed by quantitative real-time reverse transcriptase PCR (qRT-PCR).

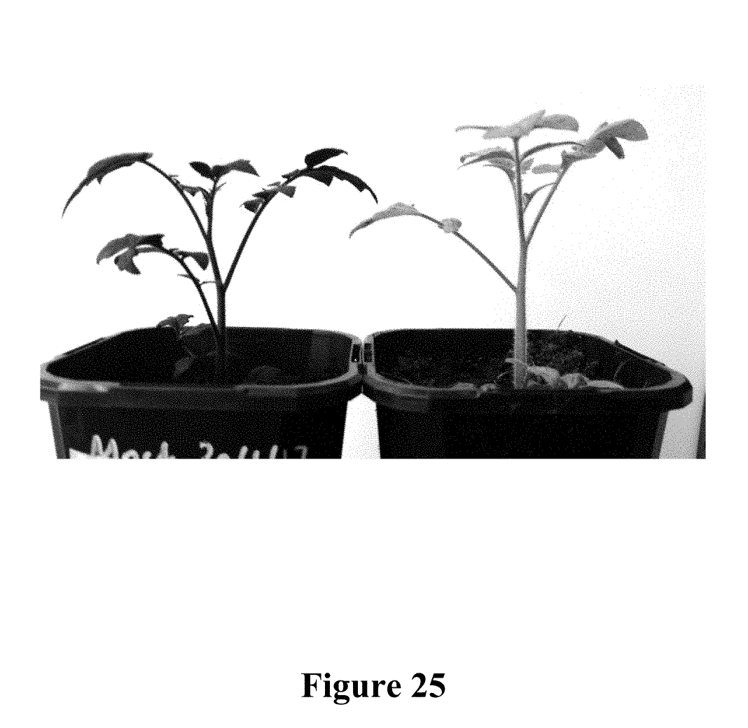

As set forth in FIG. 25, tomato plants co-transformed with pArt27 ACT:ANT1:RbcS3C 35S:nptII:NOS, showed strong anthocyanin production (left), while comparable plants without this construct (right) showed no visual signs of heightened anthocyanin production. This demonstrates that anthocyanin-producing genes are useful when co-transformed with the selectable marker, as a visual tool for the selectable marker cassette to be outcrossed in F1 generations. Genetic constructs for anthocyanin production in rice and sorghum were also produced (see Example 8) that may serve as a visual selectable marker.

To develop another endogenous (intragenic) selectable marker for intragenic plant transformation, the rice DREB1A gene was tested. To enable this method, first a kill curve was established on rice callus. Rice calli were produced for Oryza sativa cultivar Reiziq and IR64 and plants were regenerated as described in Example 7. MS basal medium supplemented with Gamborge B5 vitamins, 1 mg·L⁻¹ NAA, 2 mg·L⁻¹ BAP, 2 mg·L⁻¹ kinetin, 3% sucrose and 7% Agar 7% was determined to be most suitable as a rice regeneration medium. Six concentrations of sodium chloride (100, 150, 200, 250, 300 and 350 mM) were added to the medium. Results 4 weeks later showed that 100 mM NaCl provided the most suitable condition for selection that sufficiently suppressed regeneration for both cultivars (Reiziq and IR64). Hence, 100 mM NaCl was considered as effective selection to produce transformed rice plants.

Next, the rice DREB1A gene was tested either in combination with the rice ACTIN1 promoter and DREB1A terminator or the rice NCED3 promoter and terminator as a suitable selectable marker for rice transformation by providing salinity tolerance.

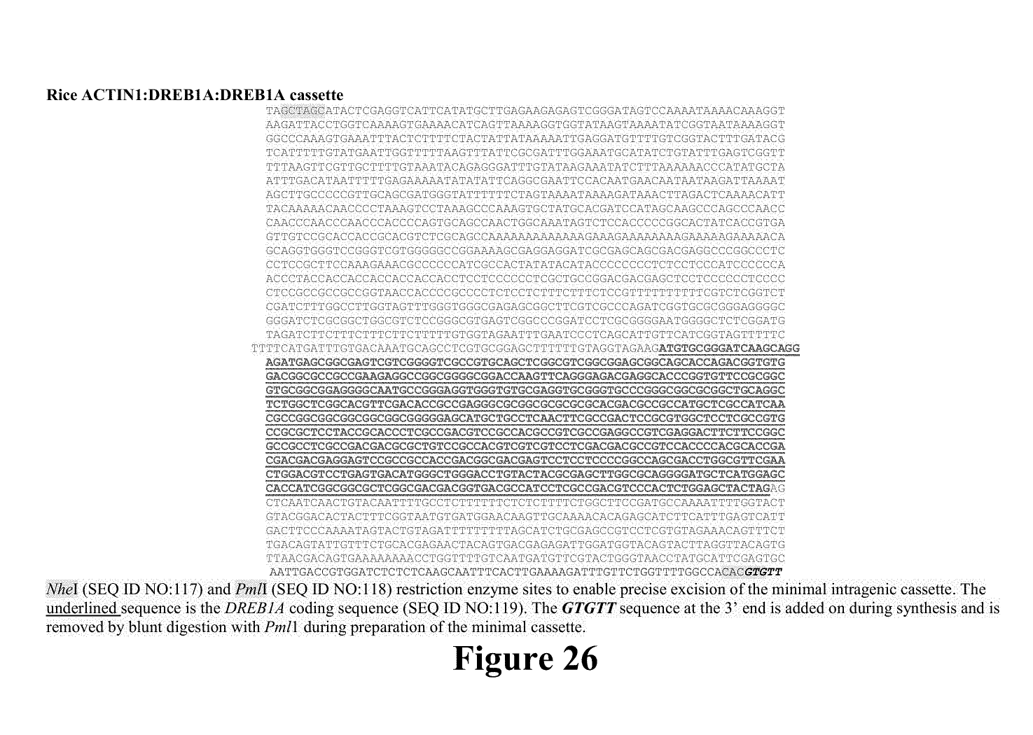

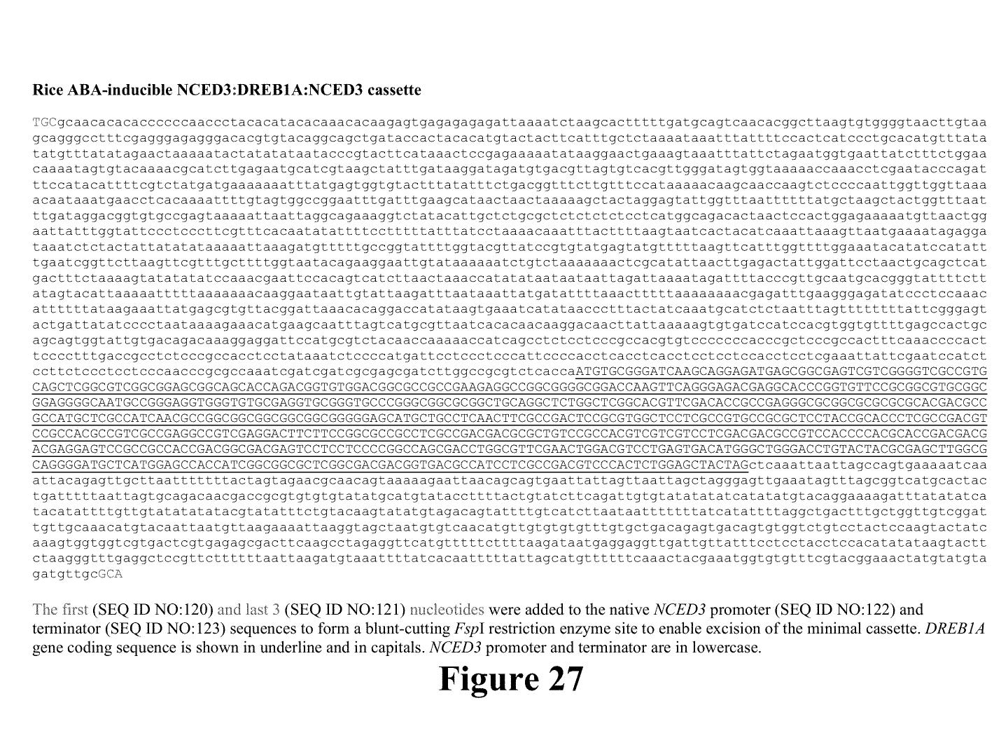

These fully intragenic constructs were produced by first synthesising expression cassettes and then inserting them into the EcoRV restriction enzyme site of the subcloning vector pUC57-KAN by the manufacturer (GenScript), although any other E. coli plasmid with a blunt end cloning site would be suitable. The ACTIN1:DREB1A:DREB1A cassette is set forth in FIG. 26 and SEQ ID NO:78. The NCED3:DREB1A:NCED3 cassette is set forth in FIG. 27 and SEQ ID NO:79. Prior to transformation of rice calli via particle bombardment, the cassettes were excised using the unique restriction enzyme sites NheI PmlI for ACTIN1:DREB1A:DREB1A, and FspI for NCED3:DREB1A:NCED3.

As set forth in FIG. 28, 9% out of 180 calli transformed with the ACTIN1:DREB1A:DREB1A cassette survived on 100 mM NaCl-containing medium after 15 days, and 19% out of 300 calli transformed with the NCED3:DREB1A:NCED3 cassette survived on 100 mM NaCl-containing medium after 15 days and most of these survived also after 1 month. By comparison, none of the untransformed control calli survived in 100 mM-containing medium.

These percentages are acceptable transformation efficiencies and therefore the DREB1A gene and the corresponding intragenic cassettes were considered suitable as fully intragenic selectable marker for use in constructs of the invention for the generation of transformed intragenic plants.

### Example 4. Co-Transformation Strategies

Co-Transformation with Independent Vector

In at least certain circumstances, it can be preferred to use intragenic constructs like those mentioned herein, in a ‘two-vector two Agrobacterium strain’ co-transformation strategy. Here, the constructs can be used in conjunction with a separate T-DNA construct that contains a selectable marker gene which would integrate at a different locus and can be crossed out in F1 or F2 generations, leaving a plant that contains no foreign sequence in its genome.

A schematic diagram of such a separate T-DNA construct and vector comprising said genetic construct suitable as a selectable marker, is set forth in FIG. 19. The sequence of this selectable marker construct is set forth in SEQ ID NO:69, and has been previously described above. It will be understood that, due to the presence of non-plant-derived regulatory and selectable marker sequences that are designed to be incorporated into the genetic material of a plant, this construct is not itself a preferred construct of the invention, although it does share certain components with such preferred constructs.

The backbone sequence of the vector set forth in FIG. 19 is the backbone sequence of the binary vector pArt27. Apart from a selectable marker gene (nptII), a visual marker gene (ANT1) for anthocyanin biosynthesis has been included to enable easy outcrossing, as hereinabove described. The genetic construct comprises sequence of an Agrobacterium RB sequence; sequence of an Agrobacterium LB sequence. Located between the RB and LB sequences are:

(i) the nucleotide sequence set forth in SEQ ID NO:5 that is of the promoter sequence of a tomato ACTIN7 gene, located adjacent to the RB sequence and operably connected with (ii);

(ii) the nucleotide sequence set forth in SEQ ID NO:35 that is of a Solanum chilense ANT1 anthocyanin gene;

(iii) the nucleotide sequence set forth in SEQ ID NO:8 that is of the terminator of a tomato RbcS3C gene, operably connected with (ii);

(iv) nucleotide sequence of the double 35S promoter sequence of Cauliflower mosaic virus, operably connected with (v);

(v) nucleotide sequence that is of a neomycin phosphotransferase II (nptII) gene;

(vi) nucleotide sequence that is of the terminator of an Agrobacterium nos gene, operably connected with (v), located adjacent to the LB sequence.

The sequence of (i) has been designed such that substantial truncation of the ACTIN promoter sequence will ablate or substantially compromise the promoter function of (i), such that the ability of (i) to drive the expression of the selectable marker sequence (ii) that is of the Solanum chilense ANT1 anthocyanin gene will be eliminated or substantially reduced.

Alternatively, another version of this vector was produced where the ACTIN promoter was replaced with the RbcS3C promoter (pArt27 RbcS3C:ANT1:RbcS3C 35S:nptII:NOS).

Co-Transformation with Independent Constructs on Single Vector

While co-transformation with two-vectors is achievable, in some cases co-transformation efficiency can be quite low. In order to avoid this issue, another preferred use of intragenic T-DNA constructs like those mentioned above, is a one-vector Agrobacterium co-transformation strategy. Here, both T-DNA constructs can be co-located on the same vector. However, as they each contain their own LB and RB sequences they also produce separate T-DNAs that integrate at a different loci. Hence the T-DNA insert that contains the selectable marker gene can be crossed out in F1 or F2 generations, leaving a plant that contains no foreign sequence in its genome.

A schematic diagram of such a dual T-DNA vector comprising said genetic constructs, is set forth in FIG. 20. The sequence of this vector is set forth in SEQ ID NO:70.

Apart from a selectable marker gene (nptII), a visual marker gene (ANT1) for anthocyanin biosynthesis has been included to enable easy outcrossing. Construction of the vector was as follows: T-DNA containing tomato partial ACTIN promoter and terminator was amplified using blank pIntrA cloning vector as a template, with primers Forward (BsiWI) CGTACGGAATGCCAGCACTCC (SEQ ID NO:132) and Reverse (BsrGI) TGTACAATCGTCAACGTTCACTTCTAAAGAAATAGC (SEQ ID NO:133) and inserted into a single-T-DNA plasmid (pArt27 RbcS3C:ANT1:RbcS3C 35S:nptII:NOS) by digestion with the BsiWI enzyme.

A desired insert can then be amplified with 5′phosphorylated primers: Forward 5′PhosGATTAAAA[start insert sequence] and Reverse 5′PhosC[reverse complement of end of insert sequence] and inserted in the resulting vector opened up with HpaI and PmlI restriction enzymes, whose sites are unique in the cloning vector sequence.

### Example 5. Sequences for Expression Comprising Small RNA Sequences for Improving Resistance to Plant Viruses

As hereinabove described, in certain preferred embodiments, genetic constructs of the invention comprise one or more nucleotide sequences for expression comprising one or more small RNA nucleotide sequences, wherein said small RNA sequences are capable of modifying or altering the expression, translation and/or replication of one or more nucleic acids of a plant pathogen. Plants genetically improved using said genetic constructs may demonstrate relatively improved or enhanced disease resistance to plant pathogens, such as plant viruses.

In the past, approaches to develop genetically improved plants with improved disease resistance to viral pathogens have used anti-viral sequences that are virus-sequence derived. These previous approaches presented a risk of recombination with the viral genome during infection, creating the possibility of new strain formation. In fact, this has been shown experimentally, e.g. Greene, A. E., 1993, Mol. Biol. 22 367, and is considered a real risk that may result in virus strains with increased virulence.

This Example demonstrates that small RNA nucleotide sequences derived from plants can be used to alter or modify the expression and/or replication of viral pathogen nucleic acids.

In the preferred embodiments of the invention described in this Example, the small RNA sequences that are derived from plants do not perfectly match the viral targets and do not encode amino acids that are required for function of the virus and should therefore not be suitable for viable recombination events within the viral genomes. However, these small RNA sequences are nevertheless capable of efficiently silencing expression of these viral targets.

For this Example, several amiRNA sequences derived from plant sequences were produced and tested. Furthermore, longer RNAi construct comprise small RNA sequences derived from plant sequences have been produced and tested.

This Example demonstrates that constructs suitable for inhibiting the expression and/or replication of nucleic acids of a plant pathogen can be derived from plant sequence. Genetic constructs of the invention comprising such sequences are expected to be useful for producing genetically improved plants with improved disease resistance. By way of example, tomato plants transformed with such a construct of the invention demonstrated improved resistance to CMV, as set forth in Example 6.

amiRNA Approach

Native (tomato cv. Moneymaker) genome-derived artificial microRNA (amiRNA) nucleotide sequences were designed and cloned to target Cucumber mosaic virus (CMV). The native miRNA156b was used (SEQ ID NO:12), into which several tomato genome-derived mature microRNA sequences that partially match CMV isolate K (CMV-K) sequences in regions conserved for various isolates of CMV were introduced.

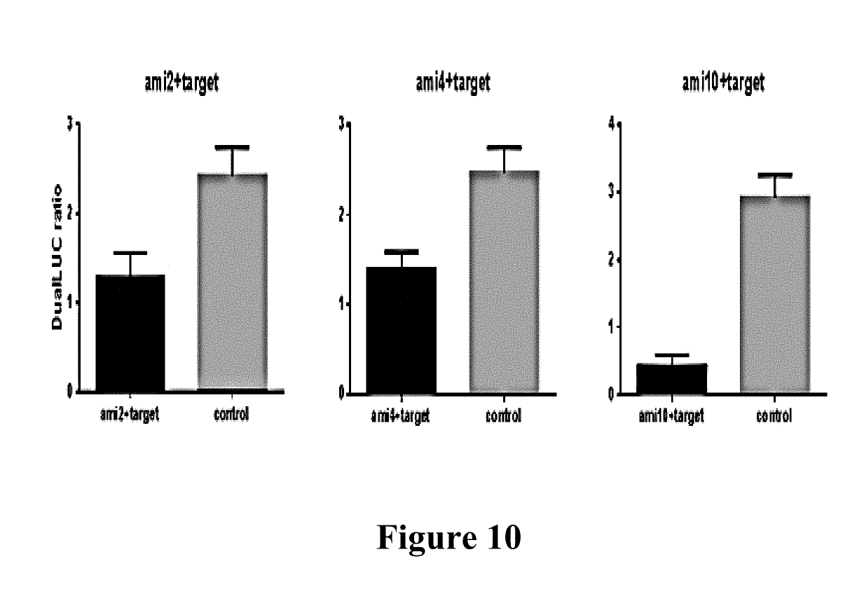

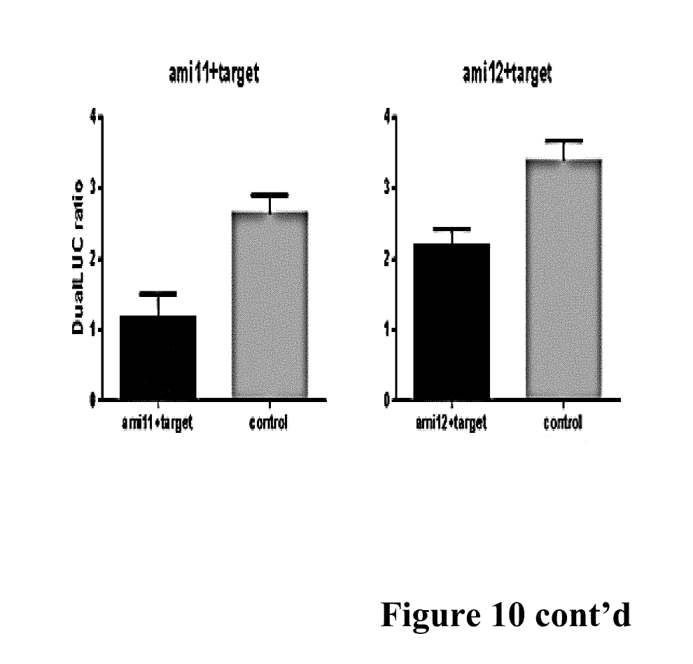

These amiRNA constructs were tested using the dual LUC assay. Approximately 25% of designed amiRNAs tested worked efficiently, such as the construct with nucleotides sequences set forth in SEQ ID NOS:13-18, causing knock-down of expression to the firefly luciferase containing the complementary viral target sequence (FIG. 10).

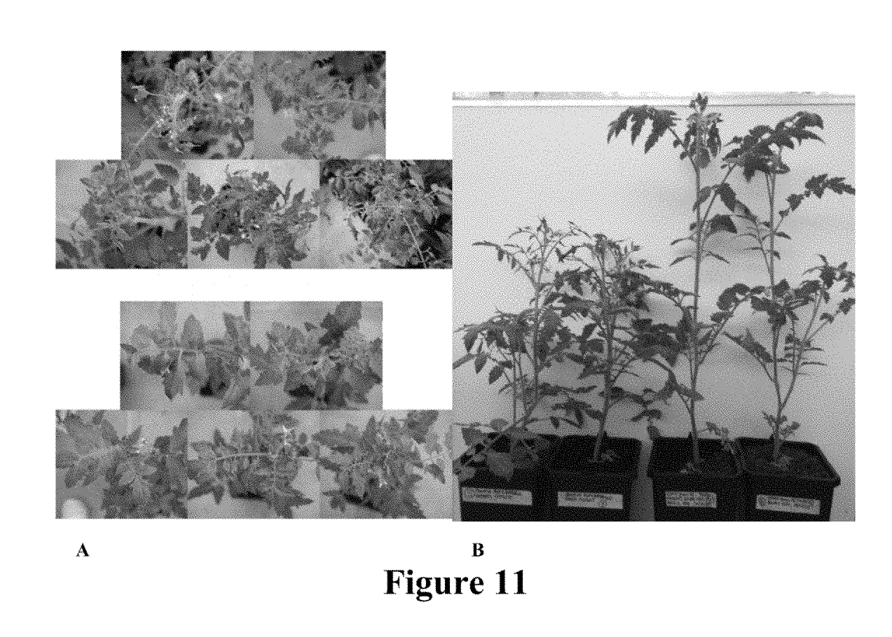

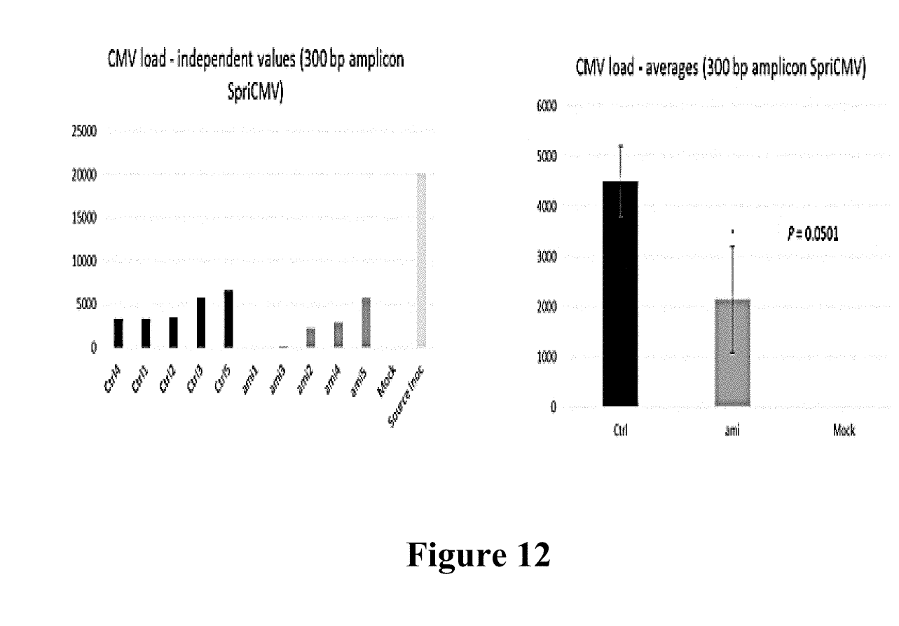

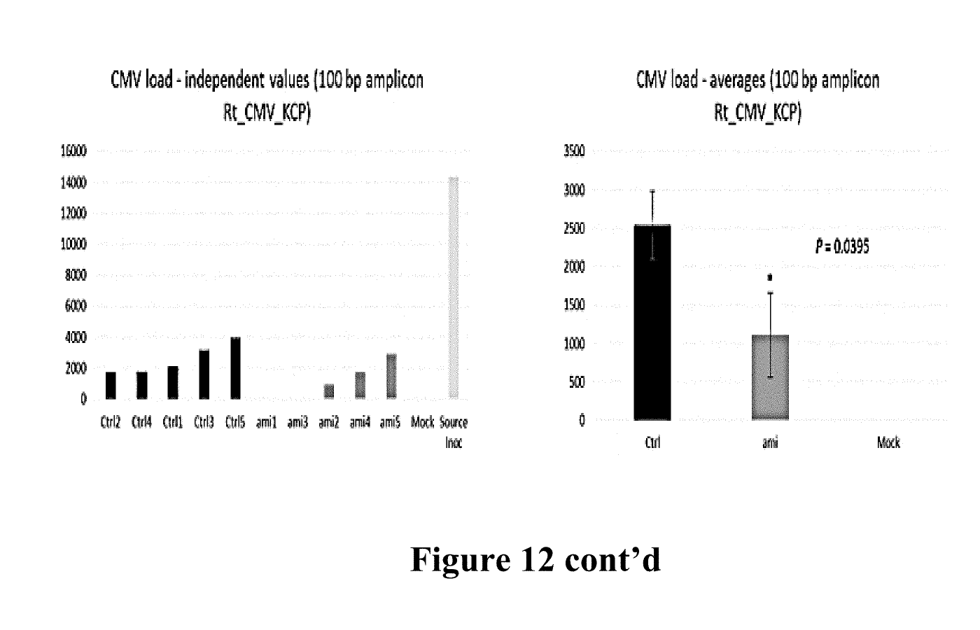

As further proof of concept, tomato plants expressing one of these amiRNA nucleotide sequences (amiRNA 10 set forth in SEQ ID NO:15) were produced by Agrobacterium-mediated transformation (following the method by Subramaniam et al., 2016, Plant Physiology, 170 1117) using a standard binary vector (pArt27 containing CaMV 35S promoter and Agrobacterium OCS terminator). As set forth in FIG. 11, these plants expressing SEQ ID NO:15 displayed improved resistance against CMV, showing decreased CMV disease symptoms as compared to corresponding wild type tomato plants. Furthermore, as set forth in FIG. 12, average CMV viral load was significantly decreased as compared to wild type plants, as assessed by qRT-PCR.

To test whether other parts of the virus can also be targeted and whether the resistance trait is heritable, tomato plants expressing a different intragenic amiRNA nucleotide sequences (amiRNA 11 set forth in SEQ ID NO:16) were produced by Agrobacterium-mediated transformation using otherwise identical conditions as above. Prior to plant transformation a transient luciferase assay was used by agroinfiltration of Nicotiana benthamina leaves, as set forth in FIG. 10. This resulted in a significant downregulation of the CMV target sequence, suggesting that amiRNA 11 would also be suitable to silence this virus in stably transformed plants. T0 plants were produced as described above and the obtained lines were tested by quantitative PCR and quantitative reverse transcriptase PCR to ensure presence and expression, respectively, of the transformed constructs. Plants from two lines (ami11-I and ami-11-II) were then grown to maturity and seeds from primary transformants were collected. Seedlings expressing homozygous or heterozygous amiRNA 11 sequence or no amiRNA 11 sequence (azygous) were identified by quantitative PCR.

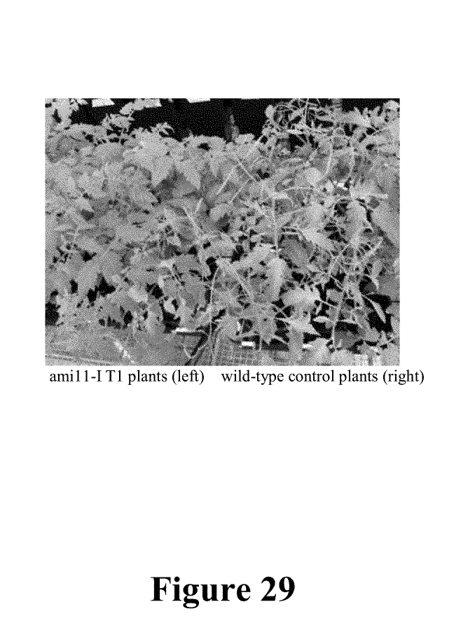

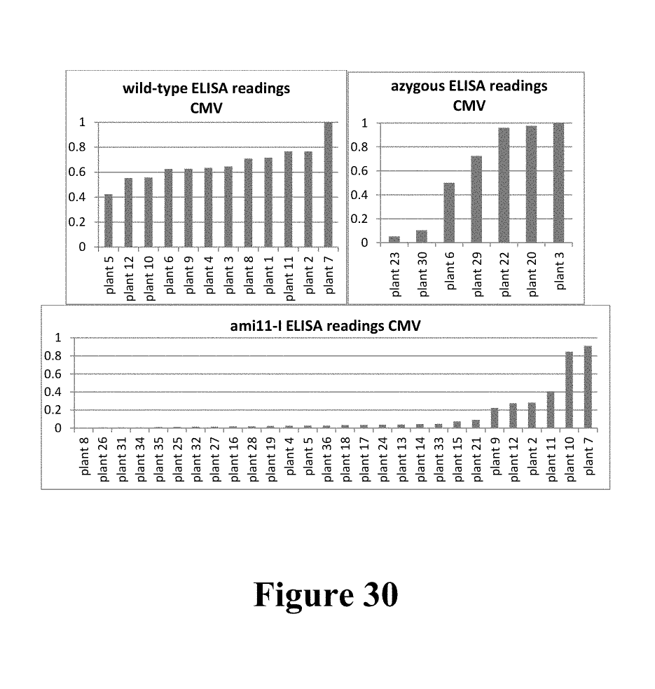

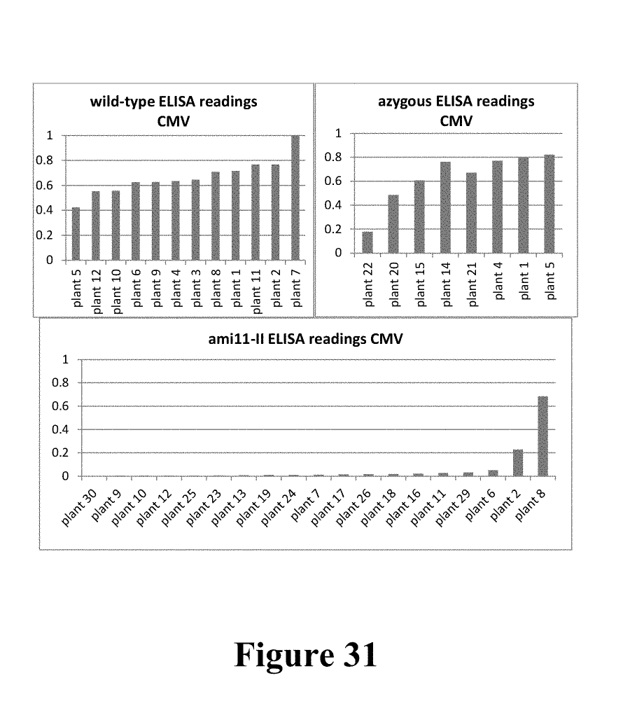

When grown to the 2-3 leaf stage (3 weeks after germination) and challenged with CMV, both homozygous and heterozygous plants harbouring amiRNA11 for both lines displayed virus resistance, while azygous plants not containing amiRNA11 showed CMV symptoms similar to wild-type plants. Examples of these plants are depicted in FIG. 29. This was consistent with results obtained when using enzyme-linked immunosorbent assays (ELISA) developed by the Queensland Department of Agriculture and Fisheries (DAF) for CMV detection. As set forth in FIGS. 30 and 31 (ami11-I and ami-11-II T1 progeny virus challenge tests), wild-type and azygous plants showed strong presence of CMV for most plants tested, while nearly all plants harbouring the ami RNA 11 construct showed little or no presence of ELISA-detectable CMV. Furthermore, routine severity scoring of symptoms of CMV-inoculated plants was carried out by DAF at two time points (3 weeks and 15 weeks after inoculation). These data are set forth in FIG. 32 and further demonstrate that ami11-I and ami-11-II T1 progeny plants showed resistance at both early and late time points compared to wild type and azygous plants that do not contain the ami11 construct. In addition, CMV inoculated wild type plants were shorter than mock-inoculated plants, but ami11-I and ami11-II plants were no shorter on average than mock-inoculated wild type plants (FIG. 32).

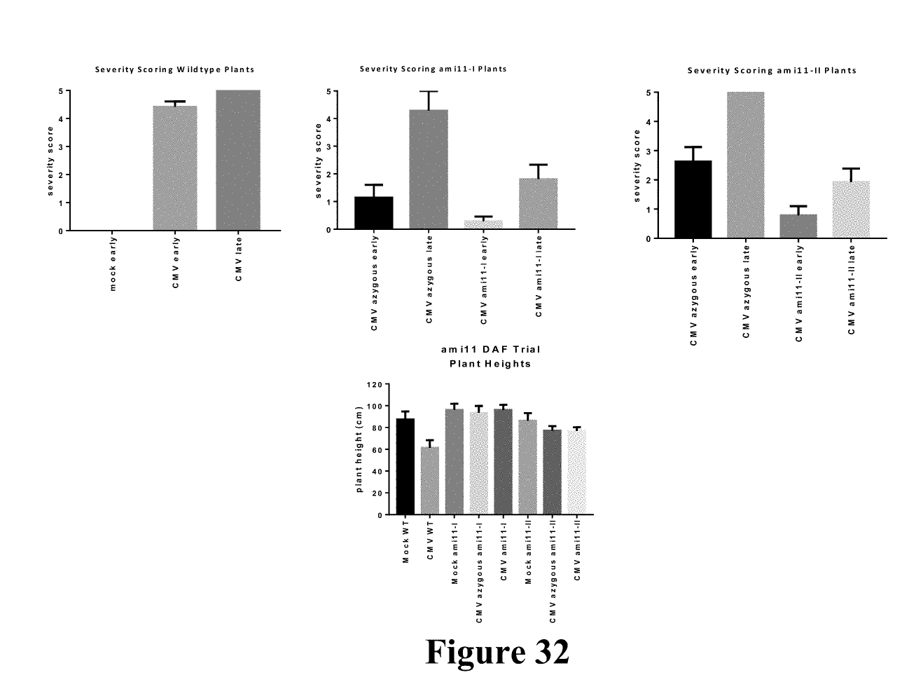

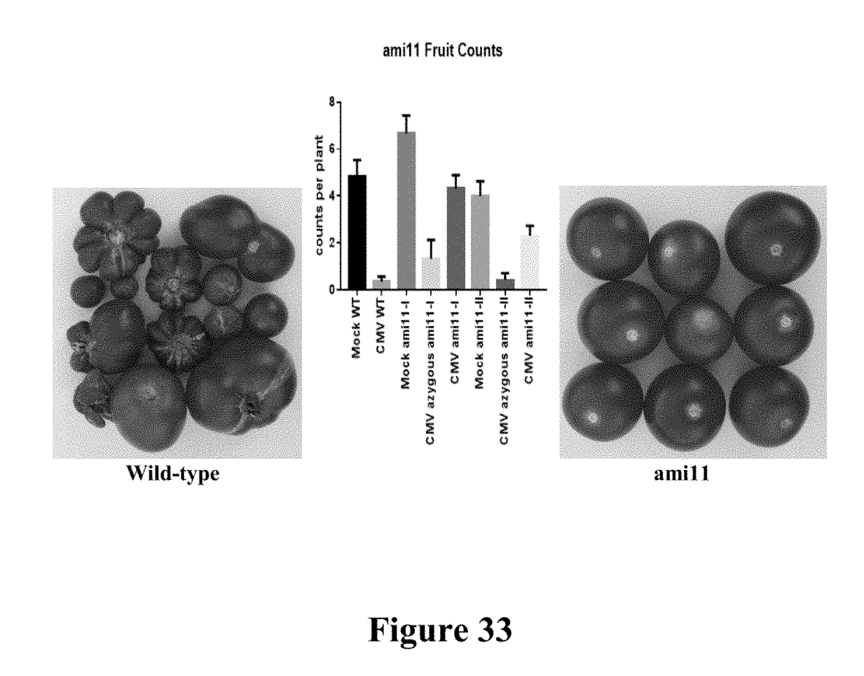

Fruit quality and quantity appeared normal and were indistinguishable from wild-type or azygous plants (FIG. 33). Fruit from CMV-challenged plants were severely affected in wild type plants but showed little or no symptoms for amiRNA 11 transformed plants.

Taken together, this demonstrates that plant genome-derived intragenic small RNA sequences can be successfully used to produce virus-resistant plants with normal yields and fruit quality and that this trait can be passed on to new generations.

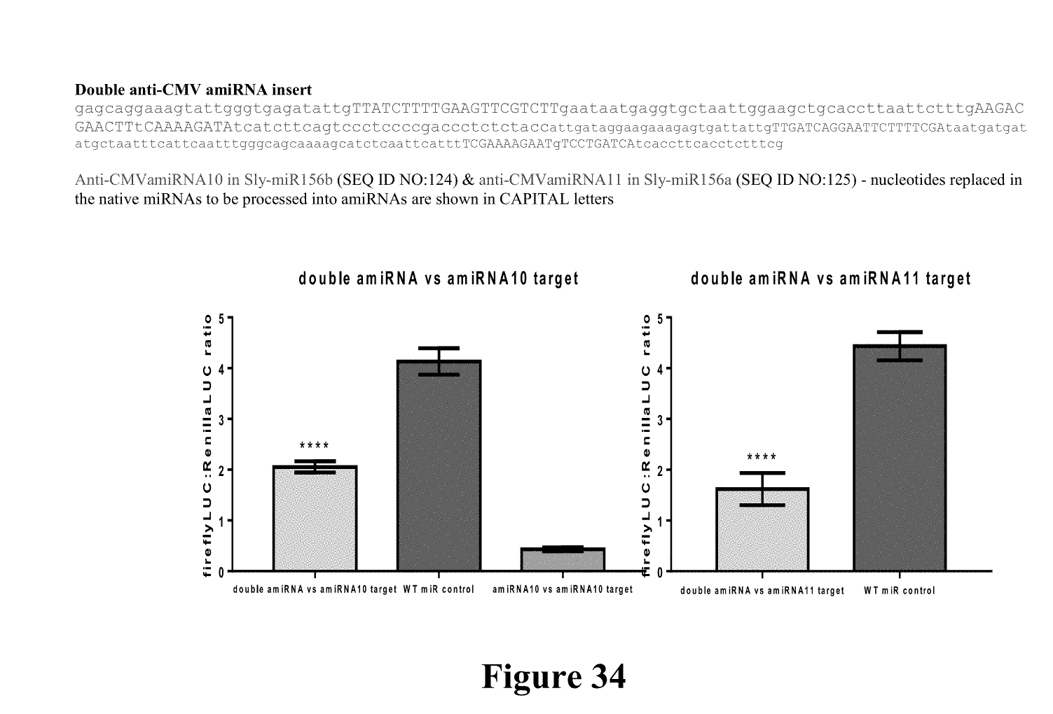

To further improve durability of virus resistance, both demonstrated amiRNA-based approaches (amiRNA10 and amiRNA11) were tested together. For this purpose both amiRNAs had to be expressed by two distinct native tomato microRNAs. Hence, nucleotides were replaced in the native Sly-miR156a and Sly-miR156b microRNAs with intragenic anti-CMV ami10 and ami11, respectively. This intragenic double ami sequence is set forth in FIG. 34 and SEQ ID NO:80. For the purpose of testing whether the construct is able to suppress the corresponding viral sequences, the dual luciferase assay using agroinfiltration of N. benthamiana plants, was employed as described above. For the purpose of this assay, the sequence was cloned into the pArt27 plasmid flanked by the CaMV 35S promoter and the OCS terminator. As set forth in FIG. 34, the construct significantly (P<0.001; Student's t test) suppressed the corresponding CMV target sequences.

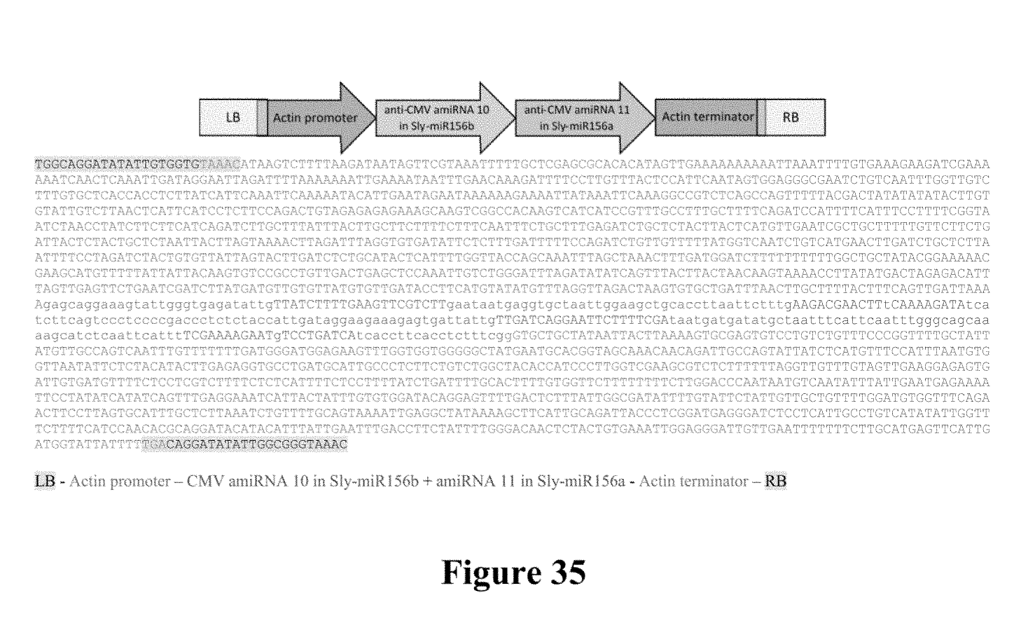

To transform plants, both demonstrated amiRNA-based approaches (amiRNA10 and amiRNA11) were combined in one fully intragenic construct. As set forth in FIG. 35 and SEQ ID NO:81, a “two-vector two Agrobacterium strain co-transformation strategy” vector was produced with pArt27 as backbone that can be used in combination with a separate selectable marker construct, that can be outcrossed at a later stage prior to commercialisation. For this purpose, the sequence (SEQ ID NO:81) was inserted into pIntrA (FIG. 18; SEQ ID NO:67). SEQ ID NO:81 was first synthesised and then amplified with F primer 5′Phos GATTAAAAGAGCAGGAAAGTATTGGGTGAGATATTG (SEQ ID NO:134) and R primer 5′Phos CcgaaagaggtgaaggtgaTGATCA (SEQ ID NO:135) to complement missing ends of the ACTIN promoter and terminator and subsequently ligated with pIntrA opened up with HpaI and PmlI. Direction of the insert was tested by sequencing.

Tomato plants were transformed with this construct (FIG. 35) as described above together with the selectable marker construct set forth in FIG. 19 and SEQ ID NO:69, as a separate vector which also harbours the tomato ANT1 gene for visual recognition of transformed plants. Regenerated plants displayed purple roots, confirming their transformation status. Further testing for double amiRNA expression and CMV resistance is currently underway.

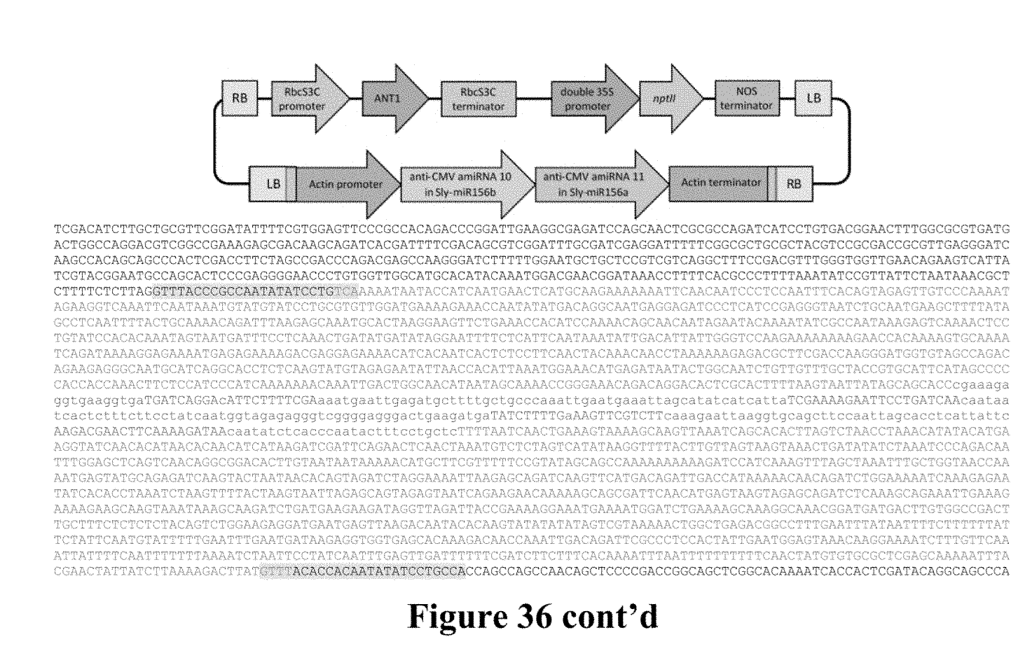

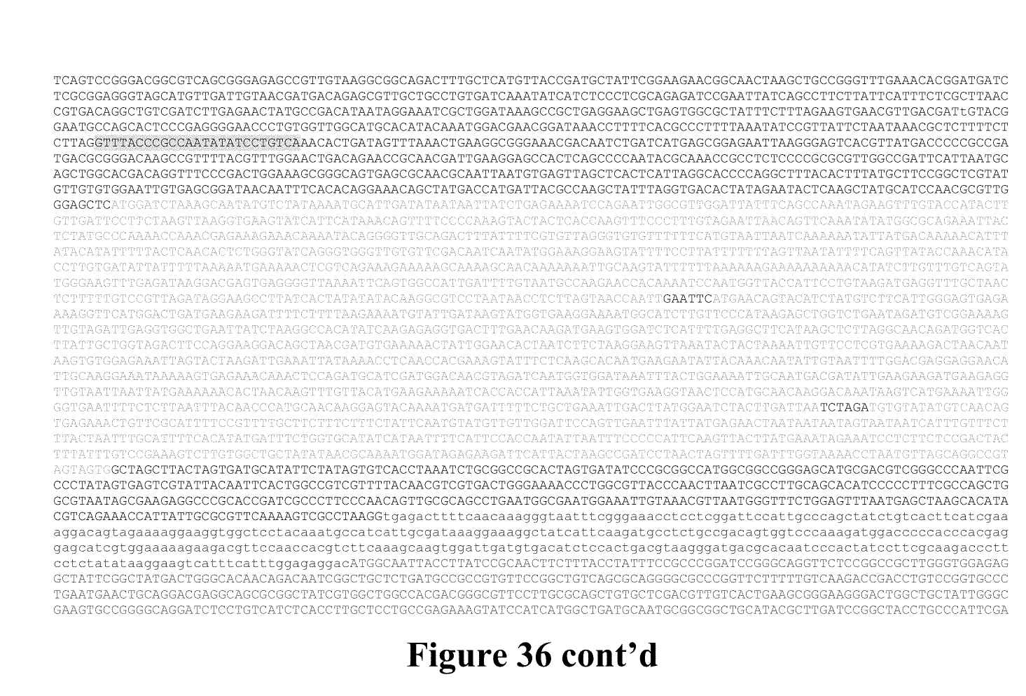

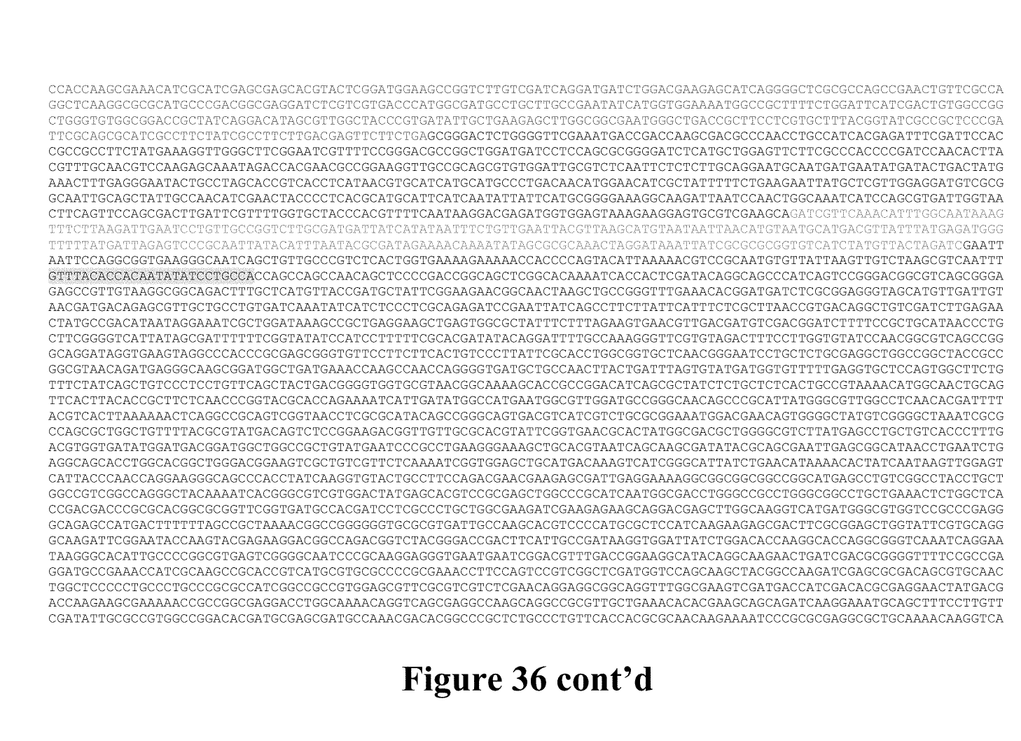

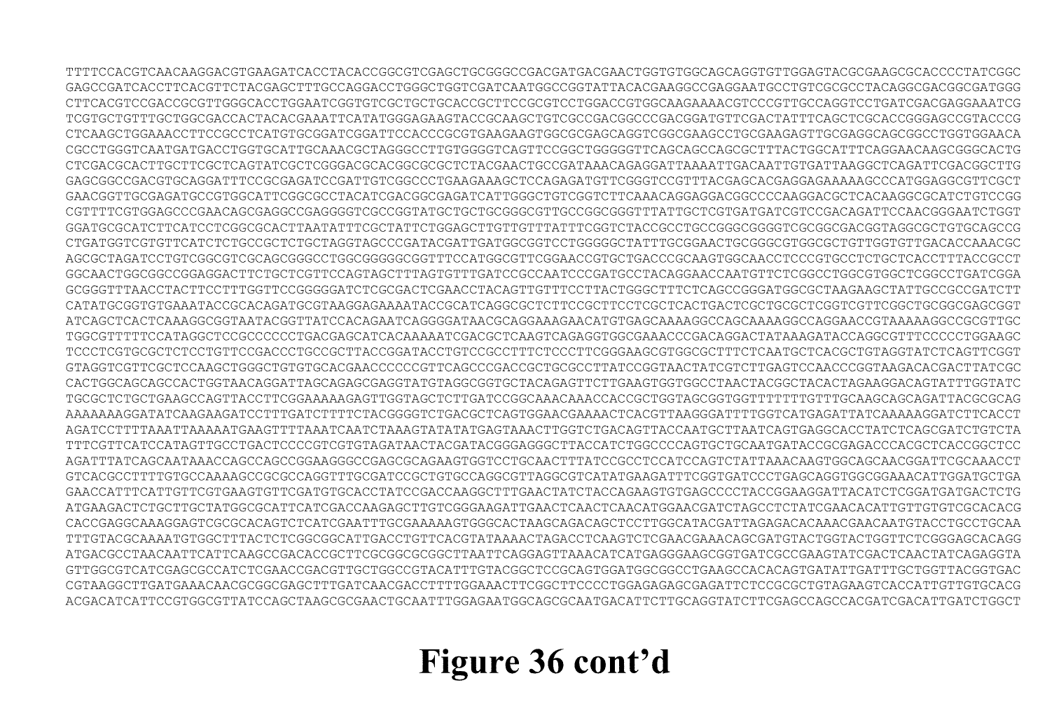

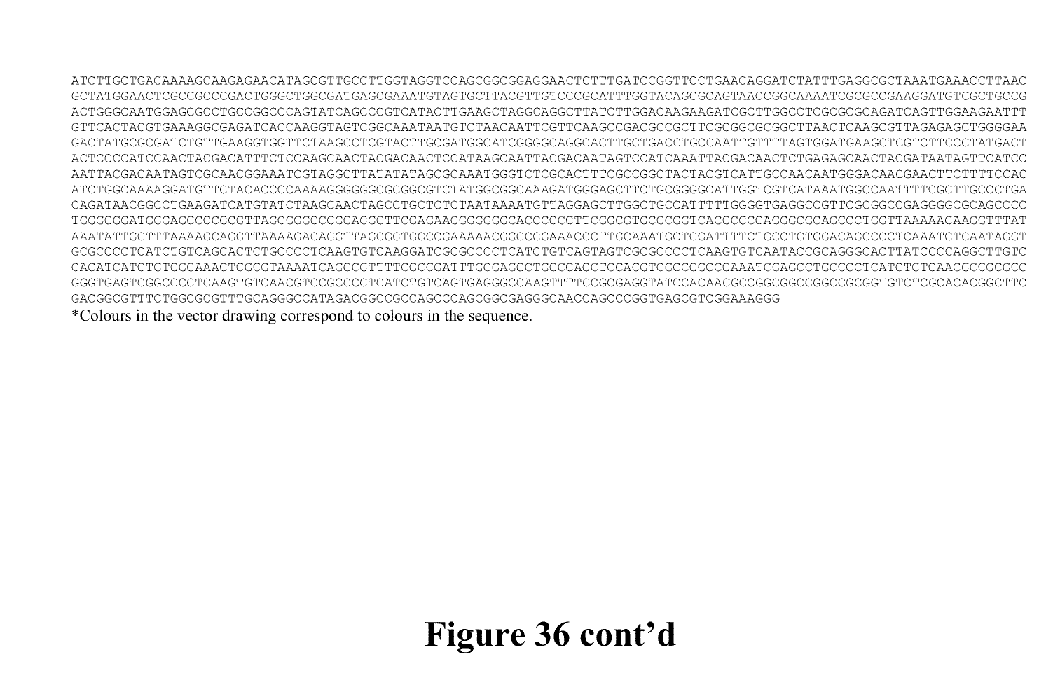

Alternatively, the double cassette (one vector containing two T-DNA cassettes) approach was used that is set forth in FIG. 20 and SEQ ID NO:70. For this purpose, the double amiRNA T-DNA cassette (SEQ ID NO:81) was inserted into pArt27 RbcS3C:ANT1:RbcS3C 35S:nptII:NOS (FIG. 19; SEQ ID NO:69). First, the double amiRNA T-DNA was amplified from the vector set forth in FIG. 35 using primers: Forward (BsiWI) CGTACGGAATGCCAGCACTCC (SEQ ID NO:136) and Reverse (BsrGI) TGTACAATCGTCAACGTTCACTTCTAAAGAAATAGC (SEQ ID NO:137) and then inserted into the single-T-DNA plasmid pArt27 RbcS3C:ANT1:RbcS3C 35S:nptII:NOS (FIG. 19; SEQ ID NO:69) opened up with the BsiWI enzyme. Tomato plant transformation, regeneration and CMV challenge experiments for this approach are currently underway. The genetic organisation and complete sequence of this double T-DNA vector for durable intragenic CMV resistance are set forth in FIG. 36 and SEQ ID NO:82.

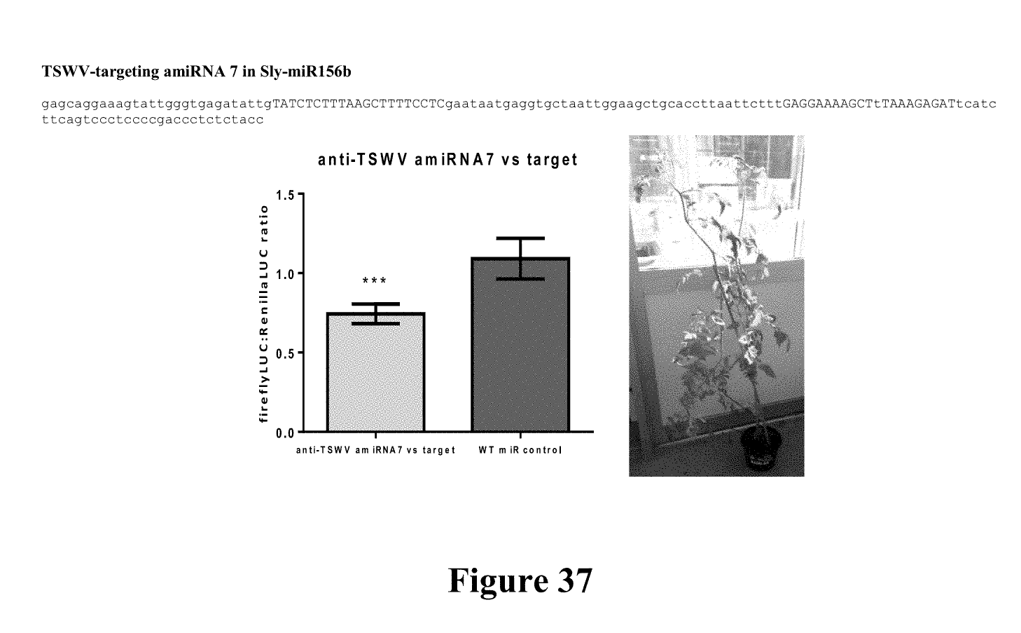

To apply the intragenic amiRNA approach also for other viruses, intragenic constructs were produced for Tomato spotted wilt virus (TSWV)-resistance in tomato, another virus that causes severe yield losses worldwide. Similar as for CMV, first intragenic sequences of sufficient length were identified that match TSWV sequence. Then, nucleotides were replaced in the native Sly-miR156b microRNA with intragenic anti-TSWV amiRNA7 sequence giving rise to intragenic sequence set forth in FIG. 37 and SEQ ID NO:83.

For the purpose of testing whether the construct is able to suppress the corresponding viral sequences, the dual luciferase assay using agroinfiltration of N. benthamiana plants, was employed as described above. For the purpose of this assay, the sequence was cloned into the pArt27 plasmid flanked by the CaMV 35S promoter and the OCS terminator. As set forth in FIG. 37, the construct significantly (P<0.001; Student's t test) suppressed the corresponding TSWV target sequence.

To transform plants, the sequence (SEQ ID NO:83) was inserted into pIntrA (FIG. 18; SEQ ID NO:67). SEQ ID NO:83 was first synthesised and then amplified with F primer 5′Phos GATTAAAAGAGCAGGAAAGTATTGGGTGAGATATTG (SEQ ID NO:138) and R primer 5′Phos CcgaaagaggtgaaggtgaTGATCA (SEQ ID NO:139) to complement missing ends of the ACTIN promoter and terminator and subsequently ligated with pIntrA opened up with HpaI and PmlI. Direction of the insert was tested by sequencing.

Tomato plants were transformed with this construct (FIG. 37) as described above, together with the selectable marker construct set forth in FIG. 19 and SEQ ID NO:69, as a separate vector which also harbours the tomato ANT1 gene for visual recognition of transformed plants. Testing for amiRNA7 presence and expression was positive for seven lines and tomatoes were harvested for seed collection. The plants had normal phenotypes, albeit growing taller than usual, they fruited and produced seeds at rates comparable to WT. TSWV resistance testing of T1 seedlings (wild type, azygous, homozygous and heterozygous) is currently underway.

To test whether this approach is also valid for other crops, intragenic amiRNA constructs were also produced for Johnson grass mosaic virus (JGMV)-, Sugarcane mosaic virus (SCMV)- and Maize dwarf mosaic virus (MDMV)-resistance in sorghum, as well as Rice tungro bacilliform virus (RTBV) resistance in rice.

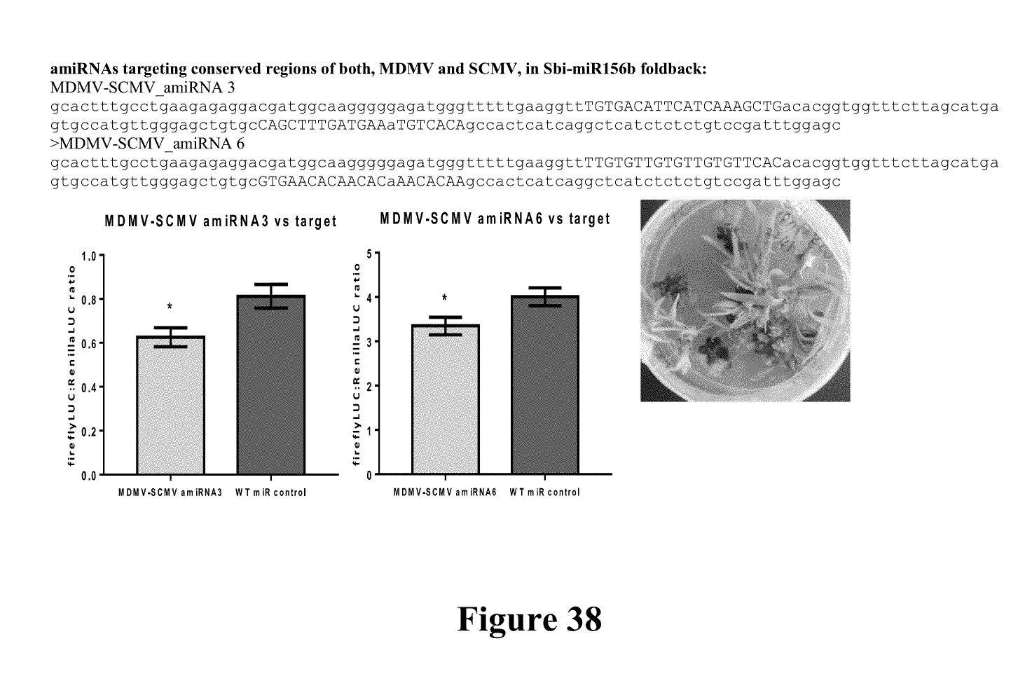

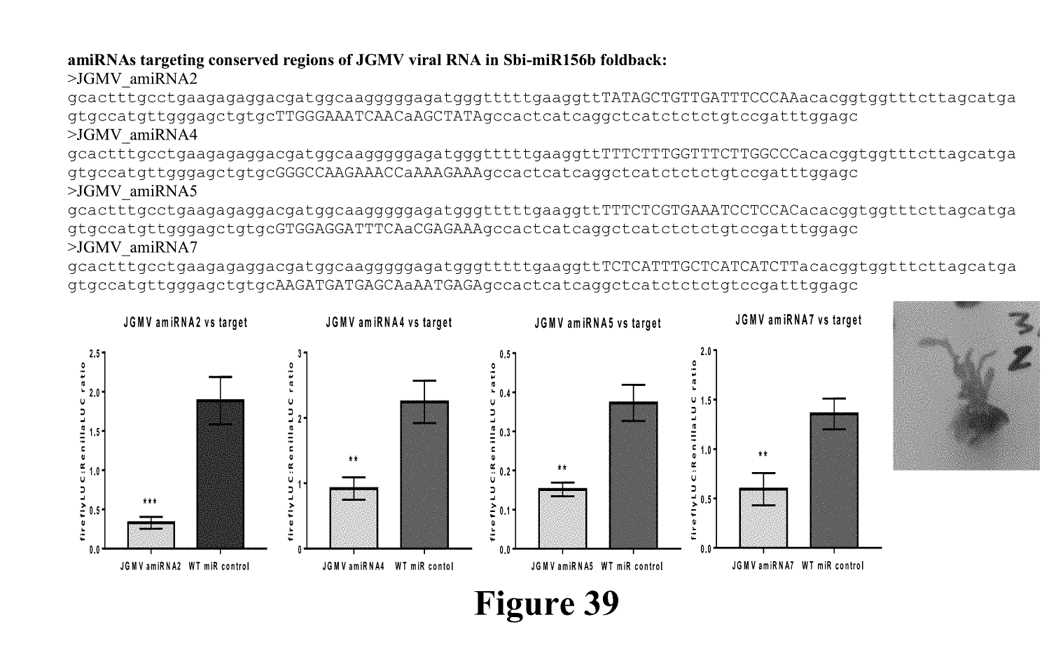

To develop this approach for multiple virus resistance in sorghum, amiRNAs were designed such that they target either multiple viruses or multiple virus isolates of the same virus. First intragenic sequences of sufficient length were identified that match JGMV, SCMV and/or MDMV in conserved regions. Then, nucleotides were replaced in the native sorghum microRNA Sbi-miR156b with various intragenic anti-viral amiRNA sequences. Some of these are set forth in FIGS. 38-39 and SEQ ID NOs:83-89. The amiRNAs were synthesised and amplified with primers F tccCTGCAGgcactttgcctgaagagaggacg (SEQ ID NO:140) and R 5′Phos gctccaaatcggacagagagatgagc (SEQ ID NO:141), digested with PstI and inserted into vector pSbiUbi1 (FIG. 21; SEQ ID NO:73) or pSbiUbi2 (FIG. 22; SEQ ID NO:74) opened up with PstI and SfoI enzymes. The resulting plasmids were cut with PmlI to obtain minimal intragenic transformation cassettes.

However, prior to plant transformation, amiRNA constructs were tested using agoinfiltration of N. benthamiana leaves. FIG. 38 shows successful testing of two anti-MDMV-SCMV amiRNA constructs using the dual luciferase assay that resulted in significant (P<0.05; Student's t test) knock down of MDMV-SCMV target sequences FIG. 39 shows successful testing of four anti-JGMV amiRNA constructs using the dual luciferase assay that resulted in significant (P<0.01; Student's t test) knock down of JGMV target sequences.

Next, sorghum plants (Sorghum bicolor cultivar Tx430) were transformed with the above amiRNAs using intragenic pSbiUbi1 and pSbiUbi2 cassettes for expression. Linear intragenic DNA cassettes were excised and used for particle bombardment of sorghum immature embryos. The sorghum transformation protocol by described by Liu et al. 2014 (IN: Cereal Genomics: Methods and Protocols, Methods in Molecular Biology, R. J. Henry & A. Furtado (eds.), Springer, New York) was used. Plants are currently regenerating and prepared for SCMV and JGMV virus challenge.

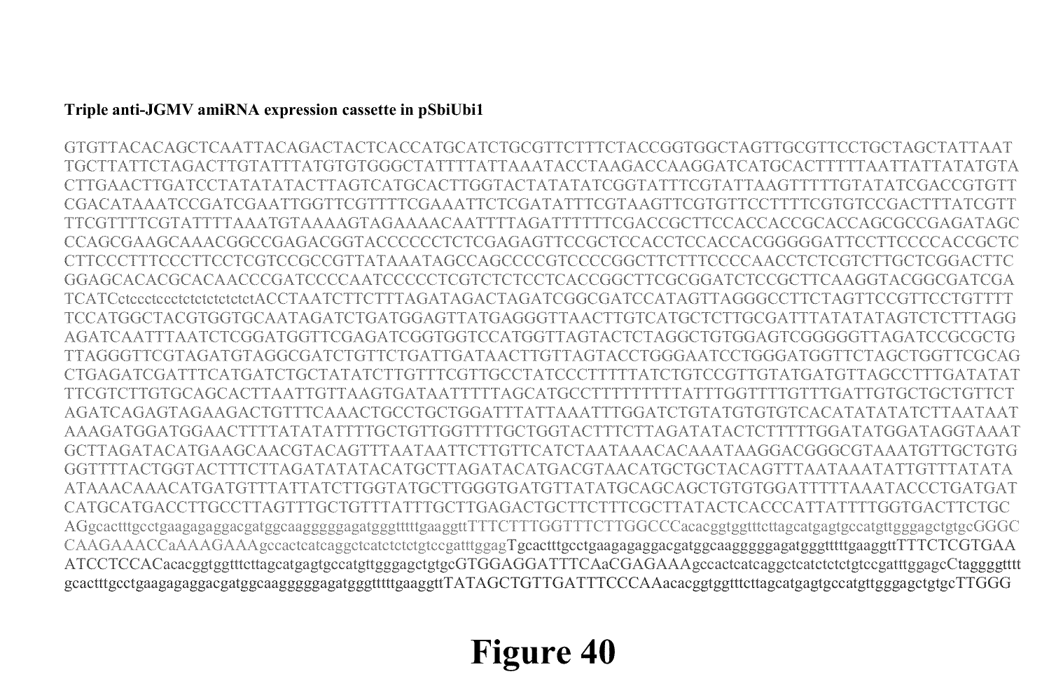

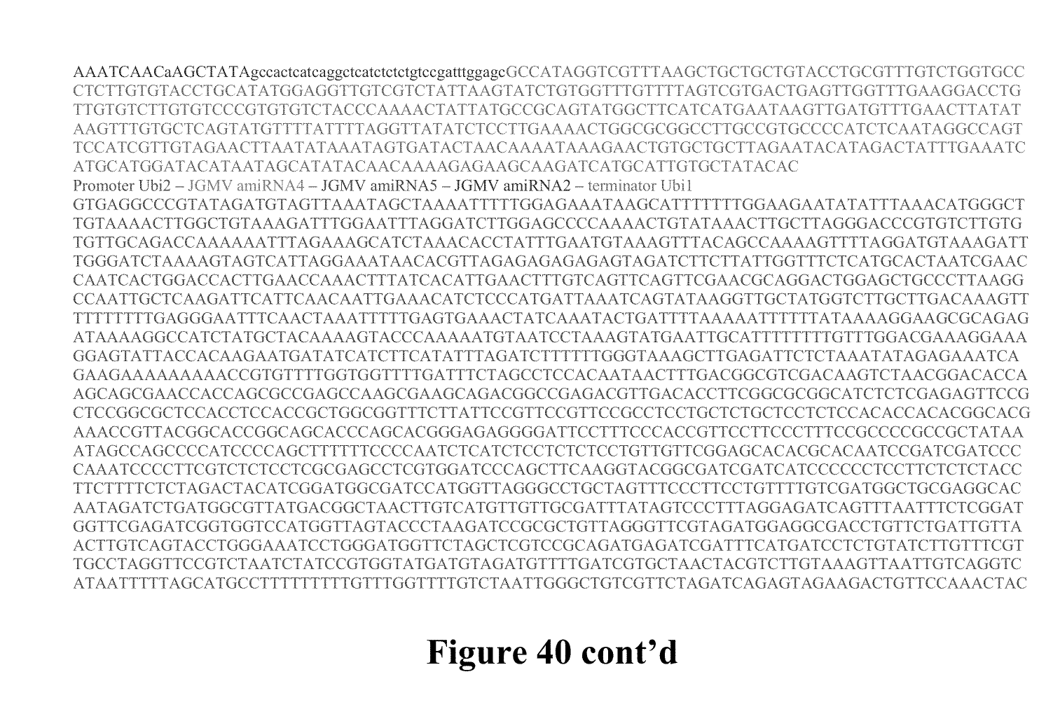

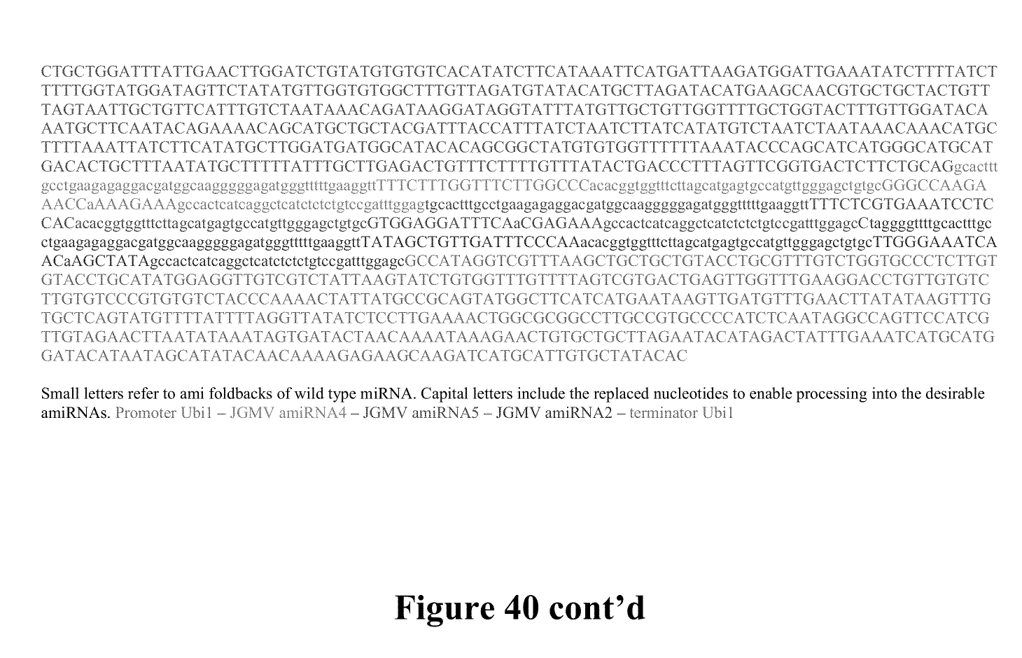

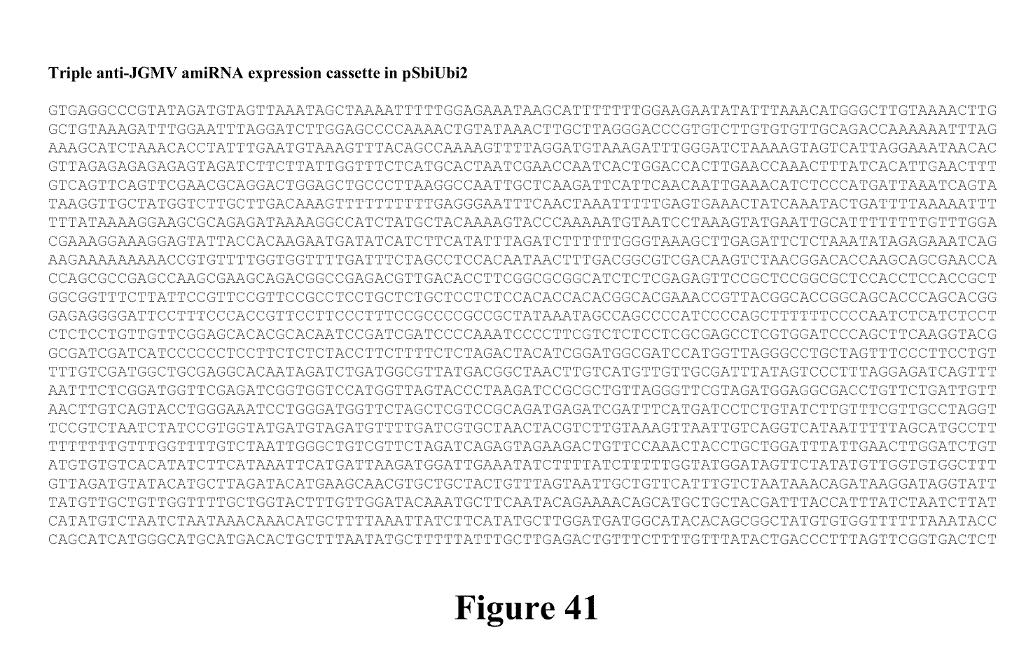

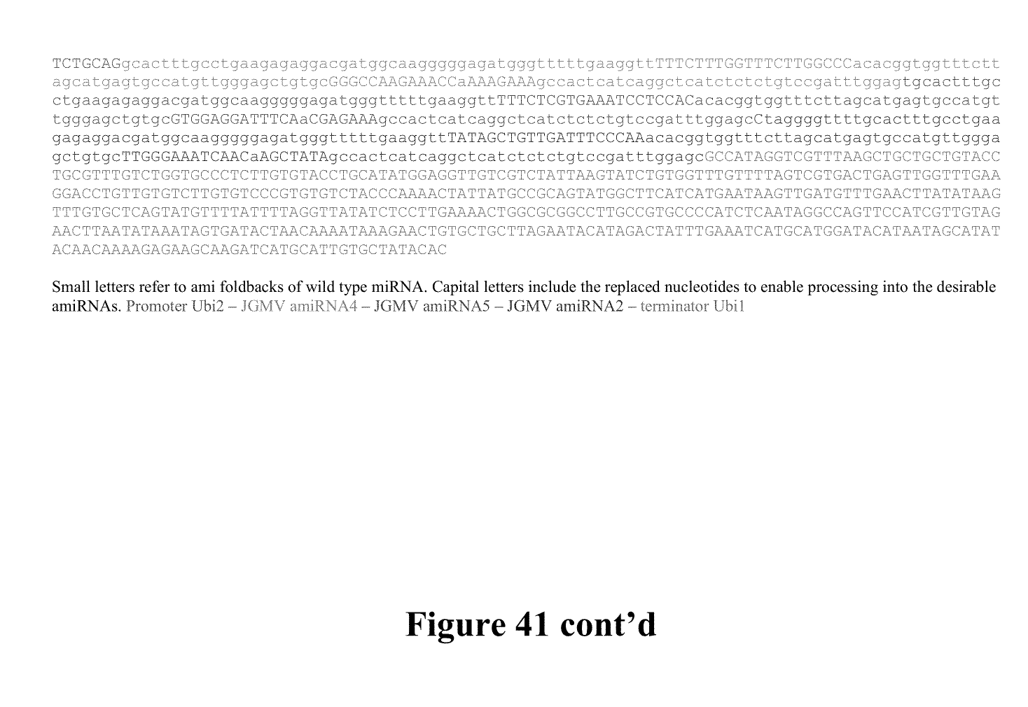

To provide multiple intragenic resistance in sorghum against JGMV, a triple amiRNA approach was used. As set forth in FIG. 40 and SEQ ID NO:90, one of these constructs contains amiRNA2 (SEQ ID NO:86), amiRNA4 (SEQ ID NO:87), and amiRNA5 (SEQ ID NO:88), in pSbiUbi1 (SEQ ID NO:73). As set forth in FIG. 41 and SEQ ID NO:91, another one of these constructs contains amiRNA2 (SEQ ID NO:86), amiRNA4 (SEQ ID NO:87), and amiRNA5 (SEQ ID NO:88), in pSbiUbi2 (SEQ ID NO:74).

The cloning strategy for these constructs was as follows: amiRNA4 was amplified with primers F tccCTGCAGgcactttgcctgaagagaggacg (SEQ ID NO:142) (adding a PstI site to the 5′ end) and R gtgcactccaaatcggacagagagatgagcc (SEQ ID NO:143) (adding an ApaLI site to the 3′ end). AmiRNA5 was amplified with primers F gtgcactttgcctgaagagaggacg (SEQ ID NO:144) (adding an ApaLI site to the 5′ end) and R aacccctaggctccaaatcggacagagagatgag (SEQ ID NO:145) (adding an AvrII site to the 3′ end). AmiRNA2 was amplified with primers F cctaggggttttgcactttgcctg (SEQ ID NO:146) (adding an AvrII site to the 5′ end) and R 5′Phos gctccaaatcggacagagagatgagc (SEQ ID NO:147). The fragments were digested with respective enzymes and ligated into either vector pSbiUbi1 or pSbiUbi2 opened up with PstI and SfoI in one reaction.

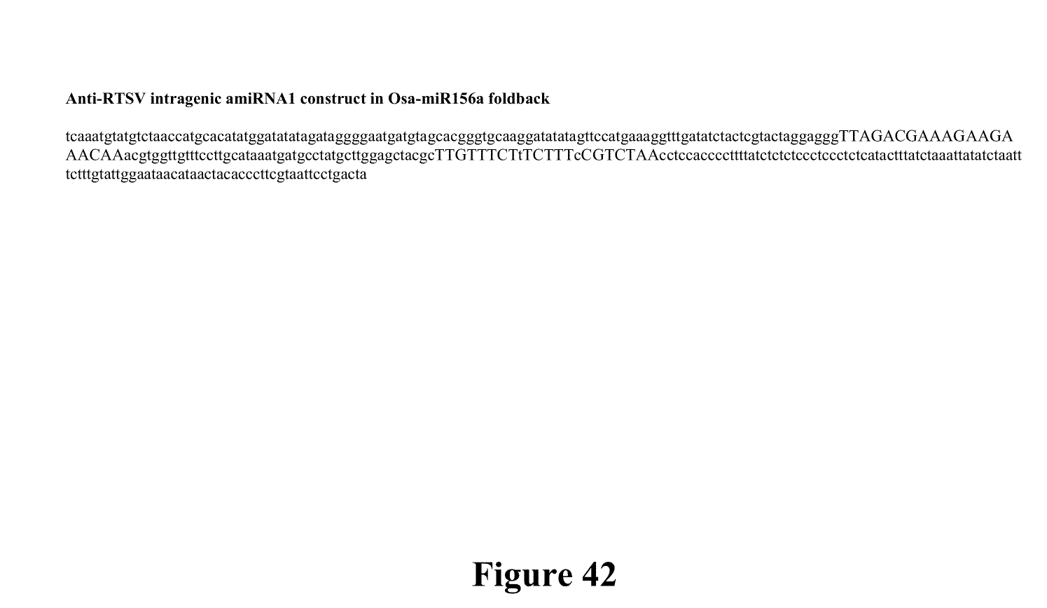

To develop the intragenic amiRNA approach for virus resistance in rice, amiRNAs were designed such that they target Rice tungro spherical virus (RTSV), a helper virus that mediates symptom severity caused by RTBV. For this purpose, nucleotides were replaced in the native rice microRNA Osa-miR156a with various intragenic anti-viral amiRNA sequences. One of these (amiRNA1) is set forth in FIG. 42 and SEQ ID NO: 93. To produce an intragenic rice transformation cassette, amiRNA1 sequence was synthesised, amplified with primers F GAGCtcaaatgtatgtctaaccatgcacatatgg (SEQ ID NO:148) (introducing nucleotides to complete SacI site to its 5′ end) and R 5′Phos tagtcaggaattacgaagggtgtagttatgttattc (SEQ ID NO:149). It was restricted with SacI and inserted into pOsaAPX (FIG. 23; SEQ ID NO:76) opened up with SacI and blunt-end cutter PsiI, the three last nucleotides of which contribute the “continuation” of native Osa-miR156a foldback identical to its overall sequence in the database. Further testing in rice plants is currently underway.

### RNAi Approach

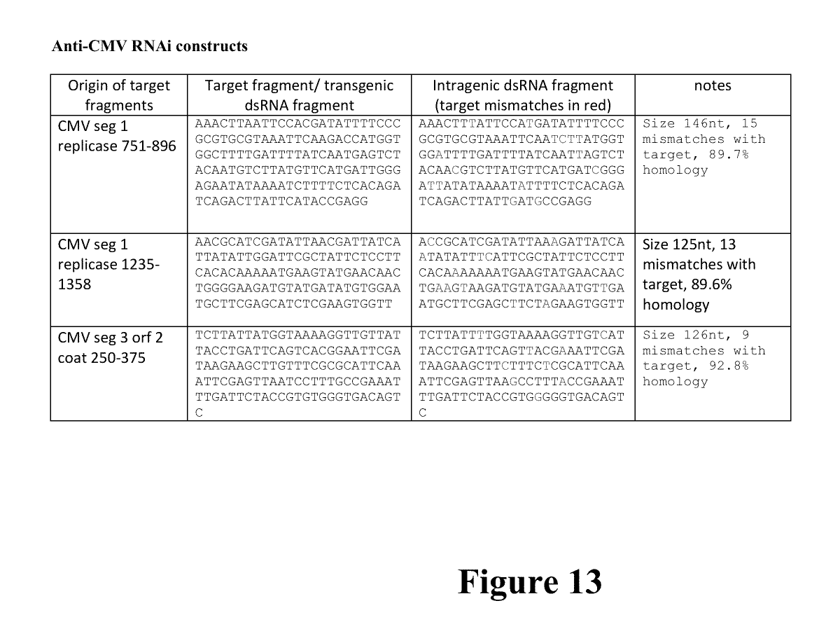

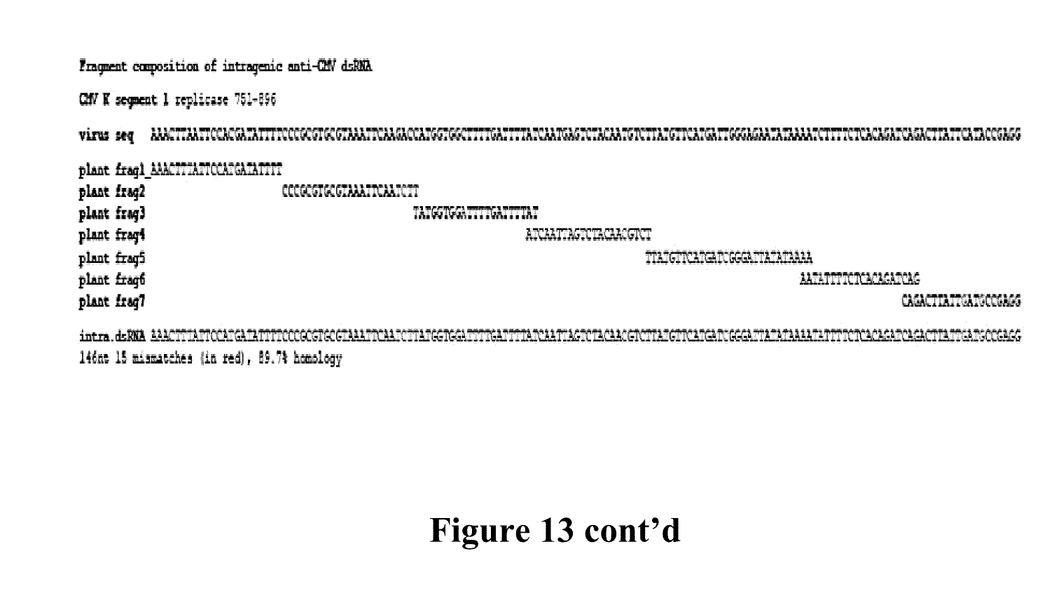

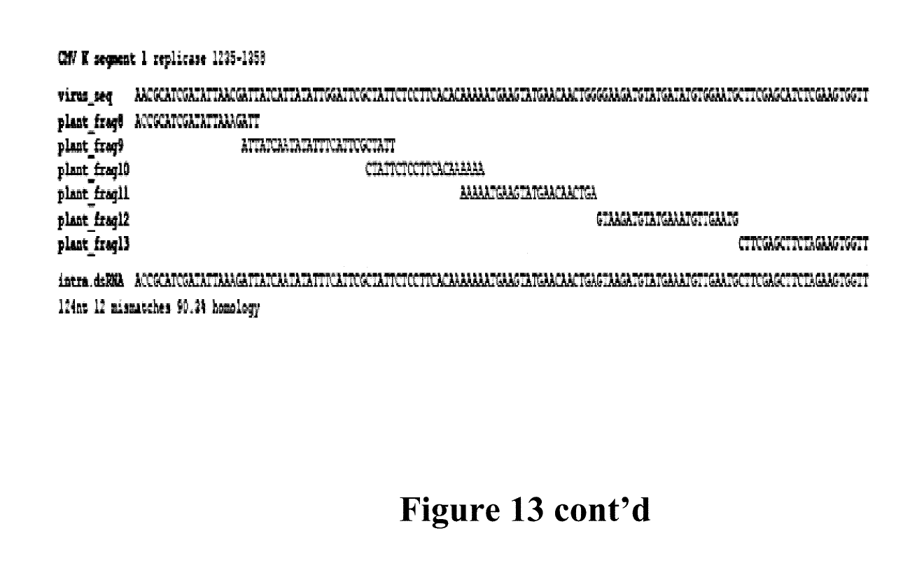

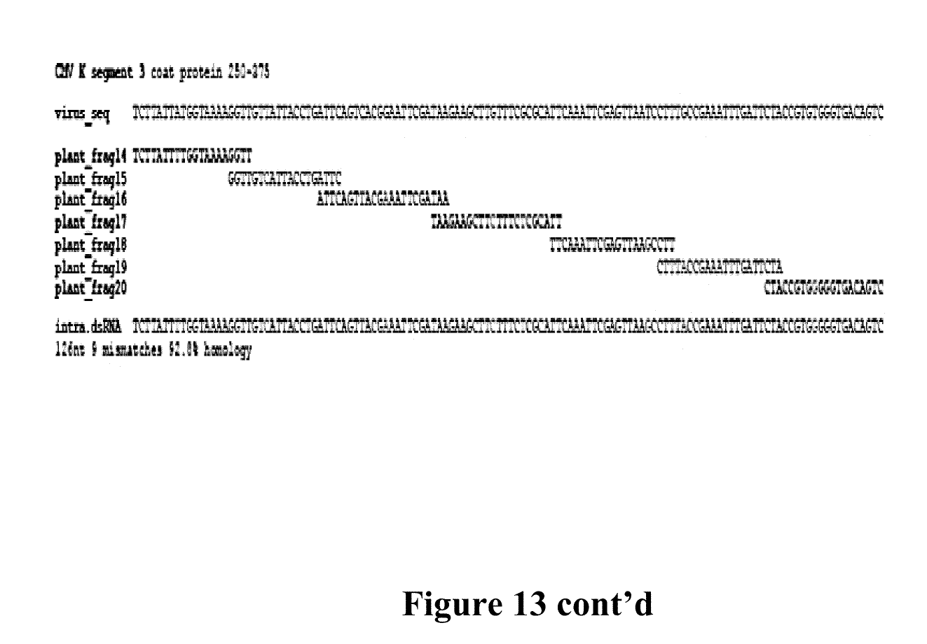

To test whether ‘traditional’ hairpin RNAi constructs comprising nucleotide sequences RNA that gives rise to dsRNA could be produced using plant-derived sequences for the invention, a long RNAi construct spanning several hundred nucleotides was designed comprising RNA sequence that targets CMV-K (SEQ ID NO:18). The intragenic RNAi sequence was created by blasting CMV-K segment sequences against the tomato genome, selecting the best matching fragments of ≥20nt in length and arranging them together with small overlaps where possible. FIG. 13 shows how tomato (cultivar Moneymaker) sequences were used and brought together to create SEQ ID NO:18, where each plant-derived sequence was at least 20 nts in length. The sequence displayed an overall match to CMV-K sequence (SEQ ID NOS:19-21) of 90%.

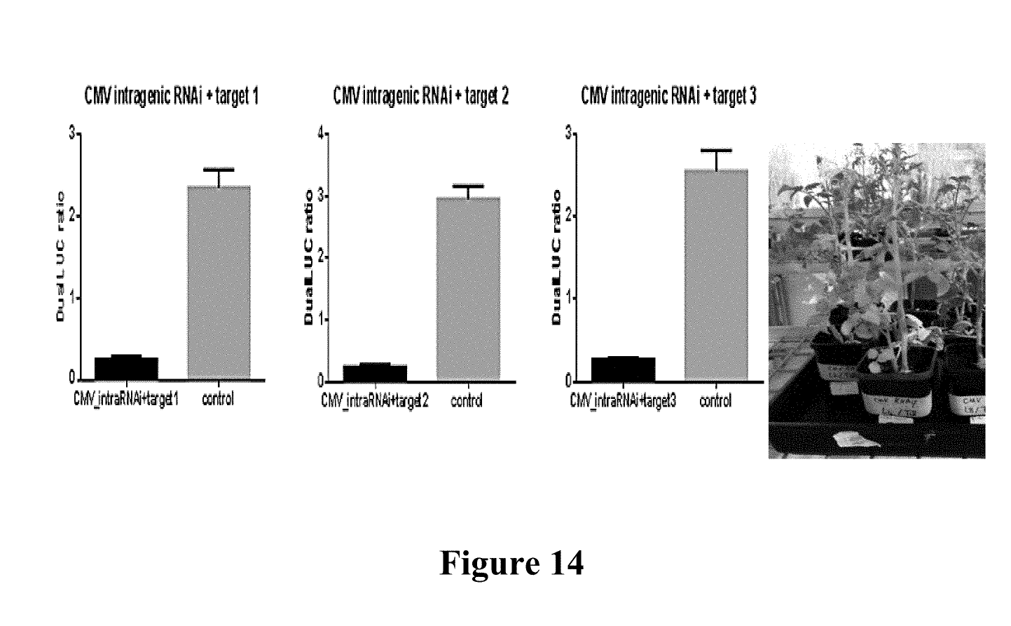

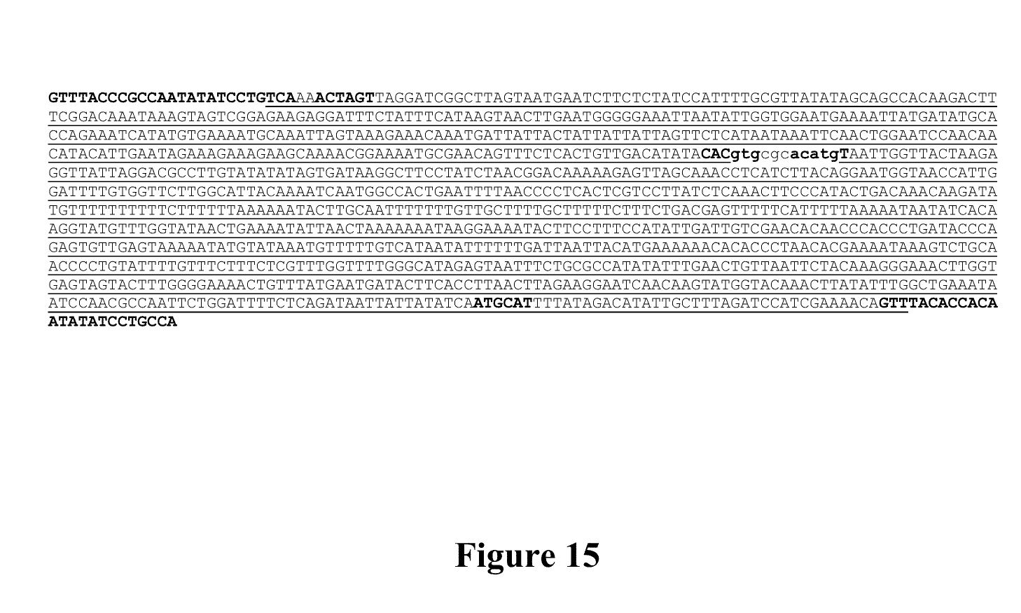

This sequence was tested for its RNAi silencing ability when brought into contact with three different corresponding CMV target sequences (using the dual LUC assay). As shown in FIG. 14, the CMV RNAi construct caused a strong knock-down of expression for all three CMV targets, relative to the control.

For tomato transformation, an intragenic RNAi construct was first built in pKannibal by including the CMV-K RNAi sequence (SEQ ID NO:18) in sense direction, followed by the PDK intron sequence as spacer and the anti-sense CMV-K RNAi sequence. The cassette was then transferred into pArt27 using SacI and SpeI sites. The complete sequence of the corresponding vector is set forth is SEQ ID NO:93. Plants were regenerated and 14 lines were confirmed to contain the intragenic construct. These had normal phenotype (FIG. 14) and are currently undergoing CMV resistance testing.

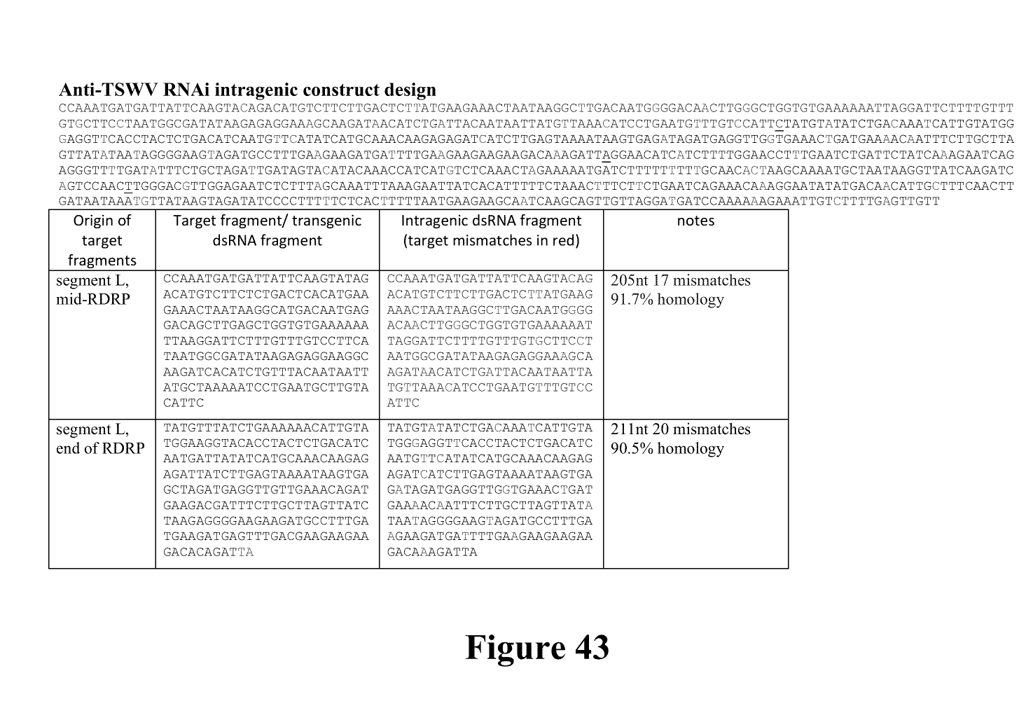

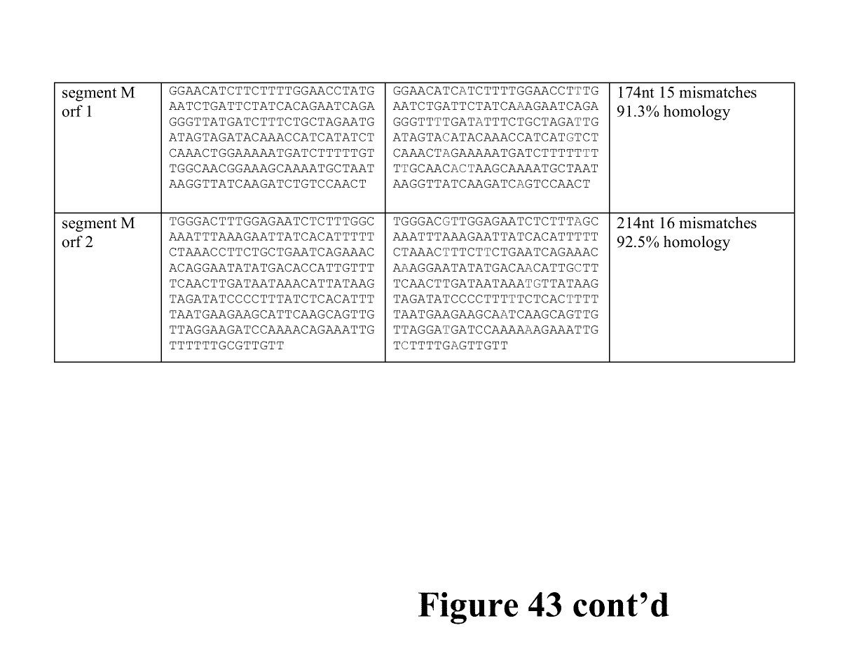

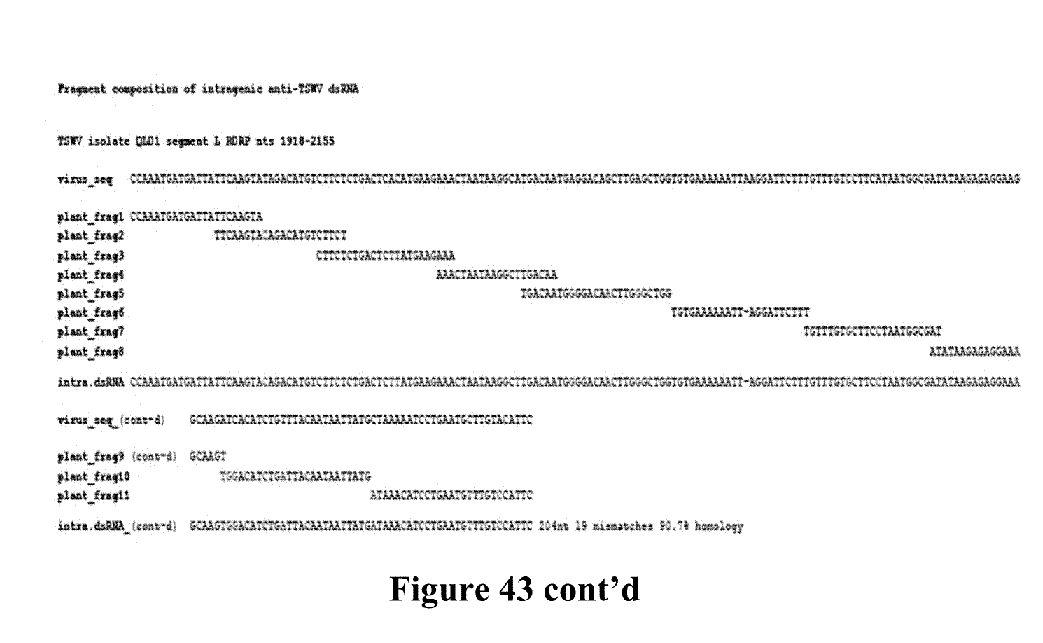

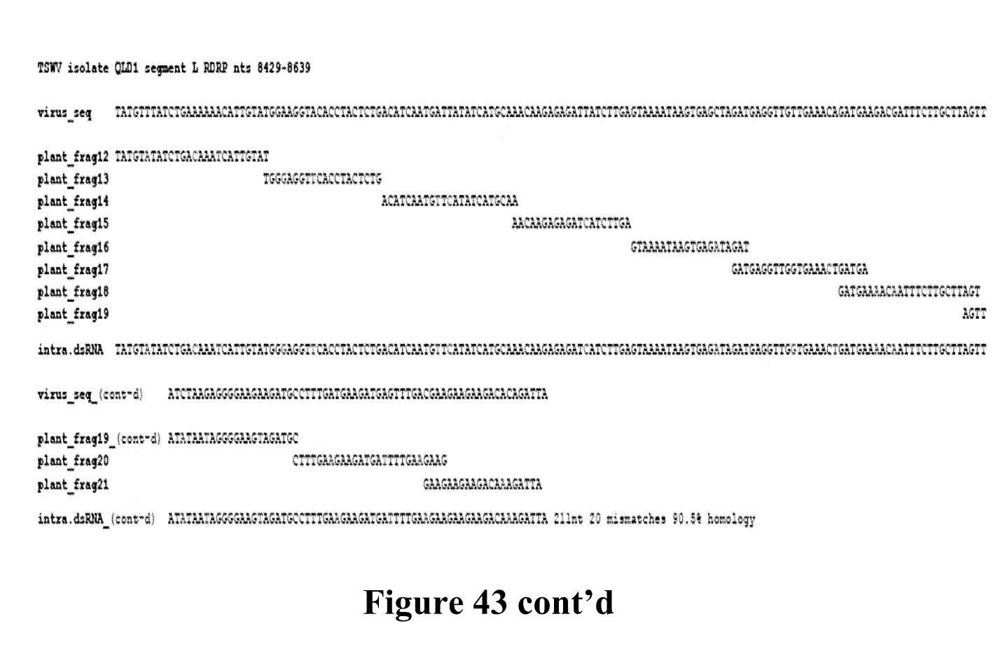

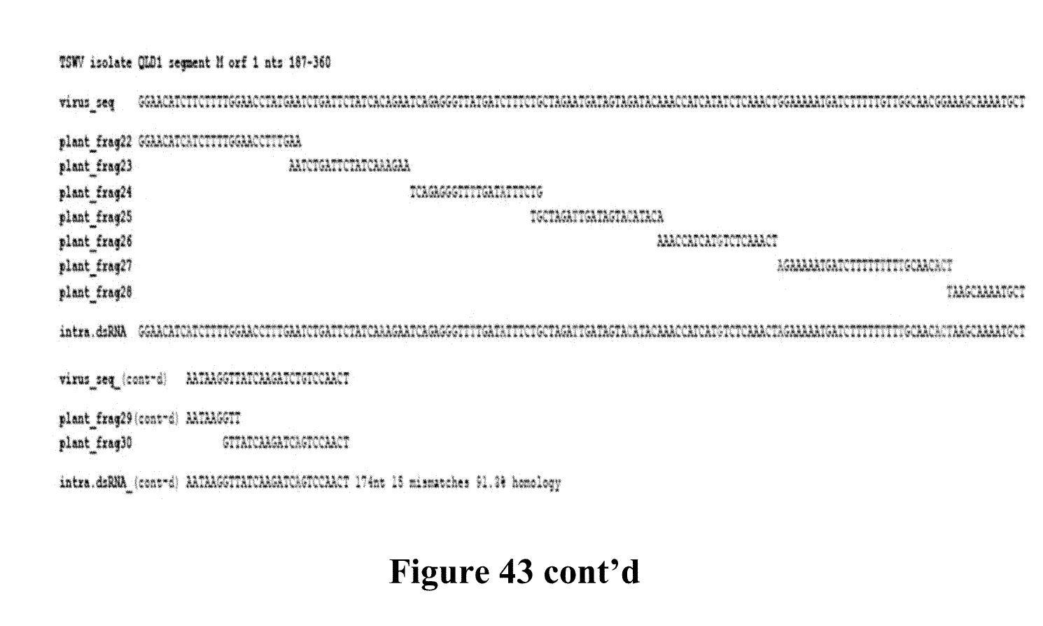

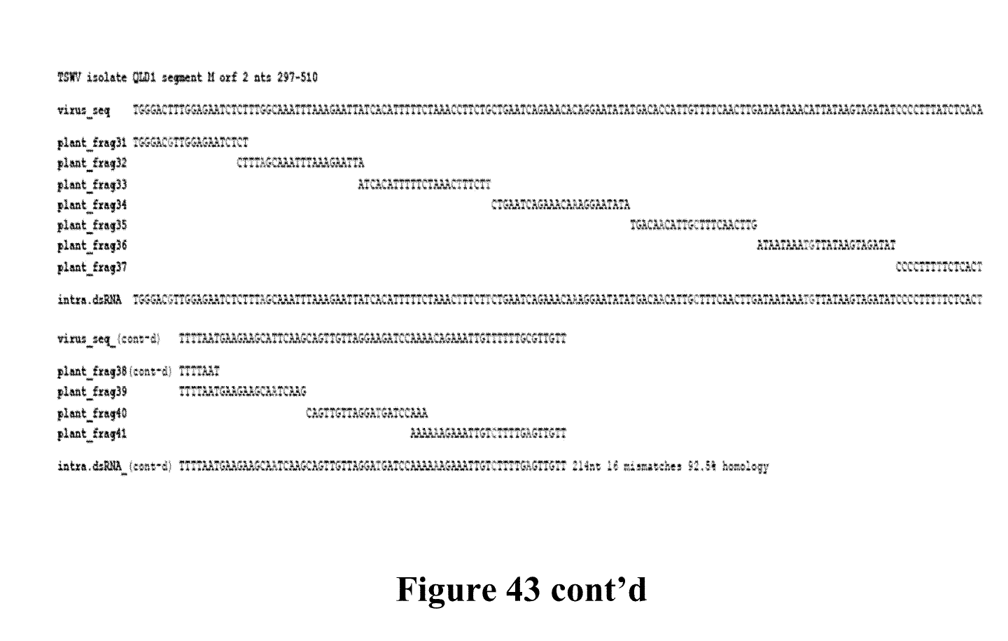

To test whether other hairpin RNAi constructs could be produced using plant-derived sequences for the invention, a long RNAi construct spanning several hundred nucleotides was designed comprising RNAi sequence that targets TSWV (SEQ ID NO:94). The intragenic RNAi sequence was created by blasting TSWV-QLD1 segment sequences against the tomato genome, selecting the best matching fragments of ≥20nt in length and arranging them together with small overlaps where possible. FIG. 43 shows how tomato (cultivar Moneymaker) sequences were used and brought together to create SEQ ID NO:94, where each plant-derived sequence was at least 20 nts in length. The sequence displayed an overall match to TSWV sequence of 91%.

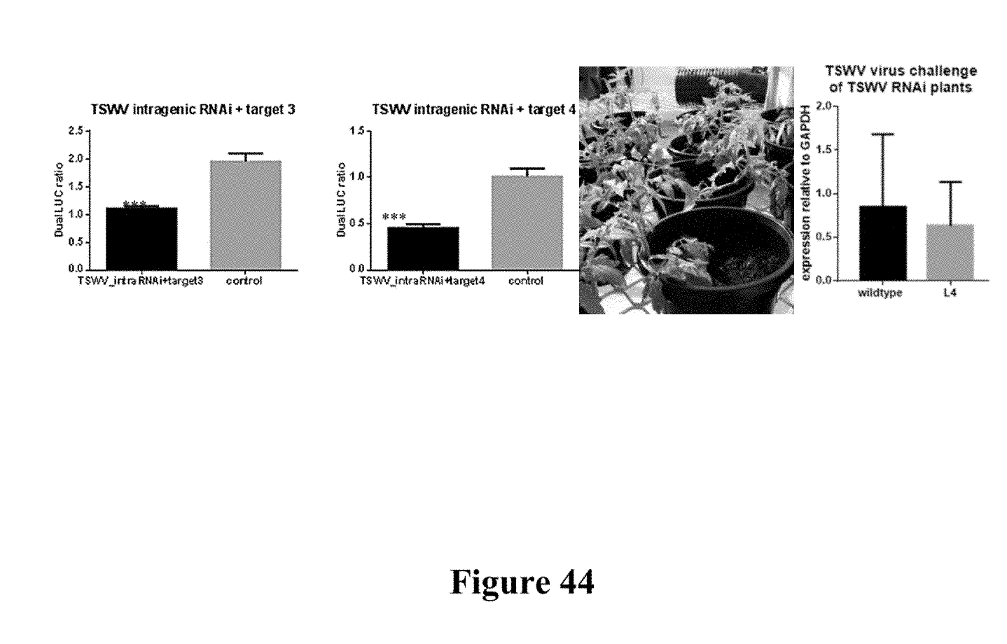

This sequence was tested for its RNAi silencing ability when brought into contact with four different corresponding TSWV target sequences (using the dual LUC assay as described herein). As shown in FIG. 44, the TSWV RNAi construct caused a strong knock-down of expression for two of the four targets, relative to the control (P<0.001; Student's t test).

For tomato transformation, an intragenic RNAi construct was first built in pKannibal by including the TSWV RNAi sequence (SEQ ID NO:94) in sense direction, followed by the PDK intron sequence as spacer and the anti-sense TSWV RNAi sequence. The cassette was then transferred into pArt27 using SacI and SpeI sites. The complete sequence of the corresponding vector is set forth is SEQ ID NO:95. Plants were regenerated and 14 lines were confirmed to contain the intragenic construct. These had normal phenotype and tomato seeds were collected for TSWV challenge testing of T1 seedlings (FIG. 44). T1 seedlings from one of these lines (L4) displayed slightly reduced levels of TSWV infection when tested by qRT-PCr (FIG. 44) and further testing of other lines is underway.

### Example 6. Developing Rapid Intragenic Strategies to Provide Useful Traits in Crop Plants Across Other Species

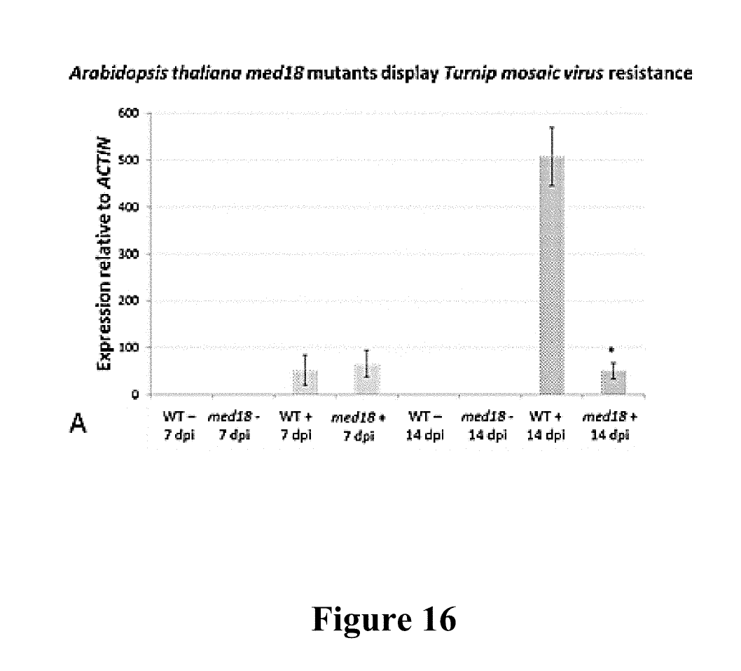

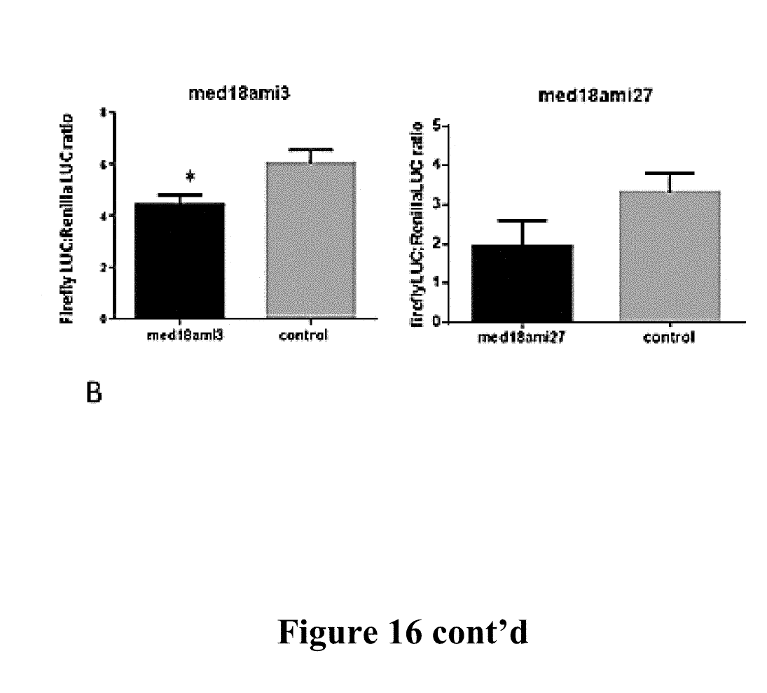

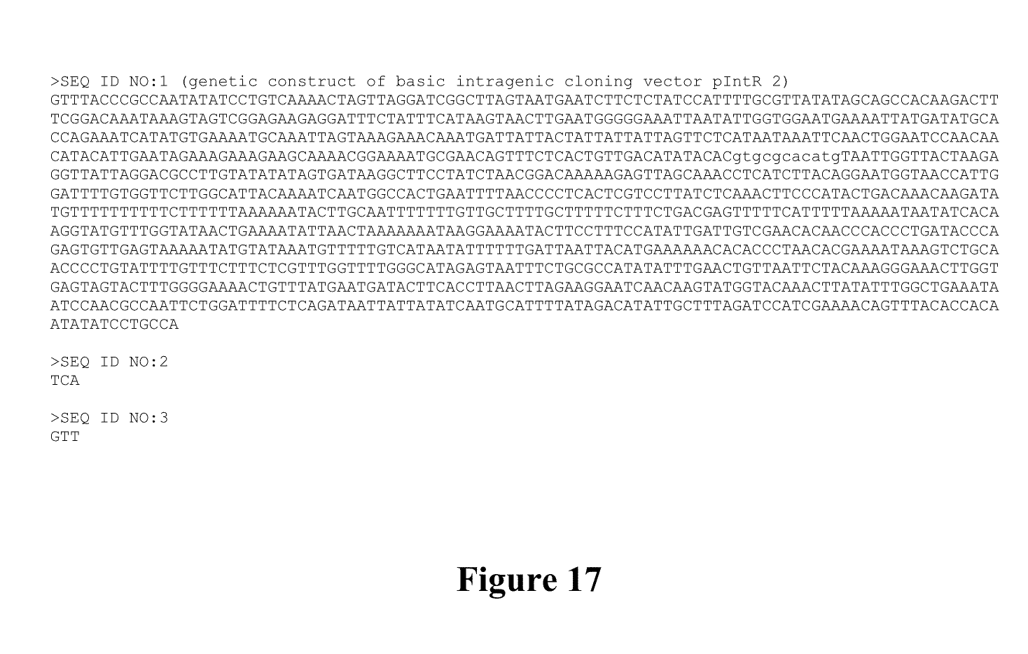

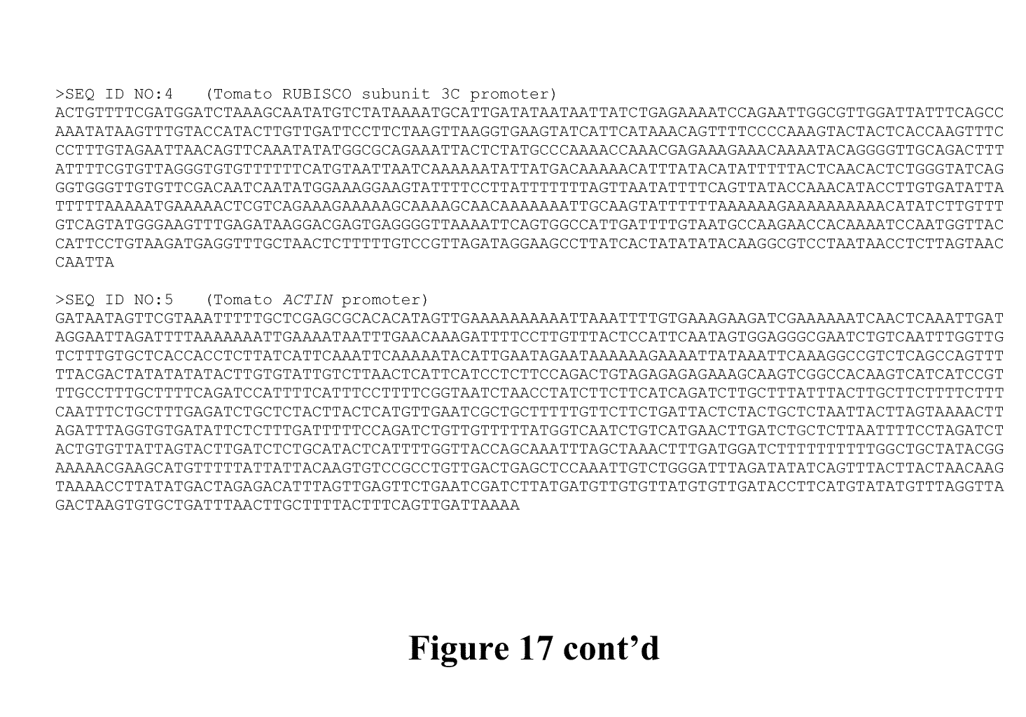

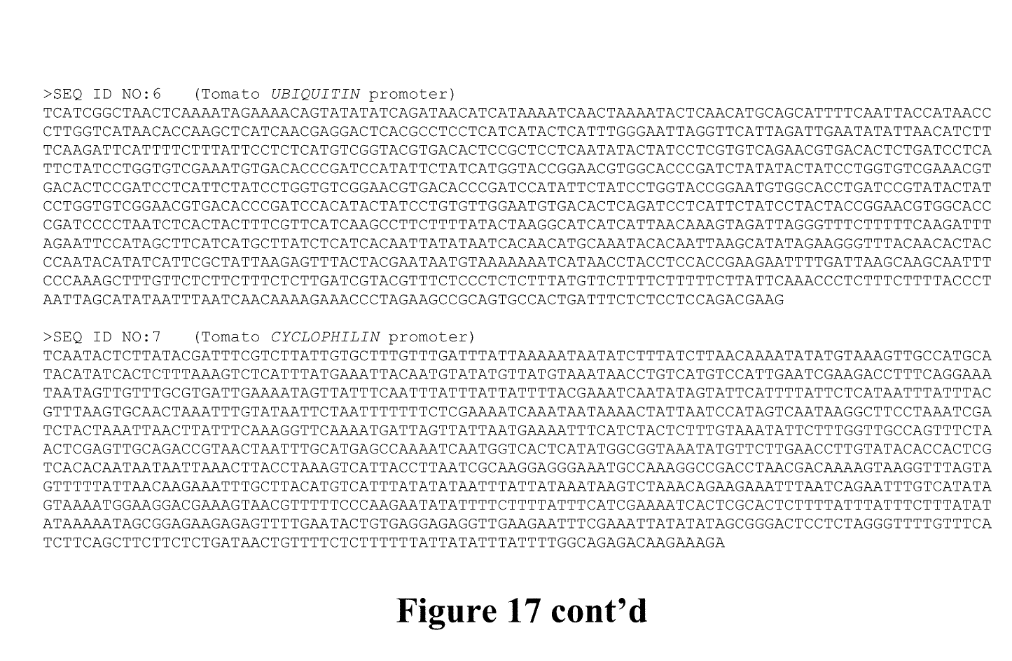

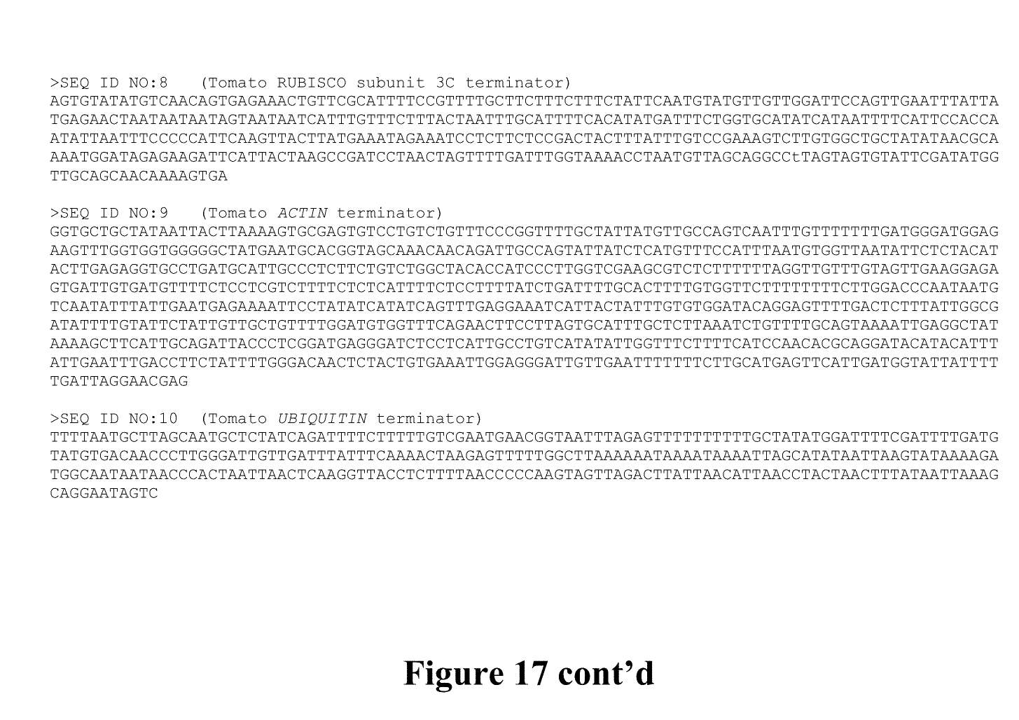

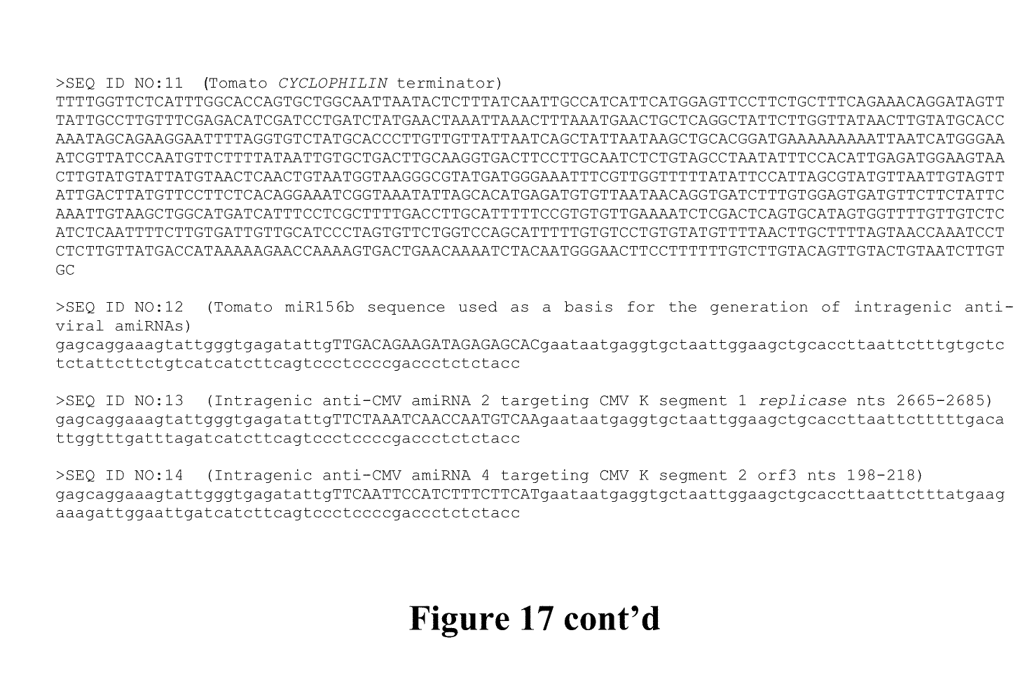

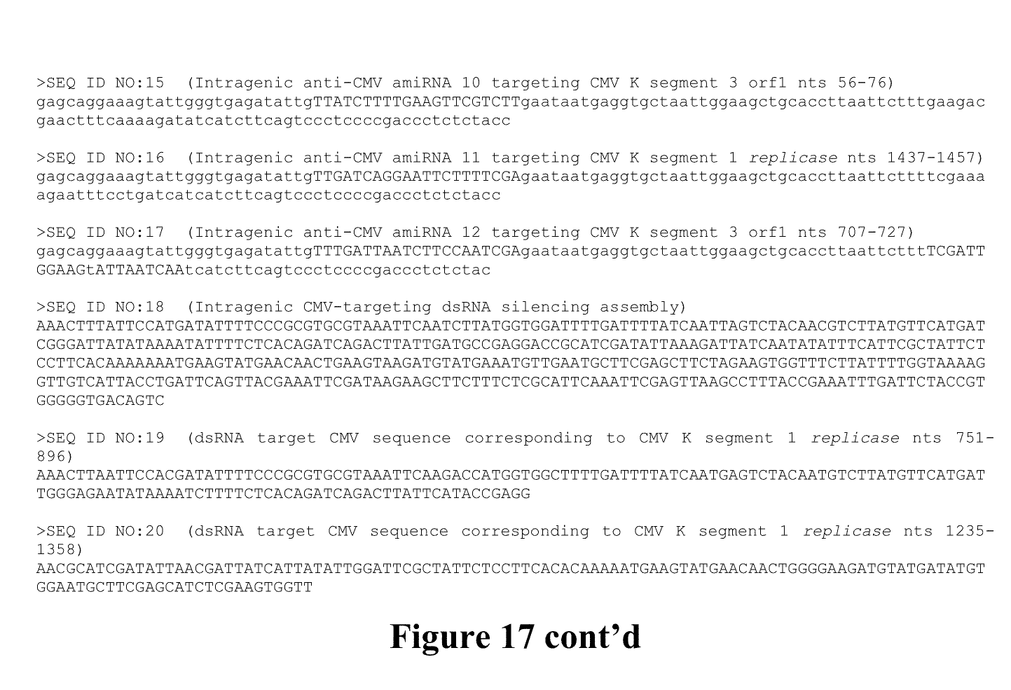

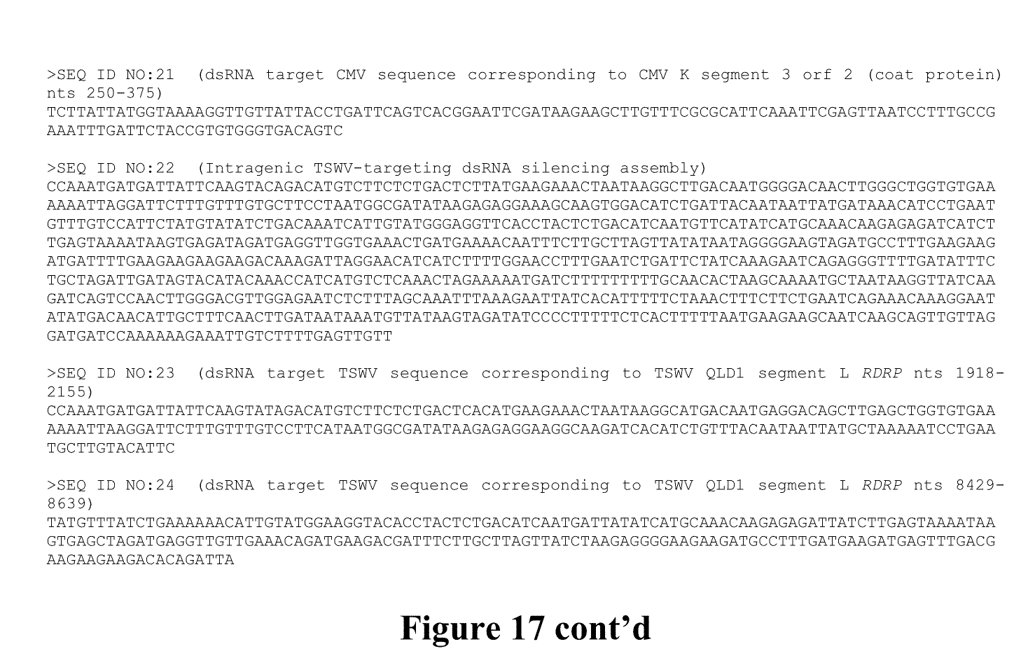

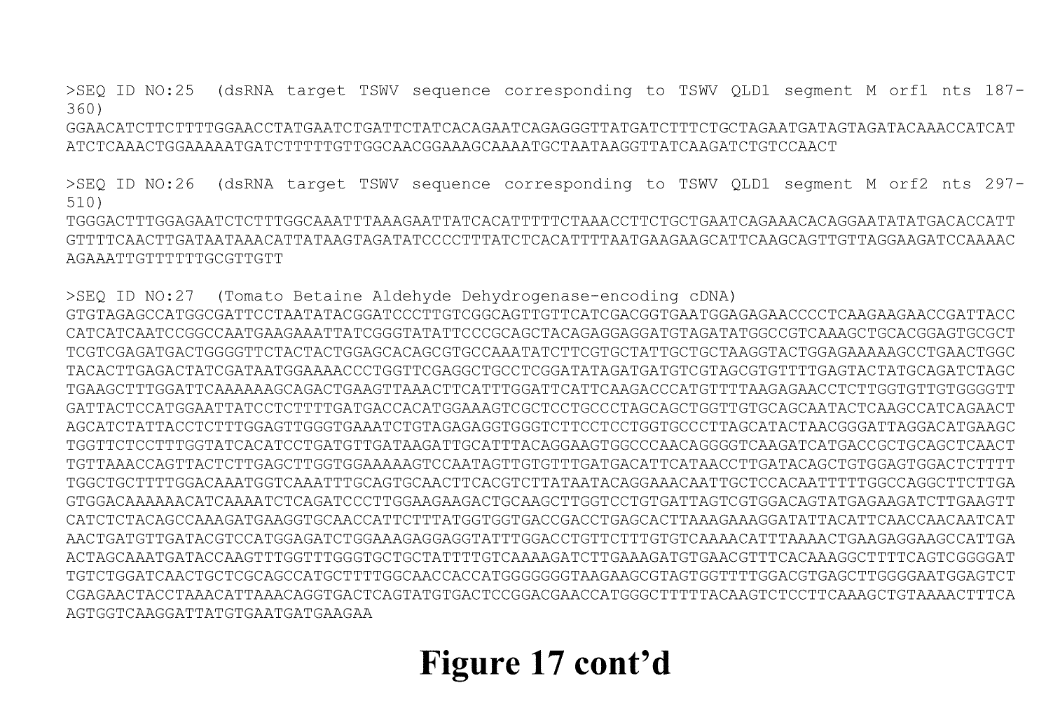

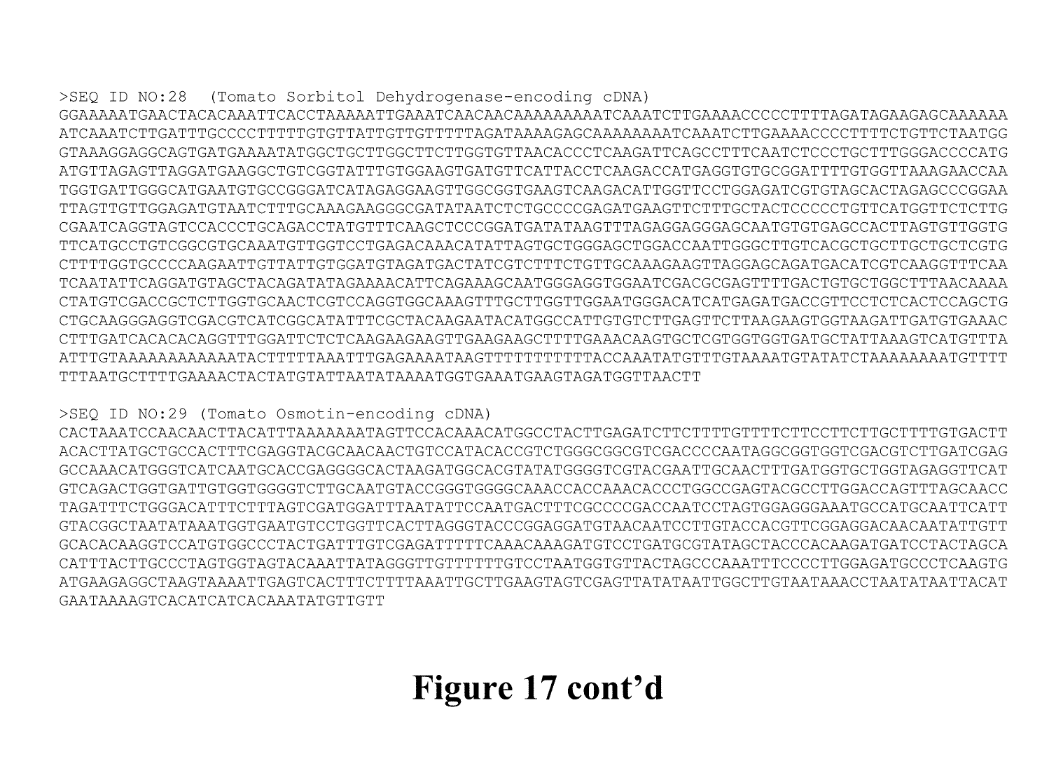

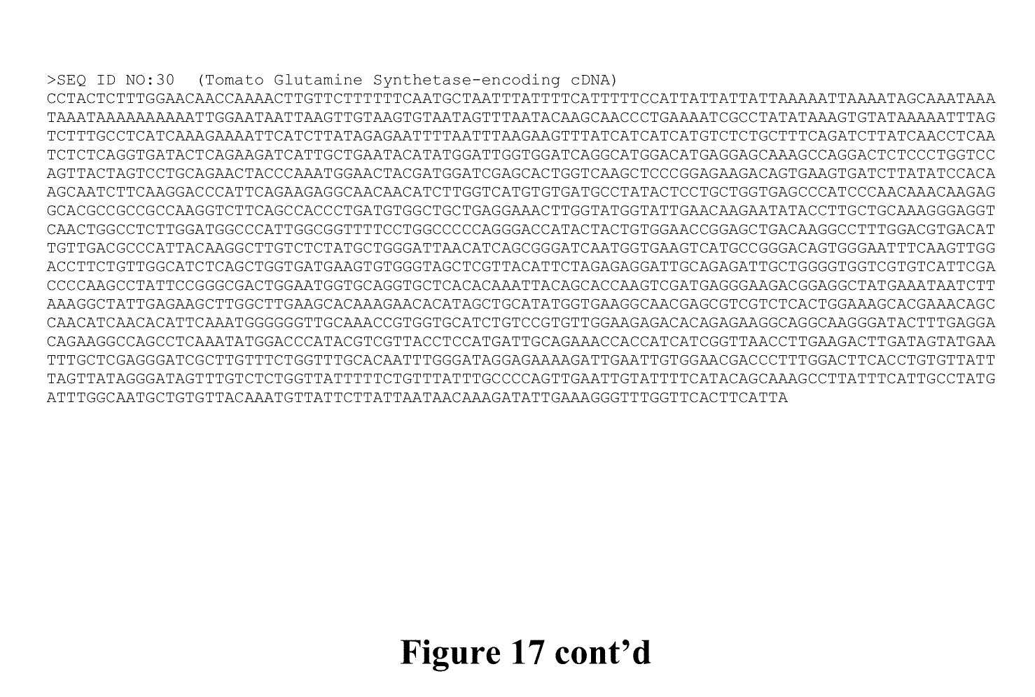

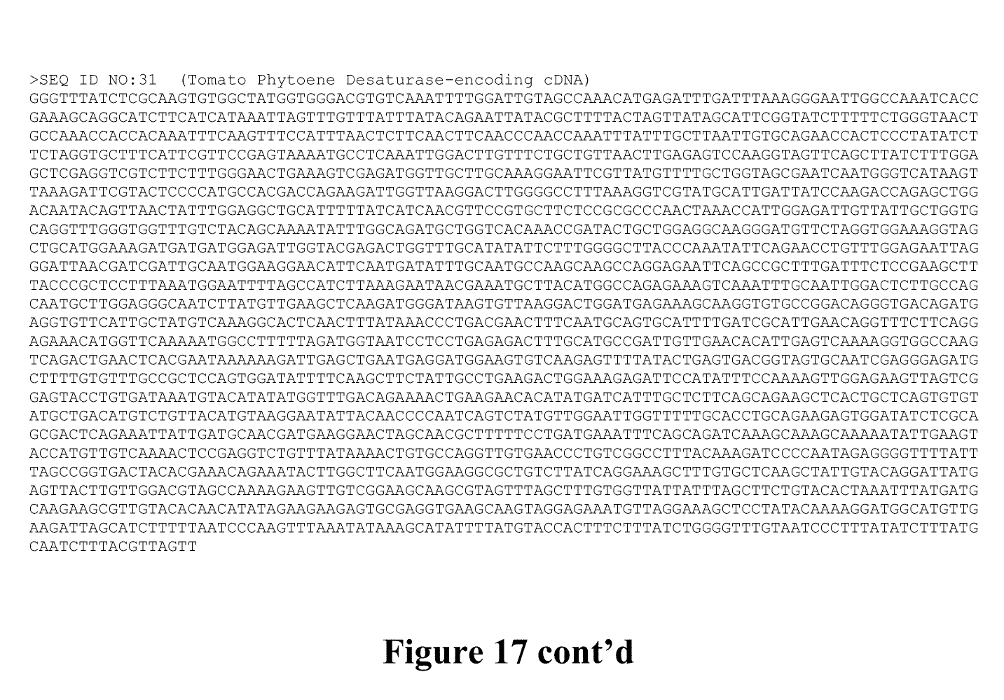

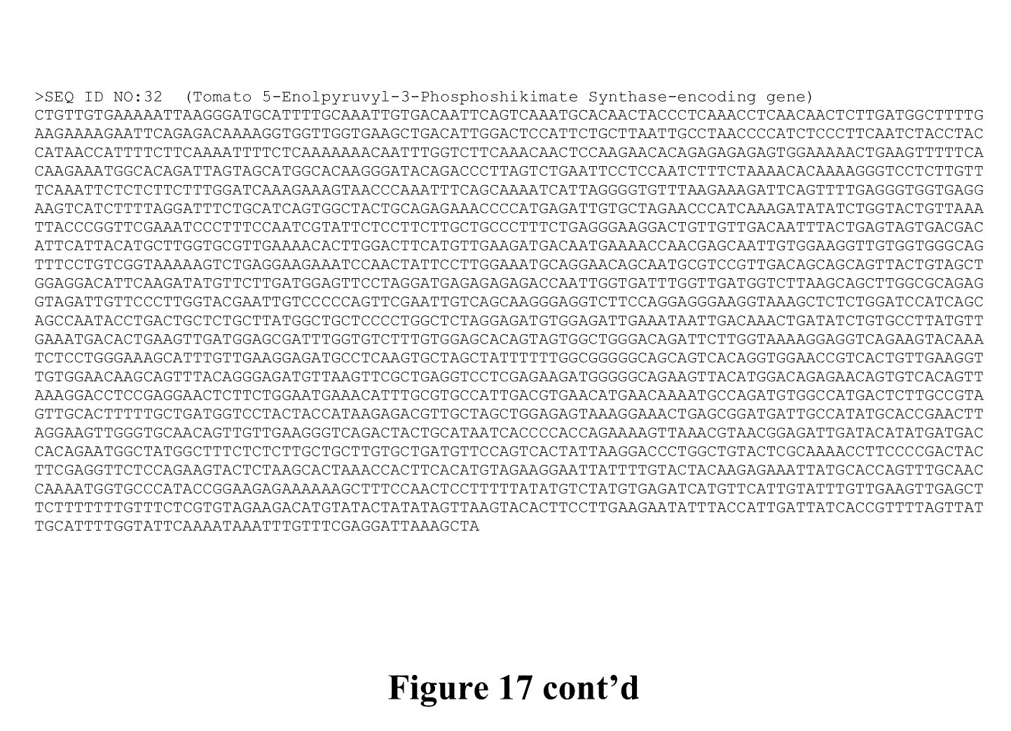

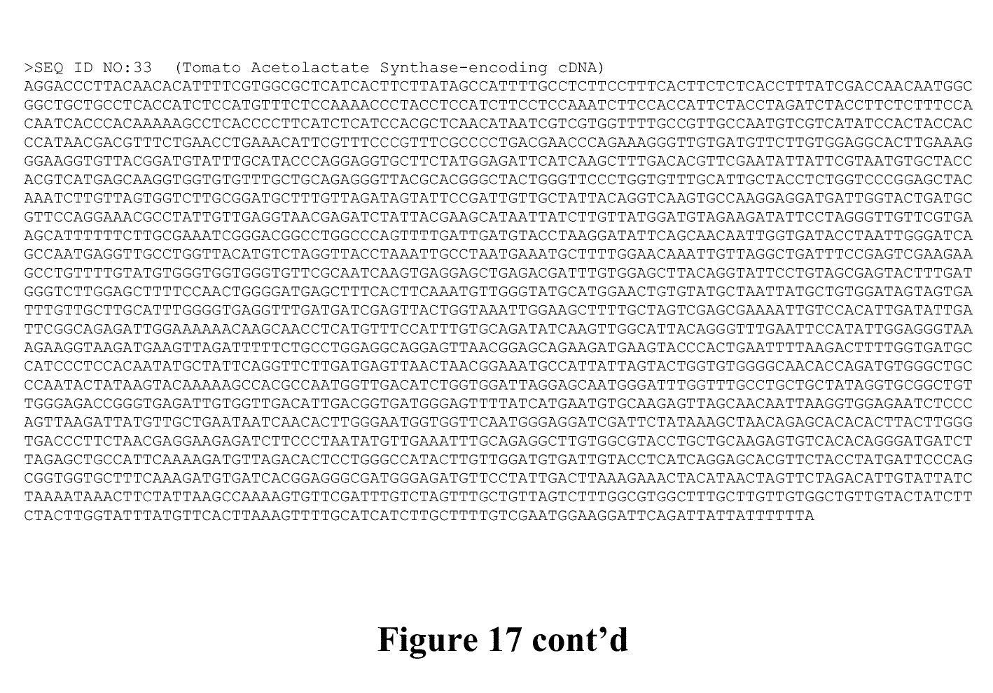

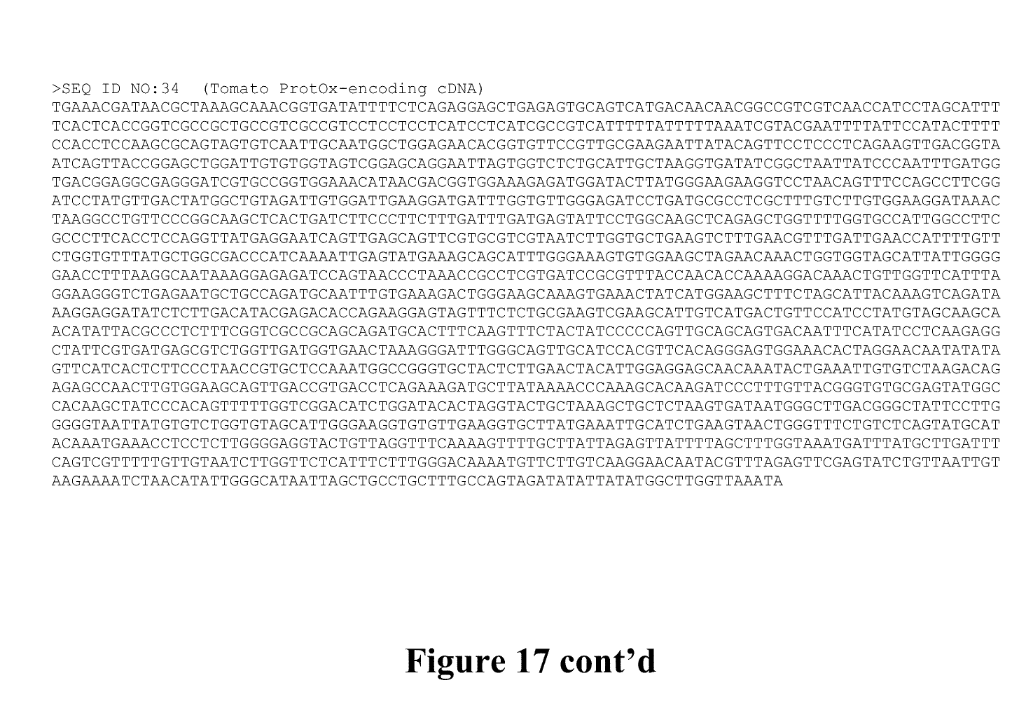

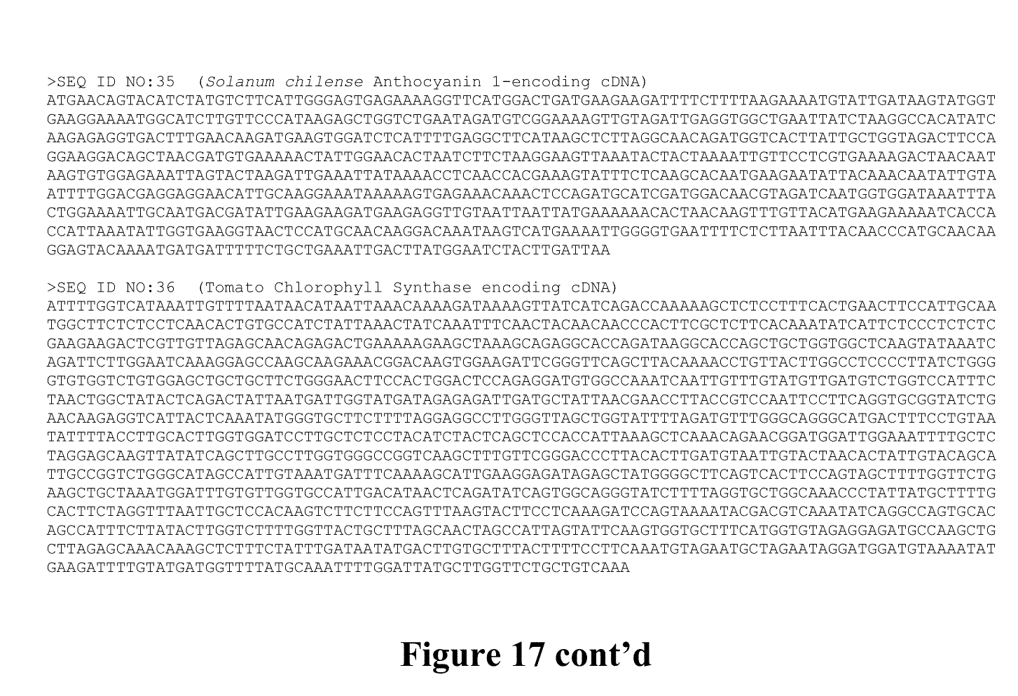

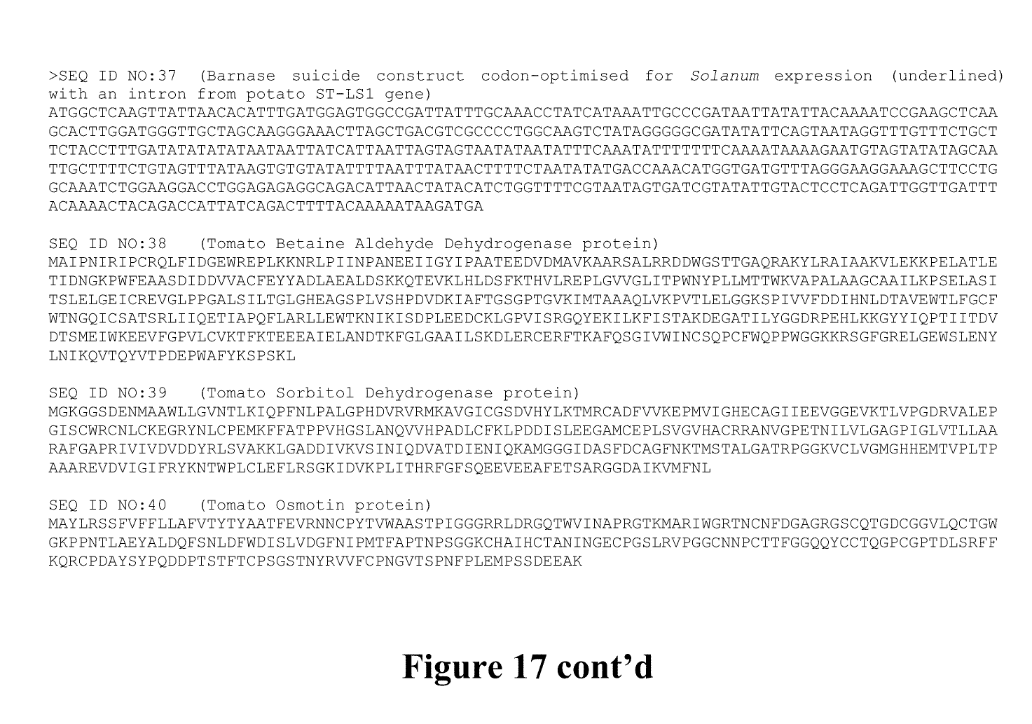

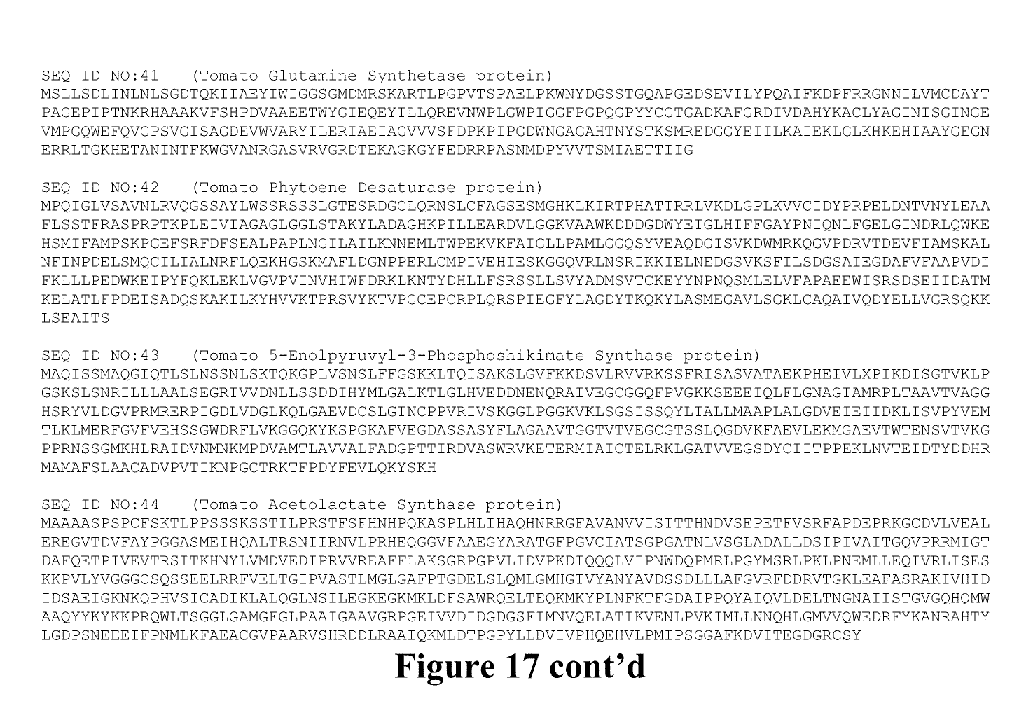

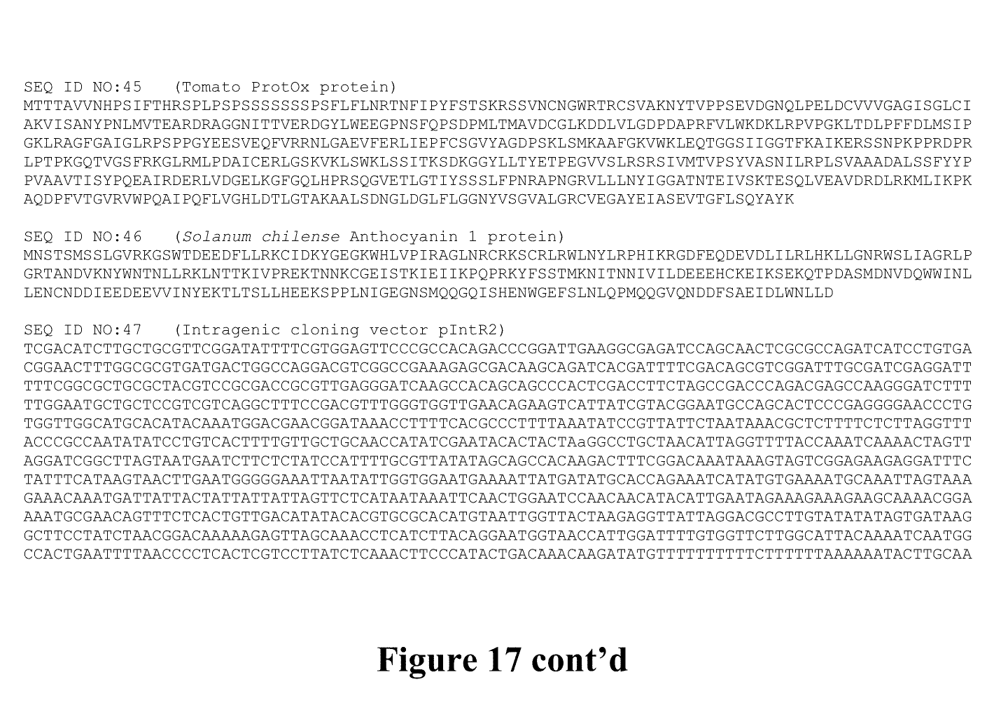

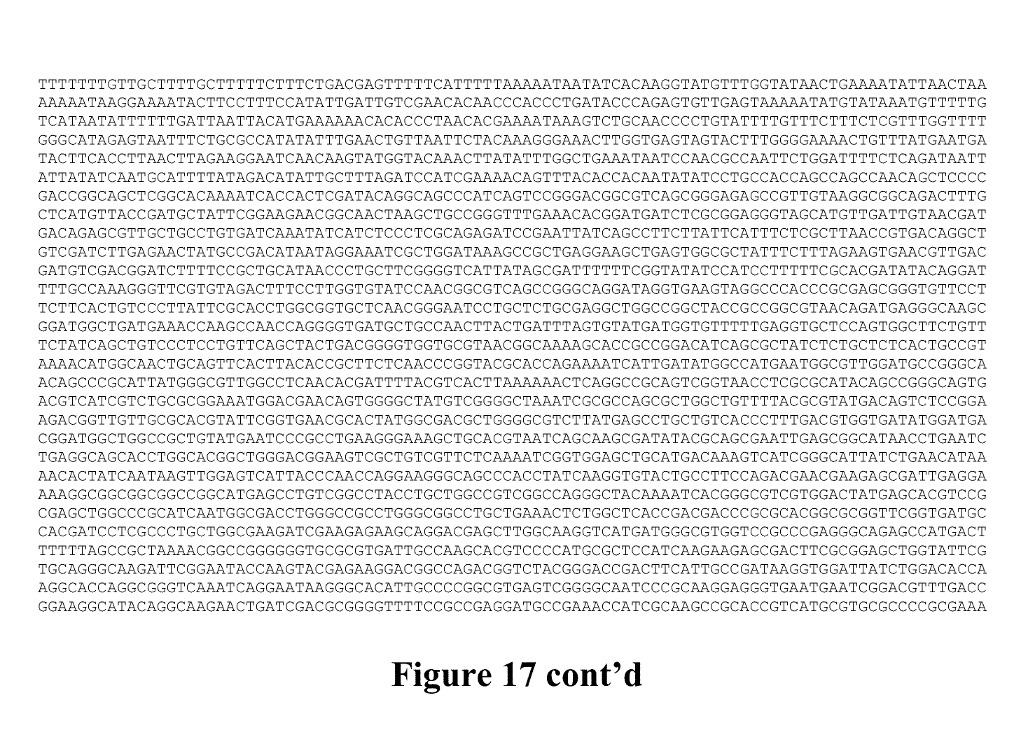

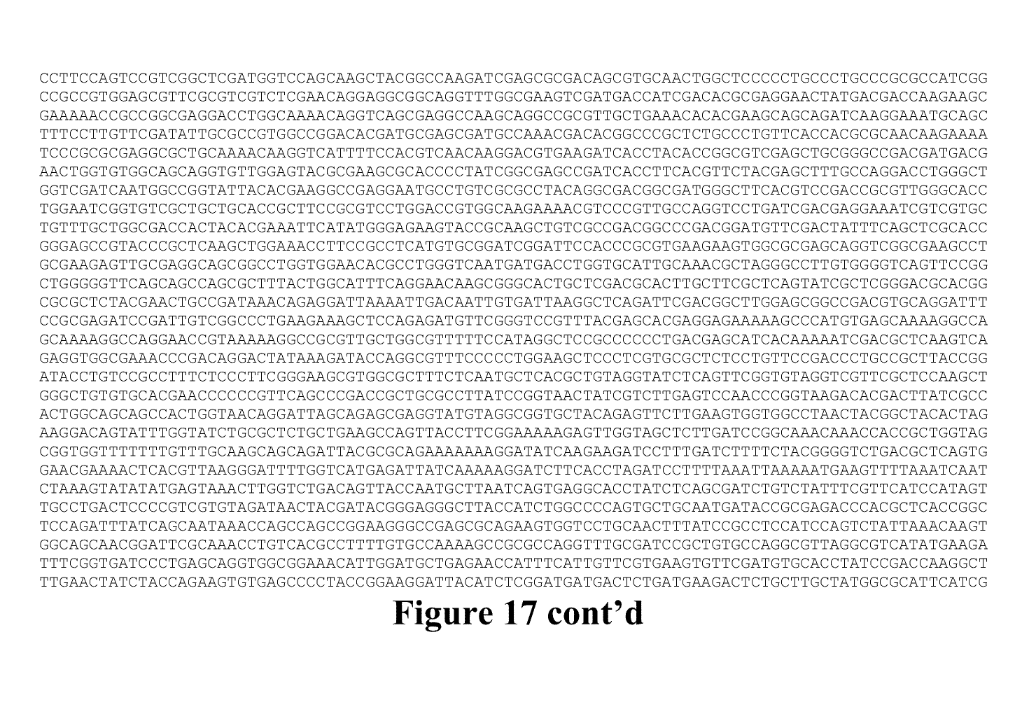

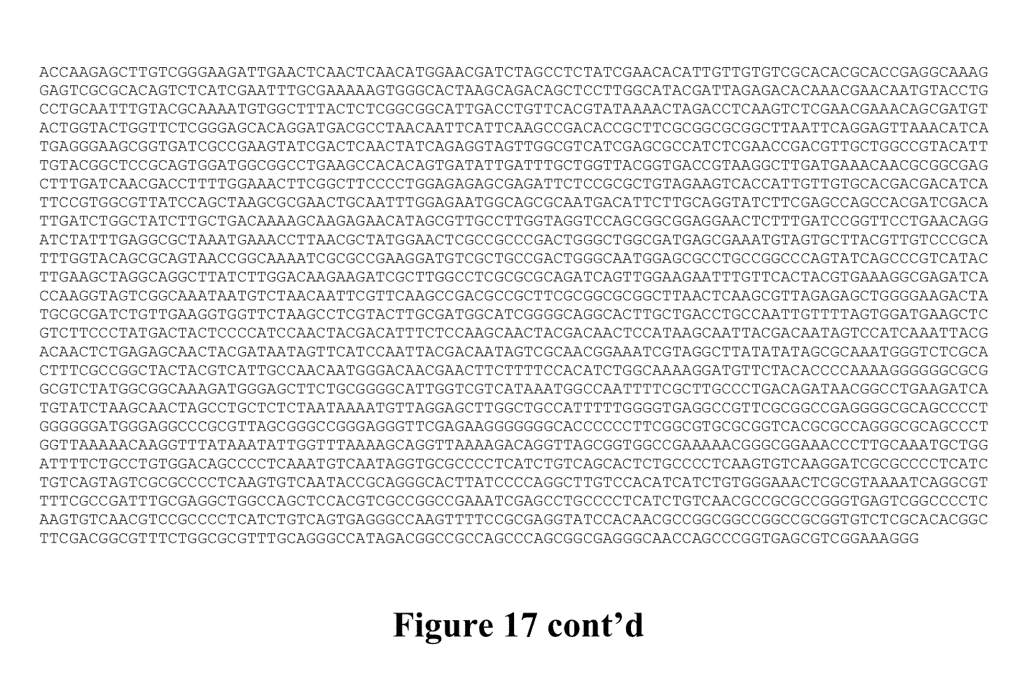

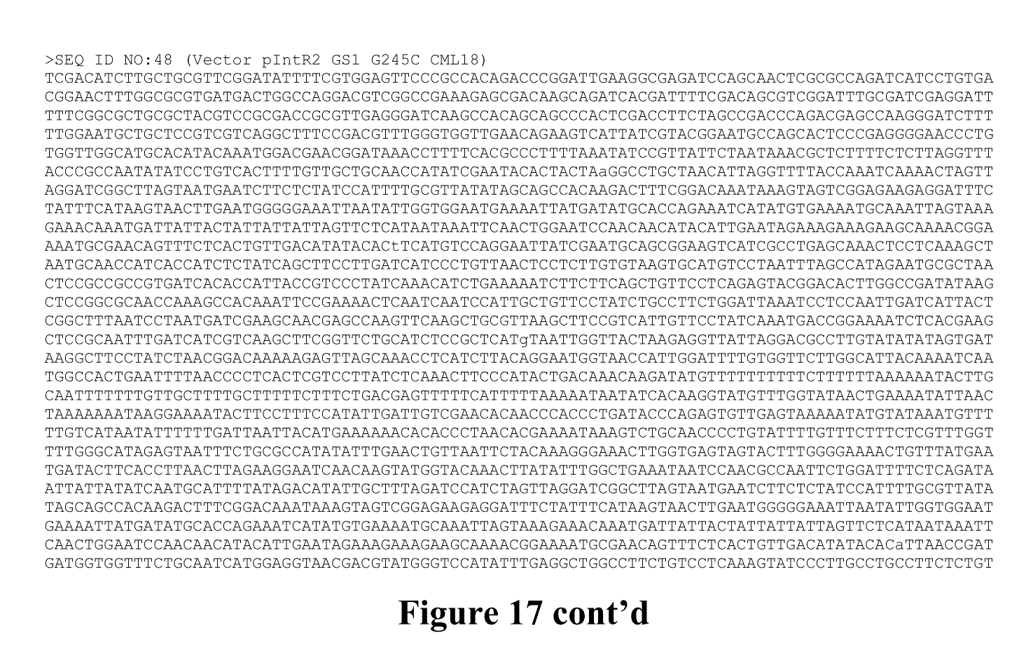

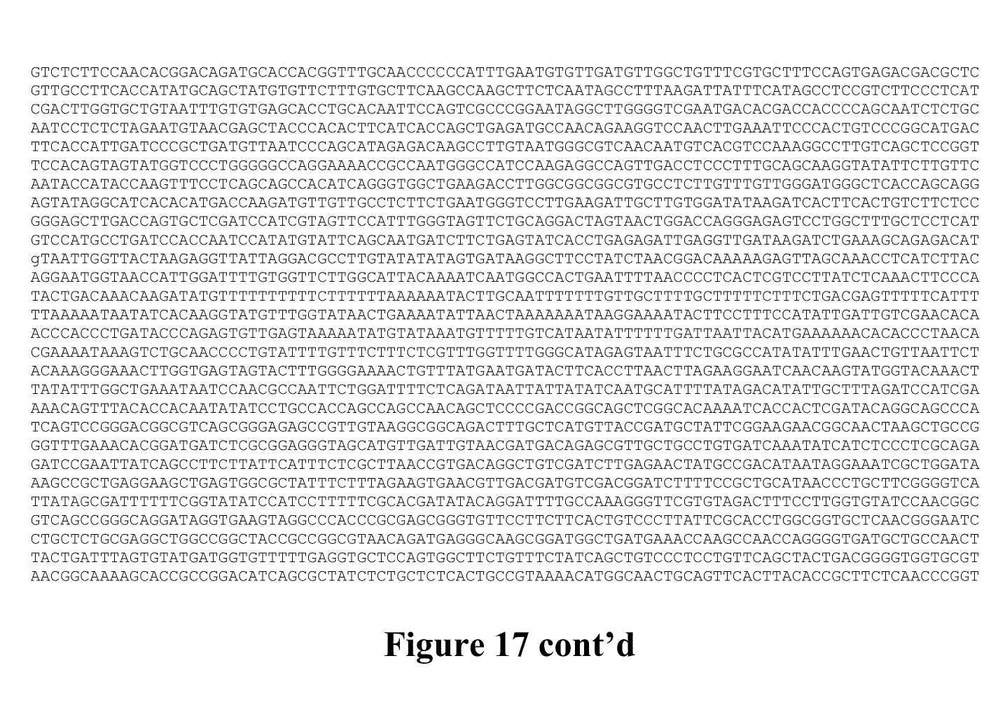

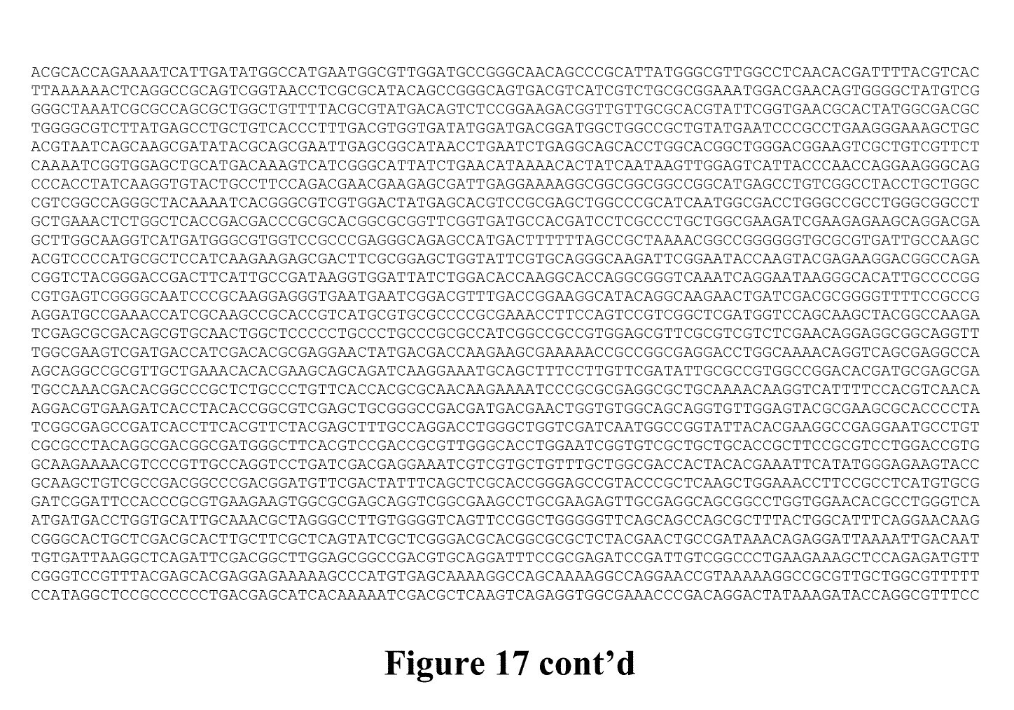

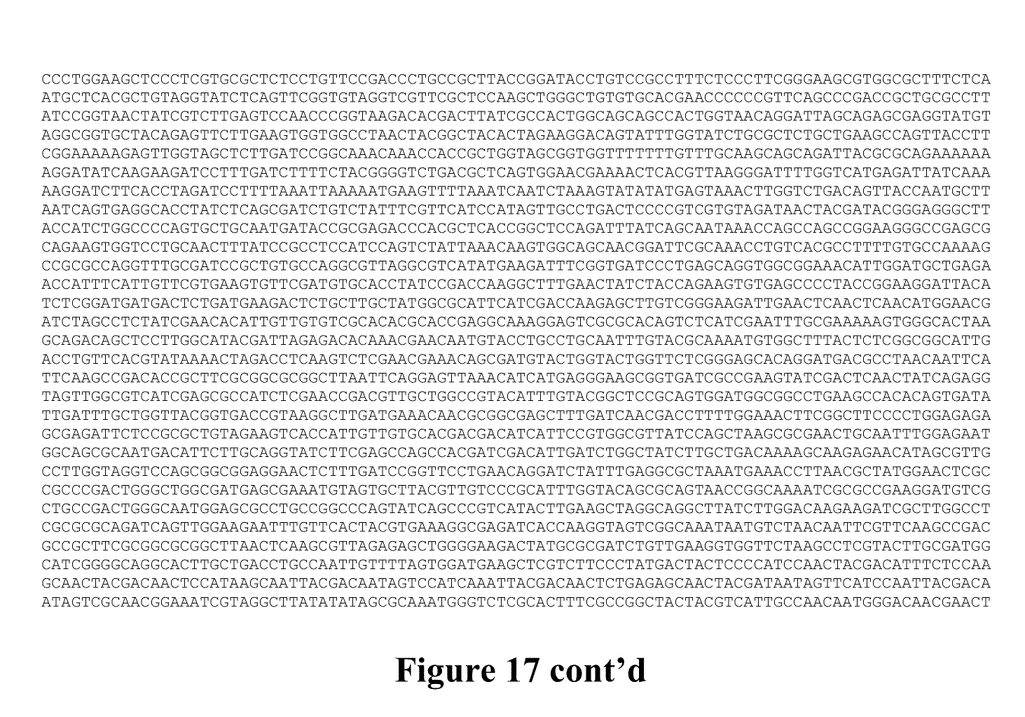

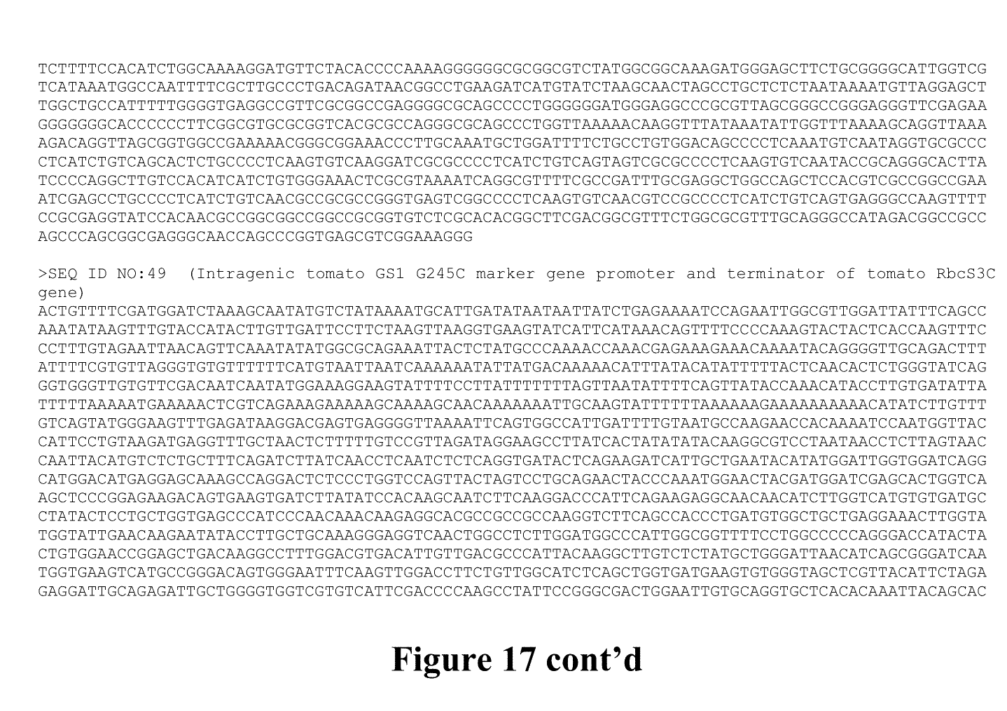

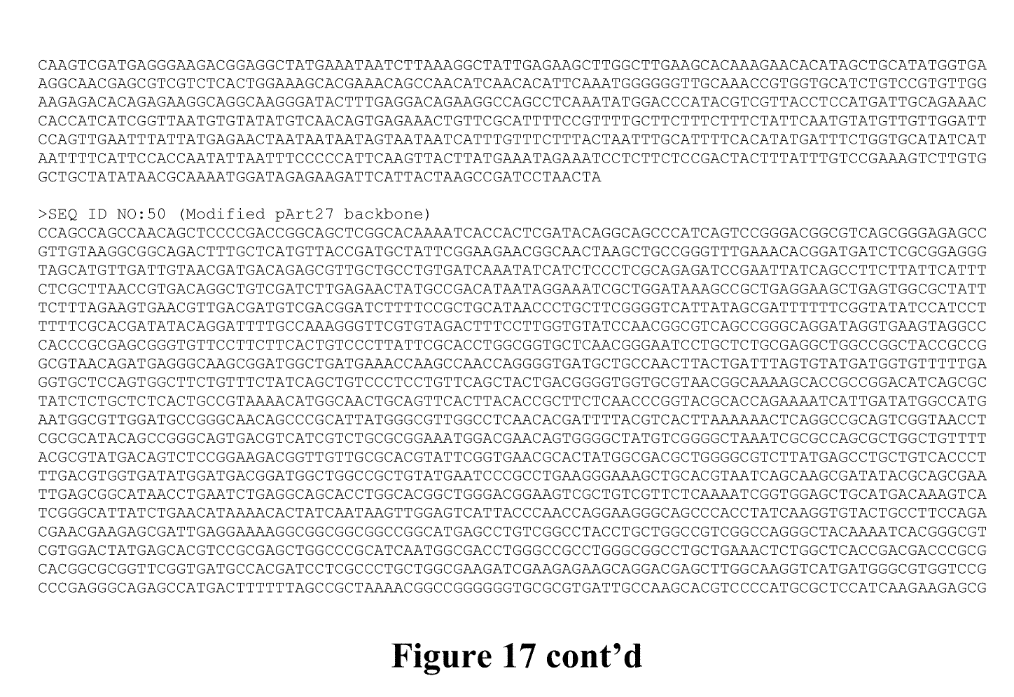

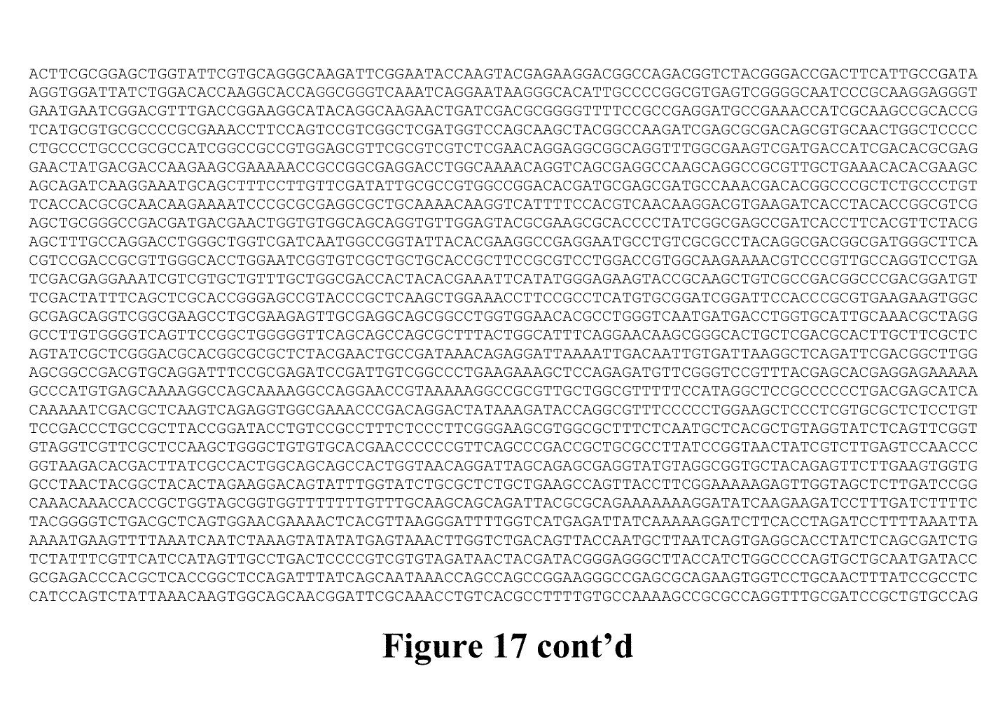

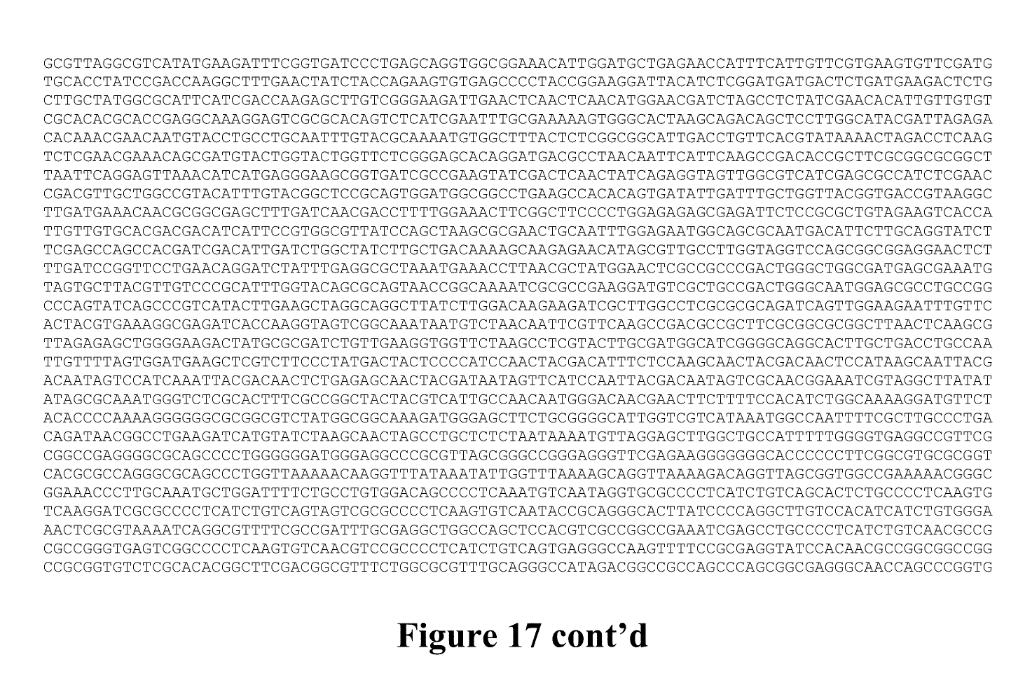

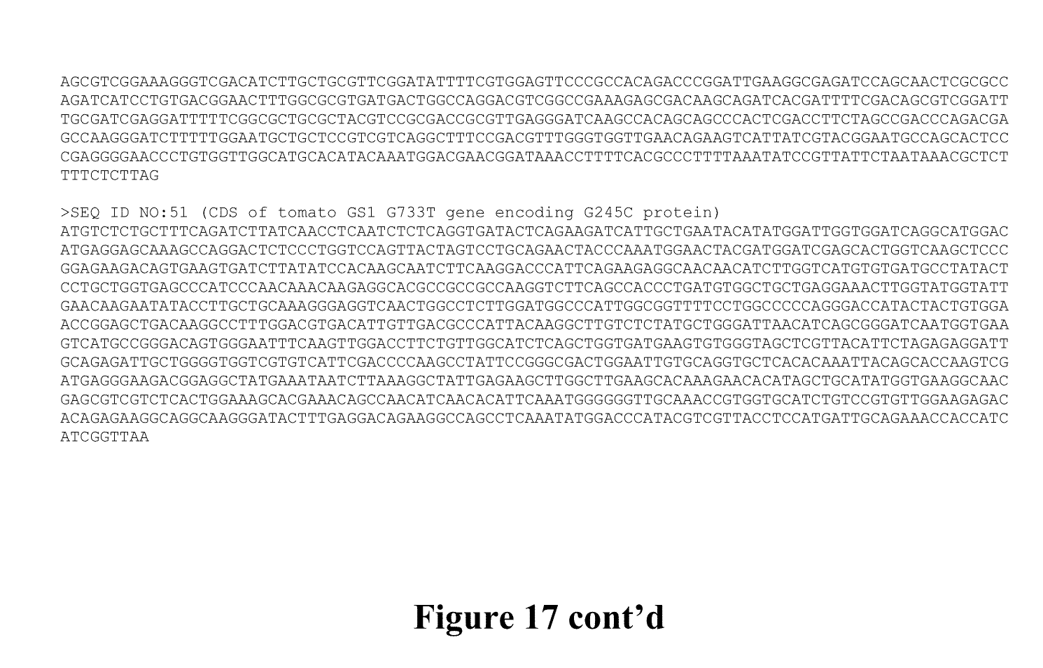

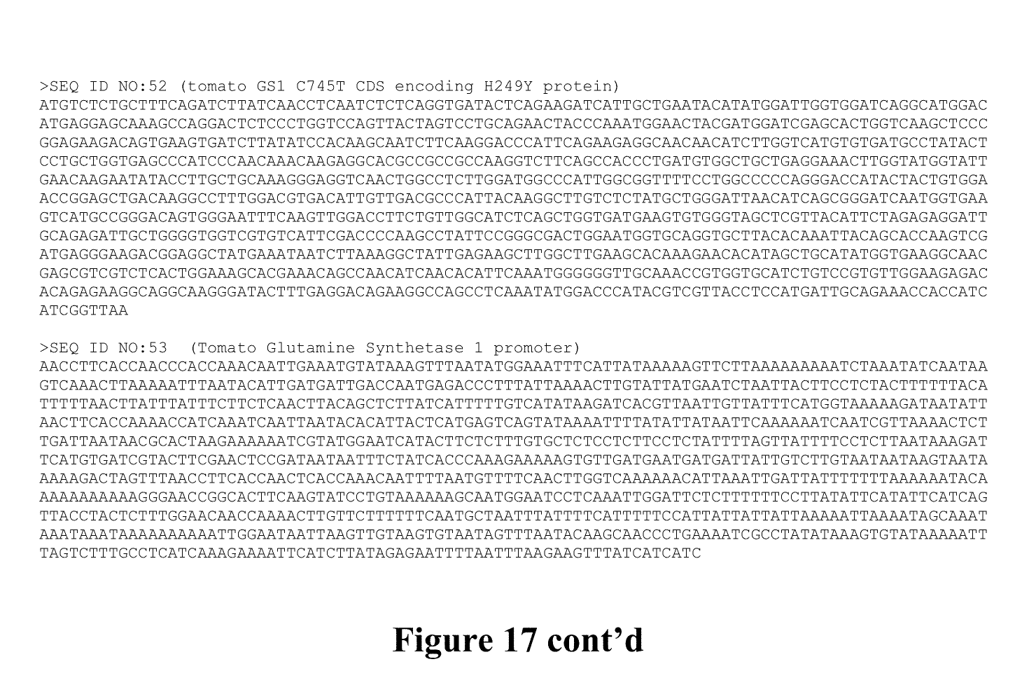

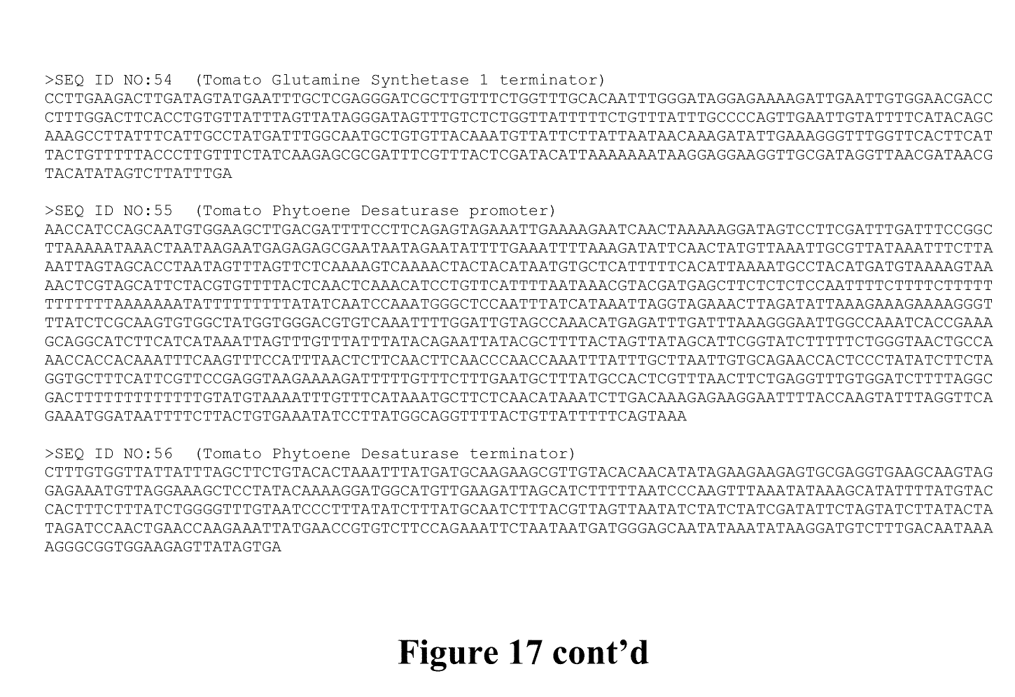

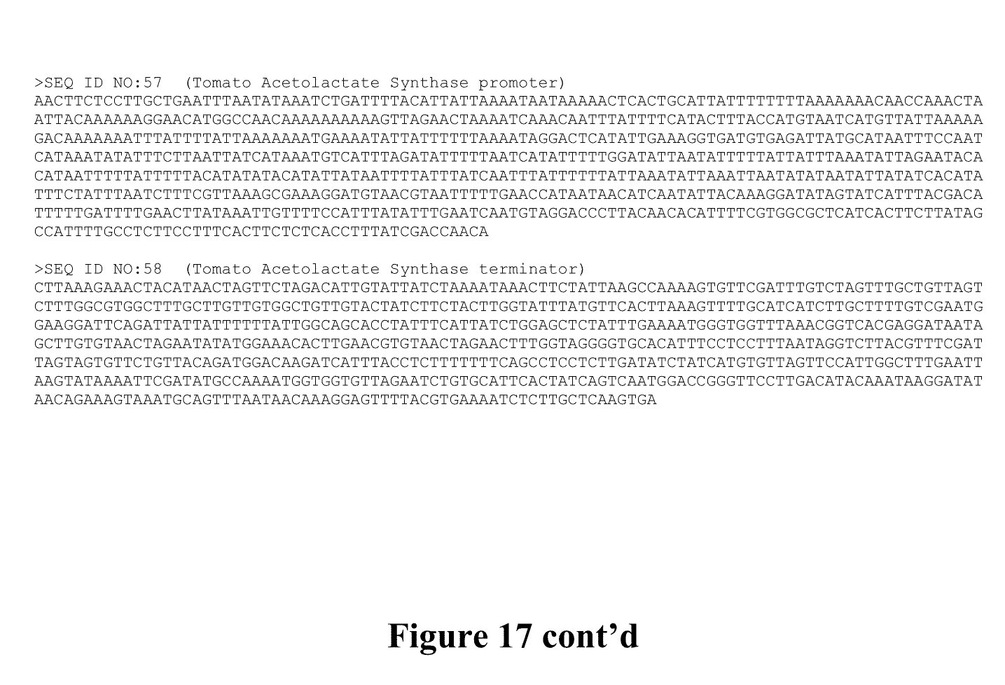

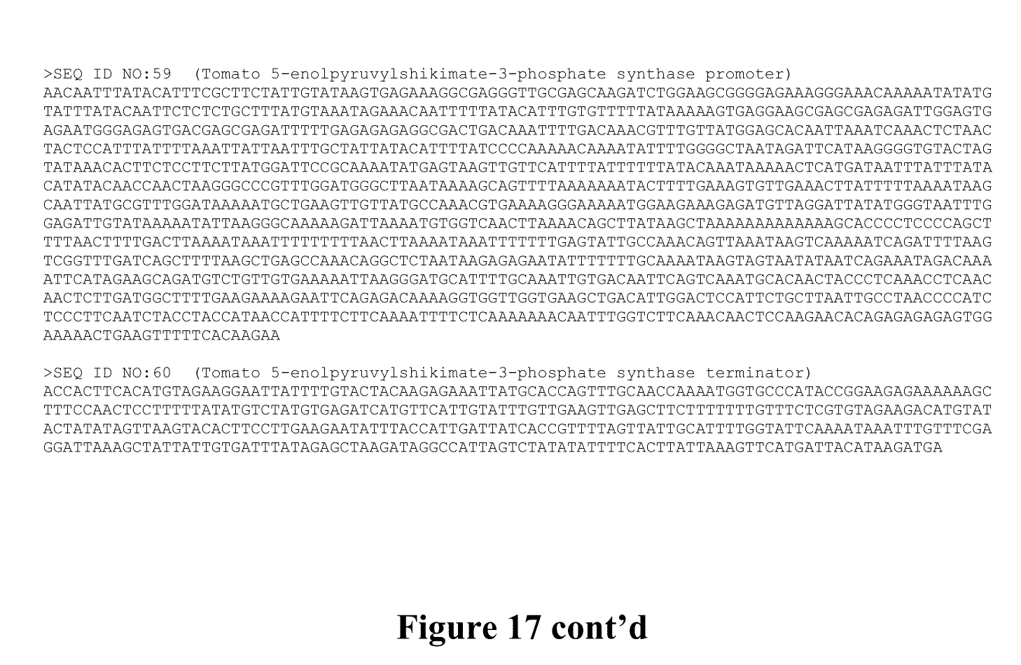

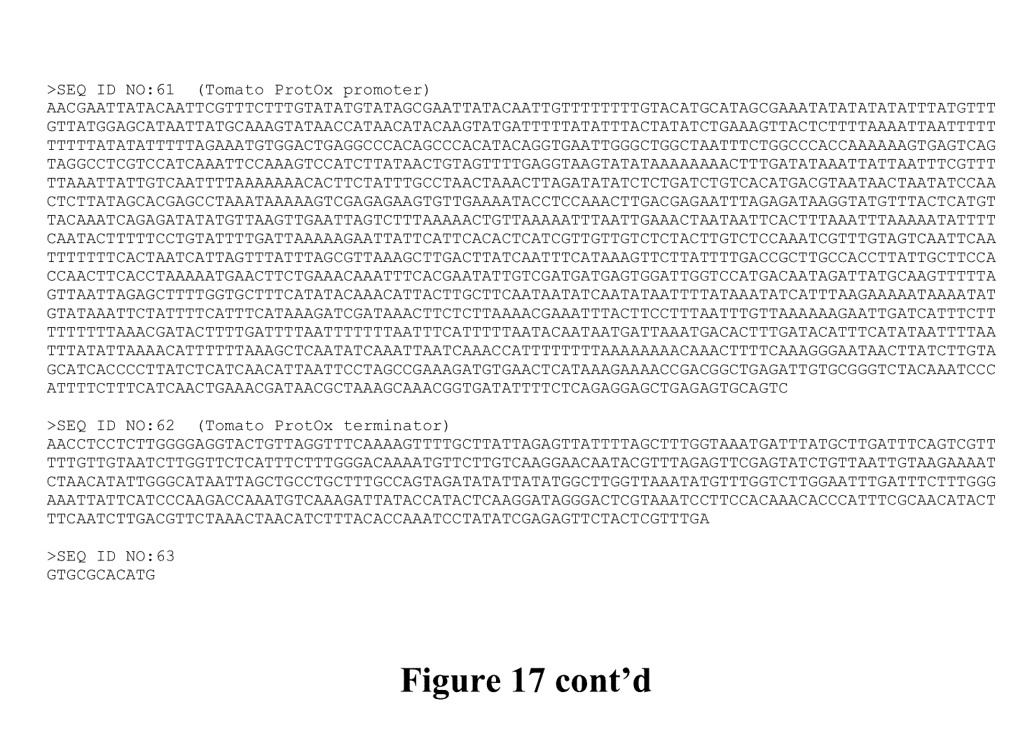

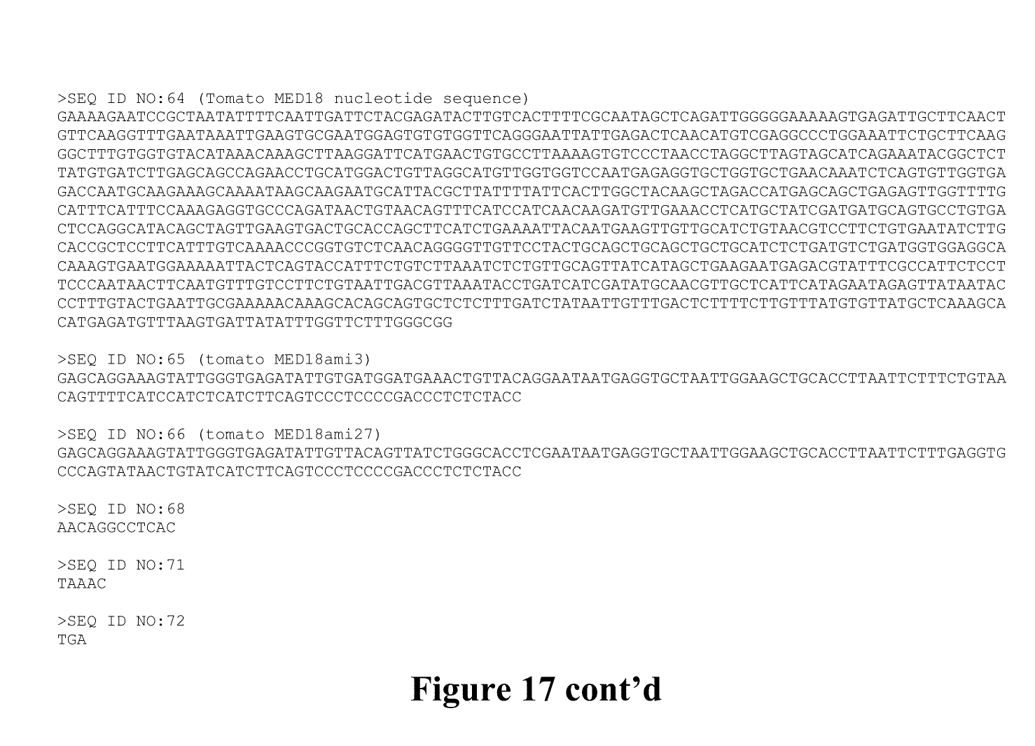

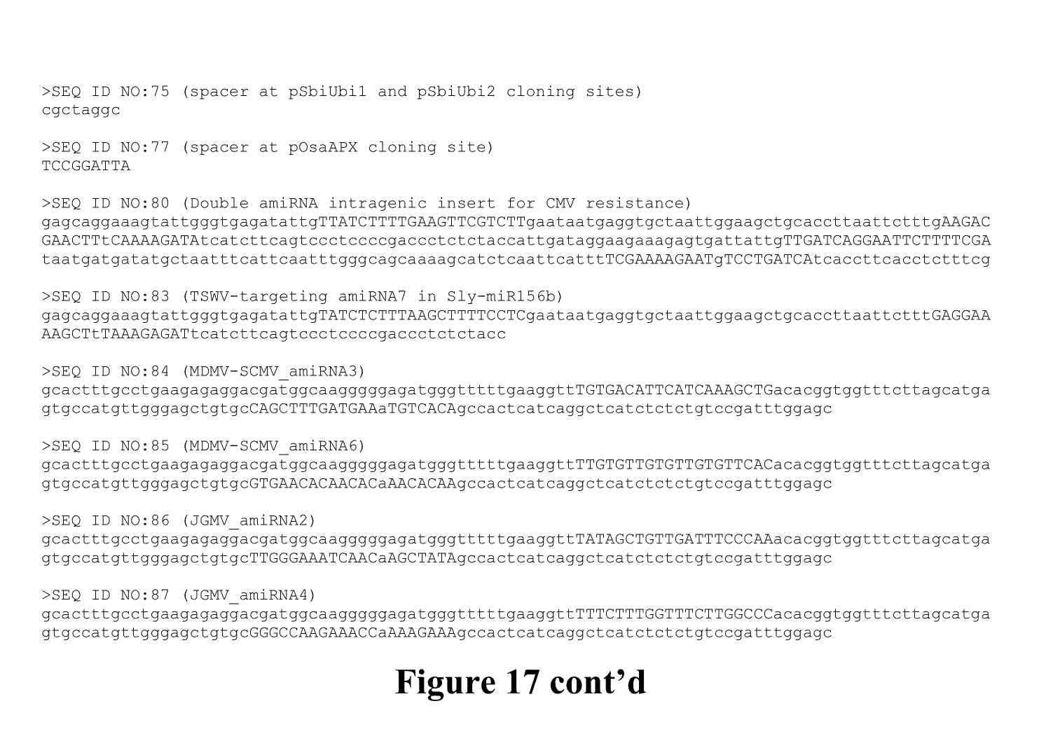

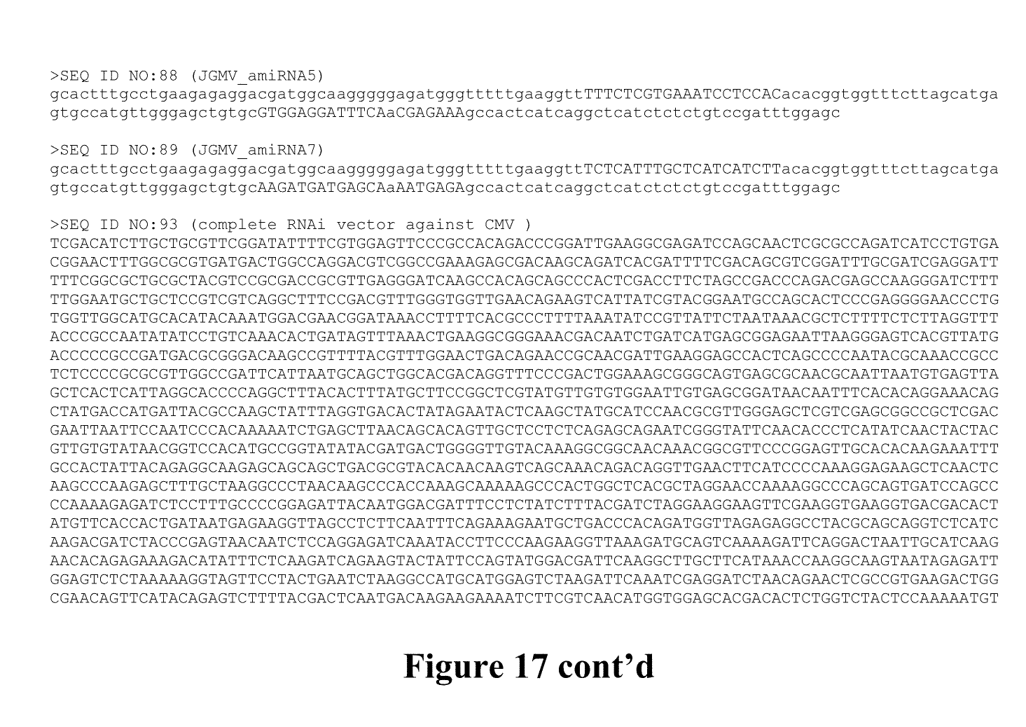

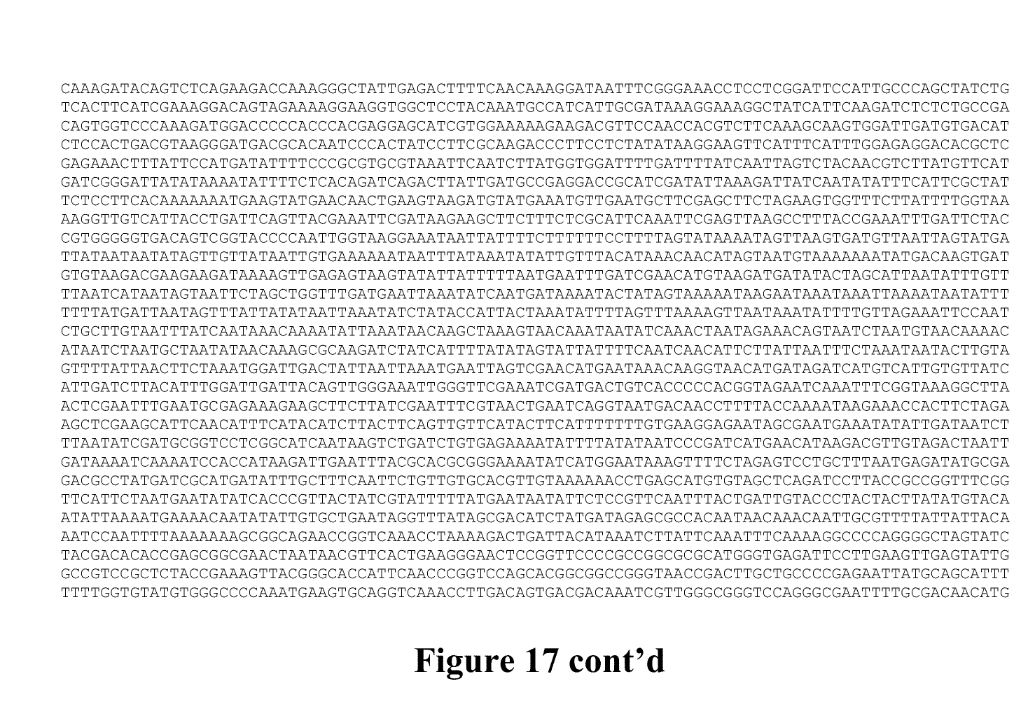

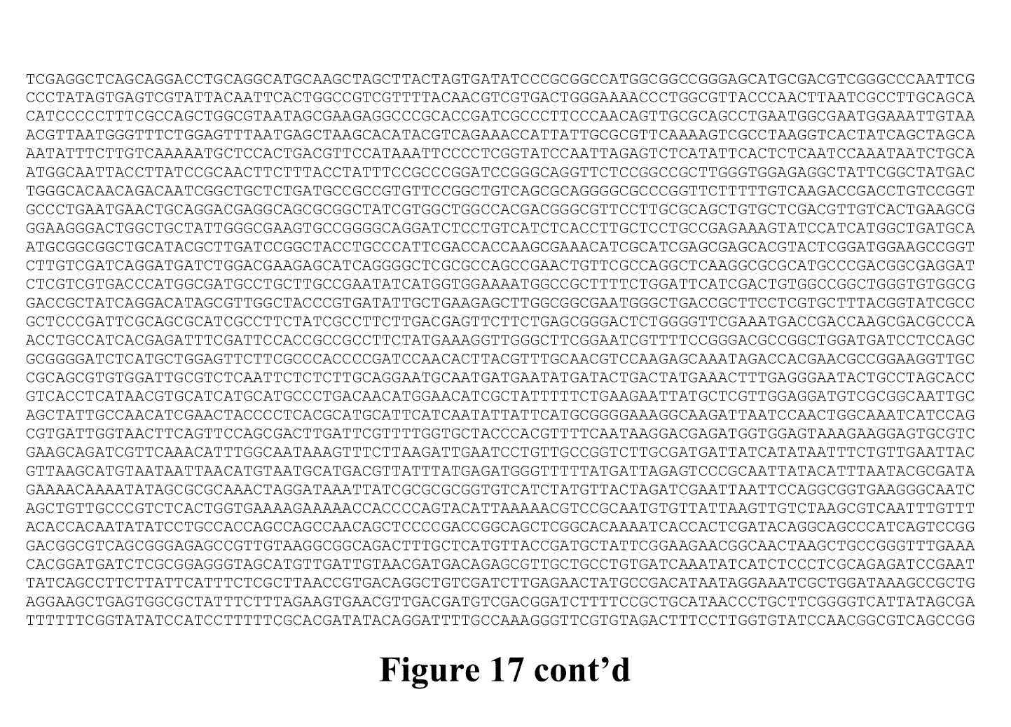

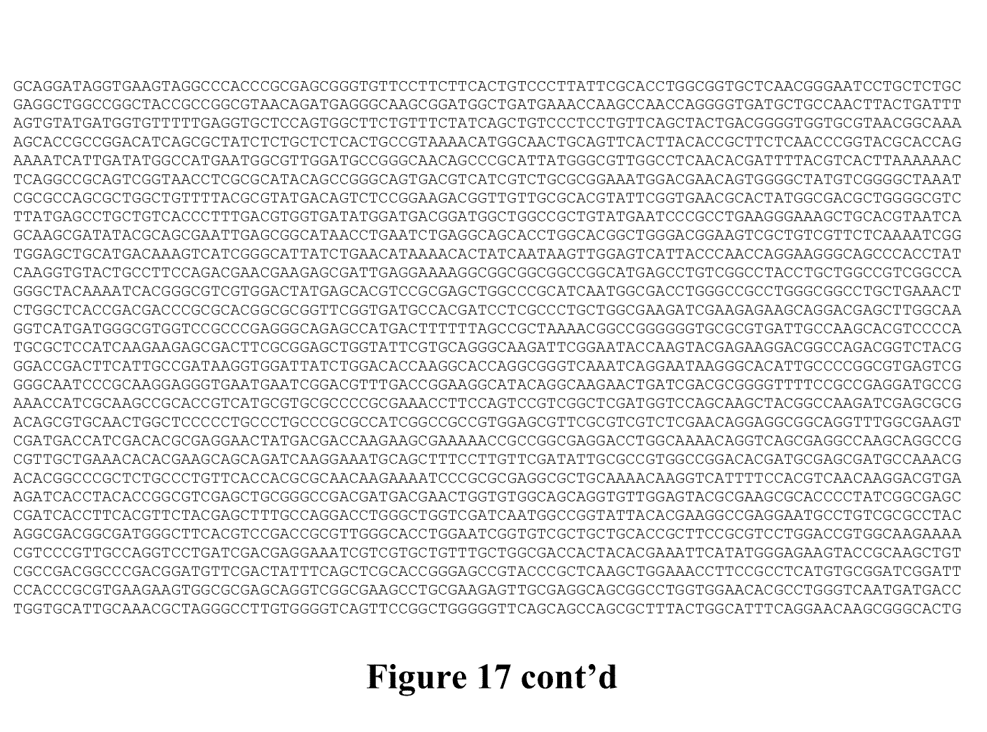

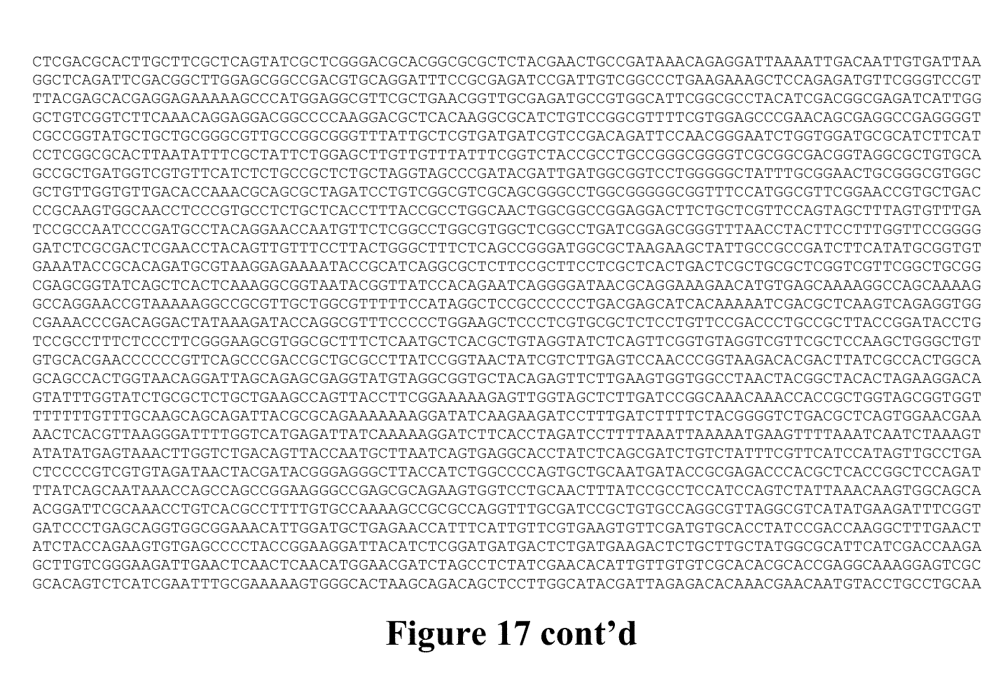

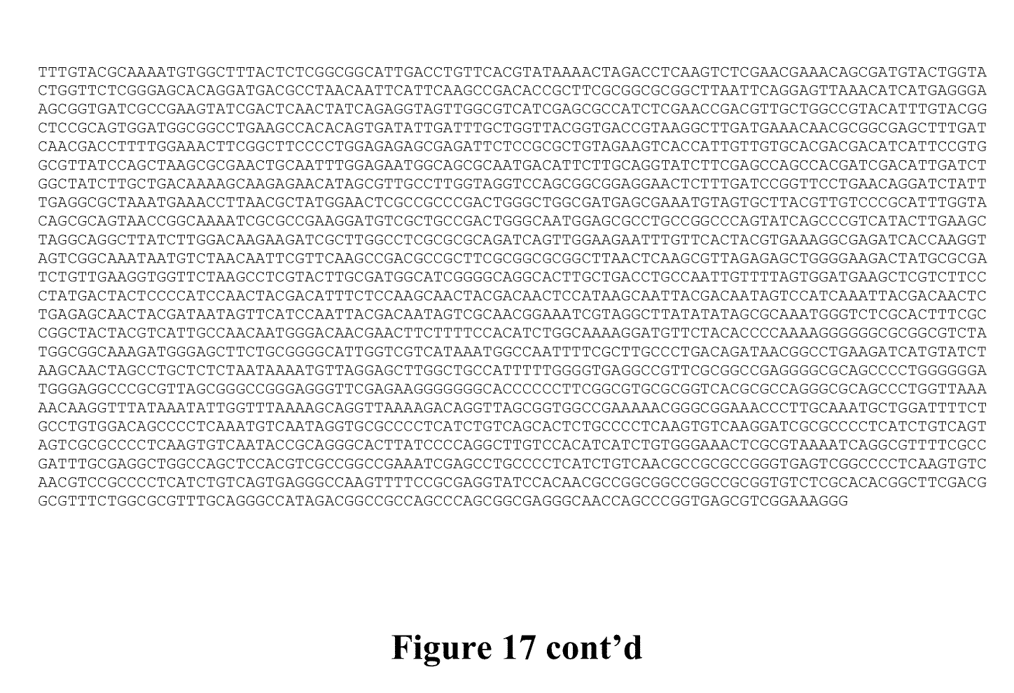

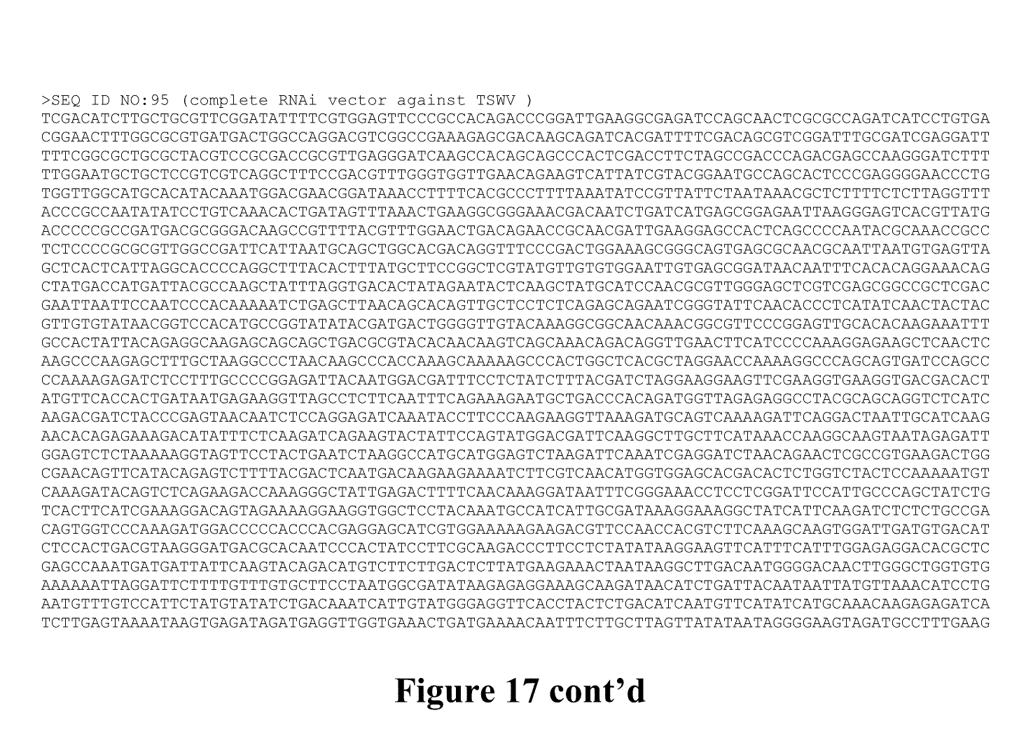

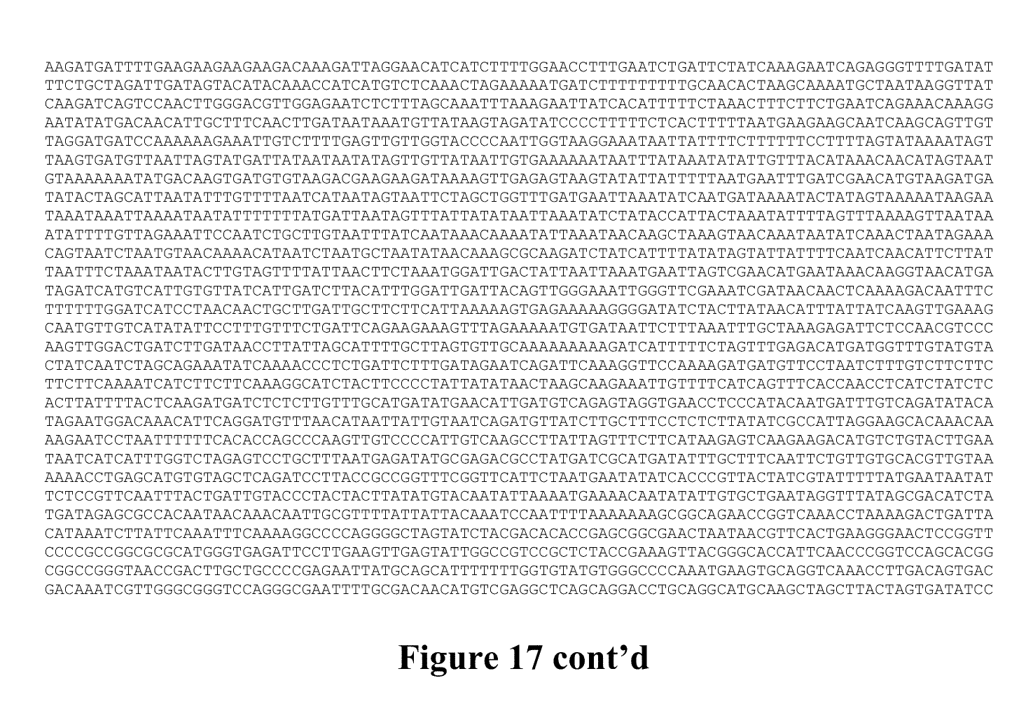

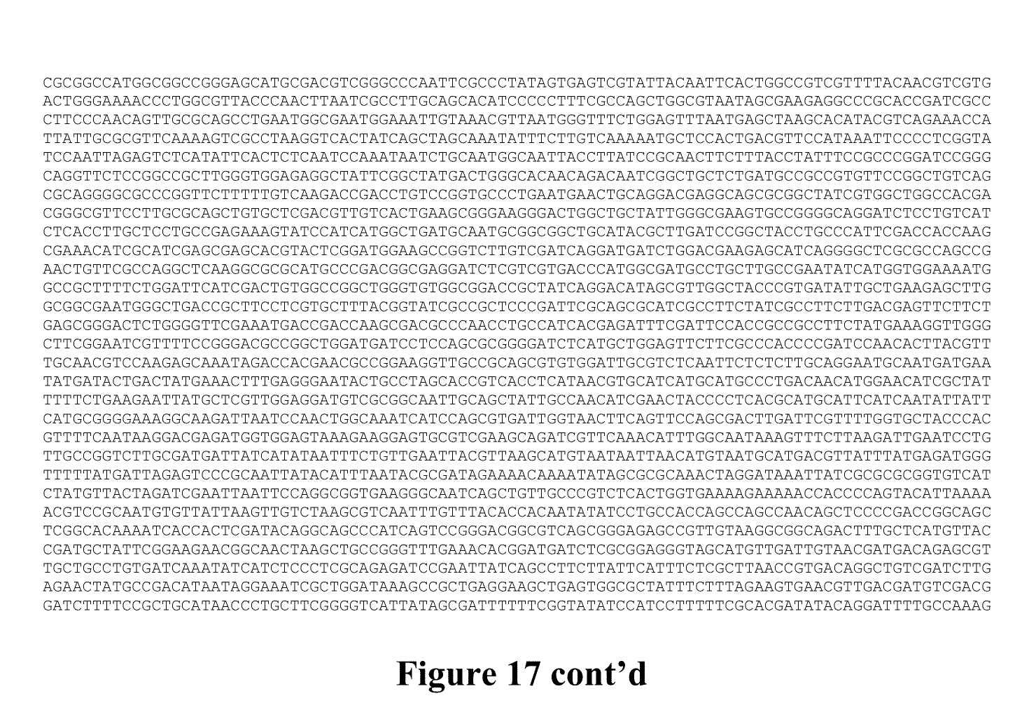

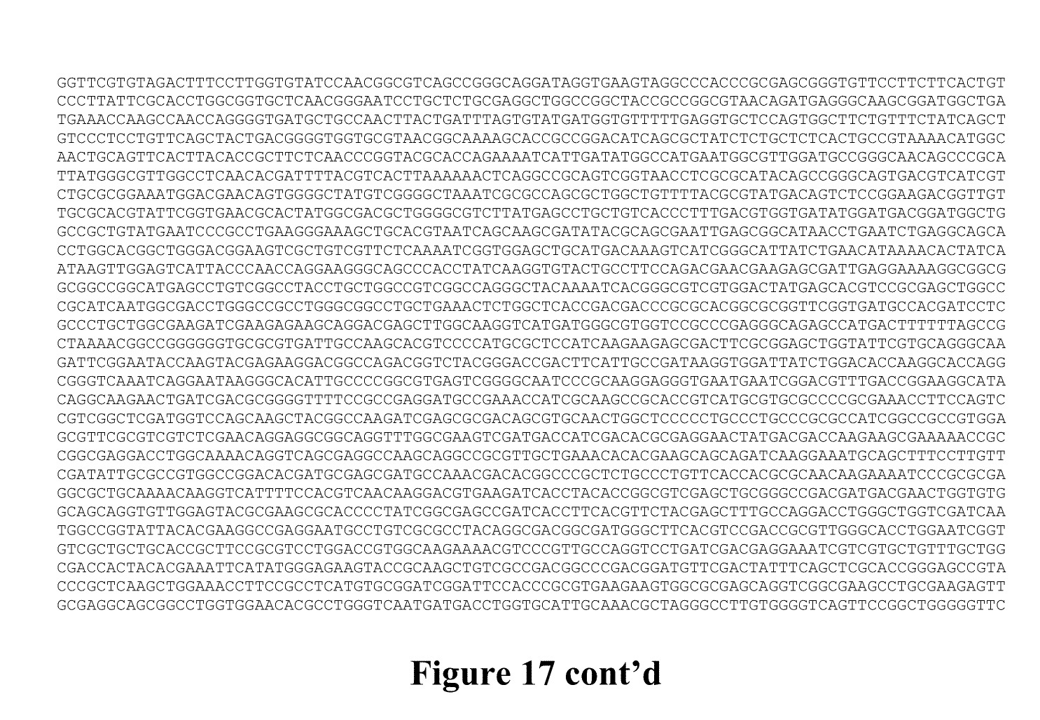

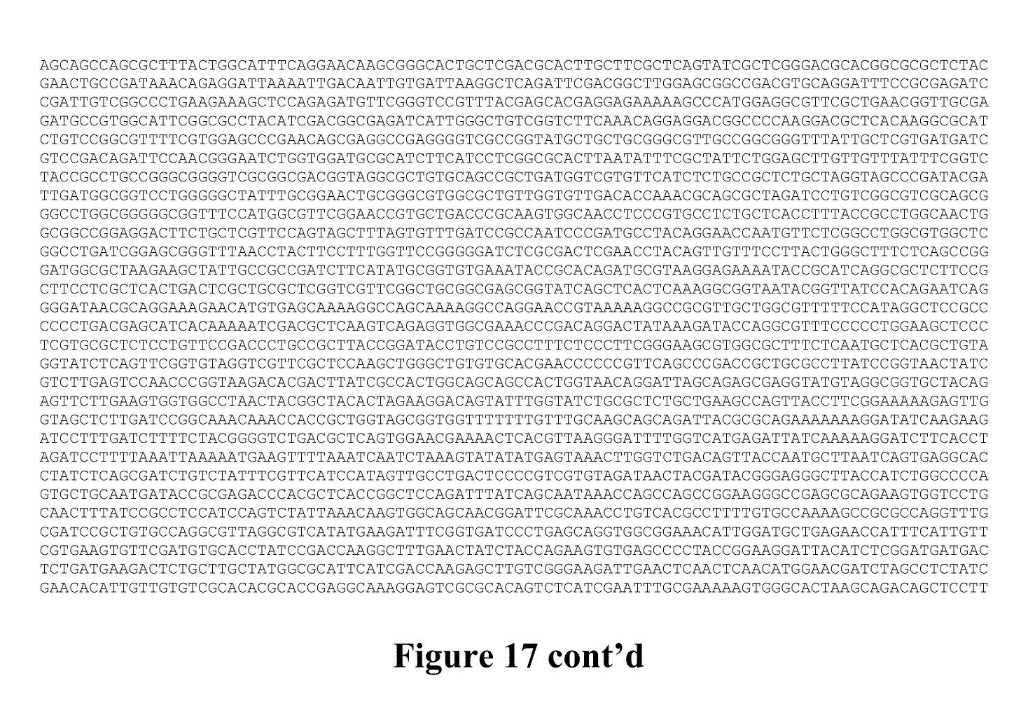

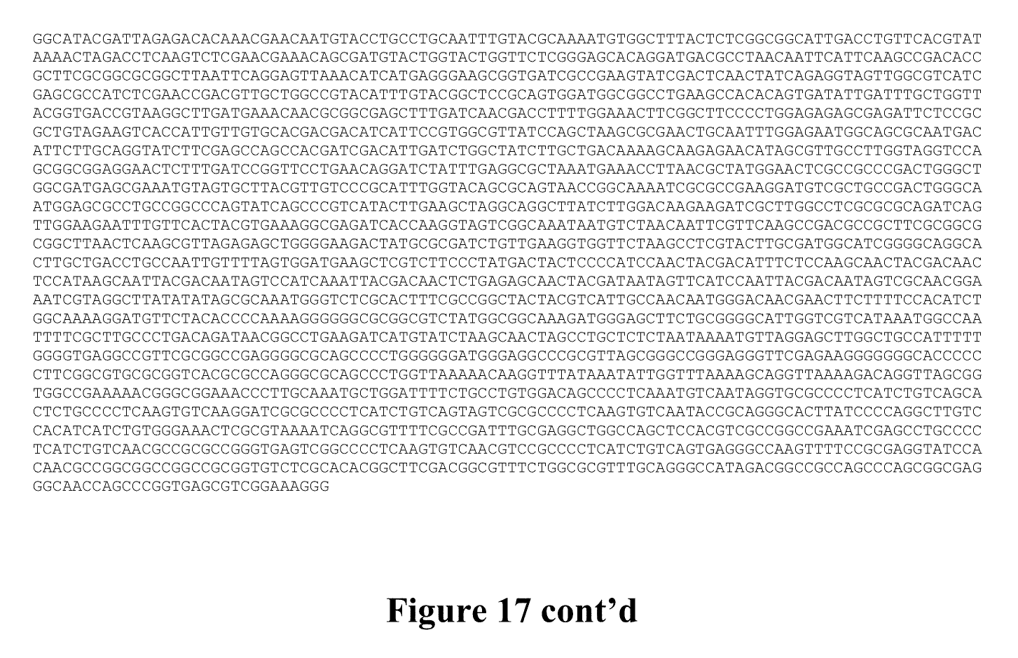

As described herein, intragenic constructs of the invention may be suitable for improving traits in crop plants, e.g. use of amiRNAs to develop disease resistance in tomato. Furthermore, constructs of the invention may facilitate trait improvement in one plants based on information obtained in another plants. By way of example, assessment of an intragenic strategy developed using the model plants Arabidopsis for use in the crop plant tomato is described herein, with reference to FIG. 16.

In developing this strategy, it was hypothesised that plant virus resistance could be achieved by activation of the salicylic acid (SA) pathway in plants. This pathway, when activated, can rapidly recognise biotrophic pathogens, mount an oxidative burst by production of reactive oxygen species, which then lead to a local hypersensitive response at the site of infection and localised programmed cell death (Mur et al., 1997, Plant J. 12 1113). As a result, biotrophic pathogens which rely on live cells, cannot proliferate and the plant is resistant. However, SA signalling is compromised by jasmonic acid (JA) signalling which typically antagonises the SA pathway, and many plant pathogens appear to hijack and activate one pathway to compromise the other, and facilitate disease progression (Thatcher et al., 2009, Plant J. 58 927). Therefore a new strategy was developed to suppress the JA pathway to upregulate the SA pathway in an attempt to induce plant resistance against biotrophic pathogens, such as viruses.

Mediator subunits control various physiological pathways in plants and the example presented herein in Arabidopsis shows that suppression of JA signalling and concurrent upregulation of SA signalling can be achieved by mutating the MED18 MEDIATOR subunit gene. In this Example it is shown that Agrobacterium-mediated T-DNA insertional mutant plants (med18) with dysfunctional Mediator 18 subunit displayed virus resistance when challenged with Turnip mosaic virus (TuMV; FIG. 16A) The alteration of expression of the endogenous MED18 gene caused reduced JA—but increased SA-mediated defence signalling, leading to significant (P<0.05) virus resistance.

It will be appreciated that a mutation in MED18 or many other genes can be achieved in an intragenic manner, for example by introducing Agrobacterium tumefaciens T-DNA that contains only endogenous (genome-derived sequence) as shown in Example 3. Alternatively, an RNAi or amiRNA approach can be used in an intragenic manner as shown in Example 4 to suppress gene or protein expression.

To test whether a strategy for this useful trait (virus resistance in plants via modification of defence signalling) can be rapidly developed for other plants, the genome of tomato was searched for the presence of MED18 orthologs (SEQ ID NO:64). Two tomato-derived amiRNA sequences (SEQ ID NOS:65-66) were then tested for the suppression of tomato MED18 using a luciferase reporter gene construct transient gene expression assays by using Agroinfiltration in Nicotiana benthamiana, as described in Example 4. As shown in FIG. 16B, both constructs led to a suppression of tomato MED18, validating this strategy and providing an alternative strategy for use of genetic constructs of the invention for improving disease resistance (and potentially other traits) in crop plants (see Example 7)

Further testing of Arabidopsis med18 mutants showed resistance against three other viruses. As set forth in FIG. 16C, these include CMV, CaMV and Alternanthera mosaic virus (AltMV). Together with TuMV, this comprises four different virus families whose resistance can be potentially achieved with intragenic approaches. This demonstrates the powerful approach of using well-studied model plants, such as Arabidopsis thaliana, to rapidly develop new intragenic strategies for crop traits.

### Example 7. Modulation of Physiological Pathways to Improve Resistance to Crop Plant Viruses

As set forth in Example 6, well-studied model plants, such as Arabidopsis thaliana are useful to develop new intragenic trait developments in crops. Plant pathogens can be categorised in two groups: those that depend on living cells to extract their nutrients (biotrophic and hemibiotrophic) and those that live off nutrients from dead cells (necrotrophic). Plant viruses are obligate biotrophic pathogens. As demonstrated in Example 6 for Arabidopsis, localised programmed cell death of a virus-infected cell is a suitable response for the plant to prevent systemic infection of the plant by a biotrophic pathogen, such as different types of viruses (FIG. 16). One way for the plant to deal with pathogens is to prepare the plant by modulating plant defence pathways prior to anticipated infections. Mediator subunits control various physiological pathways in plants and the examples presented herein in Arabidopsis and tomato plants show that suppression of JA signalling and concurrent upregulation of SA signalling can be achieved by mutating or downregulating the MED18 subunit gene. Furthermore they demonstrate that this approach can lead to the rapid identification of orthologous genes.

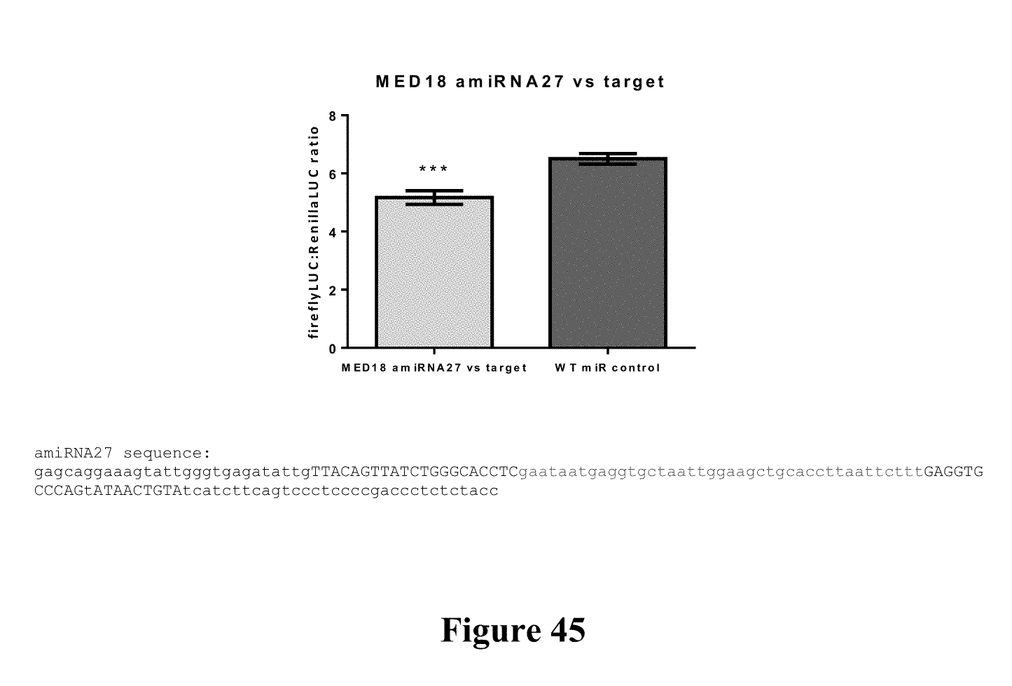

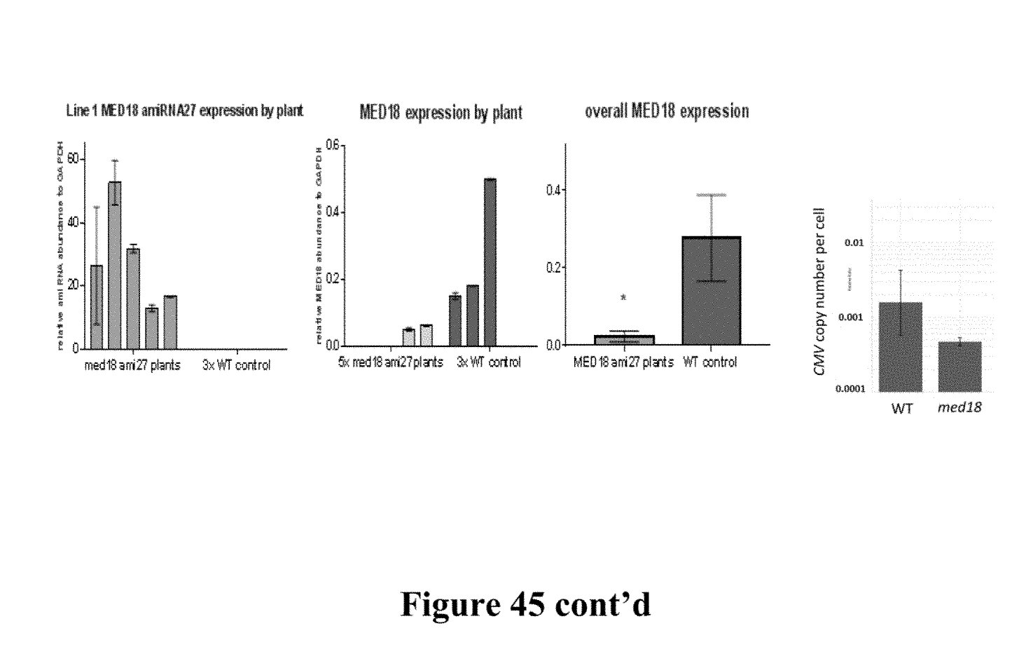

The potential ortholog identified for MED18 in tomato (SEQ ID NO:64) targeted by amiRNA27 (SEQ ID NO:66) was chosen for further development of an intragenic trait for virus resistance. First, the experiment obtained for the luciferase assay (FIG. 16B) was repeated to further increase confidence in this approach. As set forth in FIG. 45, amiRNA27 significantly (P<0.001; Student's t test) downregulated the MED18 target sequence, confirming the previous data. Next, tomato plants were transformed with the standard binary vector (pArt27 containing CaMV 35S promoter, amiRNA27 and Agrobacterium OCS terminator) to overexpress amiRNA27, using the method of Subramaniam et al., supra. A PCR-positive line was clonally propagated and the clones were tested with qRT-PCR for amiRNA27 expression and MED18 knockdown.

As set forth in FIG. 45, high amiRNA27 expression was achieved in these plants (up to 60-fold higher expression than GAPDH transcripts) and consequently MED18 expression was significantly (P<0.05; Student's t test) downregulated in the plants. Their phenotypic appearance included more vigorous growth with increased plant heights and broader leaves (FIG. 45), with normal-sized fruit but reduced seed numbers (see Examples 9 and 11). As the results in the model plant (Arabidopsis) predicted virus resistance, a detached shoot assay was developed to test for CMV resistance. Shoots of approximately 15 cm in height and at comparable developmental stages were detached from plants (wild type and MED18-compromised plants). These were mechanically inoculated with CMV as described above and subsequently kept in water-holding devices. At 2 weeks after inoculation, CMV presence was quantified in newly developed leaves by qRT-PCR. As set forth in FIG. 45, MED18-downregulated plants showed significantly lower CMV propagation than wild type plants, indicating that these plants are indeed virus resistant.

It will be appreciated that downregulation of MED18 or many other genes can be achieved in an intragenic manner, for example by introducing Agrobacterium tumefaciens T-DNA that contains only endogenous (genome-derived sequence) as shown in Example 3. Alternatively, an RNAi approach can be used in an intragenic manner as shown in Example 4 to suppress gene or protein expression.

### Example 8. Use of an Intragenic Approach to Confer Disease Resistance Against Non-Viral Pathogens

As various intragenic approaches (amiRNA, RNAi, pathway modulation) have been demonstrated for resistance against various viral pathogens in Examples 4-6, it was the aim of this invention whether this approach is feasible to be applied to confer resistance against other non-viral pathogens. One of these strategies had been set forth with the use of the model plant Arabidopsis, where it could be demonstrated that the modulation of physiological pathways can empower plants to develop rapid resistance against biotrophic pathogens.

In particular, a downregulation of the JA defence pathway can lead to the upregulation of the SA pathway, that in some aspects acts in an antagonistic fashion to JA signalling. It is believed that this decision making between pathways enables plants to mount the appropriate pathway that enables resistance (i.e. SA pathway against biotrophic/hemibiotrophic pathogens and JA pathway against necrotrophic pathogens and sucking insects). However, it appears that many pathogens hijack this hard wiring for defence signalling in plants by purposely inducing the inappropriate pathway. For example, the hemibiotrophic bacterial pathogen Pseudomonas syringae pv. tomato produces a JA mimic, coronatine, that can induce the JA defence signalling pathway in Arabidopsis and other plants. This pathway prevents or reduces the production of reactive oxygen species, a hypersensitive response and programmed cell, which normally would be the most effective response against a biotrophic pathogen.

Hence, for the purpose of this invention, it was tested whether downregulation of JA signalling (and associated upregulated SA signalling) in an intragenic manner could confer resistance against biotrophic pathogens other than plant viruses. First a detached leaf assay was developed for P. syringae pv. tomato using syringe infiltration in tomato. Disease resistance could be successfully assessed by symptom scoring and pathogen quantification using quantitative PCR at 5 days after inoculation. Next, wild type and MED18-compromised tomato plants with reduced JA signalling from Example 6 were used for P. syringae pv. tomato inoculation experiments.

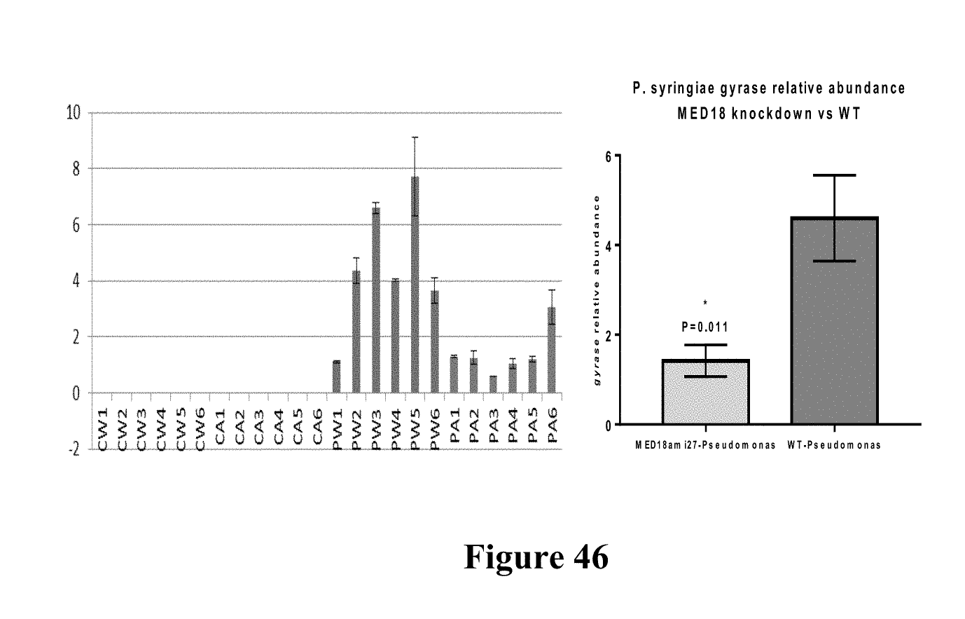

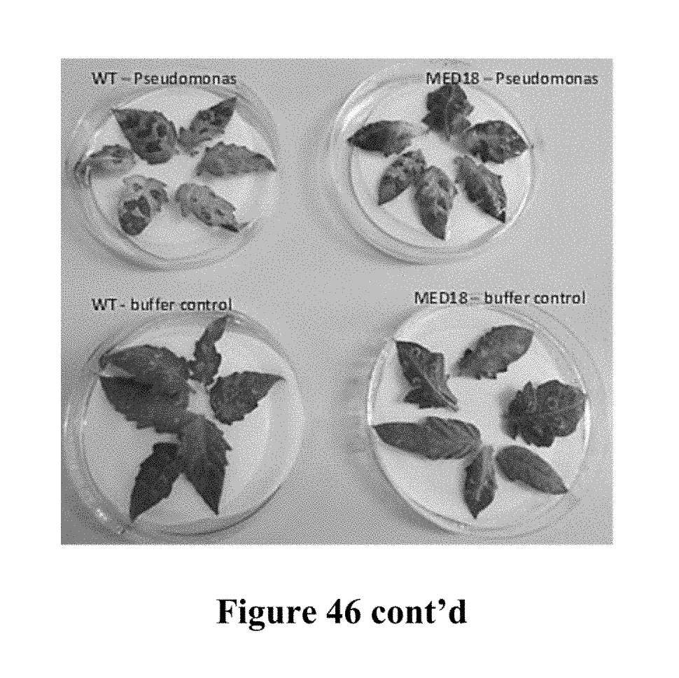

As set forth in FIG. 46, leaves with syringe-infiltrated P. syringae pv. tomato showed clear lesions and yellowing symptoms at 5 days after inoculation, while mock-inoculated leaves did not show yellowing, although some wound-induced lesions could be observed. It was noted that the wound-induced lesions in MED18-downregulated plants were clearly more prominent, confirming that these plants have the ability to mount a stronger hypersensitive response leading to programmed cell death. This is consistent with the predicted trait of heightened SA signalling ability of these plants.

P. syringae pv. tomato quantification was achieved through quantitative PCR with primers directed against the gyrase-encoding gene in P. syringae pv. tomato relative to tomato GAPDH genomic sequence. As set forth in FIG. 46, all inoculated leaves proliferated P. syringae pv. tomato while mock-inoculated leaves did not contain quantifiable amounts of these bacteria. Notably, leaves from MED18-downregualted plants showed significantly (P=0.011; Student's t test) reduced bacteria per plant cell than wild-type plants, indicating that this intragenic approach also provides a valid strategy to confer bacterial resistance to crop plants. Resistance against other biotrophic and hemibiotrophic pathogens (e.g. fungal pathogen Fusarium sp.) can be expected and testing for these pathogens is underway.

### Example 9. Use of an Intragenic Approach to Provide Abiotic Stress Tolerance in Crop Plants

Abiotic stresses in crop systems, such as salinity, drought, high temperature, chilling and flooding cause billions of dollars in yield losses annually. Such stresses also severely restrict the use of land for crop cultivation, a major issue for food security and the growing world population. For example, an increasing area of arable land is also affected by high soil salinity, often caused by excessive irrigation practices. In Australia alone, it is estimated that 12% of the land is affected by salinity and even more so by drought. There is therefore an urgent need to develop crop cultivars with increased abiotic stress tolerance.

Rice is a major crop feeding billions of people. Hence, in this Example, a salinity-tolerant rice cultivar was developed that uses only endogenous (intragenic) genomic sequence and no foreign sequence. It can be appreciated that this fully intragenic approach described in this Example can be applied to other rice cultivars and other important crops.

First, a rice variety was identified that is widely used as a commercial crop in Australia and other locations. Cultivar Oryza japonica Reiziq is popular among growers with high yield potential but lacks tolerance to abiotic stresses, in particular low temperatures and salinity. Therefore this variety was considered an ideal candidate for the intragenic introduction of abiotic stress tolerance, including the trait for salinity tolerance. Its commercialisation may lead to wider cultivation by including the many areas in the world that are affected by salinity.

For the purpose of this Example, first a new transformation protocol had to be established for the Reiziq variety. Media were as follows: Callus induction medium included LS basal medium, LS vitamins, 500 mg·L⁻¹ Glutamin, 50 mg·L⁻¹ Tryptophan, 3% sucrose, 2.5 mg·L⁻¹ 2,4-D and 5% Phytagel. Regeneration medium included MS basal medium, Gamborge B5 vitamins, 1 mg·L⁻¹ NAA, 3 mg·L⁻¹ BAP, 1 mg·L⁻¹ Kinetin, 3% sucrose and 5% Phytagel. Selection medium (1) included Regeneration medium with 200 mM NaCl. Selection medium (2) included Regeneration medium with 100 mM NaCl. Selection medium (3) included Regeneration medium with 25 mM NaCl.

The seed surface sterilisation method included dehusking the seeds, soaking of dehusked seeds in 70% ethanol and shaking for 30 s. followed by soaking and shaking the seeds in 4% (m/v) sodium hypochlorite solution containing three drops of Tween 20 for 20 min, before rinsing the seeds with sterile distilled water for 5 times to wash away the bleach.

Somatic embryogenic calli induction method included placing 15 to 20 seeds in each petri dish in the laminar airflow, pushing of the seeds slightly in the callus induction medium, and placing the petri dishes in the dark room for 3 to 4 weeks to produce somatic embryogenic calli. The somatic embryogenesis calli were then used directly for transformation or subculturing in the callus induction medium. It was found advantageous to use the 14 to 20 days old embryogenic calli for transformation.

Particle bombardment and transformation steps included preparation of the intragenic DNA fragments by cutting purified plasmid DNA with the corresponding flanking restriction sites (whose remaining nucleotides form part of the intragenic sequence), followed by fragment purification from an agarose gel subjected to electrophoresis. Alternatively, synthesised DNA can be used directly. Particle bombardment of embryogenic calli was carried out with gold particles (0.6 μm diameter) using 10 μL of 1 μg/μL linear purified DNA. For co-bombardment with two DNA fragments, 5 μg were used of each fragment. At least 10 micro calli were positioned in the centre of a plate containing Selection medium (1) and bombarded with the intragenic DNA fragment.

Selection steps included placing the plates in the dark for 3 days and subculturing of the calli to Selection medium (1). The healthy calli were then subcultured to Selection medium (2) after 10 days. The green (surviving) calli were then subcultured to Selection medium (3) until the leaves appeared. After sufficient root formation, plants were carefully transferred to soil and hardened off by placing a transparent plastic container on top of the plants.

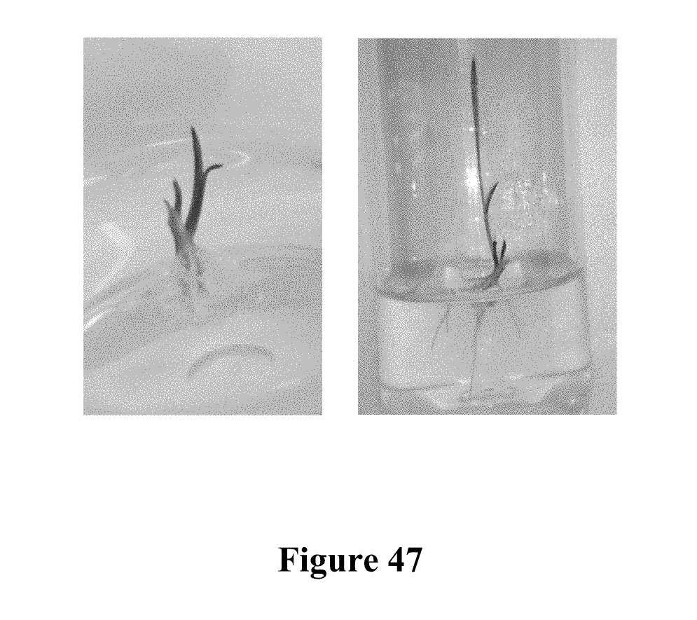

For the purpose of conferring salinity tolerance to rice plants, Reiziq embryogenic calli were transformed with intragenic DNA fragment ACTIN1:DREB1A:DREB1A set forth in SEQ ID NO:78 after cutting with restriction enzymes NheI and Pml1. Intragenic salinity tolerant rice plants were then produced and regenerated as described above. As set forth in FIG. 47, these rice plants were able to grow in 100 mM NaCl containing medium, while none of the control plants survived these conditions. The salt concentration of 100 mM corresponds to 6 ppt salt contents (or 17% seawater concentration). Current trials with this new rice cultivar are underway to determine the maximum range of salinity tolerance and how this may affect yields and grain quality. Other abiotic stress tolerance can also be expected for these plants and additional trials are planned for this purpose.

The above new rice variety harbours salinity tolerance that is mediated by a relatively strong, near-constitutive promoter (ACTIN1). For those experienced in the art, the question may arise whether the continuous activation of the DREB1A-mediated pathway in rice may lead to some yield compromises as plants need to allocate additional resources to confer salinity tolerance. To overcome this potential issue, rice transformation with another construct was trialled that included the rice ABA-inducible promoter NCED3. ABA signalling is typically activated during abiotic stress in plants, and therefore, it can be expected that no or little resources are used by the plant during growth in the absence of abiotic stress. As a result, no yield compromises would be expected when plants with NCED3 promoter-mediated ABA-inducible stress tolerance are grown under stress-free conditions.

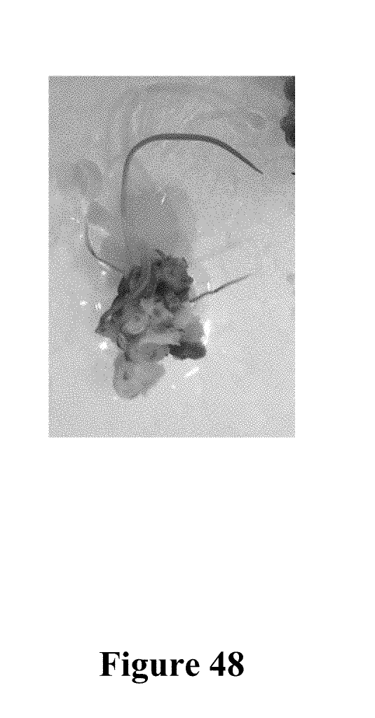

For the purpose of conferring ABA-inducible salinity tolerance to rice plants, Reiziq embryogenic calli were transformed with intragenic DNA fragment NCED3:DREB1A:NCED3 set forth in SEQ ID NO:79 after cutting with restriction enzyme FspI. Intragenic salinity tolerant rice plants were then produced and regenerated as described above. As set forth in FIG. 48, these rice plants were also able to grow in 100 mM NaCl containing medium, while none of the control plants survived these conditions. Current trials with this new rice cultivar are planned to determine the maximum range of salinity tolerance. It is expected that yield and grain quality are not compromised. Other abiotic stress tolerance can also be expected for these plants and additional trials will be carried out for this purpose.

It can be appreciated that salinity tolerance and other abiotic stress tolerance can be conferred in an intragenic manner in rice and also other crop plants by using the intragenic strategy set forth in the example above.

### Example 10. Use of an Intragenic Approach to Modify Plant Architecture and Appearance in Crop Plants

Alterations in plant architecture and appearance are desirable traits in crop plants. For example dwarf varieties for cereals enabled higher yields and earlier harvesting and formed part of the “Green Revolution”. Dwarf varieties are also desirable for many fruiting trees to enable easy harvesting, while taller, bushier varieties are desirable for other plants, such as blueberries. Forage plants are desirable that produce prolific foliage and more robust, stronger stems could provide advantages to banana plants to enable cyclone resistance. In fruits many improvements are desirable, for example increased fruit size, flavour and reduction of seeds.

Intragenic technology, as described herein, may provide options to modify plant architecture and appearance of crop plants. To explore this possibility, a suite of plant Mediator subunits was approached by intragenic amiRNA technology. The plant Mediator provides a link between RNA Polymerase II that binds to the TATA box of plant promoters and transcription factors that bind to other cis-acting elements in promoters that are typically located upstream of the TATA box. The mediator complex is comprised of approximately 30 subunits, some of which bind to various transcription factors. Hence, different Mediator subunits provide signalling and regulatory control units for various physiological pathways in plants. This feature had already been explored in Example 6 for MED18-compromised plants that displayed reduced JA signalling and increased biotic stress tolerance against viral and bacterial pathogens.

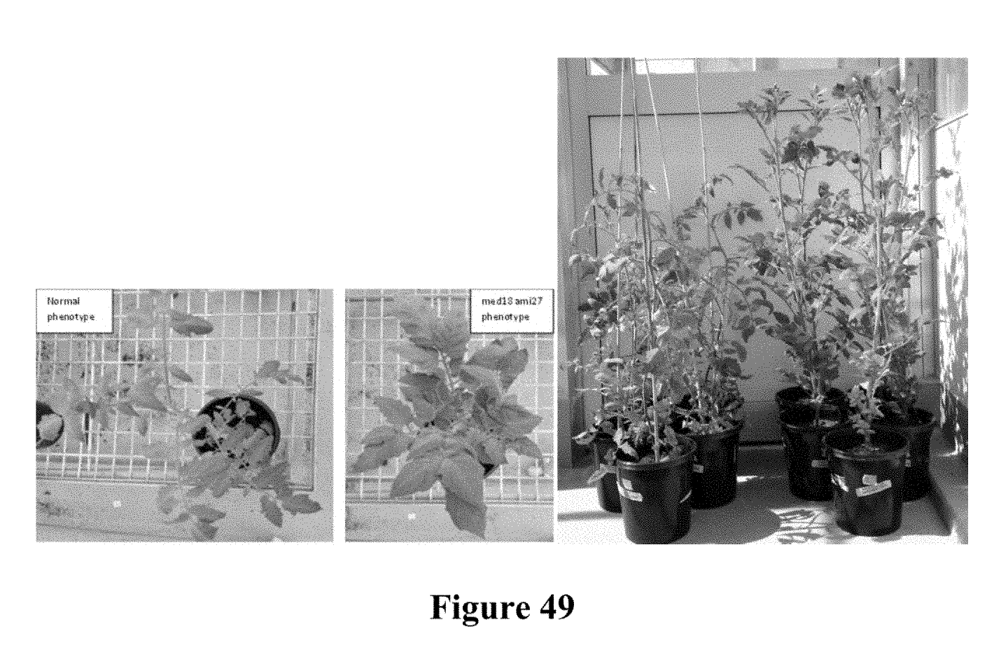

Assessment of their phenotypic appearance revealed that these plants displayed more vigorous growth with increased plant heights and broader foliage as set forth in FIG. 49. Plants produced normal-sized fruit but with reduced seed numbers. It remains untested whether these plants show variations in fruit yield at this stage, but it can be appreciated that plants with increased plant height, broader (lusher) foliage and reduced seed loads may offer some advantages to either the farmer or the consumer.

Male cytoplasmic sterility is another trait that should be explored using an intragenic approach to Mediator subunit modulation, as this is a trait that is of commercial value for seed companies who can use these plants as parental lines and who do not wish the resulting progeny to be true to type. This is a common feature of commercial tomato varieties, requiring growers to purchase seeds from seed companies.

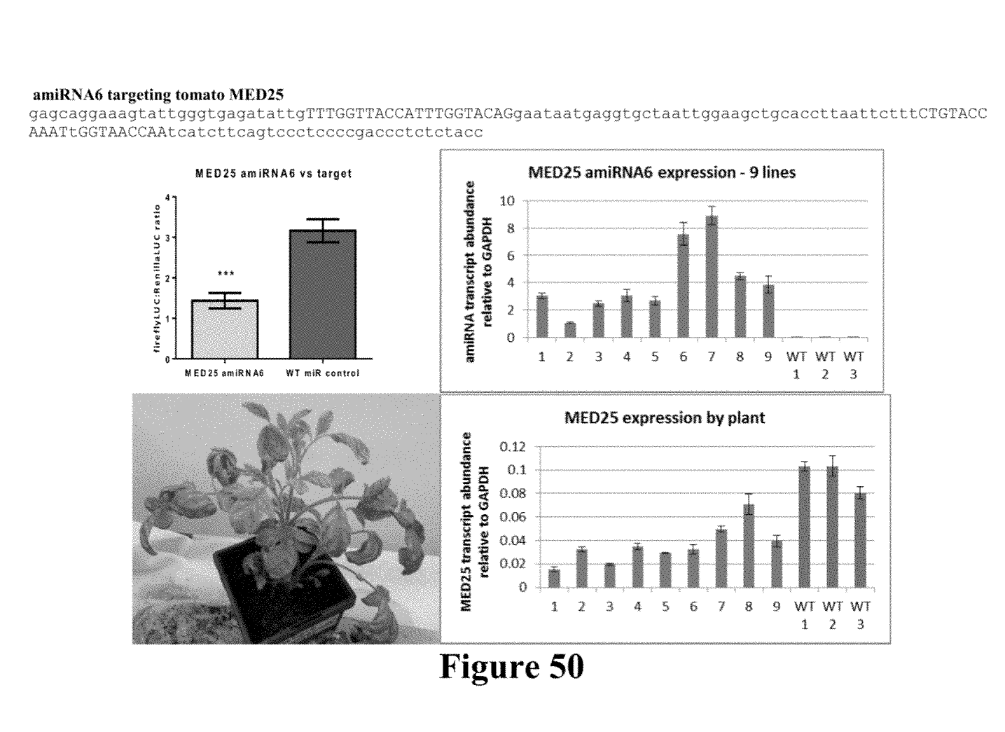

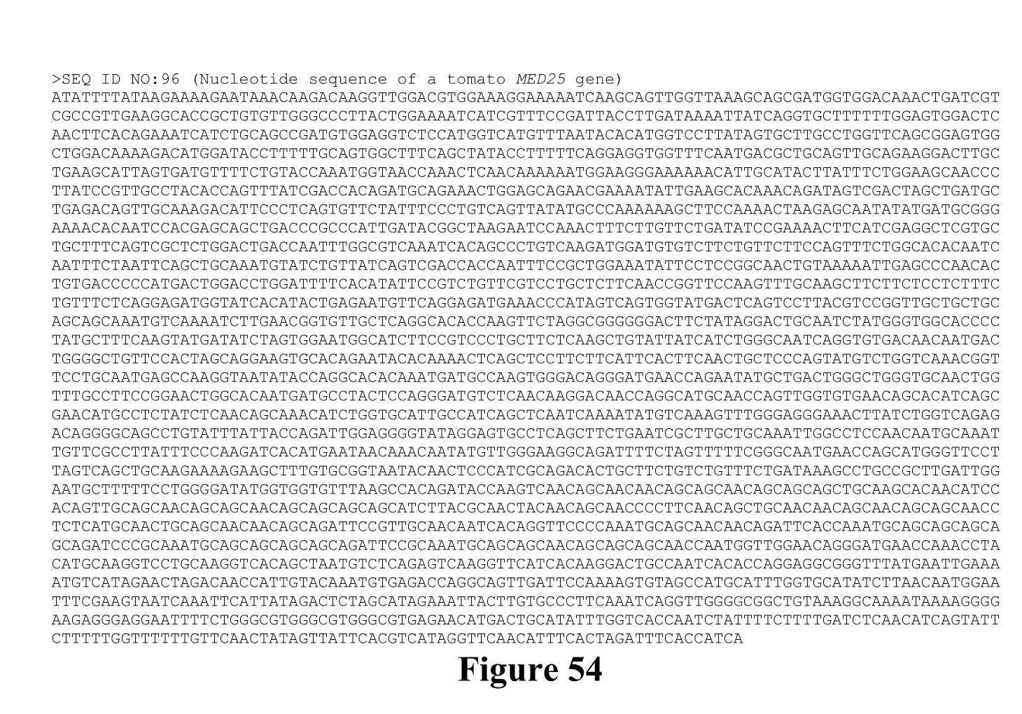

To test whether modulation of other Mediator subunits in tomato may lead to desirable plant architectural traits, the putative MED25 ortholog (SEQ ID NO:96; FIG. 54) was identified in tomato and an intragenic amiRNA (SEQ ID NO:97; FIG. 50) was designed for its downregulation. As set forth in FIG. 50, amiRNA6 was able to significantly (P<0.001; Student's t test) downregulate the tomato MED25 sequence when using the dual luciferase assay in N. benthamiana described above. AmiRNA9 was inserted into pIntrA and tomato plants were transformed as described above. Nine PCR-positive transformants (lines) were tested with qRT-PCR for amiRNA6 expression and MED25 knock-down. As set forth in FIG. 50, all nine lines expressed amiRNA6 and MED25 expression was significantly (P<0.05) reduced for all lines produced in comparison to wild type plants.

The phenotypic appearance of these plants was strikingly different than wild-type plants and included stunted plant height, bushier plants, curled broader leaves and yellow blotchiness of leaves. This demonstrates that an intragenic approach as set forth in this invention can be used to change plant architecture and appearance. It can be appreciated that altered plant architecture and appearance can be conferred in an intragenic manner in tomato and also other crop plants by using the intragenic strategy set forth in the example above.

### Example 10. Improvement of the Nutritional Value of Crop Plants

The nutritional value of plants as food sources is unquestionable a trait that is highly appreciated by consumers. Intragenic plants with improved nutritional value offer therefore direct consumer benefits and are likely to find easy acceptance. Nutritionally enhanced plants may include those with higher protein, vitamin, mineral, antioxidant, polyunsaturated fatty acid levels. One particular nutritional aspect that has been highlighted as beneficial for consumer's health is the anthocyanin content in fruit and vegetables. Some of these “superfoods” with increased anthocyanin levels include blueberries, purple carrots, beetroot and the Queen Garnet plum. Notably, higher anthocyanin levels in consumed food has led to reduced blood pressure and other cardiovascular and cancer-preventing benefits.

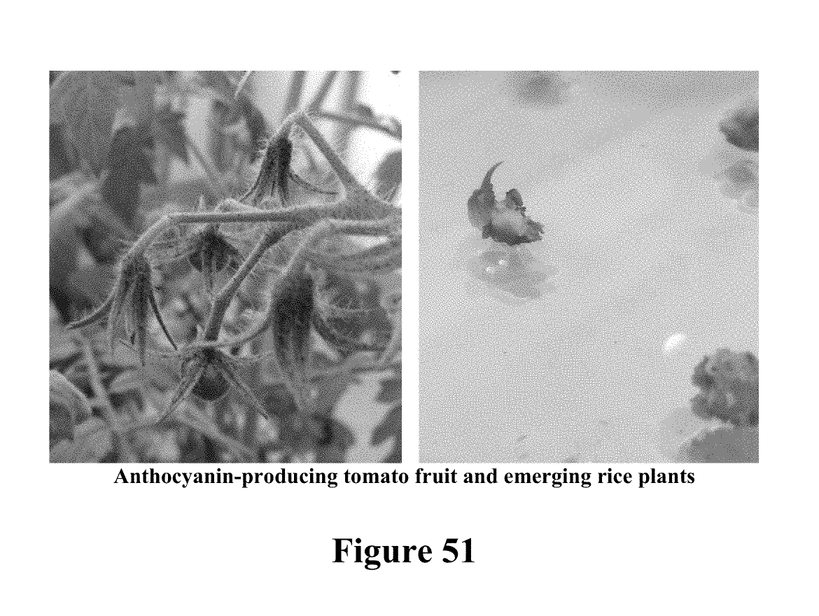

For the purpose of this invention and to increase the nutritional value of food crops, both tomato and rice plants were produced that contained higher anthocyanin levels that wild type plants. Tomato plants were transformed as described previously with the construct set forth in SEQ ID NO:69 that includes a tomato ANT1 gene flanked by the native ACTIN promoter and RbcS3C terminator. Plants were grown in the glasshouse until fruit-setting stage and their fruit colour was assessed. As set forth in FIG. 51, emerging tomato fruits had a visibly purple appearance, indicating their high anthocyanin levels.

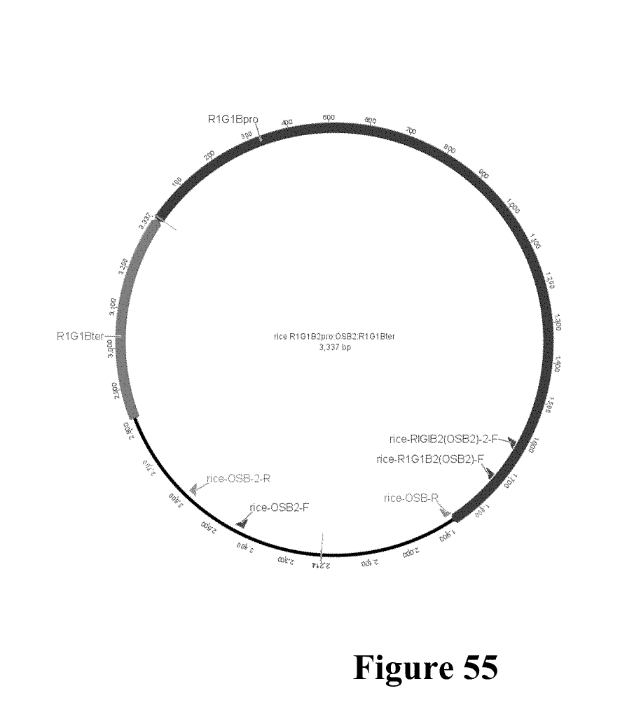

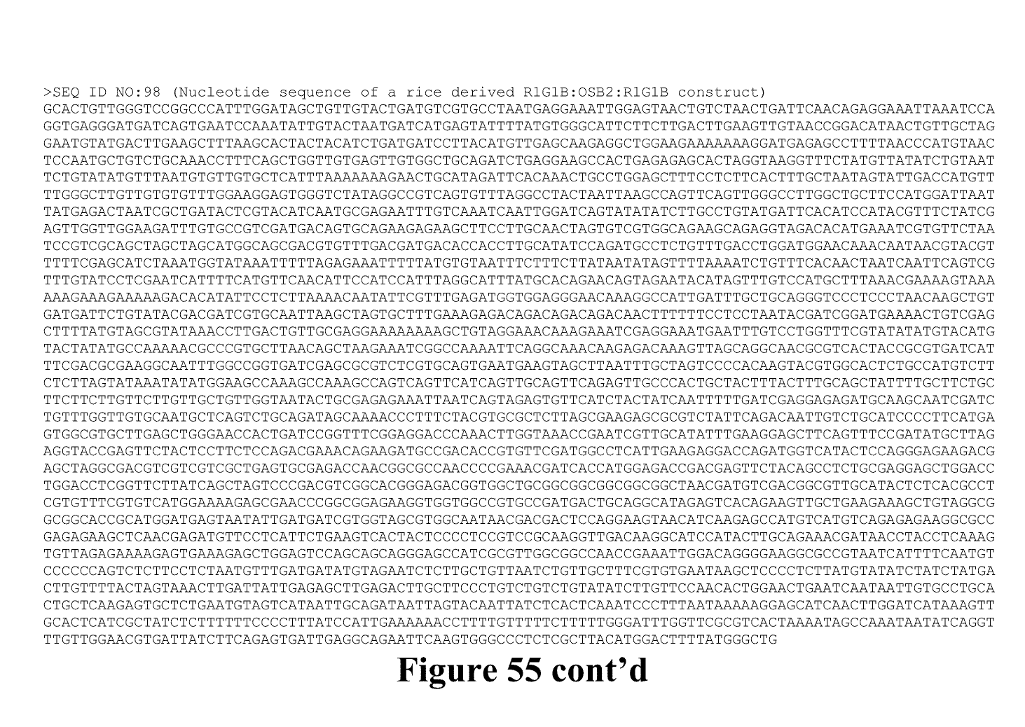

Furthermore, to improve the nutritional value of a commercial widely-consumed staple food crop, plants of a new Reiziq rice cultivar was produced that harbours a fully intragenic cassette to increase anthocyanin levels in rice grains. Rice cultivar Reiziq plants were transformed as described above with an intragenic construct comprising the sequence set forth in SEQ ID NO:98 (FIG. 55) that includes a rice OSB2 gene flanked by the native R1G1B promoter and terminator in addition to the ACTIN1:DREB1A:DREB1A cassette. Prior to particle bombardment the intragenic OSB2 cassette was excised and purified by cutting with FspI and Apa1I restriction enzymes. The rice R1G1B promoter and terminator cassette was chosen as the corresponding gene expresses strongest in the endosperm of mature rice grains. Plants were successfully produced as set forth in FIG. 51 and are currently grown to maturity to measure anthocyanin levels in rice grains. It is anticipated that consumer acceptance of these plants would be high as these plants offer direct consumer benefits and are fully intragenic. In addition, they are likely to display improved abiotic stress tolerance mediated by the intragenic DREB1A cassette that may benefit the growers of this variety. Future crosses with other varieties can be anticipated as these plants are integrated into breeding programs.

It can be appreciated that higher anthocyanin levels and other improved nutritional values can be conferred in an intragenic manner in tomato, rice and also other crop plants by using the intragenic strategies set forth in the example above.

### Example 11. Other Consumer-Friendly Traits

The benefit of new crop cultivars may be best appreciated by consumers if they experience an improvement to existing plant products. For the purpose of this invention and to make the case that intragenic technology as set forth in this patent is useful by providing direct benefits to the consumer experience, two improved crop varieties were produced. These include heart-shaped tomatoes and fragrant rice.

Heart-shaped tomatoes may prove popular to consumers based on their colour and original shape. As they have potential to enhance the consumer's experience there is a potential market for this product. Fragrant (jasmine) rice is already popular with consumers who based on the volatiles that are released after cooking are prepared to pay a higher price for this rice. Therefore these consumer-friendly traits were chosen as examples for intragenic technology described in this invention.

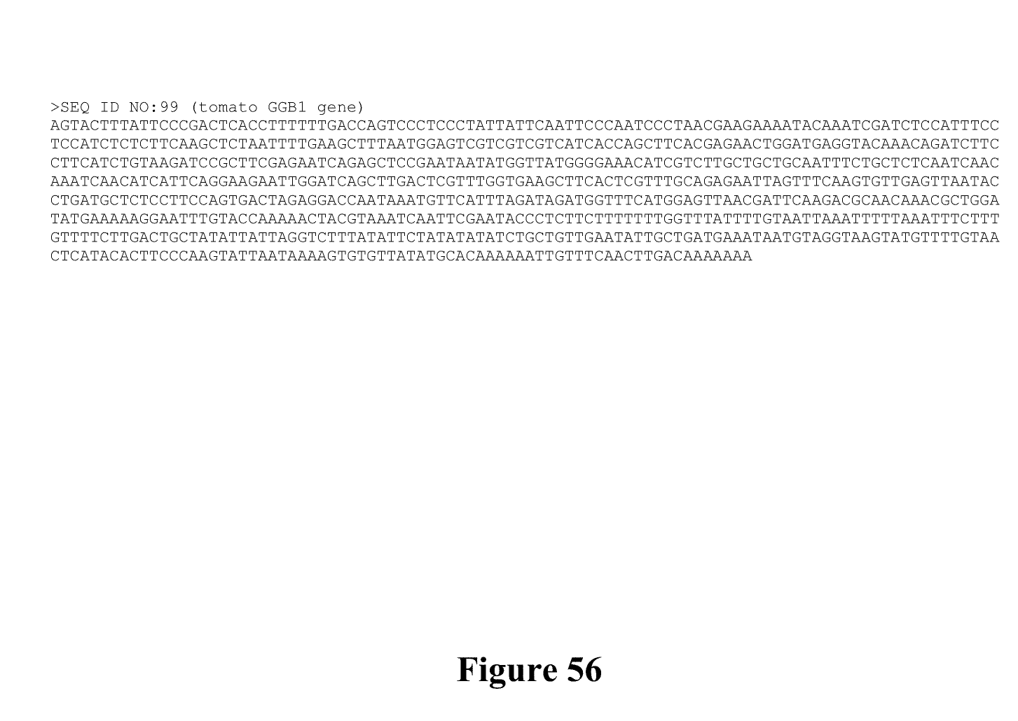

Plants producing heart-shaped tomatoes were generated by RNAi-mediated downregulation of the tomato gene encoding the γ-subunit of the type B heterotrimeric G protein (GGB1). Downregulation of this gene in a transgenic manner has recently been described for MicroTom tomatoes where it resulted in pointy fruits (Subramaniam et al., supra). The transcript sequence of this gene is set forth in SEQ ID NO:99 (FIG. 56).

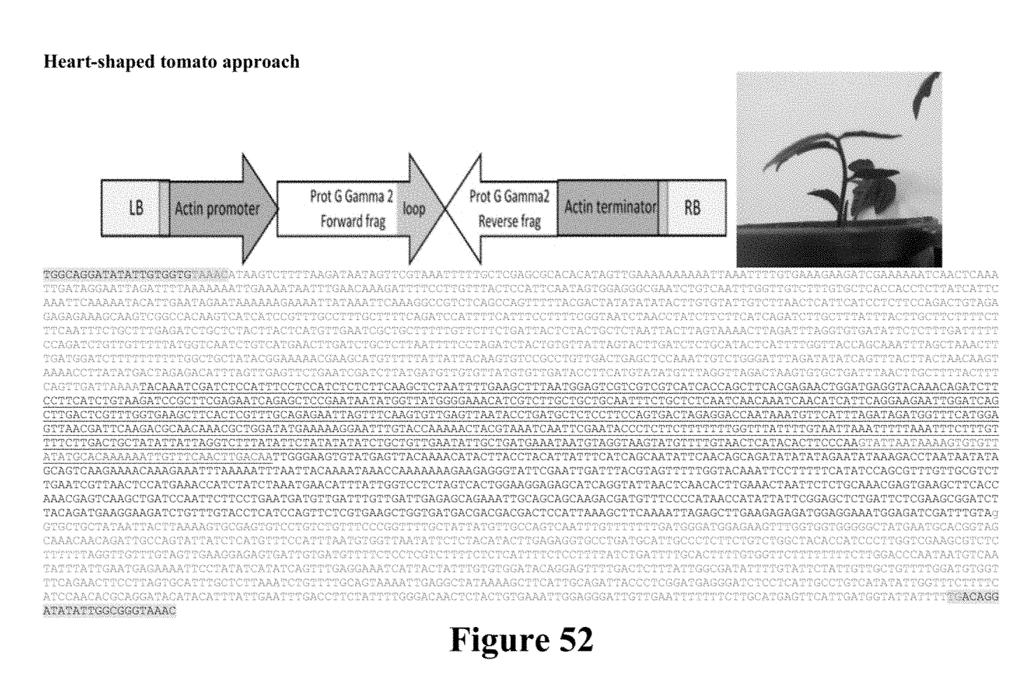

To produce an intragenic RNAi construct in the ACTIN promoter-terminator expression cassette (pIntraA), first the long “Forward” fragment was amplified with F primer 5′PhosGATTAAAATACAAATCGATCTCCATTTCCTCCATC (SEQ ID NO:150) complementing the end of the ACTIN promoter and R primer tcccaaTTGTCAAGTTGAAACAATTTTTTGTGCATATAAC (SEQ ID NO:151) adding three nucleotides to create a temporary MfeI restriction enzyme site. The shorter “Reverse” fragment was amplified with F primer tcccaaTTGGGAAGTGTATGAGTTACAAAACATACTTACCT (SEQ ID NO:152) adding three nucleotides to create a temporary MfeI restriction enzyme site and R primer 5′PhosCTACAAATCGATCTCCATTTCCTCCATC (SEQ ID NO:153) complementing the start of the ACTIN terminator. The fragments were restricted with MfeI and assembled in one ligation with pIntrA opened up with HpaI and PmlI. As the MfeI site is ligated between the long and short fragments, half of it belongs to the long fragment and the other half to the short fragment. The direction of the insert was verified for the complementation of promoter and terminator. The complete intragenic construct encompassing LB and RB fragments is set forth in SEQ ID NO:100 and FIG. 52.

Tomato transformation (cv. Moneymaker) was performed by co-transforming the construct in SEQ ID NO:100 with the marker gene cassette containing both ANT1 and NPTII genes for selection of transformed plants, as described in the examples above. Purple plants (indicating their positive transformation status) were selected and further tested for gene expression by qRT-PCR. Other plants without expression of the ANT1 gene were also selected. Tomato fruit produced by these plants are expected to be of pointy and heart-shaped appearance with either purple or red fruit colour, respectively.

Rice is a major staple food crop. For the purpose of developing a consumer-friendly in an intragenic manner, a high fragrance rice cultivar was developed from a popular Australian variety (Reiziq) that does not currently possess this trait. It can be appreciated that the intragenic approach described in this invention to achieve this trait can be applied to other rice cultivars and possibly other important crops.

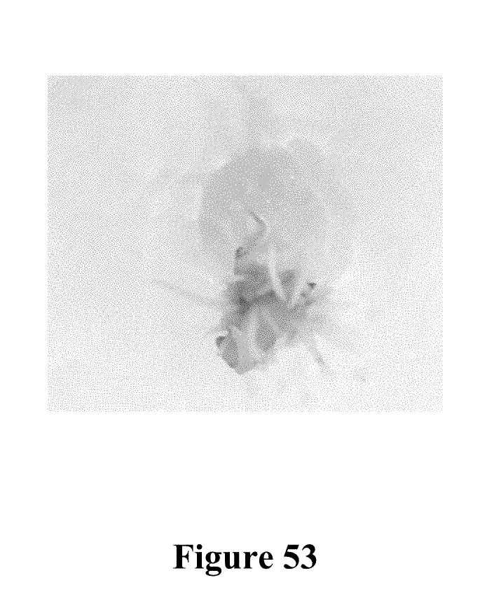

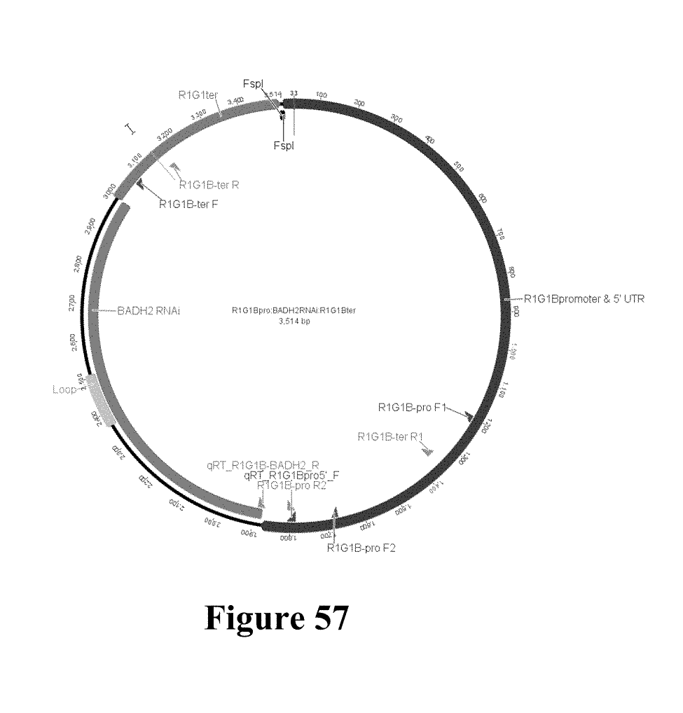

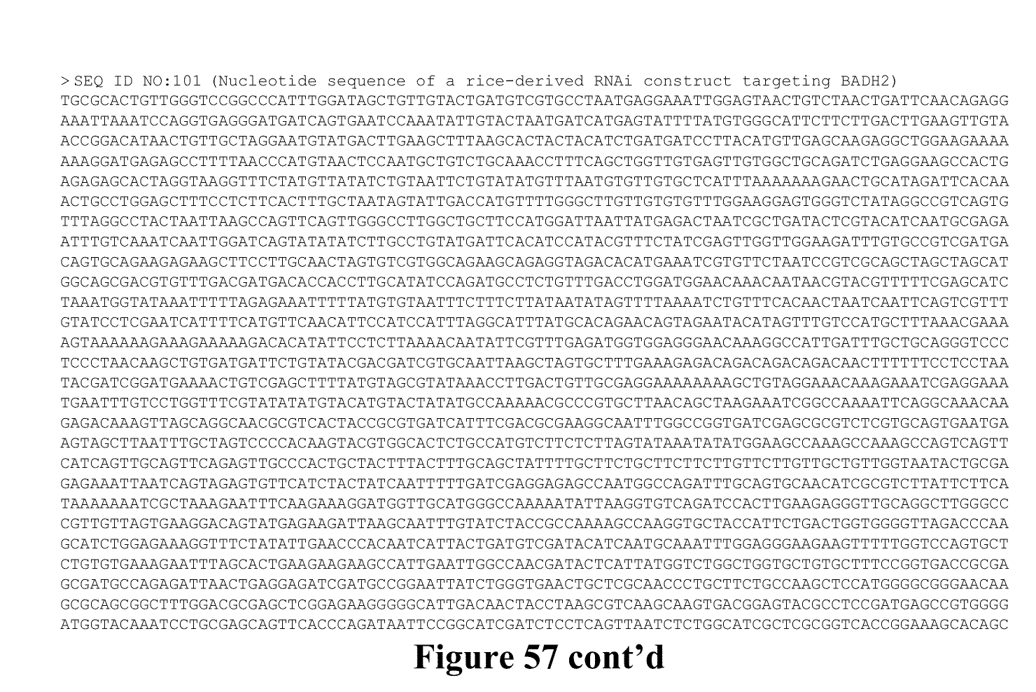

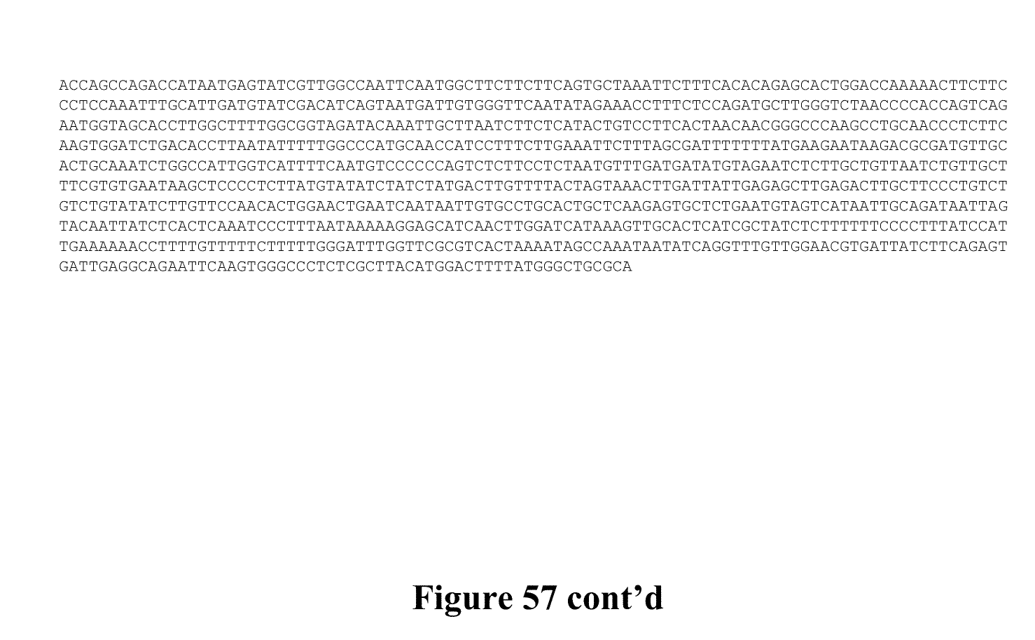

Cultivar Oryza japonica Reiziq is popular among growers with high yield potential but lacks fragrance that is typically found for jasmine (fragrant) rices. Fragrance in rice can be achieved by disrupting expression of the BADH2 gene in rice. Hence a BADH2 RNAi cassette with endogenous R1G1B promoter and terminator that expresses in rice endosperm was constructed. The complete cassette is set forth in SEQ ID NO:101 and FIG. 57. Excision of this DNA cassette prior to particle bombardment of rice calli has been achieved using FspI restriction enzyme and agarose gel electrophoresis size fragmentation. Developing intragenic rice plants with potential fragrance are set forth in FIG. 53.

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

1. A recombinant genetic construct comprising one or more nucleic acid fragments adapted for insertion into the genetic material of a plant to alter or modify a trait of the plant selected from the group consisting of a disease resistance trait, an abiotic stress tolerance trait, and a morphological trait, wherein each of said one or more nucleic acid fragments consists of a plurality of nucleotide sequences of at least 20 nucleotides in length derived from one or more plants, and wherein upon insertion of said one or more nucleic acid fragments into the genetic material of a plant, nucleotide sequence that is introduced into the genetic material of the plant consists of said plant derived nucleotide sequence.

2. The recombinant genetic construct of claim 1, wherein the nucleotide sequences derived from one or more plants are derived from one plant or a plurality of plants of the same species.

3. The recombinant genetic construct of claim 1, wherein the total length of the one or more nucleic acid fragments of the genetic construct that are insertable into the genetic material of a plant is at least 100 base pairs; at least 500 base pairs; at least 1000 base pairs; at least 2000 base pairs; or at least 3000 base pairs.

4. The recombinant genetic construct of claim 1, wherein the one or more nucleic acid fragments of the genetic construct that are insertable into the genetic material of a plant comprise one or more protein coding nucleotide sequences for expression in a plant to alter or modify the trait in the plant.

5. The recombinant genetic construct of claim 1, wherein the one or more nucleic acid fragments of the genetic construct that are insertable into the genetic material of a plant comprise one or more non-protein-coding nucleotide sequences for expression in a plant to alter or modify the trait in the plant.

6. The recombinant genetic construct of claim 5, wherein the non-protein-coding nucleotide sequences comprise one or more small RNA nucleotide sequences.

7. The recombinant genetic construct of claim 1, further comprising flanking sequences of or surrounding the one or more nucleic acid fragments insertable into the genetic material of a plant, wherein said flanking sequences comprise (a) one or more restriction digest sites; and/or (b) one or more border sequences functional for Agrobacterium T-DNA-mediated plant transformation.

8. The recombinant genetic construct of claim 1, comprising:
a first border nucleotide sequence;
a second border nucleotide sequence; and
one or more additional nucleotide sequences located between the first border nucleotide sequence and the second border nucleotide sequence,
wherein said additional nucleotide sequences, and at least a portion of said first border nucleotide sequence that is adjacent to said additional nucleotide sequences, are derived from one or more plants.

9. The recombinant genetic construct of claim 1, comprising a nucleotide sequence set forth in SEQ ID NOS:1-35, 49, 51-56, 66-68, 71-92, or 94-101, or a nucleic acid encoding an amino acid sequence set forth in SEQ ID NOS:38-46, or a fragment or variant thereof.

10. The recombinant genetic construct of claim 1, wherein the one or more plants from which the nucleotide sequences are derived or derivable is or includes a grass of the Poaceae family; a Gossypium species; a berry plant; a tree; an ornamental plant; a vine; a cereal; a leguminous plant; a solanaceous plant; a brassicaceous plant; a cucurbitaceous plant; a rosaceous plant; or an asteraceous plant.

11. A method of genetically improving a plant, including the step of inserting at least a fragment of the genetic construct of claim 1 comprising one or more nucleotide sequences derived from one or more plants into the genetic material of a plant cell or plant tissue, wherein the plant that is genetically improved is of the same species as the one or more plants from which the one or more nucleotide sequences of said nucleic acid fragment of the genetic construct are derived.

12. The method of claim 11, including the further step of selecting a genetically improved plant wherein one or more traits of the plant selected from the group consisting of disease resistance; abiotic stress tolerance; and a morphological trait, are altered or modified as a result of insertion of the at least a nucleic acid fragment of the genetic construct, into the genetic material of the plant.

13. The method of claim 12, wherein the trait of the plant is relatively improved, increased, or otherwise positively altered by the expression of one or more nucleic acids in the plant from the nucleic acid fragment of the genetic construct inserted into the plant, wherein said one or more nucleic acids comprise one or more small RNA sequences and wherein said nucleic acids are capable of altering the expression and/or replication of one or more nucleic acids of a plant pathogen and/or an endogenous plant nucleic acid.

14. The method of claim 12, wherein the trait of the plant is relatively improved, increased, or otherwise positively altered by the expression of one or more proteins from the nucleic acid fragment of the genetic construct inserted into the plant.

15. The method of claim 12 wherein the trait is resistance to a disease associated with a pathogen selected from the group consisting of a plant virus; a nematode; an insect; a fungal plant pathogen; and a bacterial plant pathogen.


SEQUENCE LISTING (153 sequences)

SEQ ID NO: 1 - DNA, 1165 bp, Artificial sequence
  Synthetic
gtttacccgccaatatatcctgtcaaaactagttaggatcggcttagtaatgaatcttctctatccattttgcgttatatagcagccacaagactttcggacaaataaagtagtcggagaagaggatttctatttcataagtaacttgaatgggggaaattaatattggtggaatgaaaattatgatatgcaccagaaatcatatgtgaaaatgcaaattagtaaagaaacaaatgattattactattattattagttctcataataaattcaactggaatccaacaacatacattgaatagaaagaaagaagcaaaacggaaaatgcgaacagtttctcactgttgacatatacacgtgcgcacatgtaattggttactaagaggttattaggacgccttgtatatatagtgataaggcttcctatctaacggacaaaaagagttagcaaacctcatcttacaggaatggtaaccattggattttgtggttcttggcattacaaaatcaatggccactgaattttaacccctcactcgtccttatctcaaacttcccatactgacaaacaagatatgttttttttttcttttttaaaaaatacttgcaatttttttgttgcttttgctttttctttctgacgagtttttcatttttaaaaataatatcacaaggtatgtttggtataactgaaaatattaactaaaaaaataaggaaaatacttcctttccatattgattgtcgaacacaacccaccctgatacccagagtgttgagtaaaaatatgtataaatgtttttgtcataatattttttgattaattacatgaaaaaacacaccctaacacgaaaataaagtctgcaacccctgtattttgtttctttctcgtttggttttgggcatagagtaatttctgcgccatatatttgaactgttaattctacaaagggaaacttggtgagtagtactttggggaaaactgtttatgaatgatacttcaccttaacttagaaggaatcaacaagtatggtacaaacttatatttggctgaaataatccaacgccaattctggattttctcagataattattatatcaatgcattttatagacatattgctttagatccatcgaaaacagtttacaccacaatatatcctgcca

SEQ ID NO: 2 - DNA, 3 bp, Artificial sequence
  Synthetic
tca

SEQ ID NO: 3 - DNA, 3 bp, Artificial sequence
  Synthetic
gtt

SEQ ID NO: 4 - DNA, 774 bp, Solanum lycopersicum
actgttttcgatggatctaaagcaatatgtctataaaatgcattgatataataattatctgagaaaatccagaattggcgttggattatttcagccaaatataagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaatta

SEQ ID NO: 5 - DNA, 1007 bp, Solanum lycopersicum
gataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagttgattaaaa

SEQ ID NO: 6 - DNA, 1035 bp, Solanum lycopersicum
tcatcggctaactcaaaatagaaaacagtatatatcagataacatcataaaatcaactaaaatactcaacatgcagcattttcaattaccataacccttggtcataacaccaagctcatcaacgaggactcacgcctcctcatcatactcatttgggaattaggttcattagattgaatatattaacatctttcaagattcattttctttattcctctcatgtcggtacgtgacactccgctcctcaatatactatcctcgtgtcagaacgtgacactctgatcctcattctatcctggtgtcgaaatgtgacacccgatccatattctatcatggtaccggaacgtggcacccgatctatatactatcctggtgtcgaaacgtgacactccgatcctcattctatcctggtgtcggaacgtgacacccgatccatattctatcctggtaccggaatgtggcacctgatccgtatactatcctggtgtcggaacgtgacacccgatccacatactatcctgtgttggaatgtgacactcagatcctcattctatcctactaccggaacgtggcacccgatcccctaatctcactactttcgttcatcaagccttcttttatactaaggcatcatcattaacaaagtagattagggtttctttttcaagatttagaattccatagcttcatcatgcttatctcatcacaattatataatcacaacatgcaaatacacaattaagcatatagaagggtttacaacactacccaatacatatcattcgctattaagagtttactacgaataatgtaaaaaaatcataacctacctccaccgaagaattttgattaagcaagcaatttcccaaagctttgttctcttctttctcttgatcgtacgtttctccctctctttatgttcttttctttttcttattcaaaccctctttcttttaccctaattagcatataatttaatcaacaaaagaaaccctagaagccgcagtgccactgatttctctcctccagacgaag

SEQ ID NO: 7 - DNA, 1029 bp, Solanum lycopersicum
tcaatactcttatacgatttcgtcttattgtgctttgtttgatttattaaaaataatatctttatcttaacaaaatatatgtaaagttgccatgcatacatatcactctttaaagtctcatttatgaaattacaatgtatatgttatgtaaataacctgtcatgtccattgaatcgaagacctttcaggaaataatagttgtttgcgtgattgaaaatagttatttcaatttatttattattttacgaaatcaatatagtattcattttattctcataatttatttacgtttaagtgcaactaaatttgtataattctaatttttttctcgaaaatcaaataataaaactattaatccatagtcaataaggcttcctaaatcgatctactaaattaacttatttcaaaggttcaaaatgattagttattaatgaaaatttcatctactctttgtaaatattctttggttgccagtttctaactcgagttgcagaccgtaactaatttgcatgagccaaaatcaatggtcactcatatggcggtaaatatgttcttgaaccttgtatacaccactcgtcacacaataataattaaacttacctaaagtcattaccttaatcgcaaggagggaaatgccaaaggccgacctaacgacaaaagtaaggtttagtagtttttattaacaagaaatttgcttacatgtcatttatatataatttattataaataagtctaaacagaagaaatttaatcagaatttgtcatatagtaaaatggaaggacgaaagtaacgtttttcccaagaatatattttcttttatttcatcgaaaatcactcgcactcttttatttatttctttatatataaaaatagcggagaagagagttttgaatactgtgaggagaggttgaagaatttcgaaattatatatagcgggactcctctagggttttgtttcatcttcagcttcttctctgataactgttttctcttttttattatatttattttggcagagacaagaaaga

SEQ ID NO: 8 - DNA, 402 bp, Solanum lycopersicum
agtgtatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgcaaaatggatagagaagattcattactaagccgatcctaactagttttgatttggtaaaacctaatgttagcaggccttagtagtgtattcgatatggttgcagcaacaaaagtga

SEQ ID NO: 9 - DNA, 782 bp, Solanum lycopersicum
ggtgctgctataattacttaaaagtgcgagtgtcctgtctgtttcccggttttgctattatgttgccagtcaatttgtttttttgatgggatggagaagtttggtggtgggggctatgaatgcacggtagcaaacaacagattgccagtattatctcatgtttccatttaatgtggttaatattctctacatacttgagaggtgcctgatgcattgccctcttctgtctggctacaccatcccttggtcgaagcgtctcttttttaggttgtttgtagttgaaggagagtgattgtgatgttttctcctcgtcttttctctcattttctccttttatctgattttgcacttttgtggttcttttttttcttggacccaataatgtcaatatttattgaatgagaaaattcctatatcatatcagtttgaggaaatcattactatttgtgtggatacaggagttttgactctttattggcgatattttgtattctattgttgctgttttggatgtggtttcagaacttccttagtgcatttgctcttaaatctgttttgcagtaaaattgaggctataaaagcttcattgcagattaccctcggatgagggatctcctcattgcctgtcatatattggtttcttttcatccaacacgcaggatacatacatttattgaatttgaccttctattttgggacaactctactgtgaaattggagggattgttgaatttttttcttgcatgagttcattgatggtattatttttgattaggaacgag

SEQ ID NO: 10 - DNA, 299 bp, Solanum lycopersicum
ttttaatgcttagcaatgctctatcagattttctttttgtcgaatgaacggtaatttagagttttttttttgctatatggattttcgattttgatgtatgtgacaacccttgggattgttgatttatttcaaaactaagagtttttggcttaaaaaataaaataaaattagcatataattaagtataaaagatggcaataataacccactaattaactcaaggttacctcttttaacccccaagtagttagacttattaacattaacctactaactttataattaaagcaggaatagtc

SEQ ID NO: 11 - DNA, 866 bp, Solanum lycopersicum
ttttggttctcatttggcaccagtgctggcaattaatactctttatcaattgccatcattcatggagttccttctgctttcagaaacaggatagtttattgccttgtttcgagacatcgatcctgatctatgaactaaattaaactttaaatgaactgctcaggctattcttggttataacttgtatgcaccaaatagcagaaggaattttaggtgtctatgcacccttgttgttattaatcagctattaataagctgcacggatgaaaaaaaaattaatcatgggaaatcgttatccaatgttcttttataattgtgctgacttgcaaggtgacttccttgcaatctctgtagcctaatatttccacattgagatggaagtaacttgtatgtattatgtaactcaactgtaatggtaagggcgtatgatgggaaatttcgttggtttttatattccattagcgtatgttaattgtagttattgacttatgttccttctcacaggaaatcggtaaatattagcacatgagatgtgttaataacaggtgatctttgtggagtgatgttcttctattcaaattgtaagctggcatgatcatttcctcgcttttgaccttgcatttttccgtgtgttgaaaatctcgactcagtgcatagtggttttgttgtctcatctcaattttcttgtgattgttgcatccctagtgttctggtccagcatttttgtgtcctgtgtatgttttaacttgcttttagtaaccaaatcctctcttgttatgaccataaaaagaaccaaaagtgactgaacaaaatctacaatgggaacttccttttttgtcttgtacagttgtactgtaatcttgtgc

SEQ ID NO: 12 - DNA, 143 bp, Solanum lycopersicum
gagcaggaaagtattgggtgagatattgttgacagaagatagagagcacgaataatgaggtgctaattggaagctgcaccttaattctttgtgctctctattcttctgtcatcatcttcagtccctccccgaccctctctacc

SEQ ID NO: 13 - DNA, 143 bp, Artificial sequence
  Synthetic
gagcaggaaagtattgggtgagatattgttctaaatcaaccaatgtcaagaataatgaggtgctaattggaagctgcaccttaattctttttgacattggtttgatttagatcatcttcagtccctccccgaccctctctacc

SEQ ID NO: 14 - DNA, 143 bp, Artificial sequence
  Synthetic
gagcaggaaagtattgggtgagatattgttcaattccatctttcttcatgaataatgaggtgctaattggaagctgcaccttaattctttatgaagaaagattggaattgatcatcttcagtccctccccgaccctctctacc

SEQ ID NO: 15 - DNA, 144 bp, Artificial sequence
  Synthetic
gagcaggaaagtattgggtgagatattgttatcttttgaagttcgtcttgaataatgaggtgctaattggaagctgcaccttaattctttgaagacgaactttcaaaagatatcatcttcagtccctccccgaccctctctacc

SEQ ID NO: 16 - DNA, 143 bp, Artificial sequence
  Synthetic
gagcaggaaagtattgggtgagatattgttgatcaggaattcttttcgagaataatgaggtgctaattggaagctgcaccttaattcttttcgaaaagaatttcctgatcatcatcttcagtccctccccgaccctctctacc

SEQ ID NO: 17 - DNA, 142 bp, Artificial sequence
  Synthetic
gagcaggaaagtattgggtgagatattgtttgattaatcttccaatcgagaataatgaggtgctaattggaagctgcaccttaattcttttcgattggaagtattaatcaatcatcttcagtccctccccgaccctctctac

SEQ ID NO: 18 - DNA, 397 bp, Artificial sequence
  Synthetic
aaactttattccatgatattttcccgcgtgcgtaaattcaatcttatggtggattttgattttatcaattagtctacaacgtcttatgttcatgatcgggattatataaaatattttctcacagatcagacttattgatgccgaggaccgcatcgatattaaagattatcaatatatttcattcgctattctccttcacaaaaaaatgaagtatgaacaactgaagtaagatgtatgaaatgttgaatgcttcgagcttctagaagtggtttcttattttggtaaaaggttgtcattacctgattcagttacgaaattcgataagaagcttctttctcgcattcaaattcgagttaagcctttaccgaaatttgattctaccgtgggggtgacagtc

SEQ ID NO: 19 - DNA, 146 bp, Artificial sequence
  Synthetic
aaacttaattccacgatattttcccgcgtgcgtaaattcaagaccatggtggcttttgattttatcaatgagtctacaatgtcttatgttcatgattgggagaatataaaatcttttctcacagatcagacttattcataccgagg

SEQ ID NO: 20 - DNA, 124 bp, Artificial sequence
  Synthetic
aacgcatcgatattaacgattatcattatattggattcgctattctccttcacacaaaaatgaagtatgaacaactggggaagatgtatgatatgtggaatgcttcgagcatctcgaagtggtt

SEQ ID NO: 21 - DNA, 126 bp, Artificial sequence
  Synthetic
tcttattatggtaaaaggttgttattacctgattcagtcacggaattcgataagaagcttgtttcgcgcattcaaattcgagttaatcctttgccgaaatttgattctaccgtgtgggtgacagtc

SEQ ID NO: 22 - DNA, 803 bp, Artificial sequence
  Synthetic
ccaaatgatgattattcaagtacagacatgtcttctctgactcttatgaagaaactaataaggcttgacaatggggacaacttgggctggtgtgaaaaaattaggattctttgtttgtgcttcctaatggcgatataagagaggaaagcaagtggacatctgattacaataattatgataaacatcctgaatgtttgtccattctatgtatatctgacaaatcattgtatgggaggttcacctactctgacatcaatgttcatatcatgcaaacaagagagatcatcttgagtaaaataagtgagatagatgaggttggtgaaactgatgaaaacaatttcttgcttagttatataataggggaagtagatgcctttgaagaagatgattttgaagaagaagaagacaaagattaggaacatcatcttttggaacctttgaatctgattctatcaaagaatcagagggttttgatatttctgctagattgatagtacatacaaaccatcatgtctcaaactagaaaaatgatctttttttttgcaacactaagcaaaatgctaataaggttatcaagatcagtccaacttgggacgttggagaatctctttagcaaatttaaagaattatcacatttttctaaactttcttctgaatcagaaacaaaggaatatatgacaacattgctttcaacttgataataaatgttataagtagatatcccctttttctcactttttaatgaagaagcaatcaagcagttgttaggatgatccaaaaaagaaattgtcttttgagttgtt

SEQ ID NO: 23 - DNA, 205 bp, Artificial sequence
  Synthetic
ccaaatgatgattattcaagtatagacatgtcttctctgactcacatgaagaaactaataaggcatgacaatgaggacagcttgagctggtgtgaaaaaattaaggattctttgtttgtccttcataatggcgatataagagaggaaggcaagatcacatctgtttacaataattatgctaaaaatcctgaatgcttgtacattc

SEQ ID NO: 24 - DNA, 211 bp, Artificial sequence
  Synthetic
tatgtttatctgaaaaaacattgtatggaaggtacacctactctgacatcaatgattatatcatgcaaacaagagagattatcttgagtaaaataagtgagctagatgaggttgttgaaacagatgaagacgatttcttgcttagttatctaagaggggaagaagatgcctttgatgaagatgagtttgacgaagaagaagacacagatta

SEQ ID NO: 25 - DNA, 174 bp, Artificial sequence
  Synthetic
ggaacatcttcttttggaacctatgaatctgattctatcacagaatcagagggttatgatctttctgctagaatgatagtagatacaaaccatcatatctcaaactggaaaaatgatctttttgttggcaacggaaagcaaaatgctaataaggttatcaagatctgtccaact

SEQ ID NO: 26 - DNA, 214 bp, Artificial sequence
  Synthetic
tgggactttggagaatctctttggcaaatttaaagaattatcacatttttctaaaccttctgctgaatcagaaacacaggaatatatgacaccattgttttcaacttgataataaacattataagtagatatcccctttatctcacattttaatgaagaagcattcaagcagttgttaggaagatccaaaacagaaattgttttttgcgttgtt

SEQ ID NO: 27 - DNA, 1567 bp, Solanum lycopersicum
gtgtagagccatggcgattcctaatatacggatcccttgtcggcagttgttcatcgacggtgaatggagagaacccctcaagaagaaccgattacccatcatcaatccggccaatgaagaaattatcgggtatattcccgcagctacagaggaggatgtagatatggccgtcaaagctgcacggagtgcgcttcgtcgagatgactggggttctactactggagcacagcgtgccaaatatcttcgtgctattgctgctaaggtactggagaaaaagcctgaactggctacacttgagactatcgataatggaaaaccctggttcgaggctgcctcggatatagatgatgtcgtagcgtgttttgagtactatgcagatctagctgaagctttggattcaaaaaagcagactgaagttaaacttcatttggattcattcaagacccatgttttaagagaacctcttggtgttgtggggttgattactccatggaattatcctcttttgatgaccacatggaaagtcgctcctgccctagcagctggttgtgcagcaatactcaagccatcagaactagcatctattacctctttggagttgggtgaaatctgtagagaggtgggtcttcctcctggtgcccttagcatactaacgggattaggacatgaagctggttctcctttggtatcacatcctgatgttgataagattgcatttacaggaagtggcccaacaggggtcaagatcatgaccgctgcagctcaacttgttaaaccagttactcttgagcttggtggaaaaagtccaatagttgtgtttgatgacattcataaccttgatacagctgtggagtggactctttttggctgcttttggacaaatggtcaaatttgcagtgcaacttcacgtcttataatacaggaaacaattgctccacaatttttggccaggcttcttgagtggacaaaaaacatcaaaatctcagatcccttggaagaagactgcaagcttggtcctgtgattagtcgtggacagtatgagaagatcttgaagttcatctctacagccaaagatgaaggtgcaaccattctttatggtggtgaccgacctgagcacttaaagaaaggatattacattcaaccaacaatcataactgatgttgatacgtccatggagatctggaaagaggaggtatttggacctgttctttgtgtcaaaacatttaaaactgaagaggaagccattgaactagcaaatgataccaagtttggtttgggtgctgctattttgtcaaaagatcttgaaagatgtgaacgtttcacaaaggcttttcagtcggggattgtctggatcaactgctcgcagccatgcttttggcaaccaccatgggggggtaagaagcgtagtggttttggacgtgagcttggggaatggagtctcgagaactacctaaacattaaacaggtgactcagtatgtgactccggacgaaccatgggctttttacaagtctccttcaaagctgtaaaactttcaagtggtcaaggattatgtgaatgatgaagaa

SEQ ID NO: 28 - DNA, 1409 bp, Solanum lycopersicum
ggaaaaatgaactacacaaattcacctaaaaattgaaatcaacaacaaaaaaaaatcaaatcttgaaaacccccttttagatagaagagcaaaaaaatcaaatcttgatttgcccctttttgtgttattgttgtttttagataaaagagcaaaaaaaatcaaatcttgaaaaccccttttctgttctaatgggtaaaggaggcagtgatgaaaatatggctgcttggcttcttggtgttaacaccctcaagattcagcctttcaatctccctgctttgggaccccatgatgttagagttaggatgaaggctgtcggtatttgtggaagtgatgttcattacctcaagaccatgaggtgtgcggattttgtggttaaagaaccaatggtgattgggcatgaatgtgccgggatcatagaggaagttggcggtgaagtcaagacattggttcctggagatcgtgtagcactagagcccggaattagttgttggagatgtaatctttgcaaagaagggcgatataatctctgccccgagatgaagttctttgctactccccctgttcatggttctcttgcgaatcaggtagtccaccctgcagacctatgtttcaagctcccggatgatataagtttagaggagggagcaatgtgtgagccacttagtgttggtgttcatgcctgtcggcgtgcaaatgttggtcctgagacaaacatattagtgctgggagctggaccaattgggcttgtcacgctgcttgctgctcgtgcttttggtgccccaagaattgttattgtggatgtagatgactatcgtctttctgttgcaaagaagttaggagcagatgacatcgtcaaggtttcaatcaatattcaggatgtagctacagatatagaaaacattcagaaagcaatgggaggtggaatcgacgcgagttttgactgtgctggctttaacaaaactatgtcgaccgctcttggtgcaactcgtccaggtggcaaagtttgcttggttggaatgggacatcatgagatgaccgttcctctcactccagctgctgcaagggaggtcgacgtcatcggcatatttcgctacaagaatacatggccattgtgtcttgagttcttaagaagtggtaagattgatgtgaaacctttgatcacacacaggtttggattctctcaagaagaagttgaagaagcttttgaaacaagtgctcgtggtggtgatgctattaaagtcatgtttaatttgtaaaaaaaaaaaatactttttaaatttgagaaaataagtttttttttttaccaaatatgtttgtaaaatgtatatctaaaaaaaatgtttttttaatgcttttgaaaactactatgtattaatataaaatggtgaaatgaagtagatggttaactt

SEQ ID NO: 29 - DNA, 897 bp, Solanum lycopersicum
cactaaatccaacaacttacatttaaaaaaatagttccacaaacatggcctacttgagatcttcttttgttttcttccttcttgcttttgtgacttacacttatgctgccactttcgaggtacgcaacaactgtccatacaccgtctgggcggcgtcgaccccaataggcggtggtcgacgtcttgatcgaggccaaacatgggtcatcaatgcaccgaggggcactaagatggcacgtatatggggtcgtacgaattgcaactttgatggtgctggtagaggttcatgtcagactggtgattgtggtggggtcttgcaatgtaccgggtggggcaaaccaccaaacaccctggccgagtacgccttggaccagtttagcaacctagatttctgggacatttctttagtcgatggatttaatattccaatgactttcgccccgaccaatcctagtggagggaaatgccatgcaattcattgtacggctaatataaatggtgaatgtcctggttcacttagggtacccggaggatgtaacaatccttgtaccacgttcggaggacaacaatattgttgcacacaaggtccatgtggccctactgatttgtcgagatttttcaaacaaagatgtcctgatgcgtatagctacccacaagatgatcctactagcacatttacttgccctagtggtagtacaaattatagggttgttttttgtcctaatggtgttactagcccaaatttccccttggagatgccctcaagtgatgaagaggctaagtaaaattgagtcactttcttttaaattgcttgaagtagtcgagttatataattggcttgtaataaacctaatataattacatgaataaaagtcacatcatcacaaatatgttgtt

SEQ ID NO: 30 - DNA, 1611 bp, Solanum lycopersicum
cctactctttggaacaaccaaaacttgttcttttttcaatgctaatttattttcatttttccattattattattaaaaattaaaatagcaaataaataaataaaaaaaaaattggaataattaagttgtaagtgtaatagtttaatacaagcaaccctgaaaatcgcctatataaagtgtataaaaatttagtctttgcctcatcaaagaaaattcatcttatagagaattttaatttaagaagtttatcatcatcatgtctctgctttcagatcttatcaacctcaatctctcaggtgatactcagaagatcattgctgaatacatatggattggtggatcaggcatggacatgaggagcaaagccaggactctccctggtccagttactagtcctgcagaactacccaaatggaactacgatggatcgagcactggtcaagctcccggagaagacagtgaagtgatcttatatccacaagcaatcttcaaggacccattcagaagaggcaacaacatcttggtcatgtgtgatgcctatactcctgctggtgagcccatcccaacaaacaagaggcacgccgccgccaaggtcttcagccaccctgatgtggctgctgaggaaacttggtatggtattgaacaagaatataccttgctgcaaagggaggtcaactggcctcttggatggcccattggcggttttcctggcccccagggaccatactactgtggaaccggagctgacaaggcctttggacgtgacattgttgacgcccattacaaggcttgtctctatgctgggattaacatcagcgggatcaatggtgaagtcatgccgggacagtgggaatttcaagttggaccttctgttggcatctcagctggtgatgaagtgtgggtagctcgttacattctagagaggattgcagagattgctggggtggtcgtgtcattcgaccccaagcctattccgggcgactggaatggtgcaggtgctcacacaaattacagcaccaagtcgatgagggaagacggaggctatgaaataatcttaaaggctattgagaagcttggcttgaagcacaaagaacacatagctgcatatggtgaaggcaacgagcgtcgtctcactggaaagcacgaaacagccaacatcaacacattcaaatggggggttgcaaaccgtggtgcatctgtccgtgttggaagagacacagagaaggcaggcaagggatactttgaggacagaaggccagcctcaaatatggacccatacgtcgttacctccatgattgcagaaaccaccatcatcggttaaccttgaagacttgatagtatgaatttgctcgagggatcgcttgtttctggtttgcacaatttgggataggagaaaagattgaattgtggaacgaccctttggacttcacctgtgttatttagttatagggatagtttgtctctggttatttttctgtttatttgccccagttgaattgtattttcatacagcaaagccttatttcattgcctatgatttggcaatgctgtgttacaaatgttattcttattaataacaaagatattgaaagggtttggttcacttcatta

SEQ ID NO: 31 - DNA, 2321 bp, Solanum lycopersicum
gggtttatctcgcaagtgtggctatggtgggacgtgtcaaattttggattgtagccaaacatgagatttgatttaaagggaattggccaaatcaccgaaagcaggcatcttcatcataaattagtttgtttatttatacagaattatacgcttttactagttatagcattcggtatctttttctgggtaactgccaaaccaccacaaatttcaagtttccatttaactcttcaacttcaacccaaccaaatttatttgcttaattgtgcagaaccactccctatatcttctaggtgctttcattcgttccgagtaaaatgcctcaaattggacttgtttctgctgttaacttgagagtccaaggtagttcagcttatctttggagctcgaggtcgtcttctttgggaactgaaagtcgagatggttgcttgcaaaggaattcgttatgttttgctggtagcgaatcaatgggtcataagttaaagattcgtactccccatgccacgaccagaagattggttaaggacttggggcctttaaaggtcgtatgcattgattatccaagaccagagctggacaatacagttaactatttggaggctgcatttttatcatcaacgttccgtgcttctccgcgcccaactaaaccattggagattgttattgctggtgcaggtttgggtggtttgtctacagcaaaatatttggcagatgctggtcacaaaccgatactgctggaggcaagggatgttctaggtggaaaggtagctgcatggaaagatgatgatggagattggtacgagactggtttgcatatattctttggggcttacccaaatattcagaacctgtttggagaattagggattaacgatcgattgcaatggaaggaacattcaatgatatttgcaatgccaagcaagccaggagaattcagccgctttgatttctccgaagctttacccgctcctttaaatggaattttagccatcttaaagaataacgaaatgcttacatggccagagaaagtcaaatttgcaattggactcttgccagcaatgcttggagggcaatcttatgttgaagctcaagatgggataagtgttaaggactggatgagaaagcaaggtgtgccggacagggtgacagatgaggtgttcattgctatgtcaaaggcactcaactttataaaccctgacgaactttcaatgcagtgcattttgatcgcattgaacaggtttcttcaggagaaacatggttcaaaaatggcctttttagatggtaatcctcctgagagactttgcatgccgattgttgaacacattgagtcaaaaggtggccaagtcagactgaactcacgaataaaaaagattgagctgaatgaggatggaagtgtcaagagttttatactgagtgacggtagtgcaatcgagggagatgcttttgtgtttgccgctccagtggatattttcaagcttctattgcctgaagactggaaagagattccatatttccaaaagttggagaagttagtcggagtacctgtgataaatgtacatatatggtttgacagaaaactgaagaacacatatgatcatttgctcttcagcagaagctcactgctcagtgtgtatgctgacatgtctgttacatgtaaggaatattacaaccccaatcagtctatgttggaattggtttttgcacctgcagaagagtggatatctcgcagcgactcagaaattattgatgcaacgatgaaggaactagcaacgctttttcctgatgaaatttcagcagatcaaagcaaagcaaaaatattgaagtaccatgttgtcaaaactccgaggtctgtttataaaactgtgccaggttgtgaaccctgtcggcctttacaaagatccccaatagaggggttttatttagccggtgactacacgaaacagaaatacttggcttcaatggaaggcgctgtcttatcaggaaagctttgtgctcaagctattgtacaggattatgagttacttgttggacgtagccaaaagaagttgtcggaagcaagcgtagtttagctttgtggttattatttagcttctgtacactaaatttatgatgcaagaagcgttgtacacaacatatagaagaagagtgcgaggtgaagcaagtaggagaaatgttaggaaagctcctatacaaaaggatggcatgttgaagattagcatctttttaatcccaagtttaaatataaagcatattttatgtaccactttctttatctggggtttgtaatccctttatatctttatgcaatctttacgttagtt

SEQ ID NO: 32 - DNA, 2157 bp, Solanum lycopersicum
ctgttgtgaaaaattaagggatgcattttgcaaattgtgacaattcagtcaaatgcacaactaccctcaaacctcaacaactcttgatggcttttgaagaaaagaattcagagacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaagtaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaactattccttggaaatgcaggaacagcaatgcgtccgttgacagcagcagttactgtagctggaggacattcaagatatgttcttgatggagttcctaggatgagagagagaccaattggtgatttggttgatggtcttaagcagcttggcgcagaggtagattgttcccttggtacgaattgtcccccagttcgaattgtcagcaagggaggtcttccaggagggaaggtaaagctctctggatccatcagcagccaatacctgactgctctgcttatggctgctcccctggctctaggagatgtggagattgaaataattgacaaactgatatctgtgccttatgttgaaatgacactgaagttgatggagcgatttggtgtctttgtggagcacagtagtggctgggacagattcttggtaaaaggaggtcagaagtacaaatctcctgggaaagcatttgttgaaggagatgcctcaagtgctagctattttttggcgggggcagcagtcacaggtggaaccgtcactgttgaaggttgtggaacaagcagtttacagggagatgttaagttcgctgaggtcctcgagaagatgggggcagaagttacatggacagagaacagtgtcacagttaaaggacctccgaggaactcttctggaatgaaacatttgcgtgccattgacgtgaacatgaacaaaatgccagatgtggccatgactcttgccgtagttgcactttttgctgatggtcctactaccataagagacgttgctagctggagagtaaaggaaactgagcggatgattgccatatgcaccgaacttaggaagttgggtgcaacagttgttgaagggtcagactactgcataatcaccccaccagaaaagttaaacgtaacggagattgatacatatgatgaccacagaatggctatggctttctctcttgctgcttgtgctgatgttccagtcactattaaggaccctggctgtactcgcaaaaccttccccgactacttcgaggttctccagaagtactctaagcactaaaccacttcacatgtagaaggaattattttgtactacaagagaaattatgcaccagtttgcaaccaaaatggtgcccataccggaagagaaaaaagctttccaactcctttttatatgtctatgtgagatcatgttcattgtatttgttgaagttgagcttctttttttgtttctcgtgtagaagacatgtatactatatagttaagtacacttccttgaagaatatttaccattgattatcaccgttttagttattgcattttggtattcaaaataaatttgtttcgaggattaaagcta

SEQ ID NO: 33 - DNA, 2288 bp, Solanum lycopersicum
aggacccttacaacacattttcgtggcgctcatcacttcttatagccattttgcctcttcctttcacttctctcacctttatcgaccaacaatggcggctgctgcctcaccatctccatgtttctccaaaaccctacctccatcttcctccaaatcttccaccattctacctagatctaccttctctttccacaatcacccacaaaaagcctcaccccttcatctcatccacgctcaacataatcgtcgtggttttgccgttgccaatgtcgtcatatccactaccacccataacgacgtttctgaacctgaaacattcgtttcccgtttcgcccctgacgaacccagaaagggttgtgatgttcttgtggaggcacttgaaagggaaggtgttacggatgtatttgcatacccaggaggtgcttctatggagattcatcaagctttgacacgttcgaatattattcgtaatgtgctaccacgtcatgagcaaggtggtgtgtttgctgcagagggttacgcacgggctactgggttccctggtgtttgcattgctacctctggtcccggagctacaaatcttgttagtggtcttgcggatgctttgttagatagtattccgattgttgctattacaggtcaagtgccaaggaggatgattggtactgatgcgttccaggaaacgcctattgttgaggtaacgagatctattacgaagcataattatcttgttatggatgtagaagatattcctagggttgttcgtgaagcattttttcttgcgaaatcgggacggcctggcccagttttgattgatgtacctaaggatattcagcaacaattggtgatacctaattgggatcagccaatgaggttgcctggttacatgtctaggttacctaaattgcctaatgaaatgcttttggaacaaattgttaggctgatttccgagtcgaagaagcctgttttgtatgtgggtggtgggtgttcgcaatcaagtgaggagctgagacgatttgtggagcttacaggtattcctgtagcgagtactttgatgggtcttggagcttttccaactggggatgagctttcacttcaaatgttgggtatgcatggaactgtgtatgctaattatgctgtggatagtagtgatttgttgcttgcatttggggtgaggtttgatgatcgagttactggtaaattggaagcttttgctagtcgagcgaaaattgtccacattgatattgattcggcagagattggaaaaaacaagcaacctcatgtttccatttgtgcagatatcaagttggcattacagggtttgaattccatattggagggtaaagaaggtaagatgaagttagatttttctgcctggaggcaggagttaacggagcagaagatgaagtacccactgaattttaagacttttggtgatgccatccctccacaatatgctattcaggttcttgatgagttaactaacggaaatgccattattagtactggtgtggggcaacaccagatgtgggctgcccaatactataagtacaaaaagccacgccaatggttgacatctggtggattaggagcaatgggatttggtttgcctgctgctataggtgcggctgttgggagaccgggtgagattgtggttgacattgacggtgatgggagttttatcatgaatgtgcaagagttagcaacaattaaggtggagaatctcccagttaagattatgttgctgaataatcaacacttgggaatggtggttcaatgggaggatcgattctataaagctaacagagcacacacttacttgggtgacccttctaacgaggaagagatcttccctaatatgttgaaatttgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagcggtggtgctttcaaagatgtgatcacggagggcgatgggagatgttcctattgacttaaagaaactacataactagttctagacattgtattatctaaaataaacttctattaagccaaaagtgttcgatttgtctagtttgctgttagtctttggcgtggctttgcttgttgtggctgttgtactatcttctacttggtatttatgttcacttaaagttttgcatcatcttgcttttgtcgaatggaaggattcagattattatttttta

SEQ ID NO: 34 - DNA, 1994 bp, Solanum lycopersicum
tgaaacgataacgctaaagcaaacggtgatattttctcagaggagctgagagtgcagtcatgacaacaacggccgtcgtcaaccatcctagcattttcactcaccggtcgccgctgccgtcgccgtcctcctcctcatcctcatcgccgtcatttttatttttaaatcgtacgaattttattccatacttttccacctccaagcgcagtagtgtcaattgcaatggctggagaacacggtgttccgttgcgaagaattatacagttcctccctcagaagttgacggtaatcagttaccggagctggattgtgtggtagtcggagcaggaattagtggtctctgcattgctaaggtgatatcggctaattatcccaatttgatggtgacggaggcgagggatcgtgccggtggaaacataacgacggtggaaagagatggatacttatgggaagaaggtcctaacagtttccagccttcggatcctatgttgactatggctgtagattgtggattgaaggatgatttggtgttgggagatcctgatgcgcctcgctttgtcttgtggaaggataaactaaggcctgttcccggcaagctcactgatcttcccttctttgatttgatgagtattcctggcaagctcagagctggttttggtgccattggccttcgcccttcacctccaggttatgaggaatcagttgagcagttcgtgcgtcgtaatcttggtgctgaagtctttgaacgtttgattgaaccattttgttctggtgtttatgctggcgacccatcaaaattgagtatgaaagcagcatttgggaaagtgtggaagctagaacaaactggtggtagcattattgggggaacctttaaggcaataaaggagagatccagtaaccctaaaccgcctcgtgatccgcgtttaccaacaccaaaaggacaaactgttggttcatttaggaagggtctgagaatgctgccagatgcaatttgtgaaagactgggaagcaaagtgaaactatcatggaagctttctagcattacaaagtcagataaaggaggatatctcttgacatacgagacaccagaaggagtagtttctctgcgaagtcgaagcattgtcatgactgttccatcctatgtagcaagcaacatattacgccctctttcggtcgccgcagcagatgcactttcaagtttctactatcccccagttgcagcagtgacaatttcatatcctcaagaggctattcgtgatgagcgtctggttgatggtgaactaaagggatttgggcagttgcatccacgttcacagggagtggaaacactaggaacaatatatagttcatcactcttccctaaccgtgctccaaatggccgggtgctactcttgaactacattggaggagcaacaaatactgaaattgtgtctaagacagagagccaacttgtggaagcagttgaccgtgacctcagaaagatgcttataaaacccaaagcacaagatccctttgttacgggtgtgcgagtatggccacaagctatcccacagtttttggtcggacatctggatacactaggtactgctaaagctgctctaagtgataatgggcttgacgggctattccttgggggtaattatgtgtctggtgtagcattgggaaggtgtgttgaaggtgcttatgaaattgcatctgaagtaactgggtttctgtctcagtatgcatacaaatgaaacctcctcttggggaggtactgttaggtttcaaaagttttgcttattagagttattttagctttggtaaatgatttatgcttgatttcagtcgtttttgttgtaatcttggttctcatttctttgggacaaaatgttcttgtcaaggaacaatacgtttagagttcgagtatctgttaattgtaagaaaatctaacatattgggcataattagctgcctgctttgccagtagatatattatatggcttggttaaata

SEQ ID NO: 35 - DNA, 825 bp, Solanum chilense
atgaacagtacatctatgtcttcattgggagtgagaaaaggttcatggactgatgaagaagattttcttttaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttccaggaaggacagctaacgatgtgaaaaactattggaacactaatcttctaaggaagttaaatactactaaaattgttcctcgtgaaaagactaacaataagtgtggagaaattagtactaagattgaaattataaaacctcaaccacgaaagtatttctcaagcacaatgaagaatattacaaacaatattgtaattttggacgaggaggaacattgcaaggaaataaaaagtgagaaacaaactccagatgcatcgatggacaacgtagatcaatggtggataaatttactggaaaattgcaatgacgatattgaagaagatgaagaggttgtaattaattatgaaaaaacactaacaagtttgttacatgaagaaaaatcaccaccattaaatattggtgaaggtaactccatgcaacaaggacaaataagtcatgaaaattggggtgaattttctcttaatttacaacccatgcaacaaggagtacaaaatgatgatttttctgctgaaattgacttatggaatctacttgattaa

SEQ ID NO: 36 - DNA, 1403 bp, Solanum lycopersicum
attttggtcataaattgttttaataacataattaaacaaaagataaaagttatcatcagaccaaaaagctctcctttcactgaacttccattgcaatggcttctctcctcaacactgtgccatctattaaactatcaaatttcaactacaacaacccacttcgctcttcacaaatatcattctccctctctcgaagaagactcgttgttagagcaacagagactgaaaaagaagctaaagcagaggcaccagataaggcaccagctgctggtggctcaagtataaatcagattcttggaatcaaaggagccaagcaagaaacggacaagtggaagattcgggttcagcttacaaaacctgttacttggcctccccttatctggggtgtggtctgtggagctgctgcttctgggaacttccactggactccagaggatgtggccaaatcaattgtttgtatgttgatgtctggtccatttctaactggctatactcagactattaatgattggtatgatagagagattgatgctattaacgaaccttaccgtccaattccttcaggtgcggtatctgaacaagaggtcattactcaaatatgggtgcttcttttaggaggccttgggttagctggtattttagatgtttgggcagggcatgactttcctgtaatattttaccttgcacttggtggatccttgctctcctacatctactcagctccaccattaaagctcaaacagaacggatggattggaaattttgctctaggagcaagttatatcagcttgccttggtgggccggtcaagctttgttcgggacccttacacttgatgtaattgtactaacactattgtacagcattgccggtctgggcatagccattgtaaatgatttcaaaagcattgaaggagatagagctatggggcttcagtcacttccagtagcttttggttctgaagctgctaaatggatttgtgttggtgccattgacataactcagatatcagtggcagggtatcttttaggtgctggcaaaccctattatgcttttgcacttctaggtttaattgctccacaagtcttcttccagtttaagtacttcctcaaagatccagtaaaatacgacgtcaaatatcaggccagtgcacagccatttcttatacttggtcttttggttactgctttagcaactagccattagtattcaagtggtgctttcatggtgtagaggagatgccaagctgcttagagcaaacaaagctctttctatttgataatatgacttgtgctttacttttccttcaaatgtagaatgctagaataggatggatgtaaaatatgaagattttgtatgatggttttatgcaaattttggattatgcttggttctgctgtcaaa

SEQ ID NO: 37 - DNA, 524 bp, Artificial sequence
  Synthetic
atggctcaagttattaacacatttgatggagtggccgattatttgcaaacctatcataaattgcccgataattatattacaaaatccgaagctcaagcacttggatgggttgctagcaagggaaacttagctgacgtcgcccctggcaagtctatagggggcgatatattcagtaataggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaacatggtgatgtttagggaaggaaagcttcctggcaaatctggaaggacctggagagaggcagacattaactatacatctggttttcgtaatagtgatcgtatattgtactcctcagattggttgatttacaaaactacagaccattatcagacttttacaaaaataagatga

SEQ ID NO: 38 - protein, 505 aa, Solanum lycopersicum
MAIPNIRIPCRQLFIDGEWREPLKKNRLPIINPANEEIIGYIPAATEEDVDMAVKAARSALRRDDWGSTTGAQRAKYLRAIAAKVLEKKPELATLETIDNGKPWFEAASDIDDVVACFEYYADLAEALDSKKQTEVKLHLDSFKTHVLREPLGVVGLITPWNYPLLMTTWKVAPALAAGCAAILKPSELASITSLELGEICREVGLPPGALSILTGLGHEAGSPLVSHPDVDKIAFTGSGPTGVKIMTAAAQLVKPVTLELGGKSPIVVFDDIHNLDTAVEWTLFGCFWTNGQICSATSRLIIQETIAPQFLARLLEWTKNIKISDPLEEDCKLGPVISRGQYEKILKFISTAKDEGATILYGGDRPEHLKKGYYIQPTIITDVDTSMEIWKEEVFGPVLCVKTFKTEEEAIELANDTKFGLGAAILSKDLERCERFTKAFQSGIVWINCSQPCFWQPPWGGKKRSGFGRELGEWSLENYLNIKQVTQYVTPDEPWAFYKSPSKL

SEQ ID NO: 39 - protein, 355 aa, Solanum lycopersicum
MGKGGSDENMAAWLLGVNTLKIQPFNLPALGPHDVRVRMKAVGICGSDVHYLKTMRCADFVVKEPMVIGHECAGIIEEVGGEVKTLVPGDRVALEPGISCWRCNLCKEGRYNLCPEMKFFATPPVHGSLANQVVHPADLCFKLPDDISLEEGAMCEPLSVGVHACRRANVGPETNILVLGAGPIGLVTLLAARAFGAPRIVIVDVDDYRLSVAKKLGADDIVKVSINIQDVATDIENIQKAMGGGIDASFDCAGFNKTMSTALGATRPGGKVCLVGMGHHEMTVPLTPAAAREVDVIGIFRYKNTWPLCLEFLRSGKIDVKPLITHRFGFSQEEVEEAFETSARGGDAIKVMFNL

SEQ ID NO: 40 - protein, 246 aa, Solanum lycopersicum
MAYLRSSFVFFLLAFVTYTYAATFEVRNNCPYTVWAASTPIGGGRRLDRGQTWVINAPRGTKMARIWGRTNCNFDGAGRGSCQTGDCGGVLQCTGWGKPPNTLAEYALDQFSNLDFWDISLVDGFNIPMTFAPTNPSGGKCHAIHCTANINGECPGSLRVPGGCNNPCTTFGGQQYCCTQGPCGPTDLSRFFKQRCPDAYSYPQDDPTSTFTCPSGSTNYRVVFCPNGVTSPNFPLEMPSSDEEAK

SEQ ID NO: 41 - protein, 354 aa, Solanum lycopersicum
MSLLSDLINLNLSGDTQKIIAEYIWIGGSGMDMRSKARTLPGPVTSPAELPKWNYDGSSTGQAPGEDSEVILYPQAIFKDPFRRGNNILVMCDAYTPAGEPIPTNKRHAAAKVFSHPDVAAEETWYGIEQEYTLLQREVNWPLGWPIGGFPGPQGPYYCGTGADKAFGRDIVDAHYKACLYAGINISGINGEVMPGQWEFQVGPSVGISAGDEVWVARYILERIAEIAGVVVSFDPKPIPGDWNGAGAHTNYSTKSMREDGGYEIILKAIEKLGLKHKEHIAAYGEGNERRLTGKHETANINTFKWGVANRGASVRVGRDTEKAGKGYFEDRRPASNMDPYVVTSMIAETTIIG

SEQ ID NO: 42 - protein, 583 aa, Solanum lycopersicum
MPQIGLVSAVNLRVQGSSAYLWSSRSSSLGTESRDGCLQRNSLCFAGSESMGHKLKIRTPHATTRRLVKDLGPLKVVCIDYPRPELDNTVNYLEAAFLSSTFRASPRPTKPLEIVIAGAGLGGLSTAKYLADAGHKPILLEARDVLGGKVAAWKDDDGDWYETGLHIFFGAYPNIQNLFGELGINDRLQWKEHSMIFAMPSKPGEFSRFDFSEALPAPLNGILAILKNNEMLTWPEKVKFAIGLLPAMLGGQSYVEAQDGISVKDWMRKQGVPDRVTDEVFIAMSKALNFINPDELSMQCILIALNRFLQEKHGSKMAFLDGNPPERLCMPIVEHIESKGGQVRLNSRIKKIELNEDGSVKSFILSDGSAIEGDAFVFAAPVDIFKLLLPEDWKEIPYFQKLEKLVGVPVINVHIWFDRKLKNTYDHLLFSRSSLLSVYADMSVTCKEYYNPNQSMLELVFAPAEEWISRSDSEIIDATMKELATLFPDEISADQSKAKILKYHVVKTPRSVYKTVPGCEPCRPLQRSPIEGFYLAGDYTKQKYLASMEGAVLSGKLCAQAIVQDYELLVGRSQKKLSEAITS

SEQ ID NO: 43 - protein, 520 aa, Solanum lycopersicum
  misc_feature (84)..(84): Xaa can be any naturally occurring amino acid
MAQISSMAQGIQTLSLNSSNLSKTQKGPLVSNSLFFGSKKLTQISAKSLGVFKKDSVLRVVRKSSFRISASVATAEKPHEIVLXPIKDISGTVKLPGSKSLSNRILLLAALSEGRTVVDNLLSSDDIHYMLGALKTLGLHVEDDNENQRAIVEGCGGQFPVGKKSEEEIQLFLGNAGTAMRPLTAAVTVAGGHSRYVLDGVPRMRERPIGDLVDGLKQLGAEVDCSLGTNCPPVRIVSKGGLPGGKVKLSGSISSQYLTALLMAAPLALGDVEIEIIDKLISVPYVEMTLKLMERFGVFVEHSSGWDRFLVKGGQKYKSPGKAFVEGDASSASYFLAGAAVTGGTVTVEGCGTSSLQGDVKFAEVLEKMGAEVTWTENSVTVKGPPRNSSGMKHLRAIDVNMNKMPDVAMTLAVVALFADGPTTIRDVASWRVKETERMIAICTELRKLGATVVEGSDYCIITPPEKLNVTEIDTYDDHRMAMAFSLAACADVPVTIKNPGCTRKTFPDYFEVLQKYSKH

SEQ ID NO: 44 - protein, 659 aa, Solanum lycopersicum
MAAAASPSPCFSKTLPPSSSKSSTILPRSTFSFHNHPQKASPLHLIHAQHNRRGFAVANVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIVRLISESKKPVLYVGGGCSQSSEELRRFVELTGIPVASTLMGLGAFPTGDELSLQMLGMHGTVYANYAVDSSDLLLAFGVRFDDRVTGKLEAFASRAKIVHIDIDSAEIGKNKQPHVSICADIKLALQGLNSILEGKEGKMKLDFSAWRQELTEQKMKYPLNFKTFGDAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGDPSNEEEIFPNMLKFAEACGVPAARVSHRDDLRAAIQKMLDTPGPYLLDVIVPHQEHVLPMIPSGGAFKDVITEGDGRCSY

SEQ ID NO: 45 - protein, 558 aa, Solanum lycopersicum
MTTTAVVNHPSIFTHRSPLPSPSSSSSSSPSFLFLNRTNFIPYFSTSKRSSVNCNGWRTRCSVAKNYTVPPSEVDGNQLPELDCVVVGAGISGLCIAKVISANYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIEPFCSGVYAGDPSKLSMKAAFGKVWKLEQTGGSIIGGTFKAIKERSSNPKPPRDPRLPTPKGQTVGSFRKGLRMLPDAICERLGSKVKLSWKLSSITKSDKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLLNYIGGATNTEIVSKTESQLVEAVDRDLRKMLIKPKAQDPFVTGVRVWPQAIPQFLVGHLDTLGTAKAALSDNGLDGLFLGGNYVSGVALGRCVEGAYEIASEVTGFLSQYAYK

SEQ ID NO: 46 - protein, 274 aa, Solanum lycopersicum
MNSTSMSSLGVRKGSWTDEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKTNNKCGEISTKIEIIKPQPRKYFSSTMKNITNNIVILDEEEHCKEIKSEKQTPDASMDNVDQWWINLLENCNDDIEEDEEVVINYEKTLTSLLHEEKSPPLNIGEGNSMQQGQISHENWGEFSLNLQPMQQGVQNDDFSAEIDLWNLLD

SEQ ID NO: 47 - DNA, 9113 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcacttttgttgctgcaaccatatcgaatacactactaaggcctgctaacattaggttttaccaaatcaaaactagttaggatcggcttagtaatgaatcttctctatccattttgcgttatatagcagccacaagactttcggacaaataaagtagtcggagaagaggatttctatttcataagtaacttgaatgggggaaattaatattggtggaatgaaaattatgatatgcaccagaaatcatatgtgaaaatgcaaattagtaaagaaacaaatgattattactattattattagttctcataataaattcaactggaatccaacaacatacattgaatagaaagaaagaagcaaaacggaaaatgcgaacagtttctcactgttgacatatacacgtgcgcacatgtaattggttactaagaggttattaggacgccttgtatatatagtgataaggcttcctatctaacggacaaaaagagttagcaaacctcatcttacaggaatggtaaccattggattttgtggttcttggcattacaaaatcaatggccactgaattttaacccctcactcgtccttatctcaaacttcccatactgacaaacaagatatgttttttttttcttttttaaaaaatacttgcaatttttttgttgcttttgctttttctttctgacgagtttttcatttttaaaaataatatcacaaggtatgtttggtataactgaaaatattaactaaaaaaataaggaaaatacttcctttccatattgattgtcgaacacaacccaccctgatacccagagtgttgagtaaaaatatgtataaatgtttttgtcataatattttttgattaattacatgaaaaaacacaccctaacacgaaaataaagtctgcaacccctgtattttgtttctttctcgtttggttttgggcatagagtaatttctgcgccatatatttgaactgttaattctacaaagggaaacttggtgagtagtactttggggaaaactgtttatgaatgatacttcaccttaacttagaaggaatcaacaagtatggtacaaacttatatttggctgaaataatccaacgccaattctggattttctcagataattattatatcaatgcattttatagacatattgctttagatccatcgaaaacagtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 48 - DNA, 11756 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcacttttgttgctgcaaccatatcgaatacactactaaggcctgctaacattaggttttaccaaatcaaaactagttaggatcggcttagtaatgaatcttctctatccattttgcgttatatagcagccacaagactttcggacaaataaagtagtcggagaagaggatttctatttcataagtaacttgaatgggggaaattaatattggtggaatgaaaattatgatatgcaccagaaatcatatgtgaaaatgcaaattagtaaagaaacaaatgattattactattattattagttctcataataaattcaactggaatccaacaacatacattgaatagaaagaaagaagcaaaacggaaaatgcgaacagtttctcactgttgacatatacacttcatgtccaggaattatcgaatgcagcggaagtcatcgcctgagcaaactcctcaaagctaatgcaaccatcaccatctctatcagcttccttgatcatccctgttaactcctcttgtgtaagtgcatgtcctaatttagccatagaatgcgctaactccgccgccgtgatcacaccattaccgtccctatcaaacatctgaaaaatcttcttcagctgttcctcagagtacggacacttggccgatataagctccggcgcaaccaaagccacaaattccgaaaactcaatcaatccattgctgttcctatctgccttctggattaaatcctccaattgatcattactcggctttaatcctaatgatcgaagcaacgagccaagttcaagctgcgttaagcttccgtcattgttcctatcaaatgaccggaaaatctcacgaagctccgcaatttgatcatcgtcaagcttcggttctgcatctccgctcatgtaattggttactaagaggttattaggacgccttgtatatatagtgataaggcttcctatctaacggacaaaaagagttagcaaacctcatcttacaggaatggtaaccattggattttgtggttcttggcattacaaaatcaatggccactgaattttaacccctcactcgtccttatctcaaacttcccatactgacaaacaagatatgttttttttttcttttttaaaaaatacttgcaatttttttgttgcttttgctttttctttctgacgagtttttcatttttaaaaataatatcacaaggtatgtttggtataactgaaaatattaactaaaaaaataaggaaaatacttcctttccatattgattgtcgaacacaacccaccctgatacccagagtgttgagtaaaaatatgtataaatgtttttgtcataatattttttgattaattacatgaaaaaacacaccctaacacgaaaataaagtctgcaacccctgtattttgtttctttctcgtttggttttgggcatagagtaatttctgcgccatatatttgaactgttaattctacaaagggaaacttggtgagtagtactttggggaaaactgtttatgaatgatacttcaccttaacttagaaggaatcaacaagtatggtacaaacttatatttggctgaaataatccaacgccaattctggattttctcagataattattatatcaatgcattttatagacatattgctttagatccatctagttaggatcggcttagtaatgaatcttctctatccattttgcgttatatagcagccacaagactttcggacaaataaagtagtcggagaagaggatttctatttcataagtaacttgaatgggggaaattaatattggtggaatgaaaattatgatatgcaccagaaatcatatgtgaaaatgcaaattagtaaagaaacaaatgattattactattattattagttctcataataaattcaactggaatccaacaacatacattgaatagaaagaaagaagcaaaacggaaaatgcgaacagtttctcactgttgacatatacacattaaccgatgatggtggtttctgcaatcatggaggtaacgacgtatgggtccatatttgaggctggccttctgtcctcaaagtatcccttgcctgccttctctgtgtctcttccaacacggacagatgcaccacggtttgcaaccccccatttgaatgtgttgatgttggctgtttcgtgctttccagtgagacgacgctcgttgccttcaccatatgcagctatgtgttctttgtgcttcaagccaagcttctcaatagcctttaagattatttcatagcctccgtcttccctcatcgacttggtgctgtaatttgtgtgagcacctgcacaattccagtcgcccggaataggcttggggtcgaatgacacgaccaccccagcaatctctgcaatcctctctagaatgtaacgagctacccacacttcatcaccagctgagatgccaacagaaggtccaacttgaaattcccactgtcccggcatgacttcaccattgatcccgctgatgttaatcccagcatagagacaagccttgtaatgggcgtcaacaatgtcacgtccaaaggccttgtcagctccggttccacagtagtatggtccctgggggccaggaaaaccgccaatgggccatccaagaggccagttgacctccctttgcagcaaggtatattcttgttcaataccataccaagtttcctcagcagccacatcagggtggctgaagaccttggcggcggcgtgcctcttgtttgttgggatgggctcaccagcaggagtataggcatcacacatgaccaagatgttgttgcctcttctgaatgggtccttgaagattgcttgtggatataagatcacttcactgtcttctccgggagcttgaccagtgctcgatccatcgtagttccatttgggtagttctgcaggactagtaactggaccagggagagtcctggctttgctcctcatgtccatgcctgatccaccaatccatatgtattcagcaatgatcttctgagtatcacctgagagattgaggttgataagatctgaaagcagagacatgtaattggttactaagaggttattaggacgccttgtatatatagtgataaggcttcctatctaacggacaaaaagagttagcaaacctcatcttacaggaatggtaaccattggattttgtggttcttggcattacaaaatcaatggccactgaattttaacccctcactcgtccttatctcaaacttcccatactgacaaacaagatatgttttttttttcttttttaaaaaatacttgcaatttttttgttgcttttgctttttctttctgacgagtttttcatttttaaaaataatatcacaaggtatgtttggtataactgaaaatattaactaaaaaaataaggaaaatacttcctttccatattgattgtcgaacacaacccaccctgatacccagagtgttgagtaaaaatatgtataaatgtttttgtcataatattttttgattaattacatgaaaaaacacaccctaacacgaaaataaagtctgcaacccctgtattttgtttctttctcgtttggttttgggcatagagtaatttctgcgccatatatttgaactgttaattctacaaagggaaacttggtgagtagtactttggggaaaactgtttatgaatgatacttcaccttaacttagaaggaatcaacaagtatggtacaaacttatatttggctgaaataatccaacgccaattctggattttctcagataattattatatcaatgcattttatagacatattgctttagatccatcgaaaacagtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 49 - DNA, 2169 bp, Artificial sequence
  Synthetic
actgttttcgatggatctaaagcaatatgtctataaaatgcattgatataataattatctgagaaaatccagaattggcgttggattatttcagccaaatataagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattacatgtctctgctttcagatcttatcaacctcaatctctcaggtgatactcagaagatcattgctgaatacatatggattggtggatcaggcatggacatgaggagcaaagccaggactctccctggtccagttactagtcctgcagaactacccaaatggaactacgatggatcgagcactggtcaagctcccggagaagacagtgaagtgatcttatatccacaagcaatcttcaaggacccattcagaagaggcaacaacatcttggtcatgtgtgatgcctatactcctgctggtgagcccatcccaacaaacaagaggcacgccgccgccaaggtcttcagccaccctgatgtggctgctgaggaaacttggtatggtattgaacaagaatataccttgctgcaaagggaggtcaactggcctcttggatggcccattggcggttttcctggcccccagggaccatactactgtggaaccggagctgacaaggcctttggacgtgacattgttgacgcccattacaaggcttgtctctatgctgggattaacatcagcgggatcaatggtgaagtcatgccgggacagtgggaatttcaagttggaccttctgttggcatctcagctggtgatgaagtgtgggtagctcgttacattctagagaggattgcagagattgctggggtggtcgtgtcattcgaccccaagcctattccgggcgactggaattgtgcaggtgctcacacaaattacagcaccaagtcgatgagggaagacggaggctatgaaataatcttaaaggctattgagaagcttggcttgaagcacaaagaacacatagctgcatatggtgaaggcaacgagcgtcgtctcactggaaagcacgaaacagccaacatcaacacattcaaatggggggttgcaaaccgtggtgcatctgtccgtgttggaagagacacagagaaggcaggcaagggatactttgaggacagaaggccagcctcaaatatggacccatacgtcgttacctccatgattgcagaaaccaccatcatcggttaatgtgtatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgcaaaatggatagagaagattcattactaagccgatcctaacta

SEQ ID NO: 50 - DNA, 7882 bp, Artificial qequence
  Synthetic
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttag

SEQ ID NO: 51 - DNA, 1065 bp, Solanum lycopersicum
atgtctctgctttcagatcttatcaacctcaatctctcaggtgatactcagaagatcattgctgaatacatatggattggtggatcaggcatggacatgaggagcaaagccaggactctccctggtccagttactagtcctgcagaactacccaaatggaactacgatggatcgagcactggtcaagctcccggagaagacagtgaagtgatcttatatccacaagcaatcttcaaggacccattcagaagaggcaacaacatcttggtcatgtgtgatgcctatactcctgctggtgagcccatcccaacaaacaagaggcacgccgccgccaaggtcttcagccaccctgatgtggctgctgaggaaacttggtatggtattgaacaagaatataccttgctgcaaagggaggtcaactggcctcttggatggcccattggcggttttcctggcccccagggaccatactactgtggaaccggagctgacaaggcctttggacgtgacattgttgacgcccattacaaggcttgtctctatgctgggattaacatcagcgggatcaatggtgaagtcatgccgggacagtgggaatttcaagttggaccttctgttggcatctcagctggtgatgaagtgtgggtagctcgttacattctagagaggattgcagagattgctggggtggtcgtgtcattcgaccccaagcctattccgggcgactggaattgtgcaggtgctcacacaaattacagcaccaagtcgatgagggaagacggaggctatgaaataatcttaaaggctattgagaagcttggcttgaagcacaaagaacacatagctgcatatggtgaaggcaacgagcgtcgtctcactggaaagcacgaaacagccaacatcaacacattcaaatggggggttgcaaaccgtggtgcatctgtccgtgttggaagagacacagagaaggcaggcaagggatactttgaggacagaaggccagcctcaaatatggacccatacgtcgttacctccatgattgcagaaaccaccatcatcggttaa

SEQ ID NO: 52 - DNA, 1065 bp, Solanum lycopersicum
atgtctctgctttcagatcttatcaacctcaatctctcaggtgatactcagaagatcattgctgaatacatatggattggtggatcaggcatggacatgaggagcaaagccaggactctccctggtccagttactagtcctgcagaactacccaaatggaactacgatggatcgagcactggtcaagctcccggagaagacagtgaagtgatcttatatccacaagcaatcttcaaggacccattcagaagaggcaacaacatcttggtcatgtgtgatgcctatactcctgctggtgagcccatcccaacaaacaagaggcacgccgccgccaaggtcttcagccaccctgatgtggctgctgaggaaacttggtatggtattgaacaagaatataccttgctgcaaagggaggtcaactggcctcttggatggcccattggcggttttcctggcccccagggaccatactactgtggaaccggagctgacaaggcctttggacgtgacattgttgacgcccattacaaggcttgtctctatgctgggattaacatcagcgggatcaatggtgaagtcatgccgggacagtgggaatttcaagttggaccttctgttggcatctcagctggtgatgaagtgtgggtagctcgttacattctagagaggattgcagagattgctggggtggtcgtgtcattcgaccccaagcctattccgggcgactggaatggtgcaggtgcttacacaaattacagcaccaagtcgatgagggaagacggaggctatgaaataatcttaaaggctattgagaagcttggcttgaagcacaaagaacacatagctgcatatggtgaaggcaacgagcgtcgtctcactggaaagcacgaaacagccaacatcaacacattcaaatggggggttgcaaaccgtggtgcatctgtccgtgttggaagagacacagagaaggcaggcaagggatactttgaggacagaaggccagcctcaaatatggacccatacgtcgttacctccatgattgcagaaaccaccatcatcggttaa

SEQ ID NO: 53 - DNA, 1027 bp, Solanum lycopersicum
aaccttcaccaacccaccaaacaattgaaatgtataaagtttaatatggaaatttcattataaaaagttcttaaaaaaaaatctaaatatcaataagtcaaacttaaaaatttaatacattgatgattgaccaatgagaccctttattaaaacttgtattatgaatctaattacttcctctacttttttacatttttaacttatttatttcttctcaacttacagctcttatcatttttgtcatataagatcacgttaattgttatttcatggtaaaaagataatattaacttcaccaaaaccatcaaatcaattaatacacattactcatgagtcagtataaaattttatattataattcaaaaaatcaatcgttaaaactcttgattaataacgcactaagaaaaaatcgtatggaatcatacttctctttgtgctctcctcttcctctattttagttattttcctcttaataaagattcatgtgatcgtacttcgaactccgataataatttctatcacccaaagaaaaagtgttgatgaatgatgattattgtcttgtaataataagtaataaaaagactagtttaaccttcaccaactcaccaaacaattttaatgttttcaacttggtcaaaaaacattaaattgattatttttttaaaaaatacaaaaaaaaaaagggaaccggcacttcaagtatcctgtaaaaaagcaatggaatcctcaaattggattctcttttttccttatattcatattcatcagttacctactctttggaacaaccaaaacttgttcttttttcaatgctaatttattttcatttttccattattattattaaaaattaaaatagcaaataaataaataaaaaaaaaattggaataattaagttgtaagtgtaatagtttaatacaagcaaccctgaaaatcgcctatataaagtgtataaaaatttagtctttgcctcatcaaagaaaattcatcttatagagaattttaatttaagaagtttatcatcatc

SEQ ID NO: 54 - DNA, 403 bp, Solanum lycopersicum
ccttgaagacttgatagtatgaatttgctcgagggatcgcttgtttctggtttgcacaatttgggataggagaaaagattgaattgtggaacgaccctttggacttcacctgtgttatttagttatagggatagtttgtctctggttatttttctgtttatttgccccagttgaattgtattttcatacagcaaagccttatttcattgcctatgatttggcaatgctgtgttacaaatgttattcttattaataacaaagatattgaaagggtttggttcacttcattactgtttttacccttgtttctatcaagagcgcgatttcgtttactcgatacattaaaaaaataaggaggaaggttgcgataggttaacgataacgtacatatagtcttatttga

SEQ ID NO: 55 - DNA, 1025 bp, Solanum lycopersicum
aaccatccagcaatgtggaagcttgacgattttccttcagagtagaaattgaaaagaatcaactaaaaaggatagtccttcgatttgatttccggcttaaaaataaactaataagaatgagagagcgaataatagaatattttgaaattttaaagatattcaactatgttaaattgcgttataaatttcttaaattagtagcacctaatagtttagttctcaaaagtcaaaactactacataatgtgctcatttttcacattaaaatgcctacatgatgtaaaagtaaaactcgtagcattctacgtgttttactcaactcaaacatcctgttcattttaataaacgtacgatgagcttctctctccaattttcttttcttttttttttttaaaaaaatatttttttttatatcaatccaaatgggctccaatttatcataaattaggtagaaacttagatattaaagaaagaaaagggtttatctcgcaagtgtggctatggtgggacgtgtcaaattttggattgtagccaaacatgagatttgatttaaagggaattggccaaatcaccgaaagcaggcatcttcatcataaattagtttgtttatttatacagaattatacgcttttactagttatagcattcggtatctttttctgggtaactgccaaaccaccacaaatttcaagtttccatttaactcttcaacttcaacccaaccaaatttatttgcttaattgtgcagaaccactccctatatcttctaggtgctttcattcgttccgaggtaagaaaagatttttgtttctttgaatgctttatgccactcgtttaacttctgaggtttgtggatcttttaggcgactttttttttttttgtatgtaaaatttgtttcataaatgcttctcaacataaatcttgacaaagagaaggaattttaccaagtatttaggttcagaaatggataattttcttactgtgaaatatccttatggcaggttttactgttatttttcagtaaa

SEQ ID NO: 56 - DNA, 408 bp, Solanum lycopersicum
ctttgtggttattatttagcttctgtacactaaatttatgatgcaagaagcgttgtacacaacatatagaagaagagtgcgaggtgaagcaagtaggagaaatgttaggaaagctcctatacaaaaggatggcatgttgaagattagcatctttttaatcccaagtttaaatataaagcatattttatgtaccactttctttatctggggtttgtaatccctttatatctttatgcaatctttacgttagttaatatctatctatcgatattctagtatcttatactatagatccaactgaaccaagaaattatgaaccgtgtcttccagaaattctaataatgatgggagcaatataaatataaggatgtctttgacaataaaagggcggtggaagagttatagtga

SEQ ID NO: 57 - DNA, 718 bp, Solanum lycopersicum
aacttctccttgctgaatttaatataaatctgattttacattattaaaataataaaaactcactgcattattttttttaaaaaaacaaccaaactaattacaaaaaaggaacatggccaacaaaaaaaaaagttagaactaaaatcaaacaatttattttcatactttaccatgtaatcatgttattaaaaagacaaaaaaatttattttattaaaaaaatgaaaatattattttttaaaataggactcatattgaaaggtgatgtgagattatgcataatttccaatcataaatatatttcttaattatcataaatgtcatttagatatttttaatcatatttttggatattaatatttttattatttaaatattagaatacacataatttttatttttacatatatacatattataattttatttatcaatttattttttattaaatattaaattaatatataatattatatcacatatttctatttaatctttcgttaaagcgaaaggatgtaacgtaatttttgaaccataataacatcaatattacaaaggatatagtatcatttacgacatttttgattttgaacttataaattgttttccatttatatttgaatcaatgtaggacccttacaacacattttcgtggcgctcatcacttcttatagccattttgcctcttcctttcacttctctcacctttatcgaccaaca

SEQ ID NO: 58 - DNA, 639 bp, Solanum lycopersicum
cttaaagaaactacataactagttctagacattgtattatctaaaataaacttctattaagccaaaagtgttcgatttgtctagtttgctgttagtctttggcgtggctttgcttgttgtggctgttgtactatcttctacttggtatttatgttcacttaaagttttgcatcatcttgcttttgtcgaatggaaggattcagattattattttttattggcagcacctatttcattatctggagctctatttgaaaatgggtggtttaaacggtcacgaggataatagcttgtgtaactagaatatatggaaacacttgaacgtgtaactagaactttggtaggggtgcacatttcctcctttaataggtcttacgtttcgattagtagtgttctgttacagatggacaagatcatttacctctttttttcagcctcctcttgatatctatcatgtgttagttccattggctttgaattaagtataaaattcgatatgccaaaatggtggtgttagaatctgtgcattcactatcagtcaatggaccgggttccttgacatacaaataaggatataacagaaagtaaatgcagtttaataacaaaggagttttacgtgaaaatctcttgctcaagtga

SEQ ID NO: 59 - DNA, 1272 bp, Solanum lycopersicum
aacaatttatacatttcgcttctattgtataagtgagaaaggcgagggttgcgagcaagatctggaagcggggagaaagggaaacaaaaatatatgtatttatacaattctctctgctttatgtaaatagaaacaatttttatacatttgtgtttttataaaaagtgaggaagcgagcgagagattggagtgagaatgggagagtgacgagcgagatttttgagagagaggcgactgacaaattttgacaaacgtttgttatggagcacaattaaatcaaactctaactactccatttattttaaattattaatttgctattatacattttatccccaaaaacaaaatattttggggctaatagattcataaggggtgtactagtataaacacttctccttcttatggattccgcaaaatatgagtaagttgttcattttattttttatacaaataaaaactcatgataatttatttatacatatacaaccaactaagggcccgtttggatgggcttaataaaagcagttttaaaaaaatacttttgaaagtgttgaaacttatttttaaaataagcaattatgcgtttggataaaaatgctgaagttgttatgccaaacgtgaaaagggaaaaatggaagaaagagatgttaggattatatgggtaatttggagattgtataaaaatattaagggcaaaaagattaaaatgtggtcaacttaaaacagcttataagctaaaaaaaaaaaaagcacccctccccagcttttaacttttgacttaaaataaatttttttttaacttaaaataaatttttttgagtattgccaaacagttaaataagtcaaaaatcagattttaagtcggtttgatcagcttttaagctgagccaaacaggctctaataagagagaatatttttttgcaaaataagtagtaatataatcagaaatagacaaaattcatagaagcagatgtctgttgtgaaaaattaagggatgcattttgcaaattgtgacaattcagtcaaatgcacaactaccctcaaacctcaacaactcttgatggcttttgaagaaaagaattcagagacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaa

SEQ ID NO: 60 - DNA, 379 bp, Solanum lycopersicum
accacttcacatgtagaaggaattattttgtactacaagagaaattatgcaccagtttgcaaccaaaatggtgcccataccggaagagaaaaaagctttccaactcctttttatatgtctatgtgagatcatgttcattgtatttgttgaagttgagcttctttttttgtttctcgtgtagaagacatgtatactatatagttaagtacacttccttgaagaatatttaccattgattatcaccgttttagttattgcattttggtattcaaaataaatttgtttcgaggattaaagctattattgtgatttatagagctaagataggccattagtctatatattttcacttattaaagttcatgattacataagatga

SEQ ID NO: 61 - DNA, 1515 bp, Solanum lycopersicum
aacgaattatacaattcgtttctttgtatatgtatagcgaattatacaattgttttttttgtacatgcatagcgaaatatatatatatttatgtttgttatggagcataattatgcaaagtataaccataacatacaagtatgatttttatatttactatatctgaaagttactcttttaaaattaattttttttttatatatttttagaaatgtggactgaggcccacagcccacatacaggtgaattgggctggctaatttctggcccaccaaaaaagtgagtcagtaggcctcgtccatcaaattccaaagtccatcttataactgtagttttgaggtaagtatataaaaaaaactttgatataaattattaatttcgtttttaaattattgtcaattttaaaaaaacacttctatttgcctaactaaacttagatatatctctgatctgtcacatgacgtaataactaatatccaactcttatagcacgagcctaaataaaaagtcgagagaagtgttgaaaatacctccaaacttgacgagaatttagagataaggtatgtttactcatgttacaaatcagagatatatgttaagttgaattagtctttaaaaactgttaaaaatttaattgaaactaataattcactttaaatttaaaaatattttcaatactttttcctgtattttgattaaaaagaattattcattcacactcatcgttgttgtctctacttgtctccaaatcgtttgtagtcaattcaatttttttcactaatcattagtttatttagcgttaaagcttgacttatcaatttcataaagttcttattttgaccgcttgccaccttattgcttccaccaacttcacctaaaaatgaacttctgaaacaaatttcacgaatattgtcgatgatgagtggattggtccatgacaatagattatgcaagtttttagttaattagagcttttggtgctttcatatacaaacattacttgcttcaataatatcaatataattttataaatatcatttaagaaaaataaaatatgtataaattctattttcatttcataaagatcgataaacttctcttaaaacgaaatttacttcctttaatttgttaaaaaagaattgatcatttctttttttttaaacgatacttttgattttaatttttttaatttcatttttaatacaataatgattaaatgacactttgatacatttcatataattttaatttatattaaaacattttttaaagctcaatatcaaattaatcaaaccattttttttaaaaaaaacaaacttttcaaagggaataacttatcttgtagcatcaccccttatctcatcaacattaattcctagccgaaagatgtgaactcataaagaaaaccgacggctgagattgtgcgggtctacaaatcccattttctttcatcaactgaaacgataacgctaaagcaaacggtgatattttctcagaggagctgagagtgcagtc

SEQ ID NO: 62 - DNA, 451 bp, Solanum lycopersicum
aacctcctcttggggaggtactgttaggtttcaaaagttttgcttattagagttattttagctttggtaaatgatttatgcttgatttcagtcgtttttgttgtaatcttggttctcatttctttgggacaaaatgttcttgtcaaggaacaatacgtttagagttcgagtatctgttaattgtaagaaaatctaacatattgggcataattagctgcctgctttgccagtagatatattatatggcttggttaaatatgtttggtcttggaatttgatttctttgggaaattattcatcccaagaccaaatgtcaaagattataccatactcaaggatagggactcgtaaatccttccacaaacacccatttcgcaacatactttcaatcttgacgttctaaactaacatctttacaccaaatcctatatcgagagttctactcgtttga

SEQ ID NO: 63 - DNA, 11 bp, Artificial sequence
  Synthetic
gtgcgcacatg

SEQ ID NO: 64 - DNA, 1096 bp, Solanum lycopersicum
gaaaagaatccgctaatattttcaattgattctacgagatacttgtcacttttcgcaatagctcagattgggggaaaaagtgagattgcttcaactgttcaaggtttgaataaattgaagtgcgaatggagtgtgtggttcagggaattattgagactcaacatgtcgaggccctggaaattctgcttcaagggctttgtggtgtacataaacaaagcttaaggattcatgaactgtgccttaaaagtgtccctaacctaggcttagtagcatcagaaatacggctcttatgtgatcttgagcagccagaacctgcatggactgttaggcatgttggtggtccaatgagaggtgctggtgctgaacaaatctcagtgttggtgagaccaatgcaagaaagcaaaataagcaagaatgcattacgcttattttattcacttggctacaagctagaccatgagcagctgagagttggttttgcatttcatttccaaagaggtgcccagataactgtaacagtttcatccatcaacaagatgttgaaacctcatgctatcgatgatgcagtgcctgtgactccaggcatacagctagttgaagtgactgcaccagcttcatctgaaaattacaatgaagttgttgcatctgtaacgtccttctgtgaatatcttgcaccgctccttcatttgtcaaaacccggtgtctcaacaggggttgttcctactgcagctgcagctgctgcatctctgatgtctgatggtggaggcacaaagtgaatggaaaaattactcagtaccatttctgtcttaaatctctgttgcagttatcatagctgaagaatgagacgtatttcgccattctccttcccaataacttcaatgtttgtccttctgtaattgacgttaaatacctgatcatcgatatgcaacgttgctcattcatagaatagagttataataccctttgtactgaattgcgaaaaacaaagcacagcagtgctctctttgatctataattgtttgactcttttcttgtttatgtgttatgctcaaagcacatgagatgtttaagtgattatatttggttctttgggcgg

SEQ ID NO: 65 - DNA, 143 bp, Artificial sequence
  Synthetic
gagcaggaaagtattgggtgagatattgtgatggatgaaactgttacaggaataatgaggtgctaattggaagctgcaccttaattctttctgtaacagttttcatccatctcatcttcagtccctccccgaccctctctacc

SEQ ID NO: 66 - DNA, 143 bp, Artificial sequence
  Synthetic
gagcaggaaagtattgggtgagatattgttacagttatctgggcacctcgaataatgaggtgctaattggaagctgcaccttaattctttgaggtgcccagtataactgtatcatcttcagtccctccccgaccctctctacc

SEQ ID NO: 67 - DNA, 2334 bp, Artificial sequence
  Synthetic
cctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataagtcttttaagataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagttaacaggcctcacgtgctgctataattacttaaaagtgcgagtgtcctgtctgtttcccggttttgctattatgttgccagtcaatttgtttttttgatgggatggagaagtttggtggtgggggctatgaatgcacggtagcaaacaacagattgccagtattatctcatgtttccatttaatgtggttaatattctctacatacttgagaggtgcctgatgcattgccctcttctgtctggctacaccatcccttggtcgaagcgtctcttttttaggttgtttgtagttgaaggagagtgattgtgatgttttctcctcgtcttttctctcattttctccttttatctgattttgcacttttgtggttcttttttttcttggacccaataatgtcaatatttattgaatgagaaaattcctatatcatatcagtttgaggaaatcattactatttgtgtggatacaggagttttgactctttattggcgatattttgtattctattgttgctgttttggatgtggtttcagaacttccttagtgcatttgctcttaaatctgttttgcagtaaaattgaggctataaaagcttcattgcagattaccctcggatgagggatctcctcattgcctgtcatatattggtttcttttcatccaacacgcaggatacatacatttattgaatttgaccttctattttgggacaactctactgtgaaattggagggattgttgaatttttttcttgcatgagttcattgatggtattatttttgacaggatatattggcgggtaaacctaagagaaaagagcgtttattagaataacggatatttaaaagggcgtgaaaaggtttatccgttcgtccatttgtatgtgcatgccaaccacagggttcccctcgggagtgctggcattccgtacg

SEQ ID NO: 68 - DNA, 12 bp, Artificial sequence
  Synthetic
aacaggcctcac

SEQ ID NO: 69 - DNA, 5252 bp, Artificial sequence
  Synthetic
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagcctgataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagttgattaaaagaattcatgaacagtacatctatgtcttcattgggagtgagaaaaggttcatggactgatgaagaagattttcttttaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttccaggaaggacagctaacgatgtgaaaaactattggaacactaatcttctaaggaagttaaatactactaaaattgttcctcgtgaaaagactaacaataagtgtggagaaattagtactaagattgaaattataaaacctcaaccacgaaagtatttctcaagcacaatgaagaatattacaaacaatattgtaattttggacgaggaggaacattgcaaggaaataaaaagtgagaaacaaactccagatgcatcgatggacaacgtagatcaatggtggataaatttactggaaaattgcaatgacgatattgaagaagatgaagaggttgtaattaattatgaaaaaacactaacaagtttgttacatgaagaaaaatcaccaccattaaatattggtgaaggtaactccatgcaacaaggacaaataagtcatgaaaattggggtgaattttctcttaatttacaacccatgcaacaaggagtacaaaatgatgatttttctgctgaaattgacttatggaatctacttgattaatctagatgtgtatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgcaaaatggatagagaagattcattactaagccgatcctaactagttttgatttggtaaaacctaatgttagcaggccgtagtagtggctagcttactagtgatgcatattctatagtgtcacctaaatctgcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagtcatttcatttggagaggacatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgcca

SEQ ID NO: 70 - DNA, 16273 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaaaataataccatcaatgaactcatgcaagaaaaaaattcaacaatccctccaatttcacagtagagttgtcccaaaatagaaggtcaaattcaataaatgtatgtatcctgcgtgttggatgaaaagaaaccaatatatgacaggcaatgaggagatccctcatccgagggtaatctgcaatgaagcttttatagcctcaattttactgcaaaacagatttaagagcaaatgcactaaggaagttctgaaaccacatccaaaacagcaacaatagaatacaaaatatcgccaataaagagtcaaaactcctgtatccacacaaatagtaatgatttcctcaaactgatatgatataggaattttctcattcaataaatattgacattattgggtccaagaaaaaaaagaaccacaaaagtgcaaaatcagataaaaggagaaaatgagagaaaagacgaggagaaaacatcacaatcactctccttcaactacaaacaacctaaaaaagagacgcttcgaccaagggatggtgtagccagacagaagagggcaatgcatcaggcacctctcaagtatgtagagaatattaaccacattaaatggaaacatgagataatactggcaatctgttgtttgctaccgtgcattcatagcccccaccaccaaacttctccatcccatcaaaaaaacaaattgactggcaacataatagcaaaaccgggaaacagacaggacactcgcacttttaagtaattatagcagcacgtgaggcctgttaactgaaagtaaaagcaagttaaatcagcacacttagtctaacctaaacatatacatgaaggtatcaacacataacacaacatcataagatcgattcagaactcaactaaatgtctctagtcatataaggttttacttgttagtaagtaaactgatatatctaaatcccagacaatttggagctcagtcaacaggcggacacttgtaataataaaaacatgcttcgtttttccgtatagcagccaaaaaaaaaagatccatcaaagtttagctaaatttgctggtaaccaaaatgagtatgcagagatcaagtactaataacacagtagatctaggaaaattaagagcagatcaagttcatgacagattgaccataaaaacaacagatctggaaaaatcaaagagaatatcacacctaaatctaagttttactaagtaattagagcagtagagtaatcagaagaacaaaaagcagcgattcaacatgagtaagtagagcagatctcaaagcagaaattgaaagaaaagaagcaagtaaataaagcaagatctgatgaagaagataggttagattaccgaaaaggaaatgaaaatggatctgaaaagcaaaggcaaacggatgatgacttgtggccgacttgctttctctctctacagtctggaagaggatgaatgagttaagacaatacacaagtatatatatagtcgtaaaaactggctgagacggcctttgaatttataattttcttttttattctattcaatgtatttttgaatttgaatgataagaggtggtgagcacaaagacaaccaaattgacagattcgccctccactattgaatggagtaaacaaggaaaatctttgttcaaattattttcaatttttttaaaatctaattcctatcaatttgagttgattttttcgatcttctttcacaaaatttaattttttttttcaactatgtgtgcgctcgagcaaaaatttacgaactattatcttaaaagacttatgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgattgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctcatggatctaaagcaatatgtctataaaatgcattgatataataattatctgagaaaatccagaattggcgttggattatttcagccaaatagaagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattgaattcatgaacagtacatctatgtcttcattgggagtgagaaaaggttcatggactgatgaagaagattttcttttaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttccaggaaggacagctaacgatgtgaaaaactattggaacactaatcttctaaggaagttaaatactactaaaattgttcctcgtgaaaagactaacaataagtgtggagaaattagtactaagattgaaattataaaacctcaaccacgaaagtatttctcaagcacaatgaagaatattacaaacaatattgtaattttggacgaggaggaacattgcaaggaaataaaaagtgagaaacaaactccagatgcatcgatggacaacgtagatcaatggtggataaatttactggaaaattgcaatgacgatattgaagaagatgaagaggttgtaattaattatgaaaaaacactaacaagtttgttacatgaagaaaaatcaccaccattaaatattggtgaaggtaactccatgcaacaaggacaaataagtcatgaaaattggggtgaattttctcttaatttacaacccatgcaacaaggagtacaaaatgatgatttttctgctgaaattgacttatggaatctacttgattaatctagatgtgtatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgcaaaatggatagagaagattcattactaagccgatcctaactagttttgatttggtaaaacctaatgttagcaggccgtagtagtggctagcttactagtgatgcatattctatagtgtcacctaaatctgcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagtcatttcatttggagaggacatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 71 - DNA, 5 bp, Artificial sequence
  Synthetic
taaac

SEQ ID NO: 72 - DNA, 3 bp, Artificial sequence
  Synthetic
tga

SEQ ID NO: 73 - DNA, 5917 bp, Artificial sequence
  Synthetic
tgaccaagtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttttgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttggccaagtcggcctctaatacgactcactatagggagctcgtcgagcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctcacgtgttacacagctcaattacagactactcaccatgcatctgcgttctttctaccggtggctagttgcgttcctgctagctattaattgcttattctagacttgtatttatgtgtgggctattttattaaatacctaagaccaaggatcatgcactttttaattattatatgtacttgaacttgatcctatatatacttagtcatgcacttggtactatatatcggtatttcgtattaagtttttgtatatcgaccgtgttcgacataaatccgatcgaattggttcgttttcgaaattctcgatatttcgtaagttcgtgttccttttcgtgtccgactttatcgttttcgttttcgtattttaaatgtaaaagtagaaaacaattttagattttttcgaccgcttccaccaccgcaccagcgccgagatagcccagcgaagcaaacggccgagacggtacccccctctcgagagttccgctccacctccaccacgggggattccttccccaccgctccttccctttcccttcctcgtccgccgttataaatagccagccccgtccccggcttctttccccaacctctcgtcttgctcggacttcggagcacacgcacaacccgatccccaatccccctcgtctctcctcaccggcttcgcggatctccgcttcaaggtacggcgatcgatcatcctccctccctctctctctctctacctaatcttctttagatagactagatcggcgatccatagttagggccttctagttccgttcctgtttttccatggctacgtggtgcaatagatctgatggagttatgagggttaacttgtcatgctcttgcgatttatatatagtctctttaggagatcaatttaatctcggatggttcgagatcggtggtccatggttagtactctaggctgtggagtcgggggttagatccgcgctgttagggttcgtagatgtaggcgatctgttctgattgataacttgttagtacctgggaatcctgggatggttctagctggttcgcagctgagatcgatttcatgatctgctatatcttgtttcgttgcctatccctttttatctgtccgttgtatgatgttagcctttgatatatttcgtcttgtgcagcacttaattgttaagtgataatttttagcatgcctttttttttatttggttttgtttgattgtgctgctgttctagatcagagtagaagactgtttcaaactgcctgctggatttattaaatttggatctgtatgtgtgtcacatatatatcttaataataaagatggatggaacttttatatattttgctgttggttttgctggtactttcttagatatactctttttggatatggataggtaaatgcttagatacatgaagcaacgtacagtttaataattcttgttcatctaataaacacaaataaggacgggcgtaaatgttgctgtgggttttactggtactttcttagatatatacatgcttagatacatgacgtaacatgctgctacagtttaataaatattgtttatataataaacaaacatgatgtttattatcttggtatgcttgggtgatgttatatgcagcagctgtgtggatttttaaataccctgatgatcatgcatgaccttgccttagtttgctgtttatttgcttgagactgcttctttcgcttatactcacccattattttggtgacttctgcagcgctaggcgccataggtcgtttaagctgctgctgtacctgcgtttgtctggtgccctcttgtgtacctgcatatggaggttgtcgtctattaagtatctgtggtttgttttagtcgtgactgagttggtttgaaggacctgttgtgtcttgtgtcccgtgtgtctacccaaaactattatgccgcagtatggcttcatcatgaataagttgatgtttgaacttatataagtttgtgctcagtatgttttattttaggttatatctccttgaaaactggcgcggccttgccgtgccccatctcaataggccagttccatcgttgtagaacttaatataaatagtgatactaacaaaataaagaactgtgctgcttagaatacatagactatttgaaatcatgcatggatacataatagcatatacaacaaaagagaagcaagatcatgcattgtgctatacacgtgactagtgatgcatattctatagtgtcacctaaatctgcggccgc

SEQ ID NO: 74 - DNA, 6490 bp, Artificial sequence
  Synthetic
tgaccaagtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttttgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttggccaagtcggcctctaatacgactcactatagggagctcgtcgagcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctcacgtgaggcccgtatagatgtagttaaatagctaaaatttttggagaaataagcatttttttggaagaatatatttaaacatgggcttgtaaaacttggctgtaaagatttggaatttaggatcttggagccccaaaactgtataaacttgcttagggacccgtgtcttgtgtgttgcagaccaaaaaatttagaaagcatctaaacacctatttgaatgtaaagtttacagccaaaagttttaggatgtaaagatttgggatctaaaagtagtcattaggaaataacacgttagagagagagagtagatcttcttattggtttctcatgcactaatcgaaccaatcactggaccacttgaaccaaactttatcacattgaactttgtcagttcagttcgaacgcaggactggagctgcccttaaggccaattgctcaagattcattcaacaattgaaacatctcccatgattaaatcagtataaggttgctatggtcttgcttgacaaagttttttttttgagggaatttcaactaaatttttgagtgaaactatcaaatactgattttaaaaattttttataaaaggaagcgcagagataaaaggccatctatgctacaaaagtacccaaaaatgtaatcctaaagtatgaattgcattttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggataggtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcagcgctaggcgccataggtcgtttaagctgctgctgtacctgcgtttgtctggtgccctcttgtgtacctgcatatggaggttgtcgtctattaagtatctgtggtttgttttagtcgtgactgagttggtttgaaggacctgttgtgtcttgtgtcccgtgtgtctacccaaaactattatgccgcagtatggcttcatcatgaataagttgatgtttgaacttatataagtttgtgctcagtatgttttattttaggttatatctccttgaaaactggcgcggccttgccgtgccccatctcaataggccagttccatcgttgtagaacttaatataaatagtgatactaacaaaataaagaactgtgctgcttagaatacatagactatttgaaatcatgcatggatacataatagcatatacaacaaaagagaagcaagatcatgcattgtgctatacacgtgactagtgatgcatattctatagtgtcacctaaatctgcggccgc

SEQ ID NO: 75 - DNA, 8 bp, Artificial sequence
  Synthetic
cgctaggc

SEQ ID NO: 76 - DNA, 2475 bp, Artificial sequence
  Synthetic
gaacgttttctatgatatatgtaagggtaaattggacaaatcatatatattttgcatagtaaggtgacatggcatatctatgtggtgattttggtgggaccaaggactatatcagcccacatgacaaatttaaaggacttgtttggacaatatgaaagattaaggactaaaatgacctaggagcgaaactttagggaccatattggctattctccctttttgacacgaatgaaaaatccaatttcataacttgtctggaaaccgcgagacgaatcttttgagcctaattaatccgtcattagcacatgcgaattactgtagcacttatggttaattatggactaattaagctcaaaagattcgtcttgcgatttcctttttaactgtgtaattagtttttcttttactctatatttaatgctccatgcatatgtctaaagatttgatttaatgtttttcgaaaaaacttttggaggactaaccgggcctaacgtgacttgaagagctgtgacagcgcaaatcgtgaaacgcggatggacctagcattatggtgatgtaggaagtgccttgctggcagtggcaggtaccgtgcaagtgtaataccatagatccgttggcttatctgattacatgatgatgattactccctccgtttcacaaatataagtcattttagcatttttcacatttatattgatgttatgtctagattcattaacatcaatatgaatgtgggaaatgctagaatgacttacattgtgaaacggatcattaacatcaatatgaatgtggaaaatgctagaatgacttacactgtgaaacggagggagtatacgattatgtaatgaaaaaaggagtacaatactagtcgccgtctccccgcaaaaaaagtactagttgtcgtcaagtaggggagtaataataataataataataagggataatatacaggctgtgtttagttcgtgtgccaaatttttttaaagtatacggacaaatatttaaatattaaacatagactaataacaaaacaaattacagattccatctgtaaactgcgagacgaatctattaaacctaattaattcgttattagcaaatgtttactgtagcaccacattatcaaatcatggcgtaattagctcaaaagattcgtctcgcgatttacatgcaaaccatgcaattgatttttttttcatctacgtttagttctatgcatgtgtccaaatattcgatgtgatgaaaaaattggaaattcgaggaaaaaaatttaaatctaaacacggccacagtataaaaaaaatagtagcgttgttgtttatgaaagaggatggtaaagtaagacaagacaacgcaagggcctaaaaaagtggagacgaagaagaagacggaatatattgcattggaaaagtgagcgcttggacgagagaaaaactcggattcaagcgtccatatcagtggacaccaccaatgggaggtggccacgtgggcaggtcccgggtggaatctggcgcgttcacacgggaggttccgaaattacggcaacgccactggagtgcgaggcgcaggatgtgagatccacggcgggggctccgctactagaaacttcttctggtcgtgggtggtacgcaccctcgcgcctcgcctttatattactagtaagaagatctcatccctccttggtgaggtgaggtgagttgagttggggattgattgattgattcggattgggaagaagaagaagcaggggagctccggattataagaagcctttagagagcgggatatccgcaaaagattaatgccgatttgtattttgcgccttagagtcagtacgatcaagactgtcgtggcggttgtaataaaaattagtgtgctttgggccatctttttatgtgattccaattgtctttctcttcattcttgctttgatgctctttgtctggacctctagaccgccgtattgtactgtggagtttcaaagttaccaagctatttgctgtcaagataactatggattgaattccccttgatggatgaaccaactgttgttgtttgcccgttcttcagctttcgtttgtgcggccatcgatcgccatgcgttgcttaaacccatttctagctcccctaccctgctgcatccgccctcttctgcgcgatcgttggattgcgagtggttggctggttgcacgacttgtggagaccgaaacaaataatttttggtcaaattgatcggtggtactgtcggagcatctattttttctttagcttagatcgtataattgtaggattgggatttgtatattaatatatacaggtcgattaaaacaatgcaactattcgtgatgtcatgtgacctaaacaaatgtgtgccatttatgatatttttcaagagtggttcttatagacttcttactaacaaaaattcacgacaattggactgagcctcaaaagttaataaaaaagaatcgattc

SEQ ID NO: 77 - DNA, 9 bp, Artificial sequence
  Synthetic
tccggatta

SEQ ID NO: 78 - DNA, 2383 bp, Artificial sequence
  Synthetic
tagctagcatactcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaagatgtgcgggatcaagcaggagatgagcggcgagtcgtcggggtcgccgtgcagctcggcgtcggcggagcggcagcaccagacggtgtggacggcgccgccgaagaggccggcggggcggaccaagttcagggagacgaggcacccggtgttccgcggcgtgcggcggaggggcaatgccgggaggtgggtgtgcgaggtgcgggtgcccgggcggcgcggctgcaggctctggctcggcacgttcgacaccgccgagggcgcggcgcgcgcgcacgacgccgccatgctcgccatcaacgccggcggcggcggcggcgggggagcatgctgcctcaacttcgccgactccgcgtggctcctcgccgtgccgcgctcctaccgcaccctcgccgacgtccgccacgccgtcgccgaggccgtcgaggacttcttccggcgccgcctcgccgacgacgcgctgtccgccacgtcgtcgtcctcgacgacgccgtccaccccacgcaccgacgacgacgaggagtccgccgccaccgacggcgacgagtcctcctccccggccagcgacctggcgttcgaactggacgtcctgagtgacatgggctgggacctgtactacgcgagcttggcgcaggggatgctcatggagccaccatcggcggcgctcggcgacgacggtgacgccatcctcgccgacgtcccactctggagctactagagctcaatcaactgtacaattttgcctcttttttctctcttttctggcttccgatgccaaaattttggtactgtacggacactactttcggtaatgtgatggaacaagttgcaaaacacagagcatcttcatttgagtcattgacttcccaaaatagtactgtagatttttttttagcatctgcgagccgtcctcgtgtagaaacagtttcttgacagtattgtttctgcacgagaactacagtgacgagagattggatggtacagtacttaggttacagtgttaacgacagtgaaaaaaaacctggttttgtcaatgatgttcgtactgggtaacctatgcattcgagtgcaattgaccgtggatctctctcaagcaatttcacttgaaaagatttgttctggttttggccacacgtgtt

SEQ ID NO: 79 - DNA, 3375 bp, Artificial sequence
  Synthetic
tgcgcaacacacaccccccaaccctacacatacacaaacacaagagtgagagagagattaaaatctaagcactttttgatgcagtcaacacggcttaagtgtggggtaacttgtaagcagggcctttcgagggagagggacacgtgtacaggcagctgataccactacacatgtactacttcatttgctctaaaataaatttattttccactcatccctgcacatgtttatatatgtttatatagaactaaaaatactatatataatacccgtacttcataaactccgagaaaaatataaggaactgaaagtaaatttattctagaatggtgaattatctttctggaacaaaatagtgtacaaaacgcatcttgagaatgcatcgtaagctatttgataaggatagatgtgacgttagtgtcacgttgggatagtggtaaaaaccaaacctcgaatacccagatttccatacattttcgtctatgatgaaaaaaatttatgagtggtgtactttatatttctgacggtttcttgtttccataaaaacaagcaaccaagtctccccaattggttggttaaaacaataaatgaacctcacaaaattttgtagtggccggaatttgatttgaagcataactaactaaaaagctactaggagtattggtttaattttttatgctaagctactggtttaatttgataggacggtgtgccgagtaaaaattaattaggcagaaaggtctatacattgctctgcgctctctctctcctcatggcagacactaactccactggagaaaaatgttaactggaattatttggtattccctcccttcgtttcacaatatattttcctttttatttatcctaaaacaaatttacttttaagtaatcactacatcaaattaaagttaatgaaaatagaggataaatctctactattatatataaaaattaaagatgtttttgccggtattttggtacgttatccgtgtatgagtatgtttttaagttcatttggttttggaaatacatatccatatttgaatcggttcttaagttcgtttgcttttggtaatacagaaggaattgtataaaaaatctgtctaaaaaaactcgcatattaacttgagactattggattcctaactgcagctcatgactttctaaaagtatatatatccaaacgaattccacagtcatcttaactaaaccatatataataataattagattaaaatagattttacccgttgcaatgcacgggtattttcttatagtacattaaaaatttttaaaaaaacaaggaataattgtattaagatttaataaattatgatattttaaactttttaaaaaaaacgagatttgaagggagatatccctccaaacattttttataagaaattatgagcgtgttacggattaaacacaggaccatataagtgaaatcatataaccctttactatcaaatgcatctctaatttagttttttttattcgggagtactgattatatcccctaataaaagaaacatgaagcaatttagtcatgcgttaatcacacaacaaggacaacttattaaaaagtgtgatccatccacgtggtgttttgagccactgcagcagtggtattgtgacagacaaaggaggattccatgcgtctacaaccaaaaaccatcagcctctcctcccgccacgtgtcccccccacccgctcccgccactttcaaaccccacttcccctttgaccgcctctcccgccacctcctataaatctccccatgattcctccctcccattccccacctcacctcacctcctcctccacctcctcgaaattattcgaatccatctccttctccctcctcccaacccgcgccaaatcgatcgatcgcgagcgatcttggccgcgtctcaccaatgtgcgggatcaagcaggagatgagcggcgagtcgtcggggtcgccgtgcagctcggcgtcggcggagcggcagcaccagacggtgtggacggcgccgccgaagaggccggcggggcggaccaagttcagggagacgaggcacccggtgttccgcggcgtgcggcggaggggcaatgccgggaggtgggtgtgcgaggtgcgggtgcccgggcggcgcggctgcaggctctggctcggcacgttcgacaccgccgagggcgcggcgcgcgcgcacgacgccgccatgctcgccatcaacgccggcggcggcggcggcgggggagcatgctgcctcaacttcgccgactccgcgtggctcctcgccgtgccgcgctcctaccgcaccctcgccgacgtccgccacgccgtcgccgaggccgtcgaggacttcttccggcgccgcctcgccgacgacgcgctgtccgccacgtcgtcgtcctcgacgacgccgtccaccccacgcaccgacgacgacgaggagtccgccgccaccgacggcgacgagtcctcctccccggccagcgacctggcgttcgaactggacgtcctgagtgacatgggctgggacctgtactacgcgagcttggcgcaggggatgctcatggagccaccatcggcggcgctcggcgacgacggtgacgccatcctcgccgacgtcccactctggagctactagctcaaattaattagccagtgaaaaatcaaattacagagttgcttaatttttttactagtagaacgcaacagtaaaaagaattaacagcagtgaattattagttaattagctagggagttgaaatagtttagcggtcatgcactactgatttttaattagtgcagacaacgaccgcgtgtgtgtatatgcatgtataccttttactgtatcttcagattgtgtatatatatcatatatgtacaggaaaagatttatatatcatacatattttgttgtatatatatacgtatatttctgtacaagtatatgtagacagtattttgtcatcttaataatttttttatcatattttaggctgactttgctggttgtcggattgttgcaaacatgtacaattaatgttaagaaaattaaggtagctaatgtgtcaacatgttgtgtgtgtttgtgctgacagagtgacagtgtggtctgtcctactccaagtactatcaaagtggtggtcgtgactcgtgagagcgacttcaagcctagaggttcatgtttttcttttaagataatgaggaggttgattgttatttcctcctacctccacatatataagtacttctaagggtttgaggctccgttcttttttaattaagatgtaaattttatcacaatttttattagcatgttttttcaaactacgaaatggtgtgtttcgtacggaaactatgtatgtagatgttgcgca

SEQ ID NO: 80 - DNA, 288 bp, Artificial sequence
  Synthetic
gagcaggaaagtattgggtgagatattgttatcttttgaagttcgtcttgaataatgaggtgctaattggaagctgcaccttaattctttgaagacgaactttcaaaagatatcatcttcagtccctccccgaccctctctaccattgataggaagaaagagtgattattgttgatcaggaattcttttcgataatgatgatatgctaatttcattcaatttgggcagcaaaagcatctcaattcattttcgaaaagaatgtcctgatcatcaccttcacctctttcg

SEQ ID NO: 81 - DNA, 2126 bp, Artificial sequence
  Synthetic
tggcaggatatattgtggtgtaaacataagtcttttaagataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagttgattaaaagagcaggaaagtattgggtgagatattgttatcttttgaagttcgtcttgaataatgaggtgctaattggaagctgcaccttaattctttgaagacgaactttcaaaagatatcatcttcagtccctccccgaccctctctaccattgataggaagaaagagtgattattgttgatcaggaattcttttcgataatgatgatatgctaatttcattcaatttgggcagcaaaagcatctcaattcattttcgaaaagaatgtcctgatcatcaccttcacctctttcgggtgctgctataattacttaaaagtgcgagtgtcctgtctgtttcccggttttgctattatgttgccagtcaatttgtttttttgatgggatggagaagtttggtggtgggggctatgaatgcacggtagcaaacaacagattgccagtattatctcatgtttccatttaatgtggttaatattctctacatacttgagaggtgcctgatgcattgccctcttctgtctggctacaccatcccttggtcgaagcgtctcttttttaggttgtttgtagttgaaggagagtgattgtgatgttttctcctcgtcttttctctcattttctccttttatctgattttgcacttttgtggttcttttttttcttggacccaataatgtcaatatttattgaatgagaaaattcctatatcatatcagtttgaggaaatcattactatttgtgtggatacaggagttttgactctttattggcgatattttgtattctattgttgctgttttggatgtggtttcagaacttccttagtgcatttgctcttaaatctgttttgcagtaaaattgaggctataaaagcttcattgcagattaccctcggatgagggatctcctcattgcctgtcatatattggtttcttttcatccaacacgcaggatacatacatttattgaatttgaccttctattttgggacaactctactgtgaaattggagggattgttgaatttttttcttgcatgagttcattgatggtattatttttgacaggatatattggcgggtaaac

SEQ ID NO: 82 - DNA, 16558 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaaaataataccatcaatgaactcatgcaagaaaaaaattcaacaatccctccaatttcacagtagagttgtcccaaaatagaaggtcaaattcaataaatgtatgtatcctgcgtgttggatgaaaagaaaccaatatatgacaggcaatgaggagatccctcatccgagggtaatctgcaatgaagcttttatagcctcaattttactgcaaaacagatttaagagcaaatgcactaaggaagttctgaaaccacatccaaaacagcaacaatagaatacaaaatatcgccaataaagagtcaaaactcctgtatccacacaaatagtaatgatttcctcaaactgatatgatataggaattttctcattcaataaatattgacattattgggtccaagaaaaaaaagaaccacaaaagtgcaaaatcagataaaaggagaaaatgagagaaaagacgaggagaaaacatcacaatcactctccttcaactacaaacaacctaaaaaagagacgcttcgaccaagggatggtgtagccagacagaagagggcaatgcatcaggcacctctcaagtatgtagagaatattaaccacattaaatggaaacatgagataatactggcaatctgttgtttgctaccgtgcattcatagcccccaccaccaaacttctccatcccatcaaaaaaacaaattgactggcaacataatagcaaaaccgggaaacagacaggacactcgcacttttaagtaattatagcagcacccgaaagaggtgaaggtgatgatcaggacattcttttcgaaaatgaattgagatgcttttgctgcccaaattgaatgaaattagcatatcatcattatcgaaaagaattcctgatcaacaataatcactctttcttcctatcaatggtagagagggtcggggagggactgaagatgatatcttttgaaagttcgtcttcaaagaattaaggtgcagcttccaattagcacctcattattcaagacgaacttcaaaagataacaatatctcacccaatactttcctgctcttttaatcaactgaaagtaaaagcaagttaaatcagcacacttagtctaacctaaacatatacatgaaggtatcaacacataacacaacatcataagatcgattcagaactcaactaaatgtctctagtcatataaggttttacttgttagtaagtaaactgatatatctaaatcccagacaatttggagctcagtcaacaggcggacacttgtaataataaaaacatgcttcgtttttccgtatagcagccaaaaaaaaaagatccatcaaagtttagctaaatttgctggtaaccaaaatgagtatgcagagatcaagtactaataacacagtagatctaggaaaattaagagcagatcaagttcatgacagattgaccataaaaacaacagatctggaaaaatcaaagagaatatcacacctaaatctaagttttactaagtaattagagcagtagagtaatcagaagaacaaaaagcagcgattcaacatgagtaagtagagcagatctcaaagcagaaattgaaagaaaagaagcaagtaaataaagcaagatctgatgaagaagataggttagattaccgaaaaggaaatgaaaatggatctgaaaagcaaaggcaaacggatgatgacttgtggccgacttgctttctctctctacagtctggaagaggatgaatgagttaagacaatacacaagtatatatatagtcgtaaaaactggctgagacggcctttgaatttataattttcttttttattctattcaatgtatttttgaatttgaatgataagaggtggtgagcacaaagacaaccaaattgacagattcgccctccactattgaatggagtaaacaaggaaaatctttgttcaaattattttcaatttttttaaaatctaattcctatcaatttgagttgattttttcgatcttctttcacaaaatttaattttttttttcaactatgtgtgcgctcgagcaaaaatttacgaactattatcttaaaagacttatgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgattgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctcatggatctaaagcaatatgtctataaaatgcattgatataataattatctgagaaaatccagaattggcgttggattatttcagccaaatagaagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattgaattcatgaacagtacatctatgtcttcattgggagtgagaaaaggttcatggactgatgaagaagattttcttttaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttccaggaaggacagctaacgatgtgaaaaactattggaacactaatcttctaaggaagttaaatactactaaaattgttcctcgtgaaaagactaacaataagtgtggagaaattagtactaagattgaaattataaaacctcaaccacgaaagtatttctcaagcacaatgaagaatattacaaacaatattgtaattttggacgaggaggaacattgcaaggaaataaaaagtgagaaacaaactccagatgcatcgatggacaacgtagatcaatggtggataaatttactggaaaattgcaatgacgatattgaagaagatgaagaggttgtaattaattatgaaaaaacactaacaagtttgttacatgaagaaaaatcaccaccattaaatattggtgaaggtaactccatgcaacaaggacaaataagtcatgaaaattggggtgaattttctcttaatttacaacccatgcaacaaggagtacaaaatgatgatttttctgctgaaattgacttatggaatctacttgattaatctagatgtgtatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgcaaaatggatagagaagattcattactaagccgatcctaactagttttgatttggtaaaacctaatgttagcaggccgtagtagtggctagcttactagtgatgcatattctatagtgtcacctaaatctgcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagtcatttcatttggagaggacatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 83 - DNA, 143 bp, Artificial sequence
  Synthetic
gagcaggaaagtattgggtgagatattgtatctctttaagcttttcctcgaataatgaggtgctaattggaagctgcaccttaattctttgaggaaaagctttaaagagattcatcttcagtccctccccgaccctctctacc

SEQ ID NO: 84 - DNA, 176 bp, Artificial sequence
  Synthetic
gcactttgcctgaagagaggacgatggcaagggggagatgggtttttgaaggtttgtgacattcatcaaagctgacacggtggtttcttagcatgagtgccatgttgggagctgtgccagctttgatgaaatgtcacagccactcatcaggctcatctctctgtccgatttggagc

SEQ ID NO: 85 - DNA, 176 bp, Artificial sequence
  Synthetic
gcactttgcctgaagagaggacgatggcaagggggagatgggtttttgaaggttttgtgttgtgttgtgttcacacacggtggtttcttagcatgagtgccatgttgggagctgtgcgtgaacacaacacaaacacaagccactcatcaggctcatctctctgtccgatttggagc

SEQ ID NO: 86 - DNA, 176 bp, Artificial sequence
  Synthetic
gcactttgcctgaagagaggacgatggcaagggggagatgggtttttgaaggtttatagctgttgatttcccaaacacggtggtttcttagcatgagtgccatgttgggagctgtgcttgggaaatcaacaagctatagccactcatcaggctcatctctctgtccgatttggagc

SEQ ID NO: 87 - DNA, 176 bp, Artificial sequence
  Synthetic
gcactttgcctgaagagaggacgatggcaagggggagatgggtttttgaaggtttttctttggtttcttggcccacacggtggtttcttagcatgagtgccatgttgggagctgtgcgggccaagaaaccaaaagaaagccactcatcaggctcatctctctgtccgatttggagc

SEQ ID NO: 88 - DNA, 176 bp, Artificial sequence
  Synthetic
gcactttgcctgaagagaggacgatggcaagggggagatgggtttttgaaggtttttctcgtgaaatcctccacacacggtggtttcttagcatgagtgccatgttgggagctgtgcgtggaggatttcaacgagaaagccactcatcaggctcatctctctgtccgatttggagc

SEQ ID NO: 89 - DNA, 176 bp, Artificial sequence
  Synthetic
gcactttgcctgaagagaggacgatggcaagggggagatgggtttttgaaggtttctcatttgctcatcatcttacacggtggtttcttagcatgagtgccatgttgggagctgtgcaagatgatgagcaaaatgagagccactcatcaggctcatctctctgtccgatttggagc

SEQ ID NO: 90 - DNA, 2731 bp, Artificial sequence
  Synthetic
gtgttacacagctcaattacagactactcaccatgcatctgcgttctttctaccggtggctagttgcgttcctgctagctattaattgcttattctagacttgtatttatgtgtgggctattttattaaatacctaagaccaaggatcatgcactttttaattattatatgtacttgaacttgatcctatatatacttagtcatgcacttggtactatatatcggtatttcgtattaagtttttgtatatcgaccgtgttcgacataaatccgatcgaattggttcgttttcgaaattctcgatatttcgtaagttcgtgttccttttcgtgtccgactttatcgttttcgttttcgtattttaaatgtaaaagtagaaaacaattttagattttttcgaccgcttccaccaccgcaccagcgccgagatagcccagcgaagcaaacggccgagacggtacccccctctcgagagttccgctccacctccaccacgggggattccttccccaccgctccttccctttcccttcctcgtccgccgttataaatagccagccccgtccccggcttctttccccaacctctcgtcttgctcggacttcggagcacacgcacaacccgatccccaatccccctcgtctctcctcaccggcttcgcggatctccgcttcaaggtacggcgatcgatcatcctccctccctctctctctctctacctaatcttctttagatagactagatcggcgatccatagttagggccttctagttccgttcctgtttttccatggctacgtggtgcaatagatctgatggagttatgagggttaacttgtcatgctcttgcgatttatatatagtctctttaggagatcaatttaatctcggatggttcgagatcggtggtccatggttagtactctaggctgtggagtcgggggttagatccgcgctgttagggttcgtagatgtaggcgatctgttctgattgataacttgttagtacctgggaatcctgggatggttctagctggttcgcagctgagatcgatttcatgatctgctatatcttgtttcgttgcctatccctttttatctgtccgttgtatgatgttagcctttgatatatttcgtcttgtgcagcacttaattgttaagtgataatttttagcatgcctttttttttatttggttttgtttgattgtgctgctgttctagatcagagtagaagactgtttcaaactgcctgctggatttattaaatttggatctgtatgtgtgtcacatatatatcttaataataaagatggatggaacttttatatattttgctgttggttttgctggtactttcttagatatactctttttggatatggataggtaaatgcttagatacatgaagcaacgtacagtttaataattcttgttcatctaataaacacaaataaggacgggcgtaaatgttgctgtgggttttactggtactttcttagatatatacatgcttagatacatgacgtaacatgctgctacagtttaataaatattgtttatataataaacaaacatgatgtttattatcttggtatgcttgggtgatgttatatgcagcagctgtgtggatttttaaataccctgatgatcatgcatgaccttgccttagtttgctgtttatttgcttgagactgcttctttcgcttatactcacccattattttggtgacttctgcaggcactttgcctgaagagaggacgatggcaagggggagatgggtttttgaaggtttttctttggtttcttggcccacacggtggtttcttagcatgagtgccatgttgggagctgtgcgggccaagaaaccaaaagaaagccactcatcaggctcatctctctgtccgatttggagtgcactttgcctgaagagaggacgatggcaagggggagatgggtttttgaaggtttttctcgtgaaatcctccacacacggtggtttcttagcatgagtgccatgttgggagctgtgcgtggaggatttcaacgagaaagccactcatcaggctcatctctctgtccgatttggagcctaggggttttgcactttgcctgaagagaggacgatggcaagggggagatgggtttttgaaggtttatagctgttgatttcccaaacacggtggtttcttagcatgagtgccatgttgggagctgtgcttgggaaatcaacaagctatagccactcatcaggctcatctctctgtccgatttggagcgccataggtcgtttaagctgctgctgtacctgcgtttgtctggtgccctcttgtgtacctgcatatggaggttgtcgtctattaagtatctgtggtttgttttagtcgtgactgagttggtttgaaggacctgttgtgtcttgtgtcccgtgtgtctacccaaaactattatgccgcagtatggcttcatcatgaataagttgatgtttgaacttatataagtttgtgctcagtatgttttattttaggttatatctccttgaaaactggcgcggccttgccgtgccccatctcaataggccagttccatcgttgtagaacttaatataaatagtgatactaacaaaataaagaactgtgctgcttagaatacatagactatttgaaatcatgcatggatacataatagcatatacaacaaaagagaagcaagatcatgcattgtgctatacac

SEQ ID NO: 91 - DNA, 3304 bp, Artificial sequence
  Synthetic
gtgaggcccgtatagatgtagttaaatagctaaaatttttggagaaataagcatttttttggaagaatatatttaaacatgggcttgtaaaacttggctgtaaagatttggaatttaggatcttggagccccaaaactgtataaacttgcttagggacccgtgtcttgtgtgttgcagaccaaaaaatttagaaagcatctaaacacctatttgaatgtaaagtttacagccaaaagttttaggatgtaaagatttgggatctaaaagtagtcattaggaaataacacgttagagagagagagtagatcttcttattggtttctcatgcactaatcgaaccaatcactggaccacttgaaccaaactttatcacattgaactttgtcagttcagttcgaacgcaggactggagctgcccttaaggccaattgctcaagattcattcaacaattgaaacatctcccatgattaaatcagtataaggttgctatggtcttgcttgacaaagttttttttttgagggaatttcaactaaatttttgagtgaaactatcaaatactgattttaaaaattttttataaaaggaagcgcagagataaaaggccatctatgctacaaaagtacccaaaaatgtaatcctaaagtatgaattgcattttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggataggtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggcactttgcctgaagagaggacgatggcaagggggagatgggtttttgaaggtttttctttggtttcttggcccacacggtggtttcttagcatgagtgccatgttgggagctgtgcgggccaagaaaccaaaagaaagccactcatcaggctcatctctctgtccgatttggagtgcactttgcctgaagagaggacgatggcaagggggagatgggtttttgaaggtttttctcgtgaaatcctccacacacggtggtttcttagcatgagtgccatgttgggagctgtgcgtggaggatttcaacgagaaagccactcatcaggctcatctctctgtccgatttggagcctaggggttttgcactttgcctgaagagaggacgatggcaagggggagatgggtttttgaaggtttatagctgttgatttcccaaacacggtggtttcttagcatgagtgccatgttgggagctgtgcttgggaaatcaacaagctatagccactcatcaggctcatctctctgtccgatttggagcgccataggtcgtttaagctgctgctgtacctgcgtttgtctggtgccctcttgtgtacctgcatatggaggttgtcgtctattaagtatctgtggtttgttttagtcgtgactgagttggtttgaaggacctgttgtgtcttgtgtcccgtgtgtctacccaaaactattatgccgcagtatggcttcatcatgaataagttgatgtttgaacttatataagtttgtgctcagtatgttttattttaggttatatctccttgaaaactggcgcggccttgccgtgccccatctcaataggccagttccatcgttgtagaacttaatataaatagtgatactaacaaaataaagaactgtgctgcttagaatacatagactatttgaaatcatgcatggatacataatagcatatacaacaaaagagaagcaagatcatgcattgtgctatacac

SEQ ID NO: 92 - DNA, 309 bp, Artificial sequence
  Synthetic
tcaaatgtatgtctaaccatgcacatatggatatatagataggggaatgatgtagcacgggtgcaaggatatatagttccatgaaaggtttgatatctactcgtactaggagggttagacgaaagaagaaacaaacgtggttgtttccttgcataaatgatgcctatgcttggagctacgcttgtttctttctttccgtctaacctccaccccttttatctctctccctccctctcatactttatctaaattatatctaatttctttgtattggaataacataactacacccttcgtaattcctgacta

SEQ ID NO: 93 - DNA, 15346 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctcgtcgagcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagaaactttattccatgatattttcccgcgtgcgtaaattcaatcttatggtggattttgattttatcaattagtctacaacgtcttatgttcatgatcgggattatataaaatattttctcacagatcagacttattgatgccgaggaccgcatcgatattaaagattatcaatatatttcattcgctattctccttcacaaaaaaatgaagtatgaacaactgaagtaagatgtatgaaatgttgaatgcttcgagcttctagaagtggtttcttattttggtaaaaggttgtcattacctgattcagttacgaaattcgataagaagcttctttctcgcattcaaattcgagttaagcctttaccgaaatttgattctaccgtgggggtgacagtcggtaccccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgatgactgtcacccccacggtagaatcaaatttcggtaaaggcttaactcgaatttgaatgcgagaaagaagcttcttatcgaatttcgtaactgaatcaggtaatgacaaccttttaccaaaataagaaaccacttctagaagctcgaagcattcaacatttcatacatcttacttcagttgttcatacttcatttttttgtgaaggagaatagcgaatgaaatatattgataatctttaatatcgatgcggtcctcggcatcaataagtctgatctgtgagaaaatattttatataatcccgatcatgaacataagacgttgtagactaattgataaaatcaaaatccaccataagattgaatttacgcacgcgggaaaatatcatggaataaagttttctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 94 - DNA, 803 bp, Artificial sequence
  Synthetic
ccaaatgatgattattcaagtacagacatgtcttcttgactcttatgaagaaactaataaggcttgacaatggggacaacttgggctggtgtgaaaaaattaggattcttttgtttgtgcttcctaatggcgatataagagaggaaagcaagataacatctgattacaataattatgttaaacatcctgaatgtttgtccattctatgtatatctgacaaatcattgtatgggaggttcacctactctgacatcaatgttcatatcatgcaaacaagagagatcatcttgagtaaaataagtgagatagatgaggttggtgaaactgatgaaaacaatttcttgcttagttatataataggggaagtagatgcctttgaagaagatgattttgaagaagaagaagacaaagattaggaacatcatcttttggaacctttgaatctgattctatcaaagaatcagagggttttgatatttctgctagattgatagtacatacaaaccatcatgtctcaaactagaaaaatgatctttttttttgcaacactaagcaaaatgctaataaggttatcaagatcagtccaacttgggacgttggagaatctctttagcaaatttaaagaattatcacatttttctaaactttcttctgaatcagaaacaaaggaatatatgacaacattgctttcaacttgataataaatgttataagtagatatcccctttttctcactttttaatgaagaagcaatcaagcagttgttaggatgatccaaaaaagaaattgtcttttgagttgtt

SEQ ID NO: 95 - DNA, 16158 bp, Artificial sequence
  Synthetic
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctcgtcgagcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagccaaatgatgattattcaagtacagacatgtcttcttgactcttatgaagaaactaataaggcttgacaatggggacaacttgggctggtgtgaaaaaattaggattcttttgtttgtgcttcctaatggcgatataagagaggaaagcaagataacatctgattacaataattatgttaaacatcctgaatgtttgtccattctatgtatatctgacaaatcattgtatgggaggttcacctactctgacatcaatgttcatatcatgcaaacaagagagatcatcttgagtaaaataagtgagatagatgaggttggtgaaactgatgaaaacaatttcttgcttagttatataataggggaagtagatgcctttgaagaagatgattttgaagaagaagaagacaaagattaggaacatcatcttttggaacctttgaatctgattctatcaaagaatcagagggttttgatatttctgctagattgatagtacatacaaaccatcatgtctcaaactagaaaaatgatctttttttttgcaacactaagcaaaatgctaataaggttatcaagatcagtccaacttgggacgttggagaatctctttagcaaatttaaagaattatcacatttttctaaactttcttctgaatcagaaacaaaggaatatatgacaacattgctttcaacttgataataaatgttataagtagatatcccctttttctcactttttaatgaagaagcaatcaagcagttgttaggatgatccaaaaaagaaattgtcttttgagttgttggtaccccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgataacaactcaaaagacaatttcttttttggatcatcctaacaactgcttgattgcttcttcattaaaaagtgagaaaaaggggatatctacttataacatttattatcaagttgaaagcaatgttgtcatatattcctttgtttctgattcagaagaaagtttagaaaaatgtgataattctttaaatttgctaaagagattctccaacgtcccaagttggactgatcttgataaccttattagcattttgcttagtgttgcaaaaaaaaagatcatttttctagtttgagacatgatggtttgtatgtactatcaatctagcagaaatatcaaaaccctctgattctttgatagaatcagattcaaaggttccaaaagatgatgttcctaatctttgtcttcttcttcttcaaaatcatcttcttcaaaggcatctacttcccctattatataactaagcaagaaattgttttcatcagtttcaccaacctcatctatctcacttattttactcaagatgatctctcttgtttgcatgatatgaacattgatgtcagagtaggtgaacctcccatacaatgatttgtcagatatacatagaatggacaaacattcaggatgtttaacataattattgtaatcagatgttatcttgctttcctctcttatatcgccattaggaagcacaaacaaaagaatcctaattttttcacaccagcccaagttgtccccattgtcaagccttattagtttcttcataagagtcaagaagacatgtctgtacttgaataatcatcatttggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaaggg

SEQ ID NO: 96 - DNA, 2854 bp, Solanum lycopersicum
atattttataagaaaagaataaacaagacaaggttggacgtggaaaggaaaaatcaagcagttggttaaagcagcgatggtggacaaactgatcgtcgccgttgaaggcaccgctgtgttgggcccttactggaaaatcatcgtttccgattaccttgataaaattatcaggtgcttttttggagtggactcaacttcacagaaatcatctgcagccgatgtggaggtctccatggtcatgtttaatacacatggtccttatagtgcttgcctggttcagcggagtggctggacaaaagacatggatacctttttgcagtggctttcagctatacctttttcaggaggtggtttcaatgacgctgcagttgcagaaggacttgctgaagcattagtgatgttttctgtaccaaatggtaaccaaactcaacaaaaaatggaagggaaaaaacattgcatacttatttctggaagcaacccttatccgttgcctacaccagtttatcgaccacagatgcagaaactggagcagaacgaaaatattgaagcacaaacagatagtcgactagctgatgctgagacagttgcaaagacattccctcagtgttctatttccctgtcagttatatgcccaaaaaagcttccaaaactaagagcaatatatgatgcgggaaaacacaatccacgagcagctgacccgcccattgatacggctaagaatccaaactttcttgttctgatatccgaaaacttcatcgaggctcgtgctgctttcagtcgctctggactgaccaatttggcgtcaaatcacagccctgtcaagatggatgtgtcttctgttcttccagtttctggcacacaatcaatttctaattcagctgcaaatgtatctgttatcagtcgaccaccaatttccgctggaaatattcctccggcaactgtaaaaattgagcccaacactgtgacccccatgactggacctggattttcacatattccgtctgttcgtcctgctcttcaaccggttccaagtttgcaagcttcttctcctctttctgtttctcaggagatggtatcacatactgagaatgttcaggagatgaaacccatagtcagtggtatgactcagtccttacgtccggttgctgctgcagcagcaaatgtcaaaatcttgaacggtgttgctcaggcacaccaagttctaggcggggggacttctataggactgcaatctatgggtggcacccctatgctttcaagtatgatatctagtggaatggcatcttccgtccctgcttctcaagctgtattatcatctgggcaatcaggtgtgacaacaatgactggggctgttccactagcaggaagtgcacagaatacacaaaactcagctccttcttcattcacttcaactgctcccagtatgtctggtcaaacggttcctgcaatgagccaaggtaatataccaggcacacaaatgatgccaagtgggacagggatgaaccagaatatgctgactgggctgggtgcaactggtttgccttccggaactggcacaatgatgcctactccagggatgtctcaacaaggacaaccaggcatgcaaccagttggtgtgaacagcacatcagcgaacatgcctctatctcaacagcaaacatctggtgcattgccatcagctcaatcaaaatatgtcaaagtttgggagggaaacttatctggtcagagacaggggcagcctgtatttattaccagattggaggggtataggagtgcctcagcttctgaatcgcttgctgcaaattggcctccaacaatgcaaattgttcgccttatttcccaagatcacatgaataacaaacaatatgttgggaaggcagattttctagtttttcgggcaatgaaccagcatgggttccttagtcagctgcaagaaaagaagctttgtgcggtaatacaactcccatcgcagacactgcttctgtctgtttctgataaagcctgccgcttgattggaatgctttttcctggggatatggtggtgtttaagccacagataccaagtcaacagcaacaacagcagcaacagcagcagctgcaagcacaacatccacagttgcagcaacagcagcaacagcagcagcagcatcttacgcaactacaacagcaaccccttcaacagctgcaacaacagcaacagcagcaacctctcatgcaactgcagcaacaacagcagattccgttgcaacaatcacaggttccccaaatgcagcaacaacagattcaccaaatgcagcagcagcagcagatcccgcaaatgcagcagcagcagcagattccgcaaatgcagcagcaacagcagcagcaaccaatggttggaacagggatgaaccaaacctacatgcaaggtcctgcaaggtcacagctaatgtctcagagtcaaggttcatcacaaggactgccaatcacaccaggaggcgggtttatgaattgaaaatgtcatagaactagacaaccattgtacaaatgtgagaccaggcagttgattccaaaagtgtagccatgcatttggtgcatatcttaacaatggaatttcgaagtaatcaaattcattatagactctagcatagaaattacttgtgcccttcaaatcaggttggggcggctgtaaaggcaaaataaaaggggaagagggaggaattttctgggcgtgggcgtgggcgtgagaacatgactgcatatttggtcaccaatctattttcttttgatctcaacatcagtattctttttggttttttgttcaactatagttattcacgtcataggttcaacatttcactagatttcaccatca

SEQ ID NO: 97 - DNA, 143 bp, Artificial sequence
  Synthetic
gagcaggaaagtattgggtgagatattgtttggttaccatttggtacaggaataatgaggtgctaattggaagctgcaccttaattctttctgtaccaaattggtaaccaatcatcttcagtccctccccgaccctctctacc

SEQ ID NO: 98 - DNA, 3336 bp, Artificial sequence
  Synthetic construct
gcactgttgggtccggcccatttggatagctgttgtactgatgtcgtgcctaatgaggaaattggagtaactgtctaactgattcaacagaggaaattaaatccaggtgagggatgatcagtgaatccaaatattgtactaatgatcatgagtattttatgtgggcattcttcttgacttgaagttgtaaccggacataactgttgctaggaatgtatgacttgaagctttaagcactactacatctgatgatccttacatgttgagcaagaggctggaagaaaaaaaggatgagagccttttaacccatgtaactccaatgctgtctgcaaacctttcagctggttgtgagttgtggctgcagatctgaggaagccactgagagagcactaggtaaggtttctatgttatatctgtaattctgtatatgtttaatgtgttgtgctcatttaaaaaaagaactgcatagattcacaaactgcctggagctttcctcttcactttgctaatagtattgaccatgttttgggcttgttgtgtgtttggaaggagtgggtctataggccgtcagtgtttaggcctactaattaagccagttcagttgggccttggctgcttccatggattaattatgagactaatcgctgatactcgtacatcaatgcgagaatttgtcaaatcaattggatcagtatatatcttgcctgtatgattcacatccatacgtttctatcgagttggttggaagatttgtgccgtcgatgacagtgcagaagagaagcttccttgcaactagtgtcgtggcagaagcagaggtagacacatgaaatcgtgttctaatccgtcgcagctagctagcatggcagcgacgtgtttgacgatgacaccaccttgcatatccagatgcctctgtttgacctggatggaacaaacaataacgtacgtttttcgagcatctaaatggtataaatttttagagaaatttttatgtgtaatttctttcttataatatagttttaaaatctgtttcacaactaatcaattcagtcgtttgtatcctcgaatcattttcatgttcaacattccatccatttaggcatttatgcacagaacagtagaatacatagtttgtccatgctttaaacgaaaagtaaaaaagaaagaaaaagacacatattcctcttaaaacaatattcgtttgagatggtggagggaacaaaggccattgatttgctgcagggtccctccctaacaagctgtgatgattctgtatacgacgatcgtgcaattaagctagtgctttgaaagagacagacagacagacaacttttttcctcctaatacgatcggatgaaaactgtcgagcttttatgtagcgtataaaccttgactgttgcgaggaaaaaaaagctgtaggaaacaaagaaatcgaggaaatgaatttgtcctggtttcgtatatatgtacatgtactatatgccaaaaacgcccgtgcttaacagctaagaaatcggccaaaattcaggcaaacaagagacaaagttagcaggcaacgcgtcactaccgcgtgatcatttcgacgcgaaggcaatttggccggtgatcgagcgcgtctcgtgcagtgaatgaagtagcttaatttgctagtccccacaagtacgtggcactctgccatgtcttctcttagtataaatatatggaagccaaagccaaagccagtcagttcatcagttgcagttcagagttgcccactgctactttactttgcagctattttgcttctgcttcttcttgttcttgttgctgttggtaatactgcgagagaaattaatcagtagagtgttcatctactatcaatttttgatcgaggagagatgcaagcaatcgatctgtttggttgtgcaatgctcagtctgcagatagcaaaaccctttctacgtgcgctcttagcgaagagcgcgtctattcagacaattgtctgcatccccttcatgagtggcgtgcttgagctgggaaccactgatccggtttcggaggacccaaacttggtaaaccgaatcgttgcatatttgaaggagcttcagtttccgatatgcttagaggtaccgagttctactccttctccagacgaaacagaagatgccgacaccgtgttcgatggcctcattgaagaggaccagatggtcatactccagggagaagacgagctaggcgacgtcgtcgtcgctgagtgcgagaccaacggcgccaaccccgaaacgatcaccatggagaccgacgagttctacagcctctgcgaggagctggacctggacctcggttcttatcagctagtcccgacgtcggcacgggagacggtggctgcggcggcggcggcggctaacgatgtcgacggcgttgcatactctcacgcctcgtgtttcgtgtcatggaaaagagcgaacccggcggagaaggtggtggccgtgccgatgactgcaggcatagagtcacagaagttgctgaagaaagctgtaggcggcggcaccgcatggatgagtaatattgatgatcgtggtagcgtggcaataacgacgactccaggaagtaacatcaagagccatgtcatgtcagagagaaggcgccgagagaagctcaacgagatgttcctcattctgaagtcactactcccctccgtccgcaaggttgacaaggcatccatacttgcagaaacgataacctacctcaaagtgttagagaaaagagtgaaagagctggagtccagcagcagggagccatcgcgttggcggccaaccgaaattggacaggggaaggcgccgtaatcattttcaatgtccccccagtctcttcctctaatgtttgatgatatgtagaatctcttgctgttaatctgttgctttcgtgtgaataagctcccctcttatgtatatctatctatgacttgttttactagtaaacttgattattgagagcttgagacttgcttccctgtctgtctgtatatcttgttccaacactggaactgaatcaataattgtgcctgcactgctcaagagtgctctgaatgtagtcataattgcagataattagtacaattatctcactcaaatccctttaataaaaaggagcatcaacttggatcataaagttgcactcatcgctatctcttttttcccctttatccattgaaaaaaccttttgtttttctttttgggatttggttcgcgtcactaaaatagccaaataatatcaggtttgttggaacgtgattatcttcagagtgattgaggcagaattcaagtgggccctctcgcttacatggacttttatgggctg

SEQ ID NO: 99 - DNA, 743 bp, Solanum lycopersicum
agtactttattcccgactcaccttttttgaccagtccctccctattattcaattcccaatccctaacgaagaaaatacaaatcgatctccatttcctccatctctcttcaagctctaattttgaagctttaatggagtcgtcgtcgtcatcaccagcttcacgagaactggatgaggtacaaacagatcttccttcatctgtaagatccgcttcgagaatcagagctccgaataatatggttatggggaaacatcgtcttgctgctgcaatttctgctctcaatcaacaaatcaacatcattcaggaagaattggatcagcttgactcgtttggtgaagcttcactcgtttgcagagaattagtttcaagtgttgagttaatacctgatgctctccttccagtgactagaggaccaataaatgttcatttagatagatggtttcatggagttaacgattcaagacgcaacaaacgctggatatgaaaaaggaatttgtaccaaaaactacgtaaatcaattcgaataccctcttctttttttggtttattttgtaattaaatttttaaatttctttgttttcttgactgctatattattaggtctttatattctatatatatctgctgttgaatattgctgatgaaataatgtaggtaagtatgttttgtaactcatacacttcccaagtattaataaaagtgtgttatatgcacaaaaaattgtttcaacttgacaaaaaaa

SEQ ID NO: 100 - DNA, 3114 bp, Artificial sequence
  Synthetic
tggcaggatatattgtggtgtaaacataagtcttttaagataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagttgattaaaatacaaatcgatctccatttcctccatctctcttcaagctctaattttgaagctttaatggagtcgtcgtcgtcatcaccagcttcacgagaactggatgaggtacaaacagatcttccttcatctgtaagatccgcttcgagaatcagagctccgaataatatggttatggggaaacatcgtcttgctgctgcaatttctgctctcaatcaacaaatcaacatcattcaggaagaattggatcagcttgactcgtttggtgaagcttcactcgtttgcagagaattagtttcaagtgttgagttaatacctgatgctctccttccagtgactagaggaccaataaatgttcatttagatagatggtttcatggagttaacgattcaagacgcaacaaacgctggatatgaaaaaggaatttgtaccaaaaactacgtaaatcaattcgaataccctcttctttttttggtttattttgtaattaaatttttaaatttctttgttttcttgactgctatattattaggtctttatattctatatatatctgctgttgaatattgctgatgaaataatgtaggtaagtatgttttgtaactcatacacttcccaagtattaataaaagtgtgttatatgcacaaaaaattgtttcaacttgacaattgggaagtgtatgagttacaaaacatacttacctacattatttcatcagcaatattcaacagcagatatatatagaatataaagacctaataatatagcagtcaagaaaacaaagaaatttaaaaatttaattacaaaataaaccaaaaaaagaagagggtattcgaattgatttacgtagtttttggtacaaattcctttttcatatccagcgtttgttgcgtcttgaatcgttaactccatgaaaccatctatctaaatgaacatttattggtcctctagtcactggaaggagagcatcaggtattaactcaacacttgaaactaattctctgcaaacgagtgaagcttcaccaaacgagtcaagctgatccaattcttcctgaatgatgttgatttgttgattgagagcagaaattgcagcagcaagacgatgtttccccataaccatattattcggagctctgattctcgaagcggatcttacagatgaaggaagatctgtttgtacctcatccagttctcgtgaagctggtgatgacgacgacgactccattaaagcttcaaaattagagcttgaagagagatggaggaaatggagatcgatttgtaggtgctgctataattacttaaaagtgcgagtgtcctgtctgtttcccggttttgctattatgttgccagtcaatttgtttttttgatgggatggagaagtttggtggtgggggctatgaatgcacggtagcaaacaacagattgccagtattatctcatgtttccatttaatgtggttaatattctctacatacttgagaggtgcctgatgcattgccctcttctgtctggctacaccatcccttggtcgaagcgtctcttttttaggttgtttgtagttgaaggagagtgattgtgatgttttctcctcgtcttttctctcattttctccttttatctgattttgcacttttgtggttcttttttttcttggacccaataatgtcaatatttattgaatgagaaaattcctatatcatatcagtttgaggaaatcattactatttgtgtggatacaggagttttgactctttattggcgatattttgtattctattgttgctgttttggatgtggtttcagaacttccttagtgcatttgctcttaaatctgttttgcagtaaaattgaggctataaaagcttcattgcagattaccctcggatgagggatctcctcattgcctgtcatatattggtttcttttcatccaacacgcaggatacatacatttattgaatttgaccttctattttgggacaactctactgtgaaattggagggattgttgaatttttttcttgcatgagttcattgatggtattatttttgacaggatatattggcgggtaaac

SEQ ID NO: 101 - DNA, 3514 bp, Artificial sequence
  Synthetic construct
tgcgcactgttgggtccggcccatttggatagctgttgtactgatgtcgtgcctaatgaggaaattggagtaactgtctaactgattcaacagaggaaattaaatccaggtgagggatgatcagtgaatccaaatattgtactaatgatcatgagtattttatgtgggcattcttcttgacttgaagttgtaaccggacataactgttgctaggaatgtatgacttgaagctttaagcactactacatctgatgatccttacatgttgagcaagaggctggaagaaaaaaaggatgagagccttttaacccatgtaactccaatgctgtctgcaaacctttcagctggttgtgagttgtggctgcagatctgaggaagccactgagagagcactaggtaaggtttctatgttatatctgtaattctgtatatgtttaatgtgttgtgctcatttaaaaaaagaactgcatagattcacaaactgcctggagctttcctcttcactttgctaatagtattgaccatgttttgggcttgttgtgtgtttggaaggagtgggtctataggccgtcagtgtttaggcctactaattaagccagttcagttgggccttggctgcttccatggattaattatgagactaatcgctgatactcgtacatcaatgcgagaatttgtcaaatcaattggatcagtatatatcttgcctgtatgattcacatccatacgtttctatcgagttggttggaagatttgtgccgtcgatgacagtgcagaagagaagcttccttgcaactagtgtcgtggcagaagcagaggtagacacatgaaatcgtgttctaatccgtcgcagctagctagcatggcagcgacgtgtttgacgatgacaccaccttgcatatccagatgcctctgtttgacctggatggaacaaacaataacgtacgtttttcgagcatctaaatggtataaatttttagagaaatttttatgtgtaatttctttcttataatatagttttaaaatctgtttcacaactaatcaattcagtcgtttgtatcctcgaatcattttcatgttcaacattccatccatttaggcatttatgcacagaacagtagaatacatagtttgtccatgctttaaacgaaaagtaaaaaagaaagaaaaagacacatattcctcttaaaacaatattcgtttgagatggtggagggaacaaaggccattgatttgctgcagggtccctccctaacaagctgtgatgattctgtatacgacgatcgtgcaattaagctagtgctttgaaagagacagacagacagacaacttttttcctcctaatacgatcggatgaaaactgtcgagcttttatgtagcgtataaaccttgactgttgcgaggaaaaaaaagctgtaggaaacaaagaaatcgaggaaatgaatttgtcctggtttcgtatatatgtacatgtactatatgccaaaaacgcccgtgcttaacagctaagaaatcggccaaaattcaggcaaacaagagacaaagttagcaggcaacgcgtcactaccgcgtgatcatttcgacgcgaaggcaatttggccggtgatcgagcgcgtctcgtgcagtgaatgaagtagcttaatttgctagtccccacaagtacgtggcactctgccatgtcttctcttagtataaatatatggaagccaaagccaaagccagtcagttcatcagttgcagttcagagttgcccactgctactttactttgcagctattttgcttctgcttcttcttgttcttgttgctgttggtaatactgcgagagaaattaatcagtagagtgttcatctactatcaatttttgatcgaggagagccaatggccagatttgcagtgcaacatcgcgtcttattcttcataaaaaaatcgctaaagaatttcaagaaaggatggttgcatgggccaaaaatattaaggtgtcagatccacttgaagagggttgcaggcttgggcccgttgttagtgaaggacagtatgagaagattaagcaatttgtatctaccgccaaaagccaaggtgctaccattctgactggtggggttagacccaagcatctggagaaaggtttctatattgaacccacaatcattactgatgtcgatacatcaatgcaaatttggagggaagaagtttttggtccagtgctctgtgtgaaagaatttagcactgaagaagaagccattgaattggccaacgatactcattatggtctggctggtgctgtgctttccggtgaccgcgagcgatgccagagattaactgaggagatcgatgccggaattatctgggtgaactgctcgcaaccctgcttctgccaagctccatggggcgggaacaagcgcagcggctttggacgcgagctcggagaagggggcattgacaactacctaagcgtcaagcaagtgacggagtacgcctccgatgagccgtggggatggtacaaatcctgcgagcagttcacccagataattccggcatcgatctcctcagttaatctctggcatcgctcgcggtcaccggaaagcacagcaccagccagaccataatgagtatcgttggccaattcaatggcttcttcttcagtgctaaattctttcacacagagcactggaccaaaaacttcttccctccaaatttgcattgatgtatcgacatcagtaatgattgtgggttcaatatagaaacctttctccagatgcttgggtctaaccccaccagtcagaatggtagcaccttggcttttggcggtagatacaaattgcttaatcttctcatactgtccttcactaacaacgggcccaagcctgcaaccctcttcaagtggatctgacaccttaatatttttggcccatgcaaccatcctttcttgaaattctttagcgatttttttatgaagaataagacgcgatgttgcactgcaaatctggccattggtcattttcaatgtccccccagtctcttcctctaatgtttgatgatatgtagaatctcttgctgttaatctgttgctttcgtgtgaataagctcccctcttatgtatatctatctatgacttgttttactagtaaacttgattattgagagcttgagacttgcttccctgtctgtctgtatatcttgttccaacactggaactgaatcaataattgtgcctgcactgctcaagagtgctctgaatgtagtcataattgcagataattagtacaattatctcactcaaatccctttaataaaaaggagcatcaacttggatcataaagttgcactcatcgctatctcttttttcccctttatccattgaaaaaaccttttgtttttctttttgggatttggttcgcgtcactaaaatagccaaataatatcaggtttgttggaacgtgattatcttcagagtgattgaggcagaattcaagtgggccctctcgcttacatggacttttatgggctgcgca

SEQ ID NO: 102 - DNA, 7 bp, Artificial sequence
  Synthetic
cctcagc

SEQ ID NO: 103 - DNA, 6 bp, Artificial sequence
  Synthetic
cgtacg

SEQ ID NO: 104 - DNA, 25 bp, Artificial sequence
  Synthetic
tgacaggatatattggcgggtaaac

SEQ ID NO: 105 - DNA, 25 bp, Artificial sequence
  Synthetic
tggcaggatatattgtggtgtaaac

SEQ ID NO: 106 - DNA, 1017 bp, Lycopersicum solanum
taaacataagtcttttaagataatagttcgtaaatttttgctcgagcgcacacatagttgaaaaaaaaaattaaattttgtgaaagaagatcgaaaaaatcaactcaaattgataggaattagattttaaaaaaattgaaaataatttgaacaaagattttccttgtttactccattcaatagtggagggcgaatctgtcaatttggttgtctttgtgctcaccacctcttatcattcaaattcaaaaatacattgaatagaataaaaaagaaaattataaattcaaaggccgtctcagccagtttttacgactatatatatacttgtgtattgtcttaactcattcatcctcttccagactgtagagagagaaagcaagtcggccacaagtcatcatccgtttgcctttgcttttcagatccattttcatttccttttcggtaatctaacctatcttcttcatcagatcttgctttatttacttgcttcttttctttcaatttctgctttgagatctgctctacttactcatgttgaatcgctgctttttgttcttctgattactctactgctctaattacttagtaaaacttagatttaggtgtgatattctctttgatttttccagatctgttgtttttatggtcaatctgtcatgaacttgatctgctcttaattttcctagatctactgtgttattagtacttgatctctgcatactcattttggttaccagcaaatttagctaaactttgatggatcttttttttttggctgctatacggaaaaacgaagcatgtttttattattacaagtgtccgcctgttgactgagctccaaattgtctgggatttagatatatcagtttacttactaacaagtaaaaccttatatgactagagacatttagttgagttctgaatcgatcttatgatgttgtgttatgtgttgataccttcatgtatatgtttaggttagactaagtgtgctgatttaacttgcttttactttcagtt

SEQ ID NO: 107 - DNA, 770 bp, Lycopersicum solanum
gtgctgctataattacttaaaagtgcgagtgtcctgtctgtttcccggttttgctattatgttgccagtcaatttgtttttttgatgggatggagaagtttggtggtgggggctatgaatgcacggtagcaaacaacagattgccagtattatctcatgtttccatttaatgtggttaatattctctacatacttgagaggtgcctgatgcattgccctcttctgtctggctacaccatcccttggtcgaagcgtctcttttttaggttgtttgtagttgaaggagagtgattgtgatgttttctcctcgtcttttctctcattttctccttttatctgattttgcacttttgtggttcttttttttcttggacccaataatgtcaatatttattgaatgagaaaattcctatatcatatcagtttgaggaaatcattactatttgtgtggatacaggagttttgactctttattggcgatattttgtattctattgttgctgttttggatgtggtttcagaacttccttagtgcatttgctcttaaatctgttttgcagtaaaattgaggctataaaagcttcattgcagattaccctcggatgagggatctcctcattgcctgtcatatattggtttcttttcatccaacacgcaggatacatacatttattgaatttgaccttctattttgggacaactctactgtgaaattggagggattgttgaatttttttcttgcatgagttcattgatggtattatttttga

SEQ ID NO: 108 - DNA, 2200 bp, Artificial sequence
  Synthetic
gtgttacacagctcaattacagactactcaccatgcatctgcgttctttctaccggtggctagttgcgttcctgctagctattaattgcttattctagacttgtatttatgtgtgggctattttattaaatacctaagaccaaggatcatgcactttttaattattatatgtacttgaacttgatcctatatatacttagtcatgcacttggtactatatatcggtatttcgtattaagtttttgtatatcgaccgtgttcgacataaatccgatcgaattggttcgttttcgaaattctcgatatttcgtaagttcgtgttccttttcgtgtccgactttatcgttttcgttttcgtattttaaatgtaaaagtagaaaacaattttagattttttcgaccgcttccaccaccgcaccagcgccgagatagcccagcgaagcaaacggccgagacggtacccccctctcgagagttccgctccacctccaccacgggggattccttccccaccgctccttccctttcccttcctcgtccgccgttataaatagccagccccgtccccggcttctttccccaacctctcgtcttgctcggacttcggagcacacgcacaacccgatccccaatccccctcgtctctcctcaccggcttcgcggatctccgcttcaaggtacggcgatcgatcatcctccctccctctctctctctctacctaatcttctttagatagactagatcggcgatccatagttagggccttctagttccgttcctgtttttccatggctacgtggtgcaatagatctgatggagttatgagggttaacttgtcatgctcttgcgatttatatatagtctctttaggagatcaatttaatctcggatggttcgagatcggtggtccatggttagtactctaggctgtggagtcgggggttagatccgcgctgttagggttcgtagatgtaggcgatctgttctgattgataacttgttagtacctgggaatcctgggatggttctagctggttcgcagctgagatcgatttcatgatctgctatatcttgtttcgttgcctatccctttttatctgtccgttgtatgatgttagcctttgatatatttcgtcttgtgcagcacttaattgttaagtgataatttttagcatgcctttttttttatttggttttgtttgattgtgctgctgttctagatcagagtagaagactgtttcaaactgcctgctggatttattaaatttggatctgtatgtgtgtcacatatatatcttaataataaagatggatggaacttttatatattttgctgttggttttgctggtactttcttagatatactctttttggatatggataggtaaatgcttagatacatgaagcaacgtacagtttaataattcttgttcatctaataaacacaaataaggacgggcgtaaatgttgctgtgggttttactggtactttcttagatatatacatgcttagatacatgacgtaacatgctgctacagtttaataaatattgtttatataataaacaaacatgatgtttattatcttggtatgcttgggtgatgttatatgcagcagctgtgtggatttttaaataccctgatgatcatgcatgaccttgccttagtttgctgtttatttgcttgagactgcttctttcgcttatactcacccattattttggtgacttctgcagcgctaggcgccataggtcgtttaagctgctgctgtacctgcgtttgtctggtgccctcttgtgtacctgcatatggaggttgtcgtctattaagtatctgtggtttgttttagtcgtgactgagttggtttgaaggacctgttgtgtcttgtgtcccgtgtgtctacccaaaactattatgccgcagtatggcttcatcatgaataagttgatgtttgaacttatataagtttgtgctcagtatgttttattttaggttatatctccttgaaaactggcgcggccttgccgtgccccatctcaataggccagttccatcgttgtagaacttaatataaatagtgatactaacaaaataaagaactgtgctgcttagaatacatagactatttgaaatcatgcatggatacataatagcatatacaacaaaagagaagcaagatcatgcattgtgctatacac

SEQ ID NO: 109 - DNA, 6 bp, Artificial sequence
  Synthetic
ctgcag

SEQ ID NO: 110 - DNA, 6 bp, Artificial sequence
  Synthetic
ggcgcc

SEQ ID NO: 111 - DNA, 2311 bp, Sorghum bicolor
gtgaggcccgtatagatgtagttaaatagctaaaatttttggagaaataagcatttttttggaagaatatatttaaacatgggcttgtaaaacttggctgtaaagatttggaatttaggatcttggagccccaaaactgtataaacttgcttagggacccgtgtcttgtgtgttgcagaccaaaaaatttagaaagcatctaaacacctatttgaatgtaaagtttacagccaaaagttttaggatgtaaagatttgggatctaaaagtagtcattaggaaataacacgttagagagagagagtagatcttcttattggtttctcatgcactaatcgaaccaatcactggaccacttgaaccaaactttatcacattgaactttgtcagttcagttcgaacgcaggactggagctgcccttaaggccaattgctcaagattcattcaacaattgaaacatctcccatgattaaatcagtataaggttgctatggtcttgcttgacaaagttttttttttgagggaatttcaactaaatttttgagtgaaactatcaaatactgattttaaaaattttttataaaaggaagcgcagagataaaaggccatctatgctacaaaagtacccaaaaatgtaatcctaaagtatgaattgcattttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggataggtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcag

SEQ ID NO: 112 - DNA, 1748 bp, Oryza sativa
ttttctatgatatatgtaagggtaaattggacaaatcatatatattttgcatagtaaggtgacatggcatatctatgtggtgattttggtgggaccaaggactatatcagcccacatgacaaatttaaaggacttgtttggacaatatgaaagattaaggactaaaatgacctaggagcgaaactttagggaccatattggctattctccctttttgacacgaatgaaaaatccaatttcataacttgtctggaaaccgcgagacgaatcttttgagcctaattaatccgtcattagcacatgcgaattactgtagcacttatggttaattatggactaattaagctcaaaagattcgtcttgcgatttcctttttaactgtgtaattagtttttcttttactctatatttaatgctccatgcatatgtctaaagatttgatttaatgtttttcgaaaaaacttttggaggactaaccgggcctaacgtgacttgaagagctgtgacagcgcaaatcgtgaaacgcggatggacctagcattatggtgatgtaggaagtgccttgctggcagtggcaggtaccgtgcaagtgtaataccatagatccgttggcttatctgattacatgatgatgattactccctccgtttcacaaatataagtcattttagcatttttcacatttatattgatgttatgtctagattcattaacatcaatatgaatgtgggaaatgctagaatgacttacattgtgaaacggatcattaacatcaatatgaatgtggaaaatgctagaatgacttacactgtgaaacggagggagtatacgattatgtaatgaaaaaaggagtacaatactagtcgccgtctccccgcaaaaaaagtactagttgtcgtcaagtaggggagtaataataataataataataagggataatatacaggctgtgtttagttcgtgtgccaaatttttttaaagtatacggacaaatatttaaatattaaacatagactaataacaaaacaaattacagattccatctgtaaactgcgagacgaatctattaaacctaattaattcgttattagcaaatgtttactgtagcaccacattatcaaatcatggcgtaattagctcaaaagattcgtctcgcgatttacatgcaaaccatgcaattgatttttttttcatctacgtttagttctatgcatgtgtccaaatattcgatgtgatgaaaaaattggaaattcgaggaaaaaaatttaaatctaaacacggccacagtataaaaaaaatagtagcgttgttgtttatgaaagaggatggtaaagtaagacaagacaacgcaagggcctaaaaaagtggagacgaagaagaagacggaatatattgcattggaaaagtgagcgcttggacgagagaaaaactcggattcaagcgtccatatcagtggacaccaccaatgggaggtggccacgtgggcaggtcccgggtggaatctggcgcgttcacacgggaggttccgaaattacggcaacgccactggagtgcgaggcgcaggatgtgagatccacggcgggggctccgctactagaaacttcttctggtcgtgggtggtacgcaccctcgcgcctcgcctttatattactagtaagaagatctcatccctccttggtgaggtgaggtgagttgagttggggattgattgattgattcggattgggaagaagaagaagcaggggagc

SEQ ID NO: 113 - DNA, 708 bp, Oryza sativa
taagaagcctttagagagcgggatatccgcaaaagattaatgccgatttgtattttgcgccttagagtcagtacgatcaagactgtcgtggcggttgtaataaaaattagtgtgctttgggccatctttttatgtgattccaattgtctttctcttcattcttgctttgatgctctttgtctggacctctagaccgccgtattgtactgtggagtttcaaagttaccaagctatttgctgtcaagataactatggattgaattccccttgatggatgaaccaactgttgttgtttgcccgttcttcagctttcgtttgtgcggccatcgatcgccatgcgttgcttaaacccatttctagctcccctaccctgctgcatccgccctcttctgcgcgatcgttggattgcgagtggttggctggttgcacgacttgtggagaccgaaacaaataatttttggtcaaattgatcggtggtactgtcggagcatctattttttctttagcttagatcgtataattgtaggattgggatttgtatattaatatatacaggtcgattaaaacaatgcaactattcgtgatgtcatgtgacctaaacaaatgtgtgccatttatgatatttttcaagagtggttcttatagacttcttactaacaaaaattcacgacaattggactgagcctcaaaagttaataaaaaagaatc

SEQ ID NO: 114 - DNA, 16 bp, Oryza sativa
gagctccggattataa

SEQ ID NO: 115 - DNA, 6 bp, Artificial sequence
  Synthetic
gaacgt

SEQ ID NO: 116 - DNA, 6 bp, Artificial sequence
  Synthetic
cgattc

SEQ ID NO: 117 - DNA, 6 bp, Artificial sequence
  Synthetic
gctagc

SEQ ID NO: 118 - DNA, 6 bp, Artificial sequence
  Synthetic
cacgtg

SEQ ID NO: 119 - DNA, 717 bp, Artificial sequence
  Synthetic
atgtgcgggatcaagcaggagatgagcggcgagtcgtcggggtcgccgtgcagctcggcgtcggcggagcggcagcaccagacggtgtggacggcgccgccgaagaggccggcggggcggaccaagttcagggagacgaggcacccggtgttccgcggcgtgcggcggaggggcaatgccgggaggtgggtgtgcgaggtgcgggtgcccgggcggcgcggctgcaggctctggctcggcacgttcgacaccgccgagggcgcggcgcgcgcgcacgacgccgccatgctcgccatcaacgccggcggcggcggcggcgggggagcatgctgcctcaacttcgccgactccgcgtggctcctcgccgtgccgcgctcctaccgcaccctcgccgacgtccgccacgccgtcgccgaggccgtcgaggacttcttccggcgccgcctcgccgacgacgcgctgtccgccacgtcgtcgtcctcgacgacgccgtccaccccacgcaccgacgacgacgaggagtccgccgccaccgacggcgacgagtcctcctccccggccagcgacctggcgttcgaactggacgtcctgagtgacatgggctgggacctgtactacgcgagcttggcgcaggggatgctcatggagccaccatcggcggcgctcggcgacgacggtgacgccatcctcgccgacgtcccactctggagctactag

SEQ ID NO: 120 - DNA, 3 bp, Artificial sequence
  Synthetic
tgc

SEQ ID NO: 121 - DNA, 3 bp, Artificial sequence
  Synthetic
gca

SEQ ID NO: 122 - DNA, 1919 bp, Oryza sativa
gcaacacacaccccccaaccctacacatacacaaacacaagagtgagagagagattaaaatctaagcactttttgatgcagtcaacacggcttaagtgtggggtaacttgtaagcagggcctttcgagggagagggacacgtgtacaggcagctgataccactacacatgtactacttcatttgctctaaaataaatttattttccactcatccctgcacatgtttatatatgtttatatagaactaaaaatactatatataatacccgtacttcataaactccgagaaaaatataaggaactgaaagtaaatttattctagaatggtgaattatctttctggaacaaaatagtgtacaaaacgcatcttgagaatgcatcgtaagctatttgataaggatagatgtgacgttagtgtcacgttgggatagtggtaaaaaccaaacctcgaatacccagatttccatacattttcgtctatgatgaaaaaaatttatgagtggtgtactttatatttctgacggtttcttgtttccataaaaacaagcaaccaagtctccccaattggttggttaaaacaataaatgaacctcacaaaattttgtagtggccggaatttgatttgaagcataactaactaaaaagctactaggagtattggtttaattttttatgctaagctactggtttaatttgataggacggtgtgccgagtaaaaattaattaggcagaaaggtctatacattgctctgcgctctctctctcctcatggcagacactaactccactggagaaaaatgttaactggaattatttggtattccctcccttcgtttcacaatatattttcctttttatttatcctaaaacaaatttacttttaagtaatcactacatcaaattaaagttaatgaaaatagaggataaatctctactattatatataaaaattaaagatgtttttgccggtattttggtacgttatccgtgtatgagtatgtttttaagttcatttggttttggaaatacatatccatatttgaatcggttcttaagttcgtttgcttttggtaatacagaaggaattgtataaaaaatctgtctaaaaaaactcgcatattaacttgagactattggattcctaactgcagctcatgactttctaaaagtatatatatccaaacgaattccacagtcatcttaactaaaccatatataataataattagattaaaatagattttacccgttgcaatgcacgggtattttcttatagtacattaaaaatttttaaaaaaacaaggaataattgtattaagatttaataaattatgatattttaaactttttaaaaaaaacgagatttgaagggagatatccctccaaacattttttataagaaattatgagcgtgttacggattaaacacaggaccatataagtgaaatcatataaccctttactatcaaatgcatctctaatttagttttttttattcgggagtactgattatatcccctaataaaagaaacatgaagcaatttagtcatgcgttaatcacacaacaaggacaacttattaaaaagtgtgatccatccacgtggtgttttgagccactgcagcagtggtattgtgacagacaaaggaggattccatgcgtctacaaccaaaaaccatcagcctctcctcccgccacgtgtcccccccacccgctcccgccactttcaaaccccacttcccctttgaccgcctctcccgccacctcctataaatctccccatgattcctccctcccattccccacctcacctcacctcctcctccacctcctcgaaattattcgaatccatctccttctccctcctcccaacccgcgccaaatcgatcgatcgcgagcgatcttggccgcgtctcacca

SEQ ID NO: 123 - DNA, 733 bp, Oryza sativa
ctcaaattaattagccagtgaaaaatcaaattacagagttgcttaatttttttactagtagaacgcaacagtaaaaagaattaacagcagtgaattattagttaattagctagggagttgaaatagtttagcggtcatgcactactgatttttaattagtgcagacaacgaccgcgtgtgtgtatatgcatgtataccttttactgtatcttcagattgtgtatatatatcatatatgtacaggaaaagatttatatatcatacatattttgttgtatatatatacgtatatttctgtacaagtatatgtagacagtattttgtcatcttaataatttttttatcatattttaggctgactttgctggttgtcggattgttgcaaacatgtacaattaatgttaagaaaattaaggtagctaatgtgtcaacatgttgtgtgtgtttgtgctgacagagtgacagtgtggtctgtcctactccaagtactatcaaagtggtggtcgtgactcgtgagagcgacttcaagcctagaggttcatgtttttcttttaagataatgaggaggttgattgttatttcctcctacctccacatatataagtacttctaagggtttgaggctccgttcttttttaattaagatgtaaattttatcacaatttttattagcatgttttttcaaactacgaaatggtgtgtttcgtacggaaactatgtatgtagatgttgc

SEQ ID NO: 124 - DNA, 144 bp, Artificial sequence; identical to SEQ ID NO: 15
  Synthetic

SEQ ID NO: 125 - DNA, 144 bp, Artificial sequence
  Synthetic
attgataggaagaaagagtgattattgttgatcaggaattcttttcgataatgatgatatgctaatttcattcaatttgggcagcaaaagcatctcaattcattttcgaaaagaatgtcctgatcatcaccttcacctctttcg

SEQ ID NO: 126 - DNA, 29 bp, Artificial sequence
  Synthetic
tccctgcagaagtcaccaaaataatgggt

SEQ ID NO: 127 - DNA, 38 bp, Artificial sequence
  Synthetic
cctcacgtgttacacagctcaattacagactactcacc

SEQ ID NO: 128 - DNA, 40 bp, Artificial sequence
  Synthetic
tccctgcagcgctaggcgccataggtcgtttaagctgctg

SEQ ID NO: 129 - DNA, 40 bp, Artificial sequence
  Synthetic
tcccactagtcacgtgtatagcacaatgcatgatcttgct

SEQ ID NO: 130 - DNA, 40 bp, Artificial sequence
  Synthetic
cctcacgtgaggcccgtatagatgtagttaaatagctaaa

SEQ ID NO: 131 - DNA, 29 bp, Artificial sequence
  Synthetic
tccctgcagaagagtcaccgaactaaagg

SEQ ID NO: 132 - DNA, 21 bp, Artificial sequence
  Synthetic
cgtacggaatgccagcactcc

SEQ ID NO: 133 - DNA, 36 bp, Artificial sequence
  Synthetic
tgtacaatcgtcaacgttcacttctaaagaaatagc

SEQ ID NO: 134 - DNA, 36 bp, Artificial sequence
  Synthetic
gattaaaagagcaggaaagtattgggtgagatattg

SEQ ID NO: 135 - DNA, 25 bp, Artificial sequence
  Synthetic
ccgaaagaggtgaaggtgatgatca

SEQ ID NO: 136 - DNA, 21 bp, Artificial sequence
  Synthetic
cgtacggaatgccagcactcc

SEQ ID NO: 137 - DNA, 36 bp, Artificial sequence; identical to SEQ ID NO: 133
  Synthetic

SEQ ID NO: 138 - DNA, 36 bp, Artificial sequence; identical to SEQ ID NO: 134
  Synthetic

SEQ ID NO: 139 - DNA, 25 bp, Artificial sequence
  Synthetic
ccgaaagaggtgaaggtgatgatca

SEQ ID NO: 140 - DNA, 32 bp, Artificial sequence
  Synthetic
tccctgcaggcactttgcctgaagagaggacg

SEQ ID NO: 141 - DNA, 26 bp, Artificial sequence
  Synthetic
gctccaaatcggacagagagatgagc

SEQ ID NO: 142 - DNA, 32 bp, Artificial sequence; identical to SEQ ID NO: 140
  Synthetic

SEQ ID NO: 143 - DNA, 31 bp, Artificial sequence
  Synthetic
gtgcactccaaatcggacagagagatgagcc

SEQ ID NO: 144 - DNA, 25 bp, Artificial sequence
  Synthetic
gtgcactttgcctgaagagaggacg

SEQ ID NO: 145 - DNA, 34 bp, Artificial sequence
  Synthetic
aacccctaggctccaaatcggacagagagatgag

SEQ ID NO: 146 - DNA, 24 bp, Artificial sequence
  Synthetic
cctaggggttttgcactttgcctg

SEQ ID NO: 147 - DNA, 26 bp, Artificial sequence
  Synthetic
gctccaaatcggacagagagatgagc

SEQ ID NO: 148 - DNA, 34 bp, Artificial sequence
  Synthetic
gagctcaaatgtatgtctaaccatgcacatatgg

SEQ ID NO: 149 - DNA, 36 bp, Artificial sequence
  Synthetic
tagtcaggaattacgaagggtgtagttatgttattc

SEQ ID NO: 150 - DNA, 35 bp, Artificial sequence
  Synthetic
gattaaaatacaaatcgatctccatttcctccatc

SEQ ID NO: 151 - DNA, 40 bp, Artificial sequence
  Synthetic
tcccaattgtcaagttgaaacaattttttgtgcatataac

SEQ ID NO: 152 - DNA, 41 bp, Artificial sequence
  Synthetic
tcccaattgggaagtgtatgagttacaaaacatacttacct

SEQ ID NO: 153 - DNA, 28 bp, Artificial sequence
  Synthetic
ctacaaatcgatctccatttcctccatc